(19) United States
(12) Patent Application Publication
(10) Pub. No.: US 2023/0061804 A1
(43) Pub. Date: Mar. 2, 2023

(54) PHASE SEPARATION SENSORS AND USES THEREOF

(71) Applicant: THE ROCKEFELLER UNIVERSITY, New York, NY (US)

(72) Inventors: Felipe GARCIA QUIROZ, New York, NY (US); Elaine FUCHS, New York, NY (US)

(21) Appl. No.: 17/794,724

(22) PCT Filed: Jan. 22, 2021
(86) PCT No.: PCT/US2021/014684

§ 371 Date: Jul. 22, 2022

Related U.S. Application Data
(60) Provisional application No. 62/964,706, filed on Jan. 23, 2020.

(51) Int. Cl. A61B 5/00; G01N 33/58

(52) U.S. Cl. CPC A61B 5/0071; A61B 5/441; G01N 33/582; G01N 2500/10

(57) ABSTRACT

The present invention provides phase separation sensors capable of targeting or associating with one or more biomolecular condensate or membraneless compartment in cells. The phase separation sensors comprise at least two domains wherein a first domain comprises one or more accessory protein or molecule and a second domain comprises an artificial client protein or intrinsically disordered sequence. The artificial client protein possesses intrinsic disorder and is capable of engaging in ultra-weak phase separation-specific interactions with one or more component protein or molecule in a biomolecular condensate. Methods and applications utilizing the sensors are provided including targeting, detecting, visualizing, manipulating, monitoring a biomolecular condensate and delivering one or more functional protein, label, drug or agent to a biomolecular condensate.

### STATEMENT OF GOVERNMENTAL SUPPORT

This invention was made with government support under RO1-AR27883 awarded by the NIH. The government has certain rights in the invention.

### FIELD OF THE INVENTION

The present invention relates generally to biomolecular condensates or membraneless compartments in cells and the design and application of phase separation sensors capable of targeting or associating with biomolecular condensates. The phase separation sensors comprise at least two domains including one or more accessory protein or molecule and an artificial client protein or intrinsically disordered sequence. The invention also relates to methods and applications of the sensors.

### BACKGROUND OF THE INVENTION

Biomolecular condensates are two- and three-dimensional compartments in eukaryotic cells that concentrate specific collections of molecules without an encapsulating lipid-based membrane. Condensate formation has emerged as a fundamental mechanism for the organization of biomolecules within the nucleus and cytosol and at membranes (Hyman A A et al Annu Rev Cell Dev Biol 30 (2014) 39-58; Banani S F et al Nat. Rev. Mol. Cell Biol. 18 (2017) 285-298; Shin Y et al Science 357 (2017); Alberti S Curr. Biol. 27 (2017) R1097-R1102). Many condensates behave as dynamic liquids and appear to form through liquid-liquid phase separation (LLPS) driven by weak, multivalent interactions between macromolecules. There are numerous manifestations of this multivalency, including sticky ultra-weak interactions between intrinsically disordered proteins (IDPs), arrays of modular protein domains (Li P et al Nature 483 (2012) 336-340; Fromm S A et al Angew Chem Int Ed Engl 53(2014) 7354-7359; Banjade S et al elife 3 (2014), e04123, doi.org/10.7554/eLife.04123;Zeng M et al Cell 166 (2017)1163-1175.e12; Su X et al Science 352 (2016) 595-599; Sun D et al Cell Res. 28 (2018) 405-415), distributed weakly adhesive motifs separated by intrinsically disordered regions (IDRs) of proteins (Lin Y et al Mo. Cell 60 (2015) 208-219; Nott T J et al Mol Cell 57 (2015) 936-947; Patel A et al Cell 162 (2015) 1066-1077; Molliex A et al Cell 163 (2015) 123-133; Murakami T et al Neuron 88 (2015) 678-690; Xiang S et al Cell 163 (2015) 829-839), and repetitive base-pairing elements in RNA and DNA (Jain A et al Nature (2017) doi.org/10.1038/nature22386; Langdon E M et al Science (2018), doi.org/10.1126/science.aar7432). Specific interactions, such as interactions between modular binding domains and nucleic acid base pairing, weaker interactions between intrinsically disorder regions, and nonspecific interactions, such as electrostatic interactions and hydrophobic interactions, influence condensate formation and composition.

Representative and recognized biomolecular condensates include PML nuclear bodies, P-bodies, stress granules, the nucleolus, and two-dimensional membrane localized LAT and nephrin clusters. Individual condensates can contain hundreds of distinct molecular components. For example, promyelocytic leukemia protein (PML) bodies can contain over 200 unique proteins (Van Damme E et al J. Int. Biol. Sci. 6 (2010) 51-67,doi.org/10.7150/ijbs.6.51), the nucleolus can contain over 4500 unique proteins (Ahmad Y et al (2009) Nucleic Acids Res 27:D181-D184), and stress granules can contain over 100 proteins as well as over 1000 RNA transcripts (Khong A et al Mol. Cell 68 (2017) 808-820.e5,doi.org/10.1016/j.molcel.2017.10.015; Jain S et al Cell 164 (2016) 487-498; Markmiller S et al Cell 172 (2018), doi.org/10.1016/j.cell.2017.12.032 (590-604.e13); Youn J et al Mol. Cell 69 (2018) 517-532). Some of these components are unique to a specific condensate, but others can be shared between different types, particularly among the various RNA-containing structures (Langdon E M et al Science (2018), doi.org/10.1126/science.aar7432; Markmiller S et al Cell 172 (2018), doi.org/10.1016/j.cell.2017.12.032 (590-604.e13); Youn J et al Mol Cell 69 (2018) 517-532; Gopal J J et al Proc Natl Acad Sci 114 (2017) E2466-E2475, doi.org/10.1073/pnas.1614462114; Buchan J R et al Mol Cell 36 (2009) 932-941). Composition can vary dramatically under different cellular conditions and can rapidly change in response to signals (Markmiller S et al Cell 172(2018), doi.org/10.1016/j.cell.2017.12.032 (590-604.e13); Youn J et al Mol. Cell 69 (2018) 517-532; Buchan J R et al Mol Cell 36 (2009) 932-941; Fong K et al J Cell Biol 203 (2013) 149-164; Weidtkamp-Peters S et al J. Cell Sci. 121 (2008) 2731-2743). Even in the absence of stimuli, many condensate components or residents rapidly exchange between the condensate and the surrounding cytoplasm or nucleoplasm (Molliex A et al Cell 163 (2015) 123-133; Brangwynne C P et al Proc Natl Acad Sci 108 (2011) 4334-4339; Woodruff J B et al Cell 169 (2017),doi.org/10.1016/j.cell.2017.05.028(1066-1077.e10); Schwarz-Romond T et al J Cell Sci 120 (2007) 2402-2412; Dundr M et al Biochem. J. 356 (2001) 297-310).

Often only a few components or residents are necessary to form the condensate and deletion or depletion of these molecules decreases the size and/or number of the structures in a cell, while overexpression can have the opposite effect (Clemson C M et al Mol Cell 33 (2009) 717-726; Ishov A M et al J Cell Biol 147 (1999) 221-234; Teixeira D et al Mol Biol Cell 18 (2007) 2274-2287; Rao B S et al Proc Natl Acad Sci USA 114 (2017) E9569-E9578,doi.org/10.1073/pnas.1712396114). These resident elements are referred to as scaffolds. PML is an example of a scaffold—knocking out PML abolishes PML nuclear body formation while increasing PML expression results in an increased number of PML nuclear bodies (Ishov A M et al J. Cell Biol. 147 (1999) 221-234; Zhong S et al Blood 95 (2000) 2748-2752; de Stanchina E et al Mol Cell 13 (2004) 523-535). Other condensate residents are concentrated within the structure, often by direct interactions with scaffolds, but are not required for condensate formation, and these are referred to as clients. Examples of clients include PML nuclear body proteins Sp100 and BLM, and it has been shown that knocking out either protein does not ablate PML nuclear body formation (Ishov A M et al J. Cell Biol. 147 (1999) 221-234; Zhong S et al Oncogene 18 (1999) 7941).

Despite remarkable progress, the study of cellular phase separation remains challenging and often relies on truncated protein mutants, reconstituted systems in non-physiological buffers, and overexpression/knockin of tagged fusions that can alter a protein's phase separation behavior (Alberti S et al Cell 176, 419-434 (2019); Schmidt H B et al Nature communications 10, 1-14 (2019); Bracha D et al Cell 175, 1467-1480.e1413 (2018).

Therefore, it should be apparent that there still exists a need in the art for methods and approaches to evaluate, detect, monitor, target, and assess cellular phase separation and biomolecular condensates. There are deficiencies in the present knowledge and available tools to be able to assess, monitor and manipulate biomolecular condensates, particularly in living cells and in vivo, especially in instances where the condensates are involved in critical aspects of cellular physiology or provide markers of or targets for disease or conditions.

The citation of references herein shall not be construed as an admission that such is prior art to the present invention.

### SUMMARY OF THE INVENTION

In its most general embodiment, the present invention extends to biomolecular condensates or membraneless compartments in cells, and the ability to detect, target, monitor, assess and modulate biomolecular condensates, including in vitro or ex vivo in cells or tissues and in vivo in animals, including humans, and in animal model systems. The invention provides novel phase separation sensors capable of targeting or associating with biomolecular condensates, including nascent or preassembled biomolecular condensates. These sensors are designed to preferentially target or associate with target biomolecular condensates. The sensors comprise at least two domains, wherein a first domain includes one or more accessory protein or molecule and a second domain includes an artificial client protein or intrinsically disordered sequence. The artificial client protein or intrinsically disordered sequence is uniquely capable of interacting with one or more component protein, particularly one or more scaffold protein, in a target biomolecular condensate.

In an initial embodiment of the invention, a phase separation sensor is provided wherein the sensor is capable of targeting or associating with a biomolecular condensate and comprises at least two protein domains, wherein the first domain comprises one or more accessory protein and the second domain comprises an artificial client protein having intrinsic disorder and capable of engaging in ultra-weak phase separation-specific amino acid interactions with one or more component protein in the condensate.

In an embodiment, the phase separation sensor lacks independent phase separation behavior when expressed in the cell. In an embodiment, the phase separation sensor lacks independent phase separation behavior when expressed in the cell at reasonably high levels.

In another embodiment, the phase separation sensor associates with the biomolecular condensate without disrupting the condensate.

In an embodiment of the invention, the artificial client protein is an intrinsically disordered protein having low complexity sequence. In an embodiment, the artificial client protein contains one or more disordered region that provides one or more or multiple weakly adhesive sequence elements. In an embodiment, the artificial client protein sequence lacks recognized protein three dimensional structural aspects. In an embodiment, the artificial client protein sequence contains repeated sequence elements. In an embodiment, the artificial client protein sequence contains low complexity sequence elements. In a particular such embodiment, the low complexity sequence elements provide basis for multivalent weakly adhesive intermolecular interactions. In a particular embodiment, the low complexity sequence elements provide basis for multivalent weakly adhesive intermolecular interactions with a target scaffold protein.

In accordance with another embodiment of the invention, the sensor artificial client protein sequence comprises similar compositional bias or comprises related sequence patterns with low sequence identity to the amino acid sequence of a naturally-occurring intrinsically disordered protein or protein region within a larger protein or target protein. In one embodiment, the larger/target protein is a component of a biomolecular condensate. In one such embodiment, this similar compositional bias or related sequence patterns contributes to or is responsible for driving assembly of said biomolecular condensate.

In an embodiment of the invention, the phase separation sensor's artificial client protein sequence is related to the native or target intrinsic disordered protein (IDP) sequence. In an embodiment of the invention, the phase separation sensor's artificial client protein sequence is related to the native or target intrinsic disordered protein (IDP) sequence by reversing native or target IDP amino acid sequence. In accordance with this embodiment, the sensor's sequence artificial client protein sequence is generated by reading the original, native or target IDP sequence in the non-natural C-terminal to N-terminal direction. This provides an absolutely distinct non-native sequence for the artificial client protein, including wherein the chirality and orientation and/or the structure of molecule in space is absolutely distinct from the target IDP amino acid sequence. In an embodiment, the native or target IDP sequence's compositional bias, overall amino acid sequence and charge is maintained in the artificial client protein. In an embodiment, the artificial client protein sequence is a randomized or jumbled sequence corresponding to or based on the sequence of the target IDP sequence.

In another and alternative embodiment, the sensor's artificial client protein sequence is generated de novo without reference to the or any target sequence. In this embodiment, the artificial client protein sequence is intrinsically disordered and may comprise a repeated sequence which is a low complexity sequence comprising a limited number of amino acids. In certain embodiments, the artificial client protein sequence is intrinsically disordered and may comprise a repeated sequence which is a low complexity sequence comprising a limited number of amino acid in a repeating sequence pattern.

The invention contemplates a sensor molecule or protein which provides a functional, active, visible or detectable label or marker. In one such embodiment, the sensor comprises a reporter molecule or protein which provides a functional, active, visible or detectable label or marker The invention contemplates a sensor molecule or protein which provides a function, including an enzymatic activity or other activity or capability. In an embodiment of the invention, the sensor comprises in a first domain, or in one or more embodiment or portion of a first domain, one or more accessory protein wherein at least one accessory protein provides a detectable or functional label.

In one or more embodiment, the at least one accessory protein may be selected from fluorescent protein, protease, nuclease, ligase, peroxidase, phosphatase, kinase and protein capable of modifying a protein or nucleic acid.

In one such embodiment, at least one accessory protein is a fluorescent protein. In embodiments thereof, the fluorescent protein may be a GFP protein. In an embodiment, the GFP protein is a GFP protein with positively-charged amino acids exposed on the protein surface. In an embodiment, the GFP protein may be +15GFP. In an embodiment, the reporter molecule is a GFP with net charge +15 and is selected from +15sfGFP (SEQ ID NO:28) and +15sfGFPK (SEQ ID NO:29).

In an embodiment, a one or more accessory protein may be an enzyme. In one embodiment the enzyme may be a protease, nuclease, ligase, peroxidase, phosphatase, kinase.

In an embodiment, one or more accessory protein may comprise a label. In an embodiment, the label may include a radioactive element. In one such embodiment, the sensor may thereby introduce a label or radioactive element into a cell, particularly into a biomolecular condensate in a cell. The label or element may then be examined by known techniques, which may vary with the nature of the label or element attached. In the instance where a radioactive label is used, it may be selected from isotopes such as the isotopes ³H, ¹⁴C, ³²P, ³⁵S, ³⁶C, ⁵¹Cr, ⁵⁷Co, ⁵⁸Co, ⁵⁹Fe, ⁹⁰Y, ¹²⁵I, ¹³¹I, and ¹⁸⁶Re.

In accordance with a further embodiment, at least one accessory protein may be capable of tagging one or more biomolecular condensate component with a detectable or functional molecule, peptide or marker.

In an embodiment of the invention, the sensor is a functionalized sensor and at least one accessory protein is capable of modifying a target component protein in the condensate.

In another embodiment, the sensor is a functionalized sensor and at least one accessory protein is capable of delivering a compound or agent to the condensate or to a target component protein in the condensate.

In embodiments of the invention, the two or more domains comprising the sensor may be directly linked or may be separated in each or any instance by one or more linker sequence. In a particular embodiment, one or more accessory protein(s) and/or the accessory protein(s) and the artificial client protein are separated by a flexible linker sequence. The flexible linker sequence may comprise between 2 and 10, 10 and 20, 20 and 40, 2 and 20, 2 and 30, 2 and 40, up to 10, up to 20, up to 30, up to 40 amino acid residues. The flexible linker sequence may comprise between 2 and 10 amino acid residues. In a preferred embodiment, one or more short flexible linkers of 2 to 10 residues in length is utilized. In an embodiment, the linker sequence lacks charged residues. In an embodiment, the linker sequence contains charged residues. In an embodiment, the linker sequence contains charged residues and is zwitterionic, having equal numbers of positive-charged and negatively-charged residues. In exemplary embodiments and sequences hereof, linkers of 2, 4 and 10 residues are utilized. In embodiments, linker sequences GSPG (SEQ ID NO: 59) and/or GRSDGVPGSG (SEQ ID NO: 60), as examples, are utilized.

In a particular embodied embodiment of the invention, a phase separation sensor is provided wherein the target component protein is a filaggrin family protein or paralog protein. In one such embodiment, the sensor artificial client protein sequence is derived from or based on a filaggrin protein sequence. In one embodiment, the artificial client protein sequence is derived from or based on human filaggrin protein sequence or on a mouse filaggrin protein sequence. In an embodiment, the artificial client protein sequence is derived from or based on a filaggrin protein sequence provided in TABLE 1, or a mouse or human filaggrin protein sequence including as provided in SEQ ID NO: 1 or SEQ ID NO: 56. In a particular embodiment, the artificial client protein sequence is derived from or based on a filaggrin protein repeat component sequence.

In embodiments of the invention, exemplary filaggrin-based or filaggrin-targeting phase separation sensors are provided herein, including in TABLE 3 and in Examples 1 and 2 hereof. These sequences include artificial client protein sequences designed based on the filaggrin target sequence and tested herein. Phase separation sensor designs and examples are provided and described herein an include SEQ ID NO: 26, 27, 50, 51, 52, 53 and 54. Phase separation sensors include Sensor A (SEQ ID NO:26), Sensor B (SEQ ID NO:27), Apex2-Sensor A (SEQ ID NO:50), Apex2-Sensor B (SEQ ID NO:51), Sensor C (SEQ ID NO:52) and Sensor D (SEQ ID NO:53). An additional phase separation sensor is provided in Sensor Apex2-excluded (SEQ ID NO: 54).

In a further embodiment, phase separation sensors are contemplated and provided herein that are directed to one or more biomolecular condensate in a cell or in vivo in an animal. The sensor(s) of the invention may target or associate with one or more biomolecular condensate in the cytoplasm of a cell and/or in the nucleus of a cell. In one such embodiment, the condensate is a keratohyalin granule (KG) in the epidermis or in one or more skin cell. In embodiments, one or more phase separation sensor is provided that targets a biomolecular condensate selected from P granule, Germ granule, Lewy bodies, synaptic condensates, stress granule, P bodies, T cell signalosome, crystalline condensates of the lens fibers, and other cytoplasmic condensates or membraneless organelles assembled through liquid-liquid phase separation. In further embodiments one or more phase separation sensor is provide that targets a biomolecular condensate in the nucleus. In an embodiment, nuclear condensates may be selected from Nucleoli, Paraspeckles, Histone Locus Bodies, Cajal Bodies, Heterochromatin, super-enhancer domains. The biomolecular condensate may be an RNA-protein granule or an RNA-containing condensate. In an embodiment, the target condensate protein may be an RNA-binding protein.

In any embodiment of the invention wherein the target condensate or condensate protein is a cytoplasmic condensate or cytoplasmic condensate protein, the phase separation sensor may include one or more nuclear export signal (NES). NES sequences are known and available to one skill in the art. NES sequences described and provided herein include LELLEDLTL (SEQ ID NO: 57) and SGLELLEDLTL (SEQ ID NO: 58). In one such embodiment, the NES prevents nuclear localization and targets the protein or sensor to the cytoplasm. In any embodiment of the invention wherein the target condensate or condensate protein is a nuclear condensate or a condensate or condensate protein located in the nucleus, the phase separation sensor may include one or more nuclear localization signal (NLS), so as to promote or limit localization to the nucleus. In an embodiment, a sensor of the invention lacks a nuclear localization signal and also lacks a nuclear export signal and thereby may function, may be expressed in, or may localize to either of or both of the nucleus and cytoplasm.

In an embodiment of the invention, a phase separation sensor is provided to investigate or assess phase separation of a putative or candidate condensate, including to determine whether a target protein is incorporated in a biomolecular condensate. In an embodiment of the invention, a phase separation sensor is provided to investigate or assess phase separation of a putative or candidate condensate, including to randomly or indirectly characterize the proteins in a putative or candidate condensate. Thus in an embodiment of the invention, a phase separation sensor is designed which generically or relatively non-specifically associates with biomolecular condensates by virtue of ultra-weak interactions and not by target sequence-based derivation. Provided that the interaction is sufficient and the accessory protein label is adequate, a condensate may be generally or generically targeted and tagged or monitored by association with the sensor. In accordance with an embodiment of the invention, a phase separation sensor of the invention may identify, monitor and characterize a biomolecular condensate of previously unknown nature, composition or purpose.

In an embodiment of the invention, a sensor is designed to generally or generically recognize and monitor the phase behavior of an intrinsic disorder protein (IDP) or sequence, including wherein the IDP is predicted to undergo liquid-liquid phase separation. In an embodiment, phase behavior can be monitored by virtue of a tag or label comprised in, provided in or with the sensor.

The invention includes compositions of the phase separation sensors provided herein. The compositions include pharmaceutical compositions, optionally further comprising one or more vehicle, carrier or diluent. In embodiments of the invention, compositions including pharmaceutical compositions, may include one or more of the phase sensors in combination with an agent or compound for a diagnostic or therapeutic purpose or intent. In an embodiment, such compositions may provide targeting or delivery of an agent or compound to a biomolecular condensate, including a target biomolecular condensate.

In another embodiment, the invention provides nucleic acids encoding a phase separation sensor hereof. In an embodiment, a sensor may comprise a nucleic acid sequence, such as an RNA or DNA sequence. DNA molecules comprising the nucleic acids are an embodiment of the invention. Further, a vector comprising the nucleic acids or DNA molecules of the invention is also provided.

In additional embodiments, methods are provided herein based on the characteristics and capabilities of the phase separation sensors. In one such embodiment, a method is provided for targeting a biomolecular condensate in a cell or tissue comprising administering to the cell or tissue or otherwise expressing in the cell or tissue one or more phase separation sensor of the invention.

In an embodiment, a method is provided for targeting a biomolecular condensate in a cell comprising transfecting or transducing the cell with a nucleic acid or with a vector comprising nucleic acid encoding a sensor of the invention or otherwise capable of expressing the sensor of the invention in a cell.

In another embodiment, a method is provided for detecting or visualizing a biomolecular condensate in a cell or tissue comprising administering to the cell or tissue or otherwise expressing in the cell or tissue one or more sensor of the invention as provided herein. In one such method embodiment, the sensor comprises at least one accessory protein comprising a detectable or functional label or marker, or a protein capable of tagging the condensate with a detectable or functional label or marker, including for example by association with or localization in the condensate. In a method embodiment, the sensor comprises at least one accessory protein suitable for tagging the condensate, such as a fluorescent protein, a radioactive dye or label, a protein that creates contrast suitable for electron microscopy, or a protein otherwise capable of tagging the condensate with a detectable or functional label or marker. In a method embodiment, the sensor comprises at least one accessory protein selected from a fluorescent protein, a protein that creates contrast suitable for electron microscopy, or a protein capable of tagging the condensate with a detectable or functional label or marker.

Another method embodiment of the invention is provided in a method for monitoring biomolecular condensates in a cell comprising administering to the cell or otherwise expressing in the cell or tissue one or more sensor described and provided herein wherein the sensor is capable of tagging the condensate with a detectable or functional label or marker. In an embodiment, the sensor is capable of tagging or labeling a protein in the condensate via a chemical interaction or enzymatic reaction. In an embodiment, the sensor is capable of tagging or labeling a protein in the condensate via ultra-weak bonding or by association with or localization in the condensate. In an embodiment, the sensor is capable of tagging the condensate with a detectable or functional label or marker without significantly altering the condensate or any condensate protein. In an embodiment, the sensor is capable of tagging the condensate with a detectable or functional label or marker without altering the condensate or any condensate protein.

A kit for evaluation of biomolecular condensates in cells or tissues is provided in another embodiment of the invention, wherein the kit comprises a phase separation sensor as described and provided herein, a nucleic acid encoding a sensor hereof, or a vector comprising a nucleic acid or otherwise capable of expressing one or more sensor hereof in a cell.

In alternative methods of the invention, the phase separation sensors provided herein may be utilized in monitoring phase separation dynamics. The sensors can monitor the formation of condensates and their disassembly, including in a cell, tissue or organ. In method embodiments, a phase separation sensor can monitor the formation and/or disassembly of a target biomolecular condensate in a cell, tissue or organ, such as in skin.

Further methods embodiments include use and application of one or more phase separation sensor to evaluate or screen compounds, drugs or agents for their effect on a condensate. This is particularly relevant wherein the formation of a condensate, the size or location of a condensate, or the component make up is altered in or associated with a disease or condition, or is involved in a cellular response in an animal, particularly in a human. In one such embodiment, the sensors are utilized in screening for drugs that promote assembly or disassembly of target condensates.

Other objects and advantages will become apparent to those skilled in the art from a review of the following description which proceeds with reference to the following illustrative drawings.

### BRIEF DESCRIPTION OF THE DRAWINGS

FIG. 1. Filaggrin proteins undergo liquid-liquid phase transitions that are disrupted by disease-associated filaggrin mutations. (A) Ultrastructure and schematic of mouse skin at embryonic day E17.5. Dotted lines delineate the basement membrane, where proliferative epidermal progenitors attach (basal layer). Periodically, progenitors initiate terminal differentiation, ceasing to divide, but transcribe the necessary genes for skin barrier formation as they flux upward through keratin filament bundle-rich spinous layers; keratohyalin granule (KGs, arrows)-rich granular layers; and dead, enucleated squames which continually slough from the skin surface (corneum), replenished by differentiating cells from beneath. (B) Domain architecture of human FLG, the major known constituent of KGs, and location of nonsense FLG mutations (colored lines) associated with skin barrier disorders (FIG. S1 shows mutants). Many mutations cluster to generate truncated variants in FLG repeat domains (labeled as mut-n0 to mut-n10). (C) Mouse and human FLG are histidine-rich, low complexity (LC) proteins with identical biases in amino acid composition, but not sequence. Mean amino acid abundance across the human proteome is shown as a gray line (filled area is the standard deviation). (D) FLG and its paralogs (FLG2, RPTN, HRNR and TCHN) share a strong preference for arginine over lysine residues [calculated as R/(R+K)], a major determinant of phase separation in LC proteins (22). The gray line marks the mean Arg-bias across the mouse and human proteome (filled area is the standard deviation). See FIG. S3 for details. (E) Proteome-wide distribution of protein size (unit length 1000 a.a.), underscoring the enormous size of FLG (x marks the 99 percentile). (F) Transfection of synthesized FLG genes into HaCATs reveals that the propensity of FLG repeat proteins to undergo phase separation is governed by the number of FLG repeats. In these experiments, genes encoding tagged-FLG variants [mRFP-(r8)n, where r8=repeat #8 and n=1-8 of these repeats] were fused C-terminal to a H2B-GFP-[p2a] construct. Co-translationally, the self-cleavable [p2a] sequence (28) ensures that each construct generates one H2B-GFP molecule for each mRFP-(r8)n molecule. Panels show cells with the same total concentration of mRFP-(8)n. Quantitatively, phase separation propensity was defined as the percent of total mRFP signal within a phase-separated granule. (G) Phase separation propensity for FLG variants spanning the repeat distribution of truncated FLG mutants (mut-n0 to mut-n8; WT-size is n=12) and across a wide range of expression levels for each variant. Dashed lines are logistic fits to data with signs of a concentration-dependent phase transition. (H) Time-lapse imaging of HaCATs expressing increasing levels of mRFP-(r8)8 (related to H2BGFP via [p2a]). Shown are the initial stages of phase separation through the formation and growth of granules (marked as g1-g3). (I) The S100 (dimerization) domain of human FLG enhances the phase separation propensity of FLG repeat proteins but fails to rescue phase behavior in disease-associated variants with ≤2 FLG repeats (mut-n0-n2 in B). Construct design and quantifications are as in (B). Dashed lines are logistic fits to the data. Images are maximum intensity projections.

FIG. 2. Nonsense human FLG mutations associated with skin barrier disorders A comprehensive list of truncating human FLG mutations [compiled from ref 27 in the main manuscript and (62)], with their relative location across the length of the FLG protein denoted by colored lines that cut/truncate the protein. Lines that project below the indicated domains highlight mutations that are most common across European and Asian patients (typically >2% of patients). Because of the wide spectrum of mutations and their apparent clustering at different locations across the protein, we grouped mutations by the number of FLG repeat domains that are spared in each group of truncated variants (mut-n0 to mut-n10, that is mutants left with 0 FLG repeats to 10 repeats). A common variant, p.K4022X, occurs at the C-terminal end of FLG, sparing all repeats but ablating the small 26 a.a. C-terminal tail domain. We refer to this mutant and others that spare all FLG repeats (note that some humans have up to 12 FLG repeats) as ‘Tail mutants’.

FIG. 3. Sequence features of FLG and its paralogs across mammalian species. (A) While both mouse and human FLG share a repeat architecture and similar non-repeat domains, their repeat units and overall repeat domain differ greatly at the sequence and organization levels. Human FLG typically has 10 near perfect copies of a 324-residues repeat (with humans having up to 12 repeats), whereas repeat length in mouse FLG typically spans 16 copies of a near perfect 250-residue repeat. Total number of FLG repeats also varies across mouse strains. Because of this divergence in the repeat domain, FLG sequences are typically identified through sequence conservation in the short S100 domain. Color variations across individual repeats point to subtle changes in repeat identity—mouse FLG has higher repeat identity than human Flg repeats (˜95% vs 99%, respective). (B) Disordered regions in human FLG. The disorder score for each residue in the protein was calculated using DISOPRED (63)—a score of 1 is characteristic of IDP regions. Note that for human FLG only the first 100 residues (corresponding to the S100 domain) are ordered (folded). (C) Amino acid abundance for all amino acid residues across mouse and human FLG. Only a handful of residues (FIG. 1C) account for the majority of their composition and these biases are well-conserved across mice and humans. The mean proteome-wide abundance of each residue in human proteins is shown as a gray line (the filled area shows the standard deviation). Abundance values are nearly identical for the mouse proteome. We also show the mean abundance (magenta line) of each residue in protein domains that are part of PhaseSePro (a database of manually curated protein drivers of liquid-liquid phase separation) (51)—the filled area (magenta) shows the standard deviation. Note that Pro, Gly and Ser are prominently enriched in FLG and in protein domains found in PhaseSePro, whereas His is uniquely enriched in FLG. (D) Proteome-wide distribution of protein size in mice and humans readily reveals that FLG is among the largest proteins in its corresponding proteome.

FIG. 4. Sequence features of FLG and its paralogs across species. Analysis of sequence-encoded features that are indicators of upper critical solution temperature (UCST) phase separation behavior in low-complexity proteins across of FLG and its paralogs (FLG2, RPNT, HRNR and TCHH) in mice, humans and other mammalian species (see TABLE 1 for additional details). These indicators were recently proposed as important sequence determinants of UCST-type phase behavior in IDPS (Ref. 18). Arginine-bias was calculated as [R/(R+K)]. Percent of aromatic residues corresponds to the sum of Y, H and F residues divided by total number of residues. Hydrophobicity was calculated using the Kyte-Doolittle hydropathy scale (52), where increasingly negative values mirror increases in hydrophilicity. As reference values for these sequence features, we have included the mean value and standard deviation of each parameter across (1) all proteins in the human proteome, (2) all protein domains in the PhaSEPro database that were manually curated (as of October 2019) as drivers of liquid-liquid phase separation (51), and (3) recently characterized UCST-exhibiting IDPs. We note that FLG is not part of PhaSEPro, and the proteins in PhaSEPro are not discriminated based on sequence-encoded mechanisms of phase separation. As a result, we do not expect PhaSEPro-derived parameters to exactly recapitulate the core sequence-features of canonical UCST-type IDPs. For instance, PhaSEPro encompasses classical LCST-exhibiting IDPs like tropoleastin (LCST is the mirror behavior to UCST and both behaviors are encoded differently in IDPs; see ref 18) as well as RNA-binding proteins that likely exhibit UCST-type behavior but whose phase separation is RNA-dependent in many cases. Despite these limitations, we observe that the sequence features of FLG and its paralogs are very similar to those of UCST-exhibiting IDPs and that sequence-biases discerned from PhaSEPro align better with UCST-IDPs and with FLG than do the overall distribution of these parameters in the human proteome.

FIG. 5. FLG and RPTN (a FLG paralog) form distinct micron-sized granules in HaCATs. Immortalized human keratinocytes (HaCATs) do not form KGs when submerged in medium. However, when cultured at the air-liquid interface for 16 days, as shown here, immunostaining against human FLG (red) or human RPTN (green) reveals endogenous granule formation. Images are close-up views of cells in the granular layer of these cultures. As a note, all other subsequent experiments in this manuscript, we used submerged culture conditions (see methods), where cells remain as progenitors.

FIG. 6. Synthesis of long repetitive DNAs encoding human FLG variants. (A) Efficient iterative synthesis of repetitive FLG-like genes and their fusions to non-repeat FLG domains and fluorescent proteins using a plasmid reconstruction approach (see TABLE 2 for sequence details) (53). These genes are based on the r8 repeat of human FLG (r8 is one of 10 near perfect repeats as shown in FIG. 1B) (B) Electrophoresis of plasmids harboring synthetic Flg genes (fusions to sfGFP in this example) with the indicated number of repeats and digested at NheI and EcoRI sites that flank the genes. Each lane shows a constant fragment that corresponds to the digested vector. The left most lane is a DNA ladder (1 Kb plus DNA ladder, Thermofisher Scientific). This gel corresponds to a 0.8% agarose gel (with SYBR Safe) ran over 1.5 h (at 130V) to improve separation between the largest repetitive genes. (C) To better show the size distribution of the synthetic FLG genes we gel purified the corresponding gene bands from (B) and performed a new electrophoresis using similar conditions as in (B). The scale bar (in Kb) shows the precise size of each construct.

FIG. 7. Phase separation properties of FLG repeat proteins. (A) We transfected HaCATs with sfGFP-tagged constructs with increasing number of FLG repeats (n=1 to n=12, where “n” is the number of repeats; see Table S2 for sequence details) and saw that their phase separation propensity (i.e., the ability to form granules or condensates in the cell) was largely dependent on the number of FLG repeats. Below n=4, well-defined granules did not form and sfGFP was cytoplasmic. At n=4, most of the sfGFP fluorescence was compartmentalized into well-defined granules, establishing two phases (dilute and dense) characteristic of phase-separated systems. Constructs with more than 4 repeats typically showed more granules with highly spherical morphologies that were reminiscent of endogenous FLG granules in culture (FIG. S4). Nuclei (nu, DAPI) of transfected cells are circled. (B) Changes in FLG repeat density within cytoplasmic granules that formed de novo in HaCATs upon transfection of mRFP1-tagged FLGs with variable numbers of the r8 human FLG repeat (see FIG. S1B). When mRFP1 fluorescence intensities were normalized according to FLG repeat number (e.g. 3× more r8 units per mRFP1 molecule in constructs with n=4 vs n=12), it was clear that the density of repeats was highest within KGs assembled from proteins with the greatest FLG repeat numbers. (B) Fluorescence recovery after photobleaching (FRAP) half-lives of granules composed of FLG repeat variants in (A). Top, representative images of a recovery event; Bottom, quantifications. Dots represent individual FRAP half-life measurements (of granules in different cells) from two experiments. All images correspond to maximum intensity projections of Z stacks spanning the volume of cells in the field of view.

FIG. 8. Critical concentration for phase separation and characterization of FLG variants. (A) Relative H2BGFP to H2BRFP brightness for HaCATs that express H2BGFP-[p2A]-H2BRFP or H2BRFP-[p2a]-H2BGFP. [p2a] is an optimized self-cleaving peptide sequence that ensures equimolar synthesis of its N- and C-terminal fusion proteins. Each dot is a measurement from an individual nucleus. The overall average ratio matches the published relative brightness of EGFP and RFP (ratio=3) irrespective of the fusion format, confirming that our strategy works well to produce equimolar amounts of [p2a]-fused constructs upon their expression in HaCATs. The shown average ratio (3.08) and standard deviation accounts for all data acquired for constructs 1 and 2. In subsequent analyses and for conversion between RFP-based and GFP-based concentration values, we use a ratio of 3.0. For clarity, throughout the manuscript, a GFP-based scale is used. (B) Critical concentration for phase separation based on logistic fits from data in FIG. 1G. Operationally, this critical concentration was defined as the EC50 of the logistic fits, that is the concentration at which most cells achieve a phase separation response of 50%—wherein the total number of molecules in the dilute phase equals the number of molecules in the high density phase. While phase separation happens with a given (low) probability below the EC50 (as can be seen in our data), the concentration fluctuations that potently drive phase separation near the true critical concentration of the system become dominant near the EC50, which justifies its definition as an experimental approximation to the critical value. (C) Critical concentration for phase separation estimated from the concentration of protein in the dilute phase (which exists at the verge of phase separation into the high density phase). Note that these estimates are in excellent agreement with values derived from logistic fits in (B). The advantage of this additional approach to estimate the critical concentration for phase separation is that in this case we only need to consider select cells that already underwent phase separation, whereas for the approach in (B) we need to sample cells that express FLG variants across the entire concentration regime (below and above the critical concentration for phase separation). Note that across the data in FIGS. 1G and 1I, as well as here in B-C, we consistently see that as the critical concentration for phase separation goes down with S100-fusion or with long repeat domains, the sharpness of the phase transition increases. (D) Purified recombinant sfGFP in PBS at different concentrations (in μM) and its respective concentration values based on fluorescence measurements (reported in arbitrary units). The measurements were done in a similar fashion as in our typical cell-based experiments to account for photobleaching in our imaging protocol. Our limit of detection is ˜1 uM. (E) Linearity is lost for very high sfGFP concentrations (in the order of magnitude found within KGs, ˜500 μM-1 mM). We use calibration curves in D-E as a rough guideline to gage the range of concentrations (in μM units) at which FLG variants undergo phase separation. We note that based on the intrinsic brightness of EGFP vs sfGFP, [sfGFP]-scale=1.6 x [H2BGFP]-scale. (F) Critical concentration for phase separation (based on data in B) but converted to μM values based on calibration curves in (D)-(E). (G) Choice of fluorescent protein has a bearing on the measured critical concentration for phase separation for fluorescently-tagged FLG variants. We see that sfGFP-based constructs enhance the phase separation behavior of FLG variants with respect to mRFP-based constructs. The extent of this enhancement is similar to the response measured for constructs with the S100 (dimerizing) domain of human FLG (FIG. 1I), which likely points to weak dimerization of sfGFP (64) as a factor that alters the biophysical properties of tagged-FLG. (H) Concentration of the filaggrin scaffold (in μM units) within KGs assembled in HaCATs by transfection of two different sfGFP-tagged FLG variants (one is WT-size, n=12, and the other one is a disease-associated variant with 4 FLG repeats). (I) Quantification of FLG density within KGs studied in FIG. 9B. Note that changes in FLG density within these KGs are not responsible for the observed changes in FRAP dynamics. N.S, not statistically significant.

FIG. 9. Filaggrin processing and disease-associated mutations alter the liquid-like behavior and material properties of KG-like membraneless compartments. (A) Fluorescence recovery after photobleaching (FRAP) half-lives of granules formed de novo in immortalized human keratinocytes following transfection of indicated mRFP1-tagged FLGs with different FLG repeat truncations. Left, representative images of a recovery event; Middle, representative FRAP recovery plot (average±SD from 7 granules); Right, quantifications. (B) FRAP half-lives after internal photobleaching of granules formed from a mRFP-FLG [WT(p), mRFP-(r8)8-Tail] in comparison to one that either lacks the 26 a.a. tail domain (Tail mut) or contains the amino (S100) domain of FLG [WT(up)]. Each dot in (A)-(B) represents an individual FRAP half-life measurement of granules from multiple cells. Data are from ≥2 experiments. (C) Tagged-FLG granules undergo liquid-like fusion events. Live imaging of a cell transfected with a cytoplasmic marker (mCherry) and a WT(p) FLG [sfGFP-r(8)12-Tail]. Arrows point to granule fusion events over time (data not shown). (D-F) Atomic force microscopy (AFM) reveals liquid-like behaviors of granules. (D) Snapshots of granule (arrows) before and with pressure application reveals liquid-like streaming behavior (data not shown). (E) Representative AFM map shows that even KGs composed of the FLG tail mutant appear to be significantly stiffer than cytoplasm (see FIG. 12 for WT-type KGs data). (F) Average stiffness (Young's Modulus) per granule for KGs assembled from the FLG variants described in (B). Each dot corresponds to measurements of a different granule (average of all pixels within the granule domain in the stiffness map) in a different cell. Nu, nucleus; Asterisks, statistically significant (p<0.05); NS, not significant.

FIG. 10. Fusion between two sfGFP-FLG granules at high temporal resolution. Related to the data described above, these are snapshots 400 ms apart between two sfGFP-tagged FLG granules in HaCATs expressing WT(p) sfGFP-tagged FLG [sfGFP-(r8)10-Ctail]. Arrows point to the resolution of the fusion event over 5 seconds.

FIG. 11. Mechanical deformation of tail mutant FLG granules with an AFM probe. Temporal evolution of granule morphology, in HaCATs cells expressing a tail mutant FLG [sfGFP-(r8)8], upon their mechanical loading with an AFM probe. The AFM probe is seen as a dark shadow over the cell. Images correspond to a combination of bright-field (DIC) and GFP fluorescence, so granules appear white. Granules in this field of view are produced by a single transfected cell, which nuclei was marked with H2B-RFP. For clarity, we outline the nucleus (nu) with a red dashed line. Arrows point to the granule morphology prior to its major deformation. The top and bottom panels show two different short time series as the same granule is pushed by the AFM probe. The bottom panel corresponds to the liquid-like streaming of a granule around the nucleus that is also shown in FIG. 9D and in data not shown.

FIG. 12. Representative AFM height and stiffness maps for granules assembled from FLG processing variants and tail FLG mutants. (A) Simultaneous bright-field (DIC) and GFP fluorescence of specific, representative granules (marked by a red square) characterized by serial force-indentation measurements using AFM. For this experiment, HaCATs were co-transfected with the indicated FLG variants and a plasmid harboring H2B-RFP. The nuclear mRFP1 signal is shown overlaid over the brightfield/GFP signal. (B) Height maps from the AFM scan readily outline granule morphology, so we used them to create granule masks (labeled in red). We applied those masks to their corresponding stiffness maps (see Methods) (C) in order to average stiffness measurements over all pixels within the granule domain. (C) Note the striking changes in granule stiffness, with tail FLG mutants being particularly soft. FLG processing is clearly important to limit stiffening of granules (see FIG. 9F for average values across several granules per condition). (D) Re-scaled stiffness maps to visualize the full distribution of stiffness values for granules assembled from a tail mutant and its WT (p) counterpart. (E) Raw AFM force measurements for an indentation event within the corresponding granule masks in B.

FIG. 13. Conventional clients have limitations as in vivo probes of endogenous phase separation behavior. (A-B) Conventional clients are typically fluorescently-tagged proteins that bind to phase-separated scaffold proteins. To generate such clients and assess their effects on KGs, we introduced the short peptide ENLYFQS, which corresponds to the canonical TEVP protease cleavage sequence (cs) (65), into a mRFP-FLG* construct. The resulting construct [mRFP-cs-(r8)8-Tail] was then expressed in HaCAT cells which were also transfected with a fluorescently-tagged, protease-dead variant of TEVP (65) (sfGFP-dTEVP, see Table S5 for sequence details). The engineered client was nicely enriched within the mRFP-cs-FLG* KG-like granules that formed (left panels). However, the partition coefficient of this conventional client (P=2.1) remained low, as compared to the very high partition coefficient (P>20) of the new class of phase separation sensors that we generated (see FIG. 14). Note that the TEVP protease cleaves ENLYFQR with very low efficiency, serving as a control with very low affinity for the scaffold. This single point mutation in the cleavage sequence was sufficient to abrogate enrichment of the client into cs-containing KGs. (B) Recovery half-lives (in seconds) after photobleaching mRFP1-cs-FLG* signal in granules with or without the dTEVP client. The enrichment of the dTEVP client within tagged-KGs slowed down the liquid-like dynamics of FLG in this system. Taken together, the data in A and B show that although sfGFP-dTEVP can function as a client for phase-separated condensates, it alters the underlying liquid-liquid like dynamics, precluding its use for studying the material properties of endogenous phase transitions in vivo. Images are maximum intensity projections. Asterisks, statistically significant (p<0.05). (C-D) The 5100 domain of FLG is processed relatively early in the epidermal differentiation program. In (C and D), we show that although an S100-based client can be used to detect (albeit weakly) unprocessed FLG assembled into KGs, once it is processed, KGs remain intact but this client no longer recognized KGs. This result points out another potential caveat in designing conventional clients to study liquid phase transitions in tissues, namely the potential modification or processing of client-bound domains in the scaffold that eliminate client-binding but are otherwise nonessential for the scaffold's phase separation behavior. (C) We created tagged-FLG* constructs containing the S100 domain from mouse FLG [mS100-mRFP-(r8)8-Tail, see TABLE 5 for sequence details], and engineered a corresponding client by fusing the same mS100 domain to −20GFP with a C-terminal nuclear export signal (mS100-n20GFP, see TABLE 5 for client details). HaCATs transfected with mS100-n20GFP and FLG* with and without the mS100 domain. Note that the enrichment of the mS100-based client, while poor, is entirely dependent on the presence of the mS100 domain in FLG*. “P” indicates the partition coefficient for mS100-n20GFP. Dotted lines mark approximate cell boundaries. (D) Live imaging of mouse epidermis in utero transduced to drive suprabasal expression of mS100-n20GFP. Note that even relatively immature middle granular cells in mouse E18 skin already exclude mS100-sfGFP, which should be otherwise enriched within mS100-containing KGs (based on the behavior observed in A).

FIG. 14. Phase separation sensors efficiently enter and detect KGs, and accurately report their liquid-like properties. (A) Concept of a genetically-encoded phase separation sensor. (B) Amino acid composition of LC Tyr-high variants of a FLG repeat (repeat 8, r8), ordered at right according to their phase separation propensity. Variants were generated according to non-pathogenic residues frequently altered in FLG repeats in humans. % I: percent sequence identity to wild-type FLG repeat. Asterisks denote the two Tyr-high variants used as phase sensors in this study. (C) Domain architecture of the two phase separation sensors. % I: percent sequence identity to sensor A. (D) Sensor partitioning into KGs in HaCATs expressing Sensor A and indicated mRFP1-FLG. Partition coefficients (P, ratio of background corrected signal inside and outside granules) reveal robust ability of Sensor A to recognize FLG in its phase-separated granules (bottom row is pseudocolored to reveal the range of fluorescent intensity values). Nu, nucleus. (E) Presence of Sensor A does not alter FRAP half-life of FLG-assembled KGs in HaCATs. NS, not statistically significant. (F) Sensor A recovery half-lives after photobleaching granules composed of the indicated mRFP1-tagged FLG variants that model patient mutations. Each dot in (E)-(F) represents an individual FRAP half-life measurement of granules from multiple cells. Data are from >2 experiments. Asterisks, statistically significant (p<0.05). See also related FIGS. 13, 15, 16 and 17.

FIG. 15. Engineering of phase separation sensors. (A) Concept and design criteria for a phase separation sensor capable of sensing the phase separation behavior of filaggrin. (B) Frequency of non-synonymous mutations for each amino acid in human FLG based on the dbSNPs database (from a total of 3743 SNPs in the FLG gene). We show that the observed SNP frequency matches the expected mutational burden from simulations of a random mutational process that targets the most abundant codons in Flg. Histidine codons, being particularly abundant in Flg, are amongst the most commonly mutated. (C) Analysis of non-synonymous mutations involving His residues in human FLG. Note that His codons in FLG are frequently mutated to Tyr (Y) codons and that the frequency of these mutations cannot be predicted from simulations of a random mutational process. At the time of our analysis we identified 87 H>Y mutations (from 405 SNPs involving non-synonymous His mutations). We used these mutations to generate Tyr-high variants of a human FLG repeat (r8 in FIG. 1B) (see FIG. 14 and Materials and Methods). (D) To qualitatively assess the phase separation propensity of a FLG repeat unit and its Tyr-high variants, we fused them to a fluorescent protein at the N-terminus and a trimerization domain (NC1 domain from human COL18A1) at their C-terminus. Multimerization is a known mechanism to augment phase separation propensity—we have also confirmed this observation with a bacterial trimerization domain (foldon), see methods and supplementary text. Transfection of HaCATs with these engineered FLG repeat variants allowed us to rank their phase separation propensity based on the morphology and number of observed granules. Note that even after trimerization, the original FLG repeat fails to form compact granules and instead forms very large phases with shapes indicative of protein domains with very low surface tension. (E) Representative photobleaching experiment confirming the liquid-like behavior of Tyr-high variants (upon their trimerization). (F) To identify phase separation sensors with ideal performance (see criteria in A), we fused the original FLG repeat and its Tyr-high variants to three different fluorescent proteins that span a wide range of surface charges (see TABLE 3 for sequence details). Here we also indicate the sequence identify of each variant with respect to the original FLG repeat (r8).

FIG. 16. Evaluation of phase separation sensor designs. (A) Live imaging data for HaCATs transfected with a myc-tagged granule forming protein (myc-r8H2-foldon; foldon is a bacterial trimerization domain) and multiple sensor designs. Negatively-charged sfGFP variants lead to low partition coefficients into engineered KGs. Super-positively charged sfGFP provide improved partitioning into myc-tagged KGs and the partition coefficient is further enhanced by selection of a sensing domain with optimal phase separation propensity (ir8H2 in this case). Images correspond to maximum intensity projections. Each row corresponds to a sensor design and to the same image under different levels of signal saturation (shown in the legend as the range of allowed values for the GFP signal). (B) Immunostaining of HaCATs transfected with a myc-tagged KG-forming protein (myc-eFlg1-foldon) and two sensor designs based on the ir8H2 sensing domain: one based on a variant of +15GFP here referred as +15GFPK (top row), in which we mutated its characteristic surface-exposed Arg residues into Lys residues (TABLE 3), and one based on +15GFP (as published, see Table S2). Note that while both Arg and Lys are positively charged residues, surface-exposed Arg residues in +15GFP are partly responsible for the outstanding partitioning of +15GFP-based sensors into phase separated FLG-like granules. (C) HaCATs exclusively transfected with Sensor A (+15GFP-NES-ir8H2) or Sensor B (+15GFP-NES-ieFlg1) show fully diffuse signal in the cytoplasm.

FIG. 17. Not all clients serve as ideal probes to study phase separation. In FIG. 13, we consider caveats of conventional clients. Here, in FIG. 17, we directly compare the behaviors of the sfGFP-dTEVP client to our new type of client, Sensor A, which recognizes FLG only as it assembles into granules, where it interacts weakly along many different contact sites along the scaffold. (A) shows that phase separation sensors will work over a wide range of concentrations. (B) To easily compare data between Sensor A and the conventional client in FIG. S11 (sfGFP-dTEVP), here we show the recovery half-lives (same data as in FIG. 13) normalized to the average half-life in the absence of dTEVP. We applied the same normalization procedure for the phase separation sensor data (taken from FIG. 14E). (C) Concentration (based on fluorescence measurements) of the dTEVP client and Sensor A within tagged-KGs analyzed in (B). In these experiments, Sensor A was enriched within tagged-KGs to higher concentrations than dTEVP clients—so even very high levels of Sensor A remain innocuous and these values are well above the concentrations we measure in skin for KGs studied in FIG. 19D-H. Generally, clients engineered to bind a specific domain in the scaffold will have their maximum concentration within condensates limited by the saturation of scaffold binding sites. Phase separation sensors, which do not bind a specific domain in the scaffold, may accumulate to higher concentrations than the scaffold itself (particularly in systems like ours in which the scaffold is exceedingly larger than the sensor). (D) Sensor or client recovery half-lives after photobleaching GFP signal within tagged-KGs. The reported affinity for TEVP to ENLYQS is 60 μM (66). Thus, despite being a relatively weak binder (μM range), at the very high concentrations of FLG* protein within tagged-KGs (˜1 mM), clients with affinities in the μM range likely exist in a predominantly-bound state. This bound state is reflected in the long FRAP Half-life for sfGFP-dTEVP within tagged-KGs, which is comparable to the FRAP Half-life of tagged-FLG itself (see data in panel B of FIG. 13), but nearly an order of magnitude higher than for sensorA. These data show that relatively to dTEVP, Sensor A within KGs interacts very weakly with tagged-FLG. Note that the observed differences are not due to changes in probe size, since sfGFP-dTEVP has a lower molecular mass (54.8 KDa) than Sensor A (63.5 KDa). Asterisks, statistically significant (p<0.05).

FIG. 18. Generation of genetically-modified mice that express phase separation sensors in the epidermis. (A) We synthesized lentiviral vectors harboring genes encoding for Sensors A and B (see FIG. 14C) and under the control of a promoter of interest (see methods). For constitutive expression, we used a PGK promoter, whereas for doxycycline-inducible expression we used a TRE promoter (TRE3G, see methods for details). These vectors also included a human U6 promoter for expression of a shRNA. In most cases, unless indicated in the main manuscript, we used a well-tested Scramble (Scr) shRNA that does not target any sequence in the human and mouse genome and that has been validated to have no off-target effects in the skin epidermis (see methods). We produced high titer lentiviruses for in utero injection into the amniotic sac of mouse embryos at 9.5 days of development (see methods). This technique exposes the single layer of unspecified epidermal progenitors to the virus, which is taken up, integrated into the progenitor genome and stably propagated into adulthood. (B) Representative example of an in utero transduced mouse embryo harvested at 18 days of development. Note bright sensor signal throughout the skin surface. Using confocal spinning disk microscopy (right panel), we resolve Sensor A signal through the epidermis within fields of view of about 0.25 mm². The region demarcated in the left panel is simply a guide to the eye. Asterisks denote lack of GFP fluorescence in some (untransduced) cells, which highlights the expected mosaic nature of our in utero lentiviral approach.

FIG. 19. Skin exhibits dramatic phase separation dynamics during barrier formation. (A) Left, schematics of sagittal and planar views. Right, corresponding views of the fluorescent Sensor A in mouse skin. Planar skin views are through early, middle and late granular layers. Nu, nucleus. Dotted lines mark approximate cell boundaries. (B) Live imaging of an early granular cell over 800 min. (C) Example of photobleaching the Sensor A signal within an endogenous KG of a granular cell in mouse skin. (D) Sensor recovery half-lives after photobleaching KGs across cells within the middle granular layer of transduced mouse skin (each point is from a different cell; two animals analyzed for each sensor). (E) Quantification of changes in KG volume over time in granular cells as they reach the upper granular layer (related to Movie S6). (F) Sensor A reveals distinct liquid phase properties within different biomolecular condensates and contexts (in vivo KGs vs. granules generated de novo from S100-mRFP-(r8)8-Tail, expressed in cultured keratinocytes). For nucleolar measurements, a Sensor A variant lacking the nuclear export signal was used. In vivo and in vitro data from >2 experiments. (G) Sensor A detects an increase in the relative viscosity of KGs that occurs during granular layer maturation. Shown are FRAP half-lives in KGs within different granular layers (morphological differences at left; data from 3 animals). (H) Sensor A reveals conserved liquid-like properties of KGs despite divergence in amino acid sequence of granule-forming proteins. Mouse KG data in (H) is the same as in (G). Human KG data are from 3 separate skin equivalents and two sources of primary human keratinocytes. Asterisks, statistically significant (p<0.05). NS, not significant.

FIG. 20. Immunostaining of mouse epidermis expressing Sensor A. (A) Sagittal view of Sensor A (GFP) fluorescence and anti-FLG immunostaining in fixed and whole-mounted mouse epidermis. FLG was detected using a mouse anti-FLG (rabbit) antibody and a conventional anti-rabbit secondary antibody conjugate (see methods). The merged image shows that anti-FLG immunostaining in this whole-mount setting predominantly labels KGs in the early granular layers. (B) Planar views across early and late granular layers marked in (A). Note that Sensor A signal localizes within the rim-like structures that are typically reported upon detection of KGs with anti-FLG antibodies. In late granular cells, however, FLG immunostaining barely outlines prominent mature granules revealed by the phase separation sensor. (C-D) Because primary-secondary antibody complexes, which are larger than the expected mesh size of KGs (3-8 nm as reported for other biomolecular condensates) (67), failed to penetrate KGs in (A-B), we directly conjugated a fluorophore to an anti-mFLG antibody (see methods) to facilitate its penetration into and labeling of KGs in whole-mounted epidermis. (C) Using this approach, we show that FLG is prominently accumulated within the very large KGs that crowd the cytoplasm of mature (late) granular cells and which are also readily revealed by the phase separation sensor. Note that these mature KGs were essentially invisible to the primary-secondary antibody complex in (A-B). Note, however, that the primary antibody conjugate still fails to penetrate many KGs and the edges of those KGs are only slightly labeled. (D) The immunostaining approach in (C) reveals the same degree of KG crowding for mouse skin that lacks expression of a phase separation sensor.

FIG. 21. Liquid-like fusions between KGs in skin. (A) Time-lapse images (3D projections) of a granular cell in mouse epidermis in which two KGs undergo fusion events (as indicated by white arrows). The top time series shows raw Sensor A fluorescence, whereas the bottom panels show 3D surface renderings of Sensor A fluorescence to better visualize the 3D morphology of granules. (B) Time-lapse images (3D projections) of a human granular cell in which several KGs undergo fusion events (as indicated by white arrows). For this experiment we transduced human primary keratinocytes with two lentiviruses: one harboring a gene that encodes Sensor A and another lentivirus harboring a gene that encodes H2B-RFP to label nuclei. To allow for stratification and FLG expression in granular cells, prior to imaging we cultured transduced keratinocytes for 6 days under differentiation conditions (see methods)—we previously optimized this protocol to trigger pronounced FLG expression.

FIG. 22. Keratin-FLG interactions stabilize KGs and structure the cytoplasm in skin. (A) HaCATs transduced with a doxycycline-induced mRFP1-K10-HaCATs lack K10-, and allowed for K10 integration into the endogenous K5/K14 filaments followed by transfection with sfGFP-FLG. These HaCATs formed liquid-like KGs (arrows) interspersed within the keratin network (bottom). (B) Live imaging of cell in (A) showing three different types of keratin-KG interactions. Uncaged KGs fuse rapidly, while caged KGs fuse rarely/slowly. Fenced KGs are impeded from fusing. Double arrows depict temporal fusion events; single arrow denotes keratin cable preventing fusion. (C) When mCherry harbors the LC domains of hK10, it acquires the ability to partially partition into KGs (P=1.6). (D) Phase separation of sfGFP-(r8)4 FLG is promoted in HaCATs that contained mRFP1-K10 fibers at the time of transfection. Critical concentrations for phase separation were estimated as in FIG. 8C (data from three experiments). (E) Density of FLG within KGs assembled in (D) is similar ±an hK10 network. (F) Planar 3D view of E18.5 granular layer from the skin of an embryo transduced in utero with a suprabasal-specific driver of mRFP1-K10 and Sensor A. Accompanying cartoon depicts protein localization patterns seen in early and mature (late) granular cells. (G) Optical sections through mature granular cells in mouse skin show prominent granules encased by thick keratin bundles. Single red channel reveals void where KGs reside, indicating that the KGs are caged. Asterisks, statistically significant (p<0.05). NS, not significant.

FIG. 23. Low complexity domains in human keratin 10 mediate interactions with FLG and its KGs. (A) Architecture of human keratin 10 drawn with the proper relative size of its domains. While the coiled-coil domain is conserved among type I keratins and is central to its dimerization with type II keratins and assembly into 10 nm filaments, the LC domains vary markedly in size and in sequence. Note the atypically large low complexity (LC)N- and C-terminal domains that flank the central coiled-coil (helical) rod domain of human K10. (B) We grafted these LC domains onto mCherry and transfected these constructs into HaCATs to assess their behavior. Similar to mCherry itself, mCherry with one or both LC domains does not exhibit phase separation upon overexpression in HaCATs. mCherry grafted with K10 LC domains is predominantly diffuse in the cytoplasm, but occasionally marks perinuclear keratin fibers. (C) Co-expression of mCherry variants and sfGFP-r(8)8-Tail (to form sfGFP-tagged KGs). Note that while mCherry alone is well excluded from KGs (partition coefficient, P=0.4), mCherry grafted with one or both K10 LC domain readily partitions into KGs. The mCherry construct with both K10 LC domains exhibited the highest partition coefficient (P=1.9 vs P=1.3). All images are maximum intensity projections from live imaging data.

FIG. 24. Dense KG arrays dynamically respond to their environment to promote nuclear changes that lead to skin barrier formation. (A) Nucleus-KG interactions in HaCATs transfected with engineered FLG variants. (B) Nucleus-KG interactions in an individual granular cell from live imaging (optical section) of E18.5 mouse skin with resolution of both nuclei (H2B-RFP) and KGs (Sensor A). Arrows point to KG-associated nuclear deformations. (C) Granular cell to squame transition, complete within hours, as depicted by live imaging (3D view) of E18.5 mouse epidermis with resolution of both nuclei and KGs as in (B) (also data not shown). Early signs include chromatin compaction (arrows) and diminished partitioning of the (GFP-labeled) sensor within KGs. Late signs include KG disassembly and enucleation. (C-D) In utero Flg knockdown depletes endogenous KGs, causes a delay in enucleation and partially compromises the skin barrier. Enucleation speeds were determined by live imaging of chromatin degradation through the granular to squame transitions. Barrier quality was measured as transepidermal water loss (TEWL) in backskin of neonates. Asterisks, statistically significant (p<0.05). (E) Effects of shifting the intracellular pH on KG dynamics of mRFP1-tagged FLG* and Sensor A, as monitored by live imaging of HaCATs and shown as maximum intensity projections. Note rapid (t=5 min) pH-triggered dissolution of KG components from granules to cytoplasm. g1 and g2 show the pH-responsive behavior of individual granules. Nu, nucleus. (F) Live imaging (3D view) of the process of enucleation/cornification in epidermis of embryos transduced to drive suprabasal expression of an organelle marker (top: sensorA/KGs; bottom: H2BRFP/Nuclei) and a compatible pH reporter whose fluorescence is lost below pH 6.5. mNectarine (top) shows that when the intracellular pH of granular cells drops below pH 6.5, KGs begin to disassemble. SEpHLuorin senses a similar pH drop and further reveals that it precedes chromatin compaction. (G) Effects of pH-induced KG dynamics in Sensor A⁺ skin explants transduced with H2B-RFP and either Scr-shRNA (top) or Flg-shRNA(bottom). Note that chromatin compaction, which takes place concomitant with a pH-instigated reduction in KGs, does not occur if KGs are missing altogether, underscoring the role for this pH-mediated disassembly of KGs in triggering the process of enucleation. See also FIGS. 25-30.

FIG. 25. KGs prominently deform the nucleus. (A-B) Nucleus-KG interactions in HaCATs transfected with engineered FLG variants. (A) HaCATs that express synthetic WT(p) FLG [sfGFP-(r8)12-Tail; similar to wild-type FLG in KGs], typically form KGs that can prominently deform the nucleus. (B) In contrast to WT(p) FLG*, mutants without the C-terminal domain [sfGFP-(r8)12] form KGs that fail to deform nuclei and instead change their shape to wet the nuclear surface. (C-D) Nucleus-KG interactions in human primary keratinocytes (C) and mouse skin (D), both of which undergo terminal differentiation and generate a granular layer replete with KGs. (C) Optical sections of individual granular cells from live imaging of primary adult human keratinocytes transduced with constructs for expression of Sensor A and H2BRFP. Primary keratinocytes were cultured for 4 days under differentiation conditions to trigger stratification and KG formation (see methods). (D) Optical sections of individual granular cells from live imaging of E18.5 mouse skin transduced with constructs for expression of Sensor A and H2B-RFP. Sensor A labels endogenous KGs and H2BRFP labels nuclei. Arrows point to KG-induced nuclear deformations.

FIG. 26. Enucleation dynamics in mouse skin. (A) We studied enucleation through live imaging of E18.5 skin explants taken from an embryo whose epidermal progenitors were transduced in utero with a lentivirus harboring a gene encoding H2B-RFP under the control of a constitutive promoter. This approach allowed us to capture a few complete enucleation events per imaging session (˜16-20 h). In some instances, nuclei were sufficiently sparse to create high quality surface renderings (shown in purple) of chromatin signal in individual (late) granular cells. (B) Using the imaging and surface rendering approach in (A), we quantified the relative changes in nuclear volume, using chromatin signal as a proxy, through the process of enucleation. Note the stereotypical and rapid decline in nuclear volume (chromatin compaction) over the span of 2 hours.

FIG. 27. KG dynamics through the initial stages of enucleation. (A-B) Live imaging of the process of enucleation in E18.5 skin explants from embryos whose epidermal progenitors had been transduced in utero to enable suprabasal expression of Sensor A and constitutive expression of H2B-RFP (to label chromatin). Similar to the behavior reported in FIG. 24C, we observed a perfect synchronization of release of the sensor from within KGs, concomitant with its accumulation in the cytoplasm and the initiation of chromatin compaction. Arrows point to nuclei undergoing degeneration/loss. The late stages and completion of these two enucleation events were also observed (data not shown).

FIG. 28. Validation of pH sensors and pH sensitivity of other fluorescent proteins. (A) We transfected HACATs with constructs encoding two published pH reporters that are highly sensitive in our pH range of interest (7.4 to 6.0): SEpHLuorin (60), a GFP-based pH reporter, and mNectarine, an RFP-based pH reporter (61). Both reporters were designed to lose brightness at low pH (˜6.0). When we switched the media pH from 7.4 to 6.3 in the presence of KCl and Nigericin (see methods) to set the intracellular pH to 6.3, both SEpHLuorin and mNectarine exhibited a sharp drop in fluorescence intensity. Average final fluorescence (relative to pH 7.4) is shown (average of 10-30 cells in each case). While sfGFP (as well as EGFP and those closely-related variants) are known to be pH sensitive at pH values approaching 6.5, which we verify here for sfGFP (drop in signal to 9.1%), +15-NES-GFP is only mildly pH sensitive and remains highly fluorescent at pH 6.3. mRFP1 is known to be pH insensitive in this pH range and our experiments confirm its pH insensitivity.

FIG. 29. pH-responsiveness of tagged-KGs. Response of tagged-KGs in HaCATs co-expressing FLG* [mRFP1-(r8)8-Tail] and Sensor A (granules appear yellow due to the high enrichment of Sensor A within mRFP-tagged KGs) to our intracellular pH buffering media (with KCl and Nigericin, see methods). (A) Detailed view of tagged-KGs (within cells), soon after (t=5 min) they are exposed to different pH shifts. Note that starting a ˜pH 6.3, FLG* begins to lose its granule-forming properties and transition to the cytoplasm. This effect is more prominent at pH values below the pKa of His (see data at pH of 4.5). In contrast, at neutral or alkaline pH (see data at a pH of 9.2), we only see swelling of tagged-KGs without release/dissolution of FLG* into the cytoplasm. Results are provided when pH is kept at ˜7.4 (B) or lowered to pH 6.3 (C). Images are maximum intensity projections that were taken 5 min after the indicated shift in pH. Note that under these conditions and in the absence of a pH shift, tagged-KGs remained largely unaffected and Sensor A stays prominently accumulated within KGs. The partition coefficients shown in the images correspond to data for Sensor A in those specific cells. The average partition coefficient for Sensor A across 3 to 4 cells prior to pH change was P=26, which dropped to P=12 at pH 7.3 and to P=2.5 at pH 6.3. These sharp transitions in partition coefficient confirm that the release of Sensor A is prominently and specifically triggered by the pH shift, in agreement with the expected decline in histidine-rich FLG's propensity to maintain its phase-separated state at low pH. (D) Same as in (C) but 5 min after reversing the intracellular pH to ˜7.4. The lower panels in (D) show a detailed view of a granule (from a different cell than in the upper panels) and only include the RFP signal to highlight the pH-triggered release of FLG* into the surrounding cytoplasm, followed by its subsequent reassembly into granules as the pH is reversed to 7.4. This experiment demonstrates that the effects of pH on both FLG* and Sensor A are dynamic and reversible. In (B-D), dotted lines mark approximate cell boundaries.

FIG. 30. pH-triggered and pH-like changes in human and murine granular cells. (A) Direct manipulation of pH in human granular cells in culture leads to a rapid reduction in the partitioning of Sensor A into endogenous KGs. Its dilution into the cytoplasm as well as the progressive dissolution of KGs are consistent with the pH-triggered dynamics observed for mRFP-tagged KGs (FIG. 24E). Primary human keratinocytes were transduced to drive expression of Sensor A and H2B-RFP and induced to differentiate over 7 days to drive their stratification and FLG expression (see methods). (B) Live imaging of the process of enucleation and cornification in mouse epidermis in utero transduced to drive suprabasal expression of Sensor B, which is smaller than Sensor A and can upon loss of nuclear integrity enter the nucleus. Note that a late granular cell with abundant and large KGs first shows signs indicative of the endogenouse pH shift (FIG. 24F top panels), becoming increasingly abundant in the cytoplasm and followed by rapid entry of Sensor B into the nuclear compartment (see arrow at t=20 min). Twenty minutes after the initiation of KG dissolution (at t=40 min), Sensor B is equally partitioned between the cytoplasm and leftover KGs (similar to what we observe for Sensor A in response to the endogenous pH shift). Starting at t=100 min, the nuclear compartment is no longer visible and the cell has acquired squame-like features (arrowheads). In the last snapshot (t=120 min), a second cell (adjoined to the first squame) has transitioned to a squame.

FIG. 31. Engineering of phase separation sensors. The accessory and sensor domains are depicted and color-coded with alternative sequences or proteins in the domains. Exemplary sensor domains include i-r8H2 and ieF1. Accessory domains can provide markers, such as for live imaging, or can be active enzymes, such as for proteomics applications, or can be contrast agents, such as for EM applications. Examples of multi-domain sensors are indicated. Sensor A is 100% identical with respect to itself, and the % identity (% I) value f 27% shown for Sensor B is % identity with respect to Sensor A. The two sensors share little sequence identity among themselves.

FIG. 32. Apex2-SensorA biotinylates KG components in mice genetically-modified to express the Apex2-SensorA (SEQ ID NO:50). Apex2 does not function with regular biotin (as normally found in our bodies), but requires a chemically-modified biotin (BP) that is added to skin prior to tissue processing/fixation. Filaggrin, a KG scaffold, was detected with a rabbit anti-Flg antibody (red). Biotin-containing proteins were detected with a fluorescently-labeled monomeric streptavidin protein (mStreptavidin; gray). Sensor signal (green) was endogenous to the GFP domain in the sensor and was not amplified. Upon harvesting mouse skin and following dispase treatment to isolate the epidermal skin layer, we added BP and Hydrogen-peroxide (H₂0₂) to trigger biotinylation of proteins in proximity (with a labeling radius of a few nm) to the Apex2-containing phase separation sensor. We saw low levels of biotinylation (above background) when BP was added and H₂0₂ was omitted (not shown). The advantage of Apex2 over BioID2 (which uses endogenous biotin) is that we control the time at which the Apex2-containing sensor becomes enzymatically-active within condensates (that is, when both BP and H₂0₂ are exogenously provided). In the absence of BP even if H₂0₂ was added (bottom panels), we saw no signs of biotinylation. In contrast, when both BP and H₂O₂ were added (top panels), we saw prominent mStreptavidin signal within KGs (outlined in red by the anti-mFlg antibody and enriched in sensor signal), which indicated high-efficiency labeling of KG components. Note that biotinylation was restricted to the components residing within KGs and spared the surrounding cytoplasm.

FIG. 33. Biotinylation by a cytoplasmic Apex2 spares Flg granules. We followed the same procedure as in FIG. 32, but this time the experiment involved mice whose skin was genetically-modified to express a fluorescently-labeled Apex2 protein lacking a phase separation sensor domain. We refer to this Apex2 construct as cytoplasmic Apex2 because we also designed it to be excluded from KGs and hence resides in the cytoplasm, outside of KGs. Upong harvesting the skin epidermis and processing the tissue as in FIG. 32, note that biotinylation (mStrepatividin signal) occurs outside of KGs, sparing KG components (which appear as black holes when not labeled. The outline of KGs was demarcated by anti-mFlg antibody signal (red). This cytoplasmic Apex2 construct may be used as a control in quantitative proteomics studies involving KG-targeted Apex2 sensors. Images correspond to mouse skin from mice genetically-modified to express the indicated phase separation sensor.

FIG. 34. Apex2-SensorB biotinylates early and late granules. We followed the same procedure as in FIG. 32, but this time the experiment involved mice whose skin was genetically-modified to express a Apex2-SensorB (SEQ ID NO:51), which features a different sensor domain (SensorB instead of SensorA). As in all other cases, the resulting tissue is mosaic, so that only a subset of cells in the epidermis express the sensor. All images correspond to tissue exposed to BP and H₂0₂ to trigger Apex2-mediated biotinylation. Top panels show sagittal views of the murine epidermis. Early granular cells (those closest to the basal side) show sensor-labeled KGs (green) that also feature prominent biotinylation (mStrepatividn signal; gray). Moving towards the skin surface, late granular cells also featured sensor-labeled KGs with prominent biotinylation. Bottom panels show planar views across different epidermal layers (spinous lacking KGs, early granular, middle granular and late granular). Each level is identified with a different color. These planar views reveal KG-restricted biotinylation as KGs mature across the entire granular layer. Images correspond to mouse skin from mice genetically-modified to express the indicated phase separation sensor.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The amino acid residues described herein are preferred to be in the “L” isomeric form. However, residues in the “D” isomeric form can be substituted for any L-amino acid residue. NH₂ refers to the free amino group present at the amino terminus of a polypeptide. COOH refers to the free carboxy group present at the carboxy terminus of a polypeptide. In keeping with standard and recognized polypeptide nomenclature, abbreviations for amino acid residues are shown in the following Table of Correspondence:

TABLE OF CORRESPONDENCE

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a sequence, including a DNA sequence, that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A nucleic acid sequence, including a DNA sequence is “operatively linked” to an expression control sequence when the expression control sequence controls and regulates the transcription and translation of that nucleic acid or DNA sequence. The term “operatively linked” may include having an appropriate start signal (e.g., ATG) in front of the nucleic acid or DNA sequence to be expressed and maintaining the correct reading frame to permit expression of the nucleic acid or DNA sequence under the control of the expression control sequence and production of the desired product encoded by the nucleic acid or DNA sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

A cell has been “transformed” or “transduced” by exogenous or heterologous nucleic acid or DNA when such nucleic acid or DNA has been introduced inside the cell. The transforming or transducing nucleic acid or DNA may or may not be integrated (covalently linked) into chromosomal DNA making up the genome of the cell. In prokaryotes, yeast, and mammalian cells for example, the transforming or transducing nucleic acid or DNA may be maintained on an episomal element such as a plasmid. With respect to eukaryotic cells, a stably transformed or transduced cell is one in which the transforming or transducing nucleic acid or DNA has become integrated into a chromosome or otherwise incorporated so that it is inherited by daughter cells through chromosome replication. This stability is demonstrated by the ability of the eukaryotic cell to establish cell lines or clones comprised of a population of daughter cells containing the transforming or transducing nucleic acid or DNA.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are nucleic acids including DNA sequences encoding a phase separation sensor hereof which code for a phase separation sensor having the same amino acid sequence as provided herein, including in the Tables and Examples or sequences provided, but which are degenerate to one another. By “degenerate to” is meant that a different three-letter codon is used to specify a particular amino acid. It is well known in the art that the following codons can be used interchangeably to code for each specific amino acid (RNA codons are provided, however as would be recognized by one skilled in the art, for a DNA sequence a T should be substituted for a U in the codon sequence):

| | |
|---|---|
| Phenylalanine (Phe or F) | UUU or UUC |
| Leucine (Leu or L) | UUA or UUG or CUU or CUC or CUA or CUG |
| Isoleucine (Ile or 1) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or AGG |
| Glycine (Gly or G) | GGU or GGC or GGA or GGG |
| Tryptophan (Trp or W) | UGG |
| Termination codon | UAA (ochre) or UAG (amber) or UGA (opal) |

The phase separation sensors of the invention extend to those proteins having the amino acid sequence data, characteristics and sequences described herein and presented in the Tables and Examples herein, and the profile of activities set forth herein and in the Claims. Accordingly, proteins displaying substantially equivalent activity are likewise contemplated. Further, proteins displaying somewhat altered activity but remaining active and capable of targeting or associating with biomolecular condensates are likewise contemplated. These modifications may be deliberate, for example, such as modifications obtained through site-directed mutagenesis, or through random mutagenesis, or may be accidental, such as those obtained through mutations in hosts. Also, the phase separation sensors, including the specific sensors exemplified by noted sequence herein, are intended to include within their scope proteins specifically recited herein as well as substantially homologous analogs and variants, including allelic variations, particularly wherein the analogs or variants remain active and capable of targeting or associating with biomolecular condensates, particularly with a target biomolecular condensate.

Mutations can be made in the phase separation sequences and nucleic acid sequences provided and contemplated herein such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein or peptide. A non-conservative change is more likely to alter the structure, activity or function of the resulting protein. The present invention should be considered to include sequences containing conservative changes which do not significantly alter the activity or binding characteristics of the resulting protein or peptide.

The following provides one example of various groupings of amino acids:

Amino acids with nonpolar R groups: Alanine, Valine, Leucine, Isoleucine, Proline, Phenylalanine, Tryptophan, Methionine; Amino acids with uncharged polar R groups: Glycine, Serine, Threonine, Cysteine, Tyrosine, Asparagine, Glutamine; Amino acids with charged polar R groups (negatively charged at pH 6.0): Aspartic acid, Glutamic acid; Basic amino acids (positively charged at pH 6.0): Lysine, Arginine, Histidine (at pH 6.0).

Another grouping may be those amino acids with phenyl groups: Phenylalanine, Tryptophan, Tyrosine

Another grouping may be according to molecular weight (i.e., size of R groups): Glycine 75; Alanine 89; Serine 105; Proline 115; Valine 117; Threonine 119; Cysteine 121; Leucine 131; Isoleucine 131; Asparagine 132; Aspartic acid 133; Glutamine 146; Lysine 146; Glutamic acid 147; Methionine 149; Histidine(at pH 6.0) 155; Phenylalanine 165; Arginine 174; Tyrosine 181; Tryptophan 204.

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces P-turns in the protein's structure.

Two amino acid sequences are “substantially homologous” when at least about 70% of the amino acid residues (preferably at least about 80%, and most preferably at least about 90 or 95%) are identical, or represent conservative substitutions.

In embodiments hereof, variant peptide sequences having substantial identity to the sequences provided herein are contemplated. Variants having different amino acid sequences, wherein the sequence has at least 75%, at least 80%, at least 85%, at least 90%, at least 95% amino acid sequence identity to a sequence provided herein are included in the invention. Variants are and can be selected for maintaining the purpose and characteristics of the parent sequence from which they are variant. Thus, suitable variant artificial client protein sequences will retain the characteristic(s) of intrinsic disorder and capable of engaging in ultra-weak phase-separation specific amino acid interactions with one or more component protein, particularly one or more target component protein in the condensate.

A “heterologous” region of a nucleic acid or of a DNA construct is an identifiable segment of nucleic acid, including DNA, within a larger nucleic acid or DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene).

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein. Fab and F(ab′)₂ portions of antibody molecules are prepared by the proteolytic reaction of papain and pepsin, respectively, on substantially intact antibody molecules by methods that are well-known.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, more preferably by at least 70 percent, most preferably by at least 90 percent, a clinically significant change in the mitotic or enzymatic activity of a target cell, or alter or modify a feature of pathology such as a characteristic of one or more biomolecular condensate or target protein or component as may attend its presence and activity.

Notably, previous conceptions of the assembly of multidomain macromolecules and biological macromolecules, including those predominating in biomolecular condensates, have focused largely on the networks created by strong, specific interactions, without consideration of the extremely weak, nonspecific interactions that govern solubility, and how they would be affected by the assembly process. In fact, as demonstrated herein, considering the coupling between the strong and weak interactions, and therefore the ability of multivalency to promote phase separation, is essential to understanding the behavior of and being in a position to specifically assess, target and modulate or modify multivalent biological molecules. Thus, in some systems, such as disordered proteins, the important or relevant interactions may be in an intermediate spot on the spectrum between strong, stereospecific contacts and weak, nonspecific contacts. As disordered polymers become less soluble and as they grow, the presence of multiple points of contact between molecules provides an important driving force for phase separation. In this invention, the more weak and nonspecific interaction in biomolecular condensates are exploited in designing and applying and enabling novel phase separation sensors comprising an accessory and an artificial client sequence whereby the sensors are incorporated via the more weak and nonspecific (yet enriched across phase-separating proteins) interaction in biomolecular condensates.

In a general embodiment, the present invention relates to biomolecular condensates or membraneless compartments in cells, and the ability to detect, target, monitor, assess and modulate biomolecular condensates, including in vitro in cells and in vivo in animals. Novel phase separation sensors are provided which are uniquely capable of targeting or associating with a biomolecular condensate, particularly a specific and target biomolecular condensate by design. The sensors comprise at least two domains, wherein a first domain includes one or more accessory protein or molecule and a second domain includes an artificial client protein or intrinsically disordered sequence. The artificial client protein or intrinsically disordered sequence is uniquely capable of interacting with one or more component protein in a target biomolecular condensate.

Phase separation sensors of the invention include wherein the sensor is capable of targeting or associating with a biomolecular condensate and wherein the sensor comprises at least two protein domains. The first domain comprises one or more accessory protein or molecule. The first domain may thus include one or more subdomains or one or more proteins or peptides. The second domain comprises an artificial client protein having intrinsic disorder and capable of engaging in ultra-weak phase separation-specific amino acid interactions with one or more component protein in the condensate.

Phase separation sensors thus comprise at least one accessory protein or molecule domain and an additional domain comprising an artificial client protein having intrinsic disorder. As described and provided herein, two-domain sensors were designed, constructed and evaluated based on a two-domain structure, with the sensors having a first domain comprising a fluorescent protein or marker (the at least one accessory protein or molecule) and a second domain comprising an IDP sensing domain or an artificial client protein sequence. A general two-domain sensor architecture is as follows:

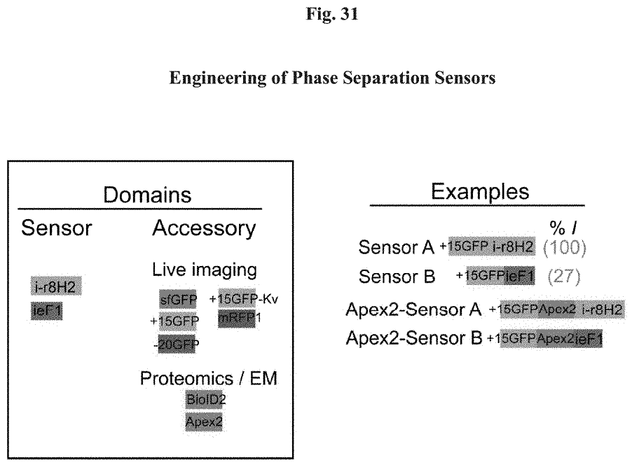

Two-domain: [Fluorescent marker/Enzyme/Cargo]-[Optional Linker]-[IDP sensing domain]
In further studies, multi-domain sensors were designed, constructed and evaluated based on a three-domain structure (see for example FIG. 31), with the sensors having a first domain comprising a fluorescent protein or marker (an accessory protein or molecule), a further domain comprising an enzyme or cargo protein (an additional accessory protein or molecule) and a final domain comprising an IDP sensing domain or an artificial client protein sequence. Linkers can optionally be utilized between the domain sequences. A general three-domain sensor architecture is as follows:
Three-domain: [Fluorescent marker]-[Optional linker]-[Enzyme/Cargo]-[Optional Linker]-[IDP sensing domain]

In particular, the phase separation sensor lacks independent phase separation behavior when expressed in the cell, including whereby the sensor associates with the biomolecular condensate without disrupting the condensate. Thus, the sensor does not independently form a condensate, but can sufficiently interact with a condensate target or target protein or sequence so as to be incorporated in a condensate.

The two or more domains comprising the sensor of the invention may be directly linked or may be separated in each or any instance by one or more linker sequence. In a particular embodiment, one or more accessory protein(s) and/or the accessory protein(s) and the artificial client protein are separated by a flexible linker sequence. The flexible linker sequence may comprise between 2 and 10, 10 and 20, 20 and 40, 2 and 20, 2 and 30, 2 and 40, up to 10, up to 20, up to 30, up to 40 amino acid residues. The flexible linker sequence may comprise between 2 and 10 amino acid residues. In a preferred embodiment, one or more short flexible linkers of 2 to 10 residues in length is utilized. In an embodiment, the linker sequence lacks charged residues. In an embodiment, the linker sequence contains charged residues. In an embodiment, the linker sequence contains charged residues and is zwitterionic, having equal numbers of positive-charged and negatively-charged residues. In exemplary embodiments and sequences hereof, linkers of 2, 4 and 10 residues are utilized. Exemplary linker sequences are provided herein, including in the Examples and alternative sequences are well known to or could be designed by those of skill in the art. For example, Chen et al describes various useful and flexible linkers (Chen X et al (2013) Adv Drug Deliv Rev 65(10):1357-1369). In embodiments, linker sequences GSPG (SEQ ID NO: 59) and/or GRSDGVPGSG (SEQ ID NO: 60), as examples, are utilized.

In a particular embodiment, a phase separation sensor is provided wherein the target component protein is a filaggrin family protein or paralog protein. In one such embodiment, the sensor artificial client protein sequence is derived from or based on a filaggrin protein sequence. The artificial client protein sequence may be derived from or based on human filaggrin protein sequence or on a mouse filaggrin protein sequence. The artificial client protein sequence may be derived from or based on a filaggrin protein sequence provided in TABLE 1, or the mouse of human filaggrin protein as provided in Table 1 or set out in SEQ ID NO: 1 or in SEQ ID NO: 56. Notably, the mouse and human filaggrin sequences when compared directly have about 34% identity in amino acid sequence. In particular, the artificial client protein sequence may be derived from or based on a filaggrin protein repeat component sequence. Exemplary filaggrin-based or filaggrin-targeting phase separation sensors are provided herein, including in TABLE 3 and in Examples 1 and 2 hereof. Phase separation sensors include Sensor A (SEQ ID NO:26), Sensor B (SEQ ID NO:27), Apex2-Sensor A (SEQ ID NO:50), Apex2-Sensor B (SEQ ID NO:51), Sensor C (SEQ ID NO:52) and Sensor D (SEQ ID NO:53).

Phase separation sensors are contemplated and provided herein that are directed to one or more biomolecular condensate in vivo in an animal. The sensor(s) of the invention may target or associate with one or more biomolecular condensate in the cytoplasm of a cell or in the nucleus of a cell. In an exemplary embodiment, the condensate is a keratohyalin granule (KG) in the epidermis or in one or more skin cell. In embodiments, one or more phase separation sensor is provided that targets a biomolecular condensate selected from P granule, Germ granule, Lewy bodies, synaptic condensates, stress granule, P bodies, T cell signalosome, crystalline condensates of the lens fibers, and other cytoplasmic condensates or membraneless organelles assembled through liquid-liquid phase separation. In further embodiments one or more phase separation sensor is provide that targets a biomolecular condensate in the nucleus. In an embodiment, nuclear condensates may be selected from Nucleoli, Paraspeckles, Histone Locus Bodies, Cajal Bodies, Heterochromatin. The biomolecular condensate may be an RNA-protein granule or an RNA-containing condensate. In an embodiment, the target condensate protein may be an RNA-binding protein.

Wherein the target condensate or condensate protein is a cytoplasmic condensate or cytoplasmic condensate protein, the phase separation sensor may include one or more nuclear export signal (NES). In particular, the NES prevents nuclear localization and targets the protein or sensor to the cytoplasm. Wherein the target condensate or condensate protein is a nuclear condensate or condensate or condensate protein located in the nucleus, the phase separation sensor may include one or more nuclear localization signal (NLS), so as to promote or limit localization to the nucleus. Exemplary NES and NLS sequences are provided herein and recognized and known in the art. Alternatively, a sensor of the invention may lack a nuclear localization signal and also lack a nuclear export signal and thereby may function, be expressed in or localize to either of or both of the nucleus and cytoplasm.

In an embodiment of the invention, a phase separation sensor is provided to investigate or access phase separation of a putative or candidate condensate, including to determine whether a target protein is incorporated in a biomolecular condensate. Thus, a phase separation sensor is provided to investigate or access phase separation of a putative or candidate condensate, including to randomly or indirectly characterize the proteins in a putative or candidate condensate. Thus, a phase separation sensor is designed which generically or relatively non-specifically associates with biomolecular condensates by virtue of ultra-weak interactions and not by target sequence-based derivation. Provided that the interaction is sufficient and the accessory protein label is adequate, a condensate may be generally or generically targeted and tagged or monitored by association with the sensor. In accordance with an embodiment of the invention, a phase separation sensor of the invention may identify, monitor and characterize a biomolecular condensate of previously unknown nature, composition or purpose. In an embodiment of the invention, a sensor is designed to generally or generically recognize and tag an intrinsic disorder protein (IDP) or sequence, including wherein the IDP is predicted to undergo liquid-liquid phase separation.

In designing and implementing a sensor for targeting or associating an unknown or non-specified biomolecular condensate, the artificial client protein sequence is designed to generically associate with one or more intrinsically disordered protein sequence, or an intrinsically disordered repeat by virtue of weak and non-specific interactions over the repeat or IDR sequence, and by selecting amino acids or a compositional character that will permit non-specific weak interactions.

The invention provides nucleic acids encoding a phase separation sensor hereof. In an embodiment, a sensor may comprise a nucleic acid sequence, such as an RNA or DNA sequence. DNA molecules comprising the nucleic acids are an embodiment of the invention. Further, a vector comprising the nucleic acids or DNA molecules of the invention is also provided.

### Accessory Proteins

An important embodiment of the phase separation sensors of the invention is that the domain component comprising an artificial client protein, by virtue of its ability to interact and associate with target component sequences, such as via intrinsic disordered sequence, thereby delivers or brings along the or a other domain component, particularly one or more accessory protein to the biomolecular condensate or within associative distance of one or more target component sequences. The other domain of the instant phase separation sensors may comprise one or more accessory protein, peptide or molecule. The accessory protein may provide a label or marker, such as a fluorescent protein, such that the biomolecular condensate can be visualized or monitored. Alternatively, or in addition, the accessory protein may provide an activity, such as an enzymatic activity, to or in the vicinity of the biomolecular condensate.

The invention contemplates a sensor molecule or protein which provides an active, useful, visible or detectable label or marker, particularly via one or more accessory protein or molecule in a first domain. The invention contemplates a sensor molecule or protein which provides a function, enzyme or capability. Thus, the sensor comprises in a first domain, or in one or more embodiment or portion of a first domain, one or more accessory protein wherein at least one accessory protein provides a detectable or functional label.

In embodiments of the invention, the at least one accessory protein is a fluorescent protein. The fluorescent protein may be selected from a protein known in the art, provided that the fluorescent protein does not detract from or interfere with the sensors ability to target or associate with a target condensate component protein or the biomolecular condensate. Numerous suitable and applicable fluorescent proteins are known and available in the art. The fluorescent protein may be selected from one or more of a blue/UV protein, a cyan protein, a green protein, a yellow protein, an orange protein, a red protein, a far-red protein, a near-IR protein, a long stokes shift protein, a photactivatible protein, a photoconvertible protein and a photo switchable protein. Examples of blue/UV fluorescent proteins include TagBFP and Sapphire. Examples of Cyan proteins include ECFP and derivatives thereof, Cerulean, TagCFP and mTFP1. Examples of green proteins include GFP and derivatives thereof, Emerald, monomeric azami green. Examples of yellow proteins include EYFP and derivatives thereof, and examples of orange proteins include monomeric kusabira orange and derivatives thereof. Red fluorescent proteins are known in the art and include for example RFP and derivatives thereof, mRaspberry, mCherry, mStrawberry, mRuby. The fluorescent protein may particularly be a GFP protein. In an embodiment, the GFP protein is a GFP protein with positively-charged amino acids exposed on the protein surface. The fluorescent protein may be a supercharged protein, wherein the protein sequence is altered, mutated or modified to have additional positively charged residues. For example, the GFP protein may be a supercharged GFP protein. Supercharged GFP proteins are described for instance in US 2011/0112040A1 and in U.S. Pat. No. 9,221,886. In a preferred embodiment and aspect, the GFP protein may be +15GFP.

The invention contemplates and includes wherein more than one phase separation sensor is introduced in a cell, whereby distinct sensors target different component proteins and/or carry different accessory proteins, such as different fluorescent proteins, such that multiple and distinct components of a biomolecular condensate are targeted and can be monitored or evaluated simultaneously.

Other relevant and useful accessory proteins in accordance with the invention are enzymes. One or more enzyme may be selected from a protease, nuclease, ligase, peroxidase, phosphatase, kinase and protein capable of modifying a protein or nucleic acid.

One or more accessory protein may comprise a label. In an embodiment, the label may include a radioactive element. In one such embodiment, the sensor may thereby introduce a label or readioactive element into a cellular sample. The label or element may then be examined by known techniques, which may vary with the nature of the label attached. In the instance where a radioactive label is used, it may be selected from isotopes such as the isotopes ³H, ¹⁴C, ³²P, ³⁵S, ³⁶Cl, ⁵¹Cr, ⁵¹Co, ⁵⁸Co ⁵⁹Fe, ⁹⁰Y ¹²⁵I, ¹³¹I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase.

In accordance with a further embodiment, at least one accessory protein may be capable of tagging one or more biomolecular condensate component with a detectable or functional molecule, peptide or marker. The examples provided herein exemplify sensors wherein one or more accessory protein is a peroxidase, including wherein the enzyme is capable of biotinylating one or more target component protein, for instance within a certain reaction distance from the enzyme protein molecule. The peroxidases Apex2 and BioID, for example, have been utilized.

In an embodiment of the invention, the sensor is a functionalized sensor and at least one accessory protein is capable of modifying a target component protein in the condensate. Said accessory protein may be capable of modifying condensate components, through covalent or non-covalent crosslinking of condensate components, to alter the material properties of the condensate. Crosslinking may be triggered by exogenous or endogenous stimuli to cells containing said condensates and accessory proteins.

In another embodiment, the sensor is a functionalized sensor and at least one accessory protein is capable of delivering a compound or agent to the condensate or to a target component protein in the condensate.

Experts in the art will further recognize, for example, that the peroxidase Apex2 domain in the phase separation sensors provided herein may be further modified to include other or alternative compounds or agents, such as enzymes or proteins of interest for example, so as to exploit said phase separation sensors as vehicles that deliver cargo of interest to biomolecular condensates. Said cargo may include but is not limited to fluorescent proteins, proteases, nucleases, ligases, peroxidases, phosphatases, kinases and other proteins capable of modifying proteins and nucleic acids.

### Artificial Client Proteins and IDP Sensing Domains

In accordance with the invention the artificial client protein aspect or domain of the phase separation sensor is an intrinsically disordered protein having low complexity sequence. Thus, the artificial client protein contains one or more disordered region that provides one or more or multiple weakly adhesive sequence elements. In an embodiment, the artificial client protein sequence lacks recognized protein three dimensional structural aspects. In an embodiment, the artificial client protein sequence contains repeated sequence elements. In an embodiment, the artificial client protein sequence contains low complexity sequence elements. In a particular such embodiment, the repeated sequence or low complexity elements provide basis for multivalent weakly adhesive intermolecular interactions.

In accordance with another embodiment of the invention, the sensor artificial client protein sequence comprises similar compositional bias or comprises related sequence patterns with low sequence identity to amino acid sequence of a naturally-occurring intrinsically disordered protein or protein region within a larger protein. This may be achieved in certain embodiments by reordering or by shuffling or randomizing the sequence of a naturally-occurring intrinsically disordered protein. In one such embodiment this similar compositional bias or related sequence patterns contributes to or is responsible for driving assembly of said biomolecular condensate.

In an embodiment of the invention, the phase separation sensor's artificial client protein sequence is related to the native or target intrinsic disordered protein (IDP) sequence by reversing its amino acid sequence. In accordance with this embodiment, the phase separation sensor sequence artificial client protein sequence is generated by reading the original, native or target IDP sequence in the non-natural C-terminal to N-terminal direction. In an embodiment, the intrinsically disordered protein sequence is reversed so as to read and be presented C terminal to N terminal in sequence order. This retains the amino acid composition but completely alters the sequence as presented per se.

In another embodiment, the artificial client protein sequence is enriched in a limited number of amino acid types. In a further embodiment, the artificial client protein sequence is enriched in a charged residues such as lysine, arginine, glutamate and aspartate. In view of the lack of sequence diversity in the artificial client protein, the sequence may be characterized by or may contain multiple short sequence repeat tracks, poly-single amino acid tracts, sequence blocks of positive or negative charge. These repetitive motifs may then contribute to interactions with the biomolecular condensate target protein(s).

A main criterion and characteristic of the artificial client protein sequence in a phase separation sensor of the invention is intrinsic disorder. It is important to note that many proteins have multi-domain architecture so that some domains are well-folded, and thus have defined/known secondary structure such as helices, sheets, etc, and some domains are intrinsically-disordered regions (IDRs). IDRs within proteins containing other domains are often responsible for their overall phase separation behavior. The phase separation sensors of the invention target the properties of those IDRs. In the case of filaggrin (FLG) (and its paralogs), for example, the overall protein is greater than 4000 amino acid residues in length and of those only the first 100 amino acids are part of a folded domain (so-called S100 domain composed of two EF-hand motifs). In other proteins, the relative size of the folded or structures and disordered domains varies widely.

Intrinsically disordered proteins (IDPs) lack stable tertiary and/or secondary structures under physiological conditions in vitro. They are highly abundant in nature and their functional repertoire complements the functions of ordered proteins. IDPs are involved in regulation, signaling, and control, where binding to multiple partners and low-specificity/low-affinity interactions play a crucial role. Intrinsic disorder is a unique structural feature that enables IDPs to participate in both one-to-many and many-to-one signaling. Numerous IDPs are associated with human diseases, including cancer, cardiovascular disease, amyloidoses, neurodegenerative diseases, and diabetes. Overall, intriguing interconnections among intrinsic disorder, cell signaling, and human diseases suggest that protein conformational diseases may result not only from protein misfolding, but also from misidentification, missignaling, and unnatural or nonnative folding. IDPs, such as α-synuclein, tau protein, p53, and BRCA1, are attractive targets for drugs modulating protein-protein interactions. From these and other examples, novel strategies for drug discovery based on IDPs are of interest and being developed (Uversky V N et al (2008) Ann Rev Biophysics 37:215-246).

The global, multi-level simplicity of IDPs/IDRs can be correlated with the character and peculiarities of their amino acid sequences, which are depleted in order-promoting residues (Trp, Cys, Ile, Val, Asn, and Leu) and enriched in disorder-promoting residues (Arg, Pro, Gln, Gly, Glu, Ser, Ala, and Lys) and commonly contain repeats (Radivojac P et al Biophys J. (2007) 92:1439-56, doi: 10.1529/biophysj.106.094045; Williams R M et al Pac Symp Biocomput. (2001) 2001:89-100; Romero P et al Proteins (2001) 42:38-48; Garner E et al Genome Inform Ser Workshop Genome Inform (1998) 9:201-13; Jorda J et al FEBS J (2010) 277:2673-82; Darling A L et al Molecules (2017) 22:27, doi: 10.3390/molecules22122027). Therefore, IDPs/IDRs are characterized by the reduced informational content of their amino acid sequences, and their amino acid alphabet is decreased in comparison with the alphabet utilized in the amino acid sequences of ordered domains and proteins. The behavior of an IDP as a highly frustrated system that does not possess a singular folded state is reflected in its free energy landscape, which is relatively flat and simple and is sensitive to different environmental changes that can modify the landscape in several different ways, lowering some energy minima while raising some energy barriers. This explains the conformational plasticity of an IDP/IDR, its extreme sensitivity to changes in the environment, its ability to interact with multiple different partners, and consequently to fold in different ways. This is then directly related to the remarkable multifunctionality of disordered proteins that are able to control, regulate, interact with, as well as be controlled and regulated by, a plethora of structurally unrelated partners (Uversky V N et al (2019) Front Phys 7(Article 10), doi:10.3389/fphy.2019.00010).

Despite their lack of a stable structure, IDPs/IDRs are involved in a multitude of crucial biological functions related to regulation, recognition, signaling, and control, where binding to multiple partners and high-specificity/low-affinity interactions plays a crucial role. Furthermore, intrinsic disorder is a unique structural feature that enables IDPs/IDRs to participate in both one-to-many and many-to-one signaling. Since they serve as general regulators of various cellular processes, IDPs/IDRs themselves are tightly controlled, however, when misexpressed, misprocessed, mismodified, or dysregulated, IDPs/IDRs are prone to engage in promiscuous, often unwanted interactions and, thus, are associated with the development of various pathological states. In fact, many human cancer-related proteins, as well as many proteins associated with neurodegeneration, diabetes, cardiovascular disease, amyloidosis, and genetic diseases, are either intrinsically disordered or contain long IDRs (Iakoucheva L M et al J Mol. Biol (2002) 323:573-584; Uversky VN Front Biosci (2014) 19:181-258; Uversky VN Front Biosci (2009) 14:5188-5238; Du Z et al Int J Mol Sci (2017) 18:10; Cheng Y et al Biochemistry (2006) 45:10448-10460; Uversky VN Curr Alzheimer Res (2008) 5:260-287; Midic U et al BMC Genomics (2009)10:1,S12). Intrinsically disordered proteins and their roles and relevance in chronic diseases is reviewed in Kulkarni P and Uversky V N (Kulkarni P and Uversky V N (2019) Biomolecules 9,147, doi:10.3390/biom9040147).

As described in the examples, phase separation sensors were designed, produced and evaluated wherein the target component protein is a filaggrin family protein or paralog protein. In certain sensors, the artificial client protein sequence was derived from or based on a filaggrin protein sequence. Artificial client protein sequences were derived from or based on human filaggrin protein sequence and on a mouse filaggrin protein sequence. Exemplary artificial client proteins based from filaggrin sequence or designed to target or associate with filaggrin protein-containing biomolecular condensates include those provided in any of SEQ ID NOs: 17-21. The sensors designed based on human filaggrin sequence were effective and active in targeting and associating with filaggrin protein in biomolecular condensates in human cells and in vivo in mice. Alternative artificial client protein sequences derived from or based on a filaggrin protein repeat component sequence, including from any of the filaggrin homologs and paralogs is contemplated and provided in the invention. Reference is made to TABLE 1 and the sequences provided and referred to therein, including mouse and human filaggrin sequences, which provides alternative FLG and paralog sequences known and provided in the art.

The invention includes compositions of the phase separation sensors provided herein. The compositions include pharmaceutical compositions, optionally further comprising one or more vehicle, carrier or diluent. The present invention further contemplates therapeutic compositions or pharmaceutical compositions useful in practicing the methods of this invention, particularly in vivo or ex vivo and in mammals or humans. A subject therapeutic composition or pharmaceutical composition includes, in admixture, a pharmaceutically acceptable excipient (such as a carrier) and one or more phase separation sensor as described herein as an active ingredient.

The preparation of therapeutic compositions or pharmaceutical compositions which contain polypeptides, analogs or active fragments as active ingredients is well understood in the art. Such compositions may be prepared as injectables, either as liquid solutions or suspensions, however, solid forms suitable for solution in, or suspension in, liquid prior to injection can also be prepared. The preparation can also be emulsified. The active ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

One or more phase separation sensor can be formulated into the therapeutic composition or pharmaceutical composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic or pharmaceutical phase separation sensor-containing compositions may be administered intravenously or intramuscularly in one embodiment, as by injection of a unit dose, for example. In another embodiment, they may be injected subcutaneously. In another embodiment, they may be administered topically through a disrupted skin barrier. Any suitable form of recognized administration may be utilized. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

Another feature of this invention is the expression of the nucleic acids, including DNA sequences, encoding one or more phase separation sensor disclosed herein. As is well known in the art, nucleic acid sequences or DNA sequences may be expressed by operatively linking them to an expression control sequence in an appropriate expression vector and employing that expression vector to transform an appropriate unicellular host.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids; phage DNAs, e.g., the numerous derivatives of phage lambda, and other phage DNA, e.g., M13 and filamentous single stranded phage DNA; yeast plasmids such as the 2 plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Any of a wide variety of expression control sequences—sequences that control the expression of a DNA sequence operatively linked to it—may be used in these vectors to express the DNA sequences of this invention. A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, insect cells, and human cells and plant cells in tissue culture.

In selecting an expression control sequence, a variety of factors will normally be considered. These include, for example, the relative strength of the system, its controllability, and its compatibility with the particular nucleic acid or DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the nucleic acid or DNA sequences to be expressed, and the ease of purification of the expression products. Considering these and other factors a person skilled in the art will be able to construct a variety of vector/expression control sequence/host combinations that will express the DNA sequences of this invention on fermentation or in large scale animal culture. The nucleic acids or DNA encoding the phase separation sensors hereof may be administered via one or more vector or DNA construct which is capable of expressing the sensor(s) in a target cell or tissue.

In additional embodiments, methods are provided herein based on the characteristics and capabilities of the phase separation sensors. Thus, methods are provided comprising administering, transfecting or transducing, or otherwise contacting a cell, tissue, sample etc with a phase separation sensor wherein the sensor is capable of targeting or associating with a biomolecular condensate and comprises at least two protein domains, wherein the first domain comprises one or more accessory protein and the second domain comprises an artificial client protein having intrinsic disorder and capable of engaging in ultra-weak phase separation-specific amino acid interactions with one or more component protein in the condensate.

In one such embodiment, a method is provided for targeting a biomolecular condensate in a cell or tissue comprising administering to the cell or tissue or otherwise expressing in the cell or tissue one or more phase separation sensor of the invention.

In an embodiment, a method is provided for targeting a biomolecular condensate in a cell comprising transfecting or transducing the cell with a vector comprising nucleic acid encoding a sensor of the invention or otherwise capable of expressing the sensor of the invention in a cell.

Biomolecular condensates refer to and include membraneless compartments in cells and are two- and three-dimensional compartments in eukaryotic cells that concentrate specific collections of molecules without an encapsulating lipid-based membrane. Biomolecular condensates may be cytoplasmic or nuclear in cell location. Biomolecular condensates include keratohyalin granule (KG), P granule, Germ granule, Lewy bodies, synaptic condensates, stress granule, P bodies, T cell signalosome, crystalline condensates of the lens fibers, Nucleoli, Paraspeckles, Histone Locus Bodies, Cajal Bodies, Heterochromatin and other cytoplasmic or nuclear condensates or membraneless organelles assembled through liquid-liquid phase separation.

In another embodiment, a method is provided for detecting or visualizing a biomolecular condensate in a cell or tissue comprising administering to the cell or tissue or otherwise expressing in the cell or tissue one or more sensor of the invention as provided herein. In one such method embodiment, the sensor comprises at least one accessory protein comprising a detectable or functional label or marker, or a protein capable of tagging the condensate with a detectable or functional label or marker, including for example by association with or localization in the condensate. In a method embodiment, the sensor comprises at least one accessory protein selected from a fluorescent protein, a protein that creates contrast suitable for electron microscopy, or a protein capable of tagging the condensate with a detectable or functional label or marker.

Another method embodiment of the invention is provided in a method for monitoring one or more biomolecular condensate(s) in a cell comprising administering to the cell or otherwise expressing in the cell or tissue one or more sensor described and provided herein wherein the sensor is capable of tagging or labeling the condensate, such as with a detectable or functional label or marker. In an embodiment, the sensor is capable of tagging or labeling a protein in the condensate via a chemical interaction or enzymatic reaction. In an embodiment, the sensor is capable of tagging or labeling a protein in the condensate via ultra-weak bonding or by association with or localization in the condensate. In an embodiment, the sensor is capable of tagging or revealing the condensate with a detectable or functional label or marker without altering the condensate or any condensate protein.

A further method embodiment provides a method for manipulating one or more biomolecular condensate(s) in a cell comprising administering to the cell or otherwise expressing in the cell or tissue one or more sensor described and provided herein wherein the sensor is capable of modifying, labeling, or altering a protein in the condensate. Thus, for example, when a cargo protein modifies protein(s) within condensates, the material properties of the condensate can be manipulated, including being altered or tuned. In one such method embodiment, covalent or noncovalent cross-linking of condensate components may alter the material properties of the condensate.

A kit for evaluation of one or more biomolecular condensate(s) in cells or tissues is provided in another distinct embodiment of the invention, wherein the kit comprises a phase separation sensor as described and provided herein, a nucleic acid encoding a sensor hereof, or a vector comprising a nucleic acid or otherwise capable of expressing one or more sensor hereof in a cell.

In alternative methods of the invention, the phase separation sensors provided herein may be utilized in monitoring phase separation dynamics. The sensors can monitor the formation of condensates and their disassembly, including in a cell, tissue or organ, including as demonstrated herein in skin.

Further methods embodiments include use and application of one or more phase separation sensor to evaluate or screen compounds, drugs or agents for their effect on a condensate. This is particularly relevant wherein the formation of a condensate, the size, the material properties or location of a condensate, or the component make up is altered in or associated with a disease or condition, or is involved in a cellular response in an animal, particularly in a human. In one such embodiment, the sensors are utilized in screening for drugs that promote assembly or disassembly of target condensates.

In accordance with the above, an assay system for screening potential drugs effective to modulate the activity of the target biomolecular condensate may be prepared. The phase specific sensor may be introduced into a test system, and the prospective drug may also be introduced into the resulting cell culture, and the culture thereafter examined to observe any changes in the biomolecular condensate in the cells or of an activity or function associated with one or more embodiment of the biomolecular condensate, due either to the addition of the prospective drug alone, or due to the effect of added quantities of the known phase separation sensor.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

### Liquid-Liquid Phase Separation Drives Skin Barrier Formation

At the body surface, the skin's stratified squamous epithelium is uniquely challenged by environmental extremes. Through processes poorly understood, enucleated surface squames derive from transcriptionally-active keratinocytes displaying filaggrin-containing keratohyalin granules (KGs) of unknown structure/function. Here we show that filaggrin assembles KGs through liquid-liquid phase separation, whose dynamics govern terminal differentiation and are disrupted by human skin barrier disease-associated mutations that compromise its critical concentration for phase separation. By engineering sensitive, innocuous fluorescent probes to interrogate endogenous phase behavior in mice, we discovered that phase transitions during epidermal differentiation crowd cellular spaces with KGs whose coalescence is restricted by keratin bundles. Strikingly, natural environmental gradients then profoundly alter KG phase dynamics to drive squame formation. Our findings expose skin as a tissue driven by phase separation. Phase separation sensors reveal abundant liquid-like organelles that are at the crux of skin barrier formation.

Liquid-liquid phase separation of biopolymers has emerged as a major driving force for assembling membraneless biomolecular condensates (1-3), including nucleoli (4), receptor signaling complexes (3, 5), germline granules (1, 6) and stress granules (7). This focus on phase separation has also unraveled unexpected insights into a range of biological processes, including genomic organization (8-10), RNA processing (11, 12), mitosis (13, 14), cell-adhesion (15) and carbon dioxide fixation in plants (16). Despite remarkable progress, the study of cellular phase separation remains challenging (17, 18), often relying upon truncated protein mutants, reconstituted systems in non-physiological buffers and overexpression/knockin of tagged fusions (17, 19) that can alter a protein's phase separation behavior.

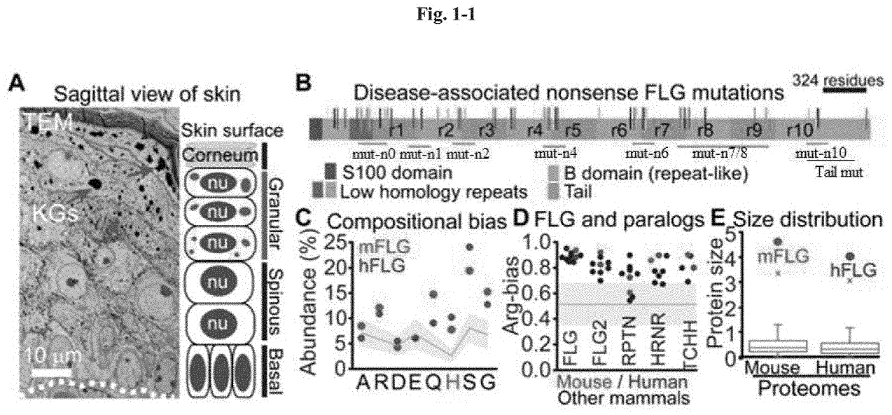

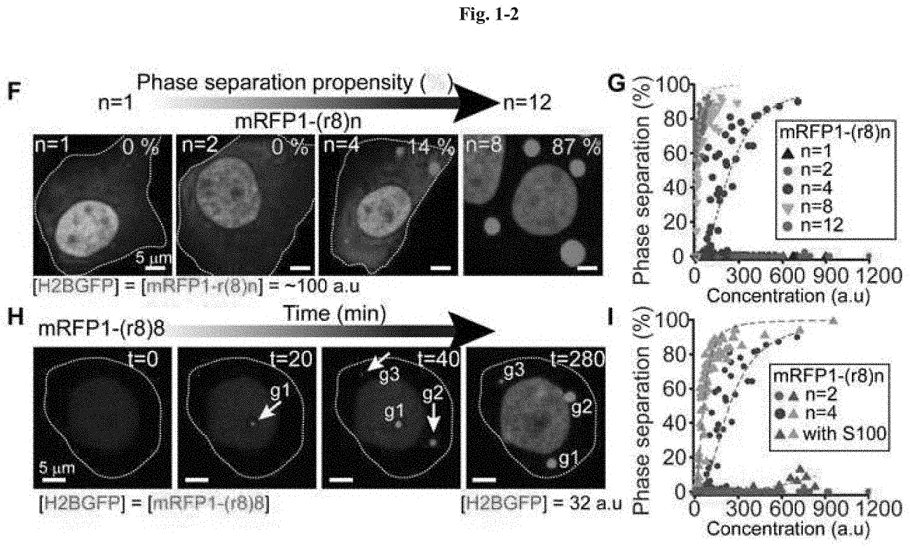

In mammalian epidermis, a self-renewing inner (basal) layer of progenitors fuels an upward flux of non-dividing keratinocytes that stratify to form the skin's surface barrier that excludes pathogens and retains body fluids (FIG. 1A) (20). In early spinous layers, terminally differentiating cells acquire an abundant network of KI and K10-containing keratin filament bundles. As keratinocytes enter the granular layers, they acquire membraneless protein deposits (‘keratohyalin granules’, KGs) of enigmatic function (21). Inexplicably, global transcription suddenly ceases and both KGs and organelles are lost, giving rise to layers of enucleated squames that seal the skin as a tight barrier to the environment.

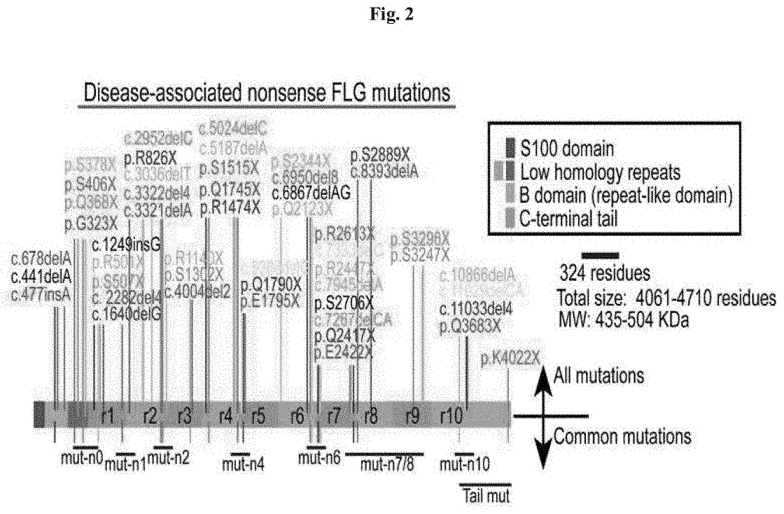

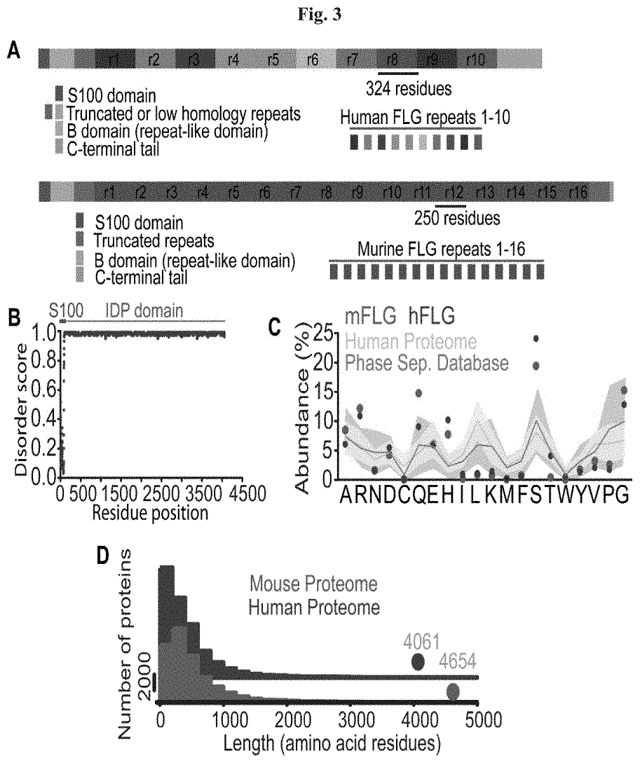

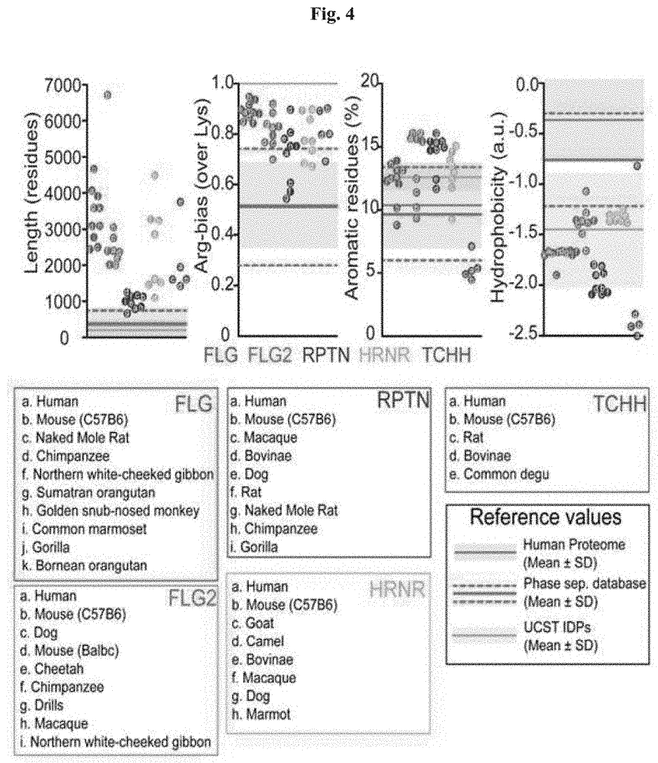

Previously, in an unbiased proteome-wide in silico search for phase transition proteins, we identified a major constituent of KGs, filaggrin (FLG) (22), whose truncating mutations are intriguingly linked to human skin barrier disorders (23) (FIG. 1B and FIG. 2). Here, we pursue the possibility that liquid-liquid phase separation might lie at the root of both epidermal differentiation and human disease. We show that filaggrin-containing KGs are liquid-like condensates and that disease-associated FLG mutations specifically perturb or abolish their critical concentration for phase separation-driven assembly. By developing innocuous and sensitive phase separation sensors that enable visualization and interrogation of endogenous liquid-liquid phase separation processes in normal skin, we discover that filaggrin's phase separation dynamics strikingly crowd and structure the cytoplasm with KGs whose liquid-like coalescence is restricted by surrounding keratin bundles. Probing deeper, we show that in addition to their physical impact on organelle integrity, liquid-like KGs sense a naturally-occurring pH shift that occurs in the final stages of terminal differentiation, just upstream of enucleation. This shift triggers dissolution of KG components to drive surface squame formation. Our findings yield unprecedented insights into the skin's barrier and contribute importantly to unearthing the physiological relevance of cellular mechanisms driven by phase separation.

Phase Separation Behavior of Filaggrin and its Paralogs in Normal and Disease States.

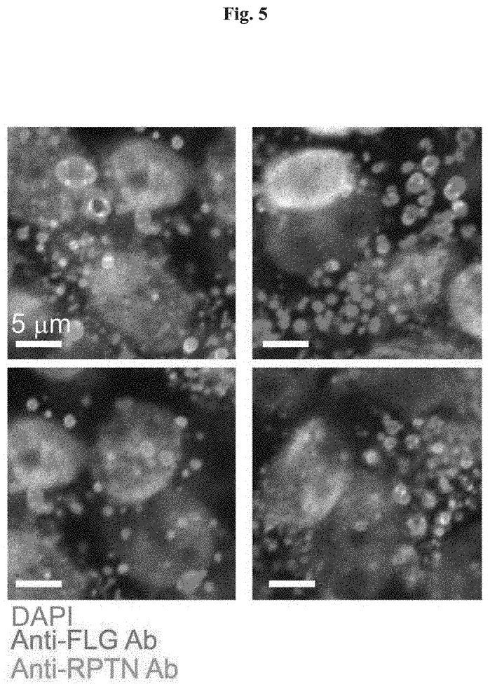

Filaggrin and its less-studied (often less-abundant) paralogs are intrinsically disordered repeat proteins with a low complexity (LC) sequence. Though their sequences are poorly conserved (24) (25, 26), mouse and human filaggrin and their paralogs share similar repeat architecture, LC biases and localization in the cell within KG-like structures (FIG. 1B-C; FIGS. 3-5 and TABLE 1).

TABLE 1
Sequence information for FLG and paralogs in all mammalian species considered in FIG. 1E and FIG. S1D.

| Protein | Species | Accession number* | Curation notes |
|---|---|---|---|
| FLG | Mouse (C57B6) | | Curated from genomic DNA and |
| | | | mRNA sequences (available |
| | | | sequence, XP_017175331.1 is low |
| | | | quality). The curated mouse Pig |
| | | | sequence (SEQ ID NO: 1) is provided |
| | | | below. |
| FLG | Human | NP_002007.1 | |
| FLG | Naked mole rat | EHB05610.1 | |
| FLG | Chimpanzee | H2R8N0 | Updated in February 2018 |
| FLG | Northern white- | XM_012511601.1 | Curated to include all of Exon3 and |
| | cheeked gibbon | | Exon2 (predicted form includes up to |
| | | | a partial B domain) |
| FLG | Sumatran orangutan | XM_003780333.2 | |
| FLG | Golden snub-nosed | XM_010352972.1 | |
| | monkey | | |
| FLG | Common marmoset | XP_008982649.1 | |
| FLG | Gorilla | BAT51076.1 | Sequence reported by Romero et al. |
| | | | lacked part of the B domain and the |
| | | | S-100 domain. |
| FLG | Bornean orangutan | BAT51075.1 | Sequence reported by Romero et al. |
| | | | lacked part of the B domain and the |
| | | | S-100 domain. |
| FLG2 | Human | NP_001014364.1 | (Based on GRCh38.p2) |
| FLG2 | Dog | XM_014120745.1 | Entry updated to XP_013976221.1 |
| FLG2 | Mouse (Balbc) | NP_001013826.1 | |
| FLG2 | Mouse (C57B6) | | Different from NP_001013826.1. |
| | | | Sequence directly curated from high |
| | | | quality gDNA available from |
| | | | GRCm38.p4 (current transcript |
| | | | annotation appears to be wrong as |
| | | | they attempted to map mRNA for the |
| | | | Balbc variant, which is much shorter) |
| FLG2 | Cheetah | XP_014943408.1 | |
| FLG2 | Chimpanzee | XP_016782784.1 | |
| FLG2 | Drill | XP_011829043.1 | |
| FLG2 | Crab-eating macaque | XP_015312597.1 | |
| FLG2 | Northern white- | XP_012366375.1 | |
| | cheeked gibbon | | |
| RPTN | Mouse (C57B6) | NP_033126.2 | |
| RPTN | Human | NP_001116437.1 | |
| RPTN | Rhesus macaque | XP_015006569.1 | |
| RPTN | Cow | XP_010801377.1 | |
| RPTN | Dog | XP_003432327.1 | Updated to XP_022260085.1 |
| RPTN | Norway rat | XP_227371.1 | |
| RPTN | Naked mole rat | XP_004854195.1 | Updated to XP_004854195.2 |
| RPTN | Chimpanzee | XP_016782772.1 | Updated to XP_016782772.2 |
| RPTN | Western lowland | XP_004026730.1 | |
| | gorilla | | |
| HRNR | Human | NP_001009931.1 | |
| HRNR | Mouse (C57B6) | NP_598459.2 | |
| HRNR | Goat | XP_013817836.1 | |
| HRNR | Bactrian camel | XP_010956837.1 | |
| HRNR | Cow | XP_010801374.1 | |
| HRNR | Rhesus monkey | XP_015007852.1 | |
| HRNR | Dog | XP_005630887.1 | |
| HRNR | Alpine marmot | XP_015358148.1 | |
| TCHH | Human | NP_009044.2 | |
| TCHH | Mouse (C57B6) | NP_001156570.1 | |
| TCHH | Norway rat | XP_006232882.1 | |
| TCHH | Cow | XP_002686080.3 | |
| TCHH | Degu | XP_012370438.1 | |

*NCBI, GenBank or Uniprot accession numbers.

Mouse Flg1 sequence (SEQ ID NO:1)—Translated from mm10 mouse Flg assembled and repaired CDS

MSALLESITSMIEIFQQYSTSDKEEETLSKEELKELLEGQLQAVLK
NPDDQDIAEVFMQMLDVDHDDKLDFAEYLLLVLKLAKAYYEASKN
ESFQTHGSNGRSKTDYKGLEEEGEEGNKQNLRRRHGGTDGKRKSD
RTRSPNGKRGKRQESRCRSEGKDKHRREPEKHRHQQDSKRKQRHG
SGSTERKDNRNKKNRQSKERNYDEIYDNGKYNEDWEASYNNCYYK
TQNTTLDQREGNRRPRADSQKEPQSSHGQADNSDSEGGRQQSHSK
PSPVRADQRRSRAGQAGSSKVSARSGSGGRGQSPDGSGRSSNRRD
RPRQPSPSQSSDSQVHSGVQVEGRRGQSSSANRRAGSSSGSGVQG
ASAGGLAADASRRSGARQGQASAQGRAGSQGQAQGRVGSSADRQG
RRGVSESQASDSEGHSDFSEGQAVGAHRQSGAGQRHEQRSSRGQH
GSRYYYEQEHSEEESDSQHQHGHQHEQQRGHQHQHEHEQPESGHR
QQQSSGRGHQGAHQEQGRDSARSRGSNQGHSSSRHQADSPRVSAR
SGSGGRGQSPDASGRSSNRRDRPRQPSPSQSSDSQVHSGVQVEGR
RGQSSSANRRAGSSSGSGVQGASAGGLAADASRRSGALQGQASAQ
GRAGSQGQAQGRVGSSADRQGRRGVSESQASDSEGHSDFSEGQAV
GAHRQSGAGQRHEQRSSRGQHGSGYYYEQEHSEEESDSQHQHGHQ
HEQQRGHQHQHQHQHEHEQPESGHRQQQSSGRGHQGAHQEQGRDS
ARSRGSNQGHSSSRHQADSPRVSARSGSGGRGQSPDASGRSSNRR
DRPRQPSPSQSSDSQVHSGVQVEGRRGQSSSANRRAGSSSGSGVQ
GTSAGGLAADASRRSGARQGQASAQGRAGSQGQAQGRVGSSADRQ
GRRGVSESQASDSEGHSDFSEGQAVGAHRQSGAGQRHEQRSSRGQ
HGSGYYYEQEHSEEESDSQHQHGHQHEQQRGHQHQHQHQHEHEQP
ESGHRQQQSSGRGNQGAHQKQGRDSARSRGSNQGHSSSRHQADSP
RVSARSGSGGRGQSPDASGRNSTKRDRPRQPSPSQSSDSHVHSGA
PDQGPRGTPSSVNRRAGSISGSGVQGASAGGLAADASRRSGARQG
QASAQGRAGSQGQAQGRVGSSADRQGRRGVSESQASDSEGHSDFS
EGQAVGAHRQSGAGQRHEQRSSRGQHGSRYYYEQEHSEEESDSQH
QHGHQHEQQRGHQHQHEHEQPESGHRQQQSSGRGHQGAHQEQGRD
SARSRGSNQGHSSSRHQADSPRVSARSGSGGRGQSPDASGRSSNR
RDRPRQPSPSQSSDSQVHSGVQVEGRRGQSSSANRRAGSSSGSGV
QGASAGGLAADASRRSGARQGQASAQGRAGSQGQAQGRIGSSADR
QGRRGVSESQASDSEGHSDFSEGQAVGAHRQSEAGQRHEQRSSRG
QHGSGYYYEQEHSEEESDSQHQHGHQHEQQRGTQHQHEHQQPESG
HRQQQSSGRGHQGTHQEQGRDSARSRGSNQGHSSSRHQADSPRVS
ARSGSGGRGQSPDASGRSSNRRDRPRQPSPSQSSDSQVHSGVQVE
GRRRQSSSANRRAGSSSGSGVQGASAGGLAADASRRSGARQGQAS
AQGRAGSQGQAQGRVGSSADRQGRRGVSESQASDSEGHSDFSEGQ
AVGAHRQSGAGQRHEQRSSRGQHGSGFYPVYYYYEQEHSEEESDS
QHQHGHQHEQQRGHQHQHQHEHEQPESGHRQQQSSGRGHQGAHQE
QGRDSARSRGSNQGHSSSRHQADSPRVSARSGSGGRGQSPDASGR
SSNRRDRPRQPSPSQSSDSQVHSGVQVEGRRGQSSSANRRAGSSS
GSGVQGASAGGLAADASRRSGALQGQASAQGRAGSQGQAQGRVGS
SADRQGRRGVSGSQASDSEGHSDFSEGQAVGAHRQSGAGQRHEQR
SSRGQHGSGYYYEQEHSEEESDSQHQHGHQHEQQRGHQHQHQHQH
EHEQPESGHRQQQFSGRGHQGAHQEQGRDSARSRGSNQGHSSSRH
QADSPRVSARSGSGGRGQSPDASGRSSNRRDRPRQPSPSQSSDSQ
VHSGVQVEGRRGQSSSANRRAGSSSGSGVQGASAGGLAADASRRS
GARQGQASAQGRAGSQGQAQGRVGSSADRQGRRGVSESQASDSEG
HSDFSEGQAVGAHRQSGAGQRHEQRSSRGQHGSGFYPVYYYYEQE
HSEEESDSQHQHGHQHEQQRGHQHQHQHQHEHEQPESGHRQQQSS
GRGHQGAHQEQGRDSARSRGSNQGHSSSRHQADSPRVSARSGSGG
RGQSPDASGRSSNRRDRPRQPSPSQSSDSQVHSGVQVEGRRGQSS
SANRRAGSSSGSGVQGASAGGLAADASRRSGARQGQASAQGRAGS
QGQAQGRVGSSADRQGRRGVSESQASDSEGHSDFSEGQAVGAHRQ
SGAGQRHEQRSSRGQHGSGYYYEQEHSEEESDSQHQHSHQHEQQR
GHQHQHQHQHEHEQPESGHRQQQFSGRGHQGAHQEQGRDSARSRG
SNQGHSSSRHQADSPRVSARSGSGGRGQSPDASGRSSNRRDRPRQ
PSPSQSSDSQVHSGVQVEGRRGQSSSANRRAGSSSGSGVQGASAG
GLAADASRRSGARQGQASAQGRAGSQGQAQGRVGSSADRQGRRGV
SESQASDSEGHSDFSEGQAVGAHRQSGAGQRHEQRSSRGQHGSGF
YPVYYYYEQEHSEEESDSQHQHGHQHEQQRGHQHQHQHQHEHEQP
ESGHRQQQSSGRGHQGAHQEQGRDSARSRGSNQGHSSSRHQADSP
RVSARSGSGGRGQSPDASGRSSNRRDRPRQPSPSQSSDSQVHSGV
QVEGRRGQSSSANRRAGSSSGSGVQGASAGGLAADASRRSGARQG
QASAQGRAGSQGQAQGRVGSSADRQGRRGVSESQASDSEGHSDFS
EGQAVGAHRQSGAGQRHEQRSSRSQHGSGYYYEQEHSEEESDSQH
QHSHQHEQQRGHQHQHQHQHEHEQPESGHRQQQSSGRGNQGAHQE
QGRDSARSRGSNQGHSSSRHQADSPRVSARSGSGGRGQSPDASGR
SSNRRDRPRQPSPSQSSDSHVHSGVQVEGRRGQSSSANRRAGSSS
GSGVQGASAGGLAADASRRSGARQGQASAQGRAGSQGQAQGRVGS
SADRQGRRGVSESQASDSEGHSDFSEGQAVGAHRQSGAGQRHEQR
SSRGQHGSGFYPVYYYYEQEHSEEESDSQHQHGHQHEQQRGHQHQ
HQHQHEHEQPESGHRQQQSSGRGHQGAHQEQGRDSARSRGSNQGH
SSSRHQADSPRVSARSGSGGRGQSPDASGRSSNRRDRSRQPSPSQ
SSDSQVHSGVQVEGRRGQSSSANRRAGSSSGSGVQGASAGGLAAD
ASRRSGARQGQASAQGRAGSQGQAQGRVGSSADRQGRRGVSESQA
SDSEGHSDFSEGQAVGAHRQSGAGQRHEQRSSRGQHGSGFYPVYY
YYEQEHSEEESDSQHQHGHQHEQQRGHQHQHQHEHEQPESGHRQQ
QSSGRGHQGAHQEQGRDSARSRGSNQGHSSSRHQADSPRVSARSG
SGGRGQSPDASGRSSNRRDRPRQPSPSQSSDSQVHSGVQVEGRRG
QSSSANRRAGSSSGSGVQGASAGGLAADASRRSGALQGQASAQGR
AGSQGQAQGRVGSSADRQGRRGVSESQASDSEGHSDFSEGQAVGA
HRQSGAGQRHEQRSSRGQHGSGYYYEQEHSEEESDSQHQHGHQHE
QQRGHQHQHQHQHEHEQPESGHRQQQSSGRGHQGAHQEQGRDSAR
SRGSNQGHSSSRHQADSPRVSARSGSGGRGQSPDASGRSSNRRDR
PRQPSPSQSSDSQVHSGVQVEGRRGQSSSANRRAGSSSGSGVQGA
SAGGLAADASRRSGARQGQASAQGRAGSQGQAQGRVGSSADRQGR
RGVSGSQASDSEGHSDFSEGQAVGAHRQSGAGQRHEQRSSRGQHG
SGYYYEQEHSEEESDSQHQHGHQHEQQRGHQHQHQHQHEHEQPES
GHRQQQFSGRGHQGAHQEQGRDSARSRGSNQGHSSSRHQADSPRV
SARSGSGGRGQSPDASGRSSNRRDRPRQPSPSQSSDSQVHSGVQV
EGRRGQSSSANRRAGSSSSSGVQGASAGGLAADASRRSGARQGQA
SAQGRAGSQGQAQGRVGSSADRQGRRGVSESQASDSEGHSDFSEG
QAVGAHRQSGAGQRHEQRSSRGQHGSGFYPVYYYYEQEHSEEESD
SQHQHGHQHEQQRGHQHQHQHQHEHEQPESGHRQQQFSGRGHQGA
HQEQGRDSARSRGSNQGHSSSRHQADSPRVSARSGSGGRGQSPDA
SGRSSNRRDRPRQPSPSQSSDSQVHSGVQVEGRRGQSSSANRRAG
SSSGSGVQGASAGGLAADASRRSGARQGQASAQGRAGSQGQAQGR
VGSSADRQGRRGVSESQASDSEGHSDFSEGQAVGAHRQSGAGQRH
EQRSSRGQHGSGYYYEQEHSEEESDSQHQQGHQHEQQRGHQHQHQ
HQHEHEQPESGHRQQQSSGRGHQGAHQEQGRDSARSRGSNQGHSS
SRHQADSPRVSARSGSGGRGQSPDGSGRSSNRRDRPRQPSASQSS
DSQVHSGVQVEAQRGQSSSANRRAGSSSGSGVQSAAASGQGGYES
IFTAKHLDFNQSHSYYYY

The human filaggrin sequence (NP_002007.1) is provided below for reference (SEQ ID NO:56):

| | |
|---|---|
| 1 | mstllenifa iinlfkqysk kdkntdtlsk |
| | kelkelleke frqilknpdd pdmvdvfmdh |
| 61 | Ididhnkkid ftefllmvfk laqayyestr |
| | kenlpisghk hrkhshhdkh ednkqeenke |
| 121 | nrkrpssler rnnrkgnkgr skspretggk |
| | rhesssekke rkgyspthre eeygknhhns |
| 181 | skkeknkten trlgdnrkrl serleekedn |
| | eegvydyent grmtqkwiqs ghiatyytiq |
| 241 | deaydttdsl leenkiyers rssdgksssq |
| | vnrsrhents qvplqesrtr krrgsrvsqd |
| 301 | rdseghseds erhsgsasrn hhgsaweqsr |
| | dgsrhprshd edrashghsa dssrqsgtrh |
| 361 | aetssrgqta ssheqarssp gerhgsghqq |
| | sadssrhsat grgqassavs drghrgssgs |
| 421 | qasdseghse nsdtqsvsgh gkaglrqqsh |
| | qestrgrsge rsgrsgssly qvstheqpds |
| 481 | ahgrtgtstg grqgshheqa rdssrhsasq |
| | egqdtirghp gssrggrqgs hheqsvnrsg |
| 541 | hsgshhshtt sqgrsdashg qsgsrsasrq |
| | trneeqsgdg trhsgsrhhe assqadssrh |
| 601 | sqvgqgqssg prtsrnqgss vsqdsdsqgh |
| | sedserwsgs asrnhhgsaq eqsrdgsrhp |
| 661 | rshhedragh ghsadssrks gtrhtqnsss |
| | gqaassheqa rssagerhgs rhqlqsadss |
| 721 | rhsgtghgqa ssavrdsghr gssgsqatds |
| | eghsedsdtq svsghgqagh hqqshqesar |
| 781 | drsgersrrs gsflyqvsth kqsesshgwt |
| | gpstgvrqgs hheqardnsr hsasqdgqdt |
| 841 | irghpgssrr grqgshheqs vdrsghsgsh |
| | hshttsqgrs dasrgqsgsr sasrttrnee |
| 901 | qsrdgsrhsg srhheassha disrhsqagq |
| | gqsegsrtsr rqgssvsqds dseghsedse |
| 961 | rwsgsasrnh rgsaqeqsrh gsrhprshhe |
| | draghghsad ssrqsgtpha etssggqaas |
| 1021 | sheqarsspg erhgsrhqqs adssrhsgip |
| | rrqassavrd sghwgssgsq asdseghsee |
| 1081 | sdtqsvsghg qdgphqqshq esardwsggr |
| | sgrsgsfiyq vstheqsesa hgrtrtstgr |
| 1141 | rqgshheqar dssrhsasqe gqdtirahpg |
| | srrggrqgsh heqsvdrsgh sgshhshtts |
| 1201 | qgrsdashgq sgsrsasrqt rkdkqsgdgs |
| | rhsgsrhhea aswadssrhs qvgqeqssgs |
| 1261 | rtsrhqgssv sqdsdserhs ddserlsgsa |
| | srnhhgssre qsrdgsrhpg fhqedrashg |
| 1321 | hsadssrqsg thhtessshg qavssheqar |
| | sspgerhgsr hqqsadssrh sgighrqass |
| 1381 | avrdsghrgs sgsqvtnseg hsedsdtqsv |
| | sahgqagphq qshkesargq sgessgrsrs |
| 1441 | flyqvssheq sesthgqtap stggrqgsrh |
| | eqarnssrhs asqdgqdtir ghpgssrggr |
| 1501 | qgsyheqsvd rsghsgyhhs httpqgrsda |
| | shgqsgprsa srqtrneeqs gdgsrhsgsr |
| 1561 | hhepstrags srhsqvgqge sagsktsrrq |
| | gssvsqdrds eghsedserr sesasrnhyg |
| 1621 | sareqsrhgs rnprshqedr ashghsaess |
| | rqsgtrhaet ssggqaassq eqarsspger |
| 1681 | hgsrhqqsad sstdsgtgrr qdssvvgdsg |
| | nrgssgsqas dseghseesd tqsvsahgqa |
| 1741 | gphqqshqes trgqsgersg rsgsflyqvs |
| | theqsesahg rtgpstggrq rsrheqards |
| 1801 | srhsasqegq dtirghpgss rggrqgshye |
| | qsvdssghsg shhshttsqe rsdvsrgqsg |
| 1861 | srsvsrqtrn ekqsgdgsrh sgsrhheass |
| | radssrhsqv gqgqssgprt srnqgssvsq |
| 1921 | dsdsqghsed serwsgsasr nhlgsaweqs |
| | rdgsrhpgsh hedraghghs adssrqsgtr |
| 1981 | htesssrgqa assheqarss agerhgshhq |
| | iqsadssrhs gighgqassa vrdsghrgys |
| 2041 | gsqasdsegh sedsdtqsvs aqgkagphqq |
| | shkesargqs gessgrsgsf lyqvstheqs |
| 2101 | esthgqsaps tggrqgshyd qaqdssrhsa |
| | sqegqdtirg hpgpsrggrq gshqeqsvdr |
| 2161 | sghsgshhsh ttsqgrsdas rgqsgsrsas |
| | rktydkeqsg dgsrhsgshh heasswadss |
| 2221 | rhslvgqgqs sgprtsrprg ssvsqdsdse |
| | ghsedserrs gsasrnhhgs aqeqsrdgsr |
| 2281 | hprshhedra ghghsaessr qsgthhaens |
| | sggqaasshe qarssagerh gshhqqsads |
| 2341 | srhsgighgq assavrdsgh rgssgsqasd |
| | seghsedsdt qsvsahgqag phqqshqest |
| 2401 | rgrsagrsgr sgsflyqvst heqsesahgr |
| | tgtstggrqg shhkqardss rhstsqegqd |
| 2461 | tihghpgsss ggrqgshyeq Ivdrsghsgs |
| | hhshttsqgr sdashghsgs rsasrqtrnd |
| 2521 | eqsgdgsrhs gsrhheassr adssghsqvg |
| | qgqsegprts rnwgssfsqd sdsqghseds |
| 2581 | erwsgsasrn hhgsaqeqlr dgsrhprshq |
| | edraghghsa dssrqsgtrh tqtssggqaa |
| 2641 | ssheqarssa gerhgshhqq sadssrhsgi |
| | ghgqassavr dsghrgysgs qasdneghse |
| 2701 | dsdtqsvsah gqagshqqsh qesargrsge |
| | tsghsgsfly qvstheqses shgwtgpstr |
| 2761 | grqgsrheqa qdssrhsasq dgqdtirghp |
| | gssrggrqgy hhehsvdssg hsgshhshtt |
| 2821 | sqgrsdasrg qsgsrsasrt trneeqsgdg |
| | srhsgsrhhe asthadisrh sqavqgqseg |
| 2881 | srrsrrqgss vsqdsdsegh sedserwsgs |
| | asrnhhgsaq eqlrdgsrhp rshqedragh |
| 2941 | ghsadssrqs gtrhtqtssg gqaassheqa |
| | rssagerhgs hhqqsadssr hsgighgqas |
| 3001 | savrdsghrg ysgsqasdne ghsedsdtqs |
| | vsahgqagsh qqshqesarg rsgetsghsg |
| 3061 | sflyqvsthe qsesshgwtg pstrgrqgsr |
| | heqaqdssrh sasqygqdti rghpgssrgg |
| 3121 | rqgyhhehsv dssghsgshh shttsqgrsd |
| | asrgqsgsrs asrttrneeq sgdssrhsvs |
| 3181 | rhheasthad isrhsqavqg qsegsrrsrr |
| | qgssvsqdsd seghsedser wsgsasrnhr |
| 3241 | gsvqeqsrhg srhprshhed raghghsadr |
| | srqsgtrhae tssggqaass heqarsspge |
| 3301 | rhgsrhqqsa dssrhsgipr gqassavrds |
| | rhwgssgsqa sdseghsees dtqsvsghgq |
| 3361 | agphqqshqe sardrsggrs grsgsflyqv |
| | stheqsesah grtrtstgrr qgshheqard |
| 3421 | ssrhsasqeg qdtirghpgs srrgrqgshy |
| | eqsvdrsghs gshhshttsq grsdasrgqs |
| 3481 | gsrsasrqtr ndeqsgdgsr hswshhheas |
| | tqadssrhsq sgqgqsagpr tsrnqgssvs |
| 3541 | qdsdsqghse dserwsgsas rnhrgsaqeq |
| | srdgsrhpts hhedraghgh saessrqsgt |
| 3601 | hhaenssggq aassheqars sagerhgshh |
| | qqsadssrhs gighgqassa vrdsghrgss |
| 3661 | gsqasdsegh sedsdtqsvs ahgqagphqq |
| | shqestrgrs agrsgrsgsf lyqvstheqs |
| 3721 | esahgragps tggrqgsrhe qardssrhsa |
| | sqegqdtirg hpgsrrggrq gsyheqsvdr |
| 3781 | sghsgshhsh ttsqgrsdas hgqsgsrsas |
| | retrneeqsg dgsrhsgsrh heastqadss |
| 3841 | rhsqsgqges agsrrsrrqg ssvsqdsdse |
| | aypedserrs esasrnhhgs sreqsrdgsr |
| 3901 | hpgsshrdta shvqsspvqs dsstakehgh |
| | fsslsqdsay hsgiqsrgsp hssssyhyqs |
| 3961 | egterqkgqs glvwrhgsyg sadydygesg |
| | frhsqhgsvs ynsnpvvfke rsdickasaf |
| 4021 | gkdhpryyat yinkdpglcg hssdiskqlg |
| | fsqsqryyyy e |

Like many proteins that drive phase separation, filaggrin family proteins across species exhibit a striking bias for arginine (over similarly charged lysine) to engage in aromatic-type interactions (22) (FIG. 1D and FIG. 4). They differ in that their only prominent aromatic residue is histidine, rather than tyrosine or phenylalanine (FIG. 3). Previously, we showed that histidine-rich, intrinsically disordered proteins (IDPs) must be large to display phase separation behaviors (22). Notably, both human (˜435-504 kDa) and mouse filaggrin are among the largest proteins across these proteomes (FIG. 1E). Interestingly, humans whose filaggrin variants have the greatest repeat numbers exhibit reduced susceptibility to skin inflammation and allergy (23).

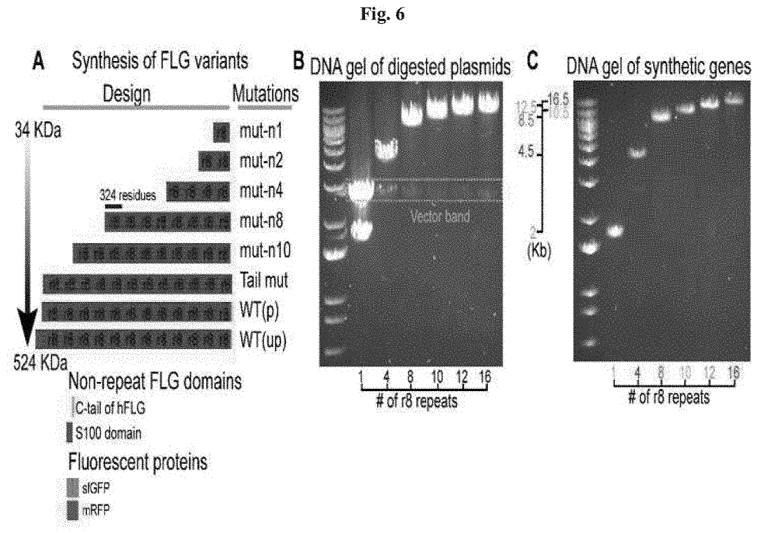

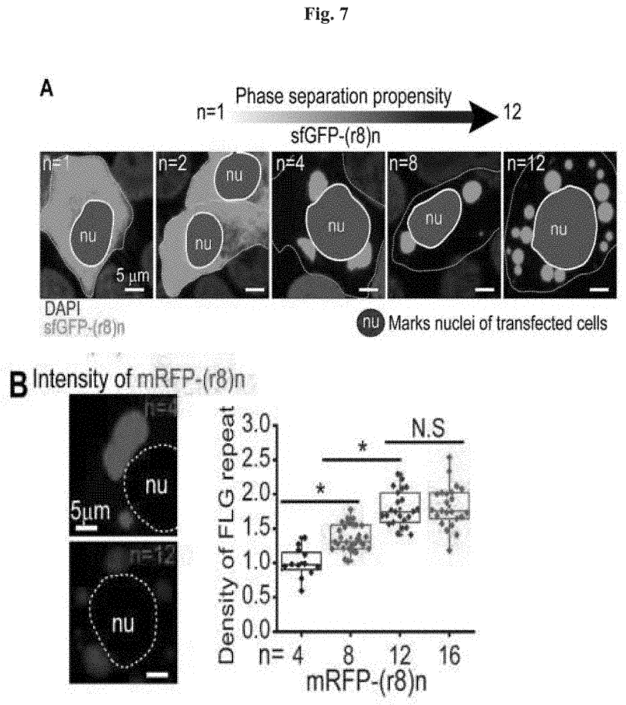

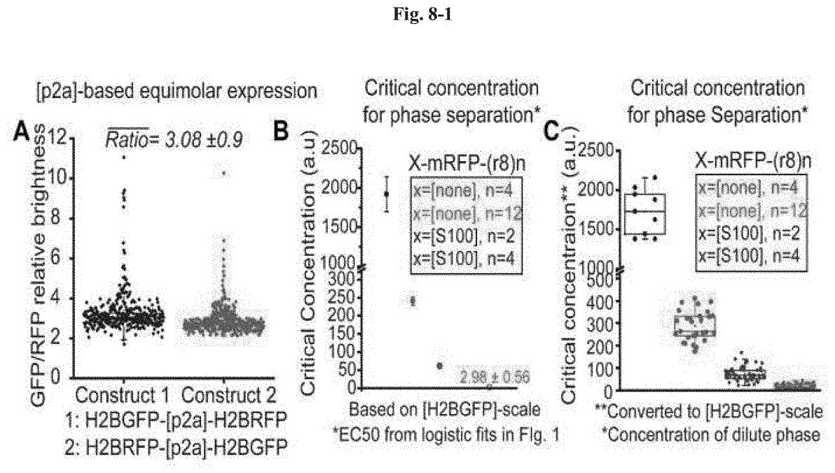

To directly interrogate filaggrin and its disease-associated variants for phase separation behavior, we first engineered expression vectors driving 1 to 16 human filaggrin repeats (humans have up to 12), each tagged with a fluorescent protein (sfGFP or mRFP)±the non-repeat domains (FIG. 6 and TABLE 2). When transfected into immortalized human keratinocytes (HaCATs) under conditions where filaggrin is not expressed, only diffuse cytoplasmic localization was observed with a single FLG repeat (FIG. 7A). By contrast, keratinocytes transfected with genes encoding variants of ≥4 repeats efficiently formed KG-like structures. Moreover, proportional to the total repeat numbers, a monotonic increase in density within KG-like granules plateaued beyond the largest known human filaggrins, suggesting that non-phenotypic filaggrins (10-12 repeats) optimally define the material properties of KGs (FIG. S6B).

TABLE 2
Sequence information for synthesized FLG
variants that appear herein. We do not
include the full sequence for the following
mRFP1-based filaggrin variants:
mRFP1-(r8)4, mRFP1- (r8)8, mRFP1-(r8)12
and mRFP1-(r8)8-Tail as their sequence
can be easily derived from the equivalent
sfGFP-based variants. We include two
mRFP1-based constructs as reference
(mRFP1-(r8)16 and S100-mRFP1-(r8)8-Tail).

| Construct | Sequence |
|---|---|
| sfGFP-(r8) | MGSKGEELFTGVVPILVELDGDVNGHKFSV |
| (SEQ ID | RGEGEGDATNGKLTLKFICTTGKLPVPWPT |
| NO: 2) | LVTTLGYGVQCFSRYPDHMKRHDFFKSAMP |
| | EGYVQERTISFKDDGTYKTRAEVKFEGDTL |
| | VNRIELKGIDFKEDGNILGHKLEYNFNSHN |
| | VYITADKQKNGIKANFKIRHNVEDGSVQLA |
| | DHYQQNTPIGDGPVLLPDNHYLSTQSVLSK |
| | DPNEKRDHMVLLEFVTAAGITHGMDELYKS |
| | PGGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYG |
| sfGFP-(r8)2 | MGSKGEELFTGVVPILVELDGDVNGHKFSV |
| (SEQ ID | RGEGEGDATNGKLTLKFICTTGKLPVPWPT |
| NO: 3) | LVTTLGYGVQCFSRYPDHMKRHDFFKSAMP |
| | EGYVQERTISFKDDGTYKTRAEVKFEGDTL |
| | VNRIELKGIDFKEDGNILGHKLEYNFNSHN |
| | VYITADKQKNGIKANFKIRHNVEDGSVQLA |
| | DHYQQNTPIGDGPVLLPDNHYLSTQSVLSK |
| | DPNEKRDHMVLLEFVTAAGITHGMDELYKS |
| | PGGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYG |
| sfGFP-(r8)4 | MGSKGEELFTGVVPILVELDGDVNGHKFSV |
| (SEQ ID | RGEGEGDATNGKLTLKFICTTGKLPVPWPT |
| NO: 4) | LVTTLGYGVQCFSRYPDHMKRHDFFKSAMP |
| | EGYVQERTISFKDDGTYKTRAEVKFEGDTL |
| | VNRIELKGIDFKEDGNILGHKLEYNFNSHN |
| | VYITADKQKNGIKANFKIRHNVEDGSVQLA |
| | DHYQQNTPIGDGPVLLPDNHYLSTQSVLSK |
| | DPNEKRDHMVLLEFVTAAGITHGMDELYKS |
| | PGGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYGQVSTHEQ |
| | SESSHGWTGPSTRGRQGSRHEQAQDSSRHS |
| | ASQDGQDTIRGHPGSSRGGRQGYHHEHSVD |
| | SSGHSGSHHSHTTSQGRSDASRGQSGSRSA |
| | SRTTRNEEQSGDGSRHSGSRHHEASTHADI |
| | SRHSQAVQGQSEGSRRSRRQGSSVSQDSDS |
| | EGHSEDSERWSGSASRNHHGSAQEQLRDGS |
| | RHPRSHQEDRAGHGHSADSSRQSGTRHTQT |
| | SSGGQAASSHEQARSSAGERHGSHHQQSAD |
| | SSRHSGIGHGQASSAVRDSGHRGYSGSQAS |
| | DNEGHSEDSDTQSVSAHGQAGSHQQSHQES |
| | ARGRSGETSGHSGSFLYGQVSTHEQSESSH |
| | GWTGPSTRGRQGSRHEQAQDSSRHSASQDG |
| | QDTIRGHPGSSRGGRQGYHHEHSVDSSGHS |
| | GSHHSHTTSQGRSDASRGQSGSRSASRTTR |
| | NEEQSGDGSRHSGSRHHEASTHADISRHSQ |
| | AVQGQSEGSRRSRRQGSSVSQDSDSEGHSE |
| | DSERWSGSASRNHHGSAQEQLRDGSRHPRS |
| | HQEDRAGHGHSADSSRQSGTRHTQTSSGGQ |
| | AASSHEQARSSAGERHGSHHQQSADSSRHS |
| | GIGHGQASSAVRDSGHRGYSGSQASDNEGH |
| | SEDSDTQSVSAHGQAGSHQQSHQESARGRS |
| | GETSGHSGSFLYG |
| sfGFP-(r8)8 | MGSKGEELFTGVVPILVELDGDVNGHKFSV |
| (SEQ ID | RGEGEGDATNGKLTLKFICTTGKLPVPWPT |
| NO: 5) | LVTTLGYGVQCFSRYPDHMKRHDFFKSAMP |
| | EGYVQERTISFKDDGTYKTRAEVKFEGDTL |
| | VNRIELKGIDFKEDGNILGHKLEYNFNSHN |
| | VYITADKQKNGIKANFKIRHNVEDGSVQLA |
| | DHYQQNTPIGDGPVLLPDNHYLSTQSVLSK |
| | DPNEKRDHMVLLEFVTAAGITHGMDELYKS |
| | PGGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYGQVSTHEQ |
| | SESSHGWTGPSTRGRQGSRHEQAQDSSRHS |
| | ASQDGQDTIRGHPGSSRGGRQGYHHEHSVD |
| | SSGHSGSHHSHTTSQGRSDASRGQSGSRSA |
| | SRTTRNEEQSGDGSRHSGSRHHEASTHADI |
| | SRHSQAVQGQSEGSRRSRRQGSSVSQDSDS |
| | EGHSEDSERWSGSASRNHHGSAQEQLRDGS |
| | RHPRSHQEDRAGHGHSADSSRQSGTRHTQT |
| | SSGGQAASSHEQARSSAGERHGSHHQQSAD |
| | SSRHSGIGHGQASSAVRDSGHRGYSGSQAS |
| | DNEGHSEDSDTQSVSAHGQAGSHQQSHQES |
| | ARGRSGETSGHSGSFLYGQVSTHEQSESSH |
| | GWTGPSTRGRQGSRHEQAQDSSRHSASQDG |
| | QDTIRGHPGSSRGGRQGYHHEHSVDSSGHS |
| | GSHHSHTTSQGRSDASRGQSGSRSASRTTR |
| | NEEQSGDGSRHSGSRHHEASTHADISRHSQ |
| | AVQGQSEGSRRSRRQGSSVSQDSDSEGHSE |
| | DSERWSGSASRNHHGSAQEQLRDGSRHPRS |
| | HQEDRAGHGHSADSSRQSGTRHTQTSSGGQ |
| | AASSHEQARSSAGERHGSHHQQSADSSRHS |
| | GIGHGQASSAVRDSGHRGYSGSQASDNEGH |
| | SEDSDTQSVSAHGQAGSHQQSHQESARGRS |
| | GETSGHSGSFLYGQVSTHEQSESSHGWTGP |
| | STRGRQGSRHEQAQDSSRHSASQDGQDTIR |
| | GHPGSSRGGRQGYHHEHSVDSSGHSGSHHS |
| | HTTSQGRSDASRGQSGSRSASRTTRNEEQS |
| | GDGSRHSGSRHHEASTHADISRHSQAVQGQ |
| | SEGSRRSRRQGSSVSQDSDSEGHSEDSERW |
| | SGSASRNHHGSAQEQLRDGSRHPRSHQEDR |
| | AGHGHSADSSRQSGTRHTQTSSGGQAASSH |
| | EQARSSAGERHGSHHQQSADSSRHSGIGHG |
| | QASSAVRDSGHRGYSGSQASDNEGHSEDSD |
| | TQSVSAHGQAGSHQQSHQESARGRSGETSG |
| | HSGSFLYGQVSTHEQSESSHGWTGPSTRGR |
| | QGSRHEQAQDSSRHSASQDGQDTIRGHPGS |
| | SRGGRQGYHHEHSVDSSGHSGSHHSHTTSQ |
| | GRSDASRGQSGSRSASRTTRNEEQSGDGSR |
| | HSGSRHHEASTHADISRHSQAVQGQSEGSR |
| | RSRRQGSSVSQDSDSEGHSEDSERWSGSAS |
| | RNHHGSAQEQLRDGSRHPRSHQEDRAGHGH |
| | SADSSRQSGTRHTQTSSGGQAASSHEQARS |
| | SAGERHGSHHQQSADSSRHSGIGHGQASSA |
| | VRDSGHRGYSGSQASDNEGHSEDSDTQSVS |
| | AHGQAGSHQQSHQESARGRSGETSGHSGSF |
| | LYGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYG |
| sfGFP-(r8)10 | MGSKGEELFTGVVPILVELDGDVNGHKFSV |
| (SEQ ID | RGEGEGDATNGKLTLKFICTTGKLPVPWPT |
| NO: 6) | LVTTLGYGVQCFSRYPDHMKRHDFFKSAMP |
| | EGYVQERTISFKDDGTYKTRAEVKFEGDTL |
| | VNRIELKGIDFKEDGNILGHKLEYNFNSHN |
| | VYITADKQKNGIKANFKIRHNVEDGSVQLA |
| | DHYQQNTPIGDGPVLLPDNHYLSTQSVLSK |
| | DPNEKRDHMVLLEFVTAAGITHGMDELYKS |
| | PGGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYGQVSTHEQ |
| | SESSHGWTGPSTRGRQGSRHEQAQDSSRHS |
| | ASQDGQDTIRGHPGSSRGGRQGYHHEHSVD |
| | SSGHSGSHHSHTTSQGRSDASRGQSGSRSA |
| | SRTTRNEEQSGDGSRHSGSRHHEASTHADI |
| | SRHSQAVQGQSEGSRRSRRQGSSVSQDSDS |
| | EGHSEDSERWSGSASRNHHGSAQEQLRDGS |
| | RHPRSHQEDRAGHGHSADSSRQSGTRHTQT |
| | SSGGQAASSHEQARSSAGERHGSHHQQSAD |
| | SSRHSGIGHGQASSAVRDSGHRGYSGSQAS |
| | DNEGHSEDSDTQSVSAHGQAGSHQQSHQES |
| | ARGRSGETSGHSGSFLYGQVSTHEQSESSH |
| | GWTGPSTRGRQGSRHEQAQDSSRHSASQDG |
| | QDTIRGHPGSSRGGRQGYHHEHSVDSSGHS |
| | GSHHSHTTSQGRSDASRGQSGSRSASRTTR |
| | NEEQSGDGSRHSGSRHHEASTHADISRHSQ |
| | AVQGQSEGSRRSRRQGSSVSQDSDSEGHSE |
| | DSERWSGSASRNHHGSAQEQLRDGSRHPRS |
| | HQEDRAGHGHSADSSRQSGTRHTQTSSGGQ |
| | AASSHEQARSSAGERHGSHHQQSADSSRHS |
| | GIGHGQASSAVRDSGHRGYSGSQASDNEGH |
| | SEDSDTQSVSAHGQAGSHQQSHQESARGRS |
| | GETSGHSGSFLYGQVSTHEQSESSHGWTGP |
| | STRGRQGSRHEQAQDSSRHSASQDGQDTIR |
| | GHPGSSRGGRQGYHHEHSVDSSGHSGSHHS |
| | HTTSQGRSDASRGQSGSRSASRTTRNEEQS |
| | GDGSRHSGSRHHEASTHADISRHSQAVQGQ |
| | SEGSRRSRRQGSSVSQDSDSEGHSEDSERW |
| | SGSASRNHHGSAQEQLRDGSRHPRSHQEDR |
| | AGHGHSADSSRQSGTRHTQTSSGGQAASSH |
| | EQARSSAGERHGSHHQQSADSSRHSGIGHG |
| | QASSAVRDSGHRGYSGSQASDNEGHSEDSD |
| | TQSVSAHGQAGSHQQSHQESARGRSGETSG |
| | HSGSFLYGQVSTHEQSESSHGWTGPSTRGR |
| | QGSRHEQAQDSSRHSASQDGQDTIRGHPGS |
| | SRGGRQGYHHEHSVDSSGHSGSHHSHTTSQ |
| | GRSDASRGQSGSRSASRTTRNEEQSGDGSR |
| | HSGSRHHEASTHADISRHSQAVQGQSEGSR |
| | RSRRQGSSVSQDSDSEGHSEDSERWSGSAS |
| | RNHHGSAQEQLRDGSRHPRSHQEDRAGHGH |
| | SADSSRQSGTRHTQTSSGGQAASSHEQARS |
| | SAGERHGSHHQQSADSSRHSGIGHGQASSA |
| | VRDSGHRGYSGSQASDNEGHSEDSDTQSVS |
| | AHGQAGSHQQSHQESARGRSGETSGHSGSF |
| | LYGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHT |
| | QTSSGGQAASSHEQARSSAGERHGSHHQQS |
| | ADSSRHSGIGHGQASSAVRDSGHRGYSGSQ |
| | ASDNEGHSEDSDTQSVSAHGQAGSHQQSHQ |
| | ESARGRSGETSGHSGSFLYGQVSTHEQSES |
| | SHGWTGPSTRGRQGSRHEQAQDSSRHSASQ |
| | DGQDTIRGHPGSSRGGRQGYHHEHSVDSSG |
| | HSGSHHSHTTSQGRSDASRGQSGSRSASRT |
| | TRNEEQSGDGSRHSGSRHHEASTHADISRH |
| | SQAVQGQSEGSRRSRRQGSSVSQDSDSEGH |
| | SEDSERWSGSASRNHHGSAQEQLRDGSRHP |
| | RSHQEDRAGHGHSADSSRQSGTRHTQTSSG |
| | GQAASSHEQARSSAGERHGSHHQQSADSSR |
| | HSGIGHGQASSAVRDSGHRGYSGSQASDNE |
| | GHSEDSDTQSVSAHGQAGSHQQSHQESARG |
| | RSGETSGHSGSFLYGQVSTHEQSESSHGWT |
| | GPSTRGRQGSRHEQAQDSSRHSASQDGQDT |
| | IRGHPGSSRGGRQGYHHEHSVDSSGHSGSH |
| | HSHTTSQGRSDASRGQSGSRSASRTTRNEE |
| | QSGDGSRHSGSRHHEASTHADISRHSQAVQ |
| | GQSEGSRRSRRQGSSVSQDSDSEGHSEDSE |
| | RWSGSASRNHHGSAQEQLRDGSRHPRSHQE |
| | DRAGHGHSADSSRQSGTRHTQTSSGGQAAS |
| | SHEQARSSAGERHGSHHQQSADSSRHSGIG |
| | HGQASSAVRDSGHRGYSGSQASDNEGHSED |
| | SDTQSVSAHGQAGSHQQSHQESARGRSGET |
| | SGHSGSFLYG |
| sfGFP-(r8)12 | MGSKGEELFTGVVPILVELDGDVNGHKFSV |
| (SEQ ID | RGEGEGDATNGKLTLKFICTTGKLPVPWPT |
| NO: 7) | LVTTLGYGVQCFSRYPDHMKRHDFFKSAMP |
| | EGYVQERTISFKDDGTYKTRAEVKFEGDTL |
| | VNRIELKGIDFKEDGNILGHKLEYNFNSHN |
| | VYITADKQKNGIKANFKIRHNVEDGSVQLA |
| | DHYQQNTPIGDGPVLLPDNHYLSTQSVLSK |
| | DPNEKRDHMVLLEFVTAAGITHGMDELYKS |
| | PGGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYGQVSTHEQ |
| | SESSHGWTGPSTRGRQGSRHEQAQDSSRHS |
| | ASQDGQDTIRGHPGSSRGGRQGYHHEHSVD |
| | SSGHSGSHHSHTTSQGRSDASRGQSGSRSA |
| | SRTTRNEEQSGDGSRHSGSRHHEASTHADI |
| | SRHSQAVQGQSEGSRRSRRQGSSVSQDSDS |
| | EGHSEDSERWSGSASRNHHGSAQEQLRDGS |
| | RHPRSHQEDRAGHGHSADSSRQSGTRHTQT |
| | SSGGQAASSHEQARSSAGERHGSHHQQSAD |
| | SSRHSGIGHGQASSAVRDSGHRGYSGSQAS |
| | DNEGHSEDSDTQSVSAHGQAGSHQQSHQES |
| | ARGRSGETSGHSGSFLYGQVSTHEQSESSH |
| | GWTGPSTRGRQGSRHEQAQDSSRHSASQDG |
| | QDTIRGHPGSSRGGRQGYHHEHSVDSSGHS |
| | GSHHSHTTSQGRSDASRGQSGSRSASRTTR |
| | NEEQSGDGSRHSGSRHHEASTHADISRHSQ |
| | AVQGQSEGSRRSRRQGSSVSQDSDSEGHSE |
| | DSERWSGSASRNHHGSAQEQLRDGSRHPRS |
| | HQEDRAGHGHSADSSRQSGTRHTQTSSGGQ |
| | AASSHEQARSSAGERHGSHHQQSADSSRHS |
| | GIGHGQASSAVRDSGHRGYSGSQASDNEGH |
| | SEDSDTQSVSAHGQAGSHQQSHQESARGRS |
| | GETSGHSGSFLYGQVSTHEQSESSHGWTGP |
| | STRGRQGSRHEQAQDSSRHSASQDGQDTIR |
| | GHPGSSRGGRQGYHHEHSVDSSGHSGSHHS |
| | HTTSQGRSDASRGQSGSRSASRTTRNEEQS |
| | GDGSRHSGSRHHEASTHADISRHSQAVQGQ |
| | SEGSRRSRRQGSSVSQDSDSEGHSEDSERW |
| | SGSASRNHHGSAQEQLRDGSRHPRSHQEDR |
| | AGHGHSADSSRQSGTRHTQTSSGGQAASSH |
| | EQARSSAGERHGSHHQQSADSSRHSGIGHG |
| | QASSAVRDSGHRGYSGSQASDNEGHSEDSD |
| | TQSVSAHGQAGSHQQSHQESARGRSGETSG |
| | HSGSFLYGQVSTHEQSESSHGWTGPSTRGR |
| | QGSRHEQAQDSSRHSASQDGQDTIRGHPGS |
| | SRGGRQGYHHEHSVDSSGHSGSHHSHTTSQ |
| | GRSDASRGQSGSRSASRTTRNEEQSGDGSR |
| | HSGSRHHEASTHADISRHSQAVQGQSEGSR |
| | RSRRQGSSVSQDSDSEGHSEDSERWSGSAS |
| | RNHHGSAQEQLRDGSRHPRSHQEDRAGHGH |
| | SADSSRQSGTRHTQTSSGGQAASSHEQARS |
| | SAGERHGSHHQQSADSSRHSGIGHGQASSA |
| | VRDSGHRGYSGSQASDNEGHSEDSDTQSVS |
| | AHGQAGSHQQSHQESARGRSGETSGHSGSF |
| | LYGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHT |
| | QTSSGGQAASSHEQARSSAGERHGSHHQQS |
| | ADSSRHSGIGHGQASSAVRDSGHRGYSGSQ |
| | ASDNEGHSEDSDTQSVSAHGQAGSHQQSHQ |
| | ESARGRSGETSGHSGSFLYGQVSTHEQSES |
| | SHGWTGPSTRGRQGSRHEQAQDSSRHSASQ |
| | DGQDTIRGHPGSSRGGRQGYHHEHSVDSSG |
| | HSGSHHSHTTSQGRSDASRGQSGSRSASRT |
| | TRNEEQSGDGSRHSGSRHHEASTHADISRH |
| | SQAVQGQSEGSRRSRRQGSSVSQDSDSEGH |
| | SEDSERWSGSASRNHHGSAQEQLRDGSRHP |
| | RSHQEDRAGHGHSADSSRQSGTRHTQTSSG |
| | GQAASSHEQARSSAGERHGSHHQQSADSSR |
| | HSGIGHGQASSAVRDSGHRGYSGSQASDNE |
| | GHSEDSDTQSVSAHGQAGSHQQSHQESARG |
| | RSGETSGHSGSFLYGQVSTHEQSESSHGWT |
| | GPSTRGRQGSRHEQAQDSSRHSASQDGQDT |
| | IRGHPGSSRGGRQGYHHEHSVDSSGHSGSH |
| | HSHTTSQGRSDASRGQSGSRSASRTTRNEE |
| | QSGDGSRHSGSRHHEASTHADISRHSQAVQ |
| | GQSEGSRRSRRQGSSVSQDSDSEGHSEDSE |
| | RWSGSASRNHHGSAQEQLRDGSRHPRSHQE |
| | DRAGHGHSADSSRQSGTRHTQTSSGGQAAS |
| | SHEQARSSAGERHGSHHQQSADSSRHSGIG |
| | HGQASSAVRDSGHRGYSGSQASDNEGHSED |
| | SDTQSVSAHGQAGSHQQSHQESARGRSGET |
| | SGHSGSFLYGQVSTHEQSESSHGWTGPSTR |
| | GRQGSRHEQAQDSSRHSASQDGQDTIRGHP |
| | GSSRGGRQGYHHEHSVDSSGHSGSHHSHTT |
| | SQGRSDASRGQSGSRSASRTTRNEEQSGDG |
| | SRHSGSRHHEASTHADISRHSQAVQGQSEG |
| | SRRSRRQGSSVSQDSDSEGHSEDSERWSGS |
| | ASRNHHGSAQEQLRDGSRHPRSHQEDRAGH |
| | GHSADSSRQSGTRHTQTSSGGQAASSHEQA |
| | RSSAGERHGSHHQQSADSSRHSGIGHGQAS |
| | SAVRDSGHRGYSGSQASDNEGHSEDSDTQS |
| | VSAHGQAGSHQQSHQESARGRSGETSGHSG |
| | SFLYGQVSTHEQSESSHGWTGPSTRGRQGS |
| | RHEQAQDSSRHSASQDGQDTIRGHPGSSRG |
| | GRQGYHHEHSVDSSGHSGSHHSHTTSQGRS |
| | DASRGQSGSRSASRTTRNEEQSGDGSRHSG |
| | SRHHEASTHADISRHSQAVQGQSEGSRRSR |
| | RQGSSVSQDSDSEGHSEDSERWSGSASRNH |
| | HGSAQEQLRDGSRHPRSHQEDRAGHGHSAD |
| | SSRQSGTRHTQTSSGGQAASSHEQARSSAG |
| | ERHGSHHQQSADSSRHSGIGHGQASSAVRD |
| | SGHRGYSGSQASDNEGHSEDSDTQSVSAHG |
| | QAGSHQQSHQESARGRSGETSGHSGSFLYG |
| sfGFP-(r8)8- | MGSKGEELFTGVVPILVELDGDVNGHKFSV |
| Tail | RGEGEGDATNGKLTLKFICTTGKLPVPWPT |
| (SEQ ID | LVTTLGYGVQCFSRYPDHMKRHDFFKSAMP |
| NO: 8) | EGYVQERTISFKDDGTYKTRAEVKFEGDTL |
| | VNRIELKGIDFKEDGNILGHKLEYNFNSHN |
| | VYITADKQKNGIKANFKIRHNVEDGSVQLA |
| | DHYQQNTPIGDGPVLLPDNHYLSTQSVLSK |
| | DPNEKRDHMVLLEFVTAAGITHGMDELYKS |
| | PGGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYGQVSTHEQ |
| | SESSHGWTGPSTRGRQGSRHEQAQDSSRHS |
| | ASQDGQDTIRGHPGSSRGGRQGYHHEHSVD |
| | SSGHSGSHHSHTTSQGRSDASRGQSGSRSA |
| | SRTTRNEEQSGDGSRHSGSRHHEASTHADI |
| | SRHSQAVQGQSEGSRRSRRQGSSVSQDSDS |
| | EGHSEDSERWSGSASRNHHGSAQEQLRDGS |
| | RHPRSHQEDRAGHGHSADSSRQSGTRHTQT |
| | SSGGQAASSHEQARSSAGERHGSHHQQSAD |
| | SSRHSGIGHGQASSAVRDSGHRGYSGSQAS |
| | DNEGHSEDSDTQSVSAHGQAGSHQQSHQES |
| | ARGRSGETSGHSGSFLYGQVSTHEQSESSH |
| | GWTGPSTRGRQGSRHEQAQDSSRHSASQDG |
| | QDTIRGHPGSSRGGRQGYHHEHSVDSSGHS |
| | GSHHSHTTSQGRSDASRGQSGSRSASRTTR |
| | NEEQSGDGSRHSGSRHHEASTHADISRHSQ |
| | AVQGQSEGSRRSRRQGSSVSQDSDSEGHSE |
| | DSERWSGSASRNHHGSAQEQLRDGSRHPRS |
| | HQEDRAGHGHSADSSRQSGTRHTQTSSGGQ |
| | AASSHEQARSSAGERHGSHHQQSADSSRHS |
| | GIGHGQASSAVRDSGHRGYSGSQASDNEGH |
| | SEDSDTQSVSAHGQAGSHQQSHQESARGRS |
| | GETSGHSGSFLYGQVSTHEQSESSHGWTGP |
| | STRGRQGSRHEQAQDSSRHSASQDGQDTIR |
| | GHPGSSRGGRQGYHHEHSVDSSGHSGSHHS |
| | HTTSQGRSDASRGQSGSRSASRTTRNEEQS |
| | GDGSRHSGSRHHEASTHADISRHSQAVQGQ |
| | SEGSRRSRRQGSSVSQDSDSEGHSEDSERW |
| | SGSASRNHHGSAQEQLRDGSRHPRSHQEDR |
| | AGHGHSADSSRQSGTRHTQTSSGGQAASSH |
| | EQARSSAGERHGSHHQQSADSSRHSGIGHG |
| | QASSAVRDSGHRGYSGSQASDNEGHSEDSD |
| | TQSVSAHGQAGSHQQSHQESARGRSGETSG |
| | HSGSFLYGQVSTHEQSESSHGWTGPSTRGR |
| | QGSRHEQAQDSSRHSASQDGQDTIRGHPGS |
| | SRGGRQGYHHEHSVDSSGHSGSHHSHTTSQ |
| | GRSDASRGQSGSRSASRTTRNEEQSGDGSR |
| | HSGSRHHEASTHADISRHSQAVQGQSEGSR |
| | RSRRQGSSVSQDSDSEGHSEDSERWSGSAS |
| | RNHHGSAQEQLRDGSRHPRSHQEDRAGHGH |
| | SADSSRQSGTRHTQTSSGGQAASSHEQARS |
| | SAGERHGSHHQQSADSSRHSGIGHGQASSA |
| | VRDSGHRGYSGSQASDNEGHSEDSDTQSVS |
| | AHGQAGSHQQSHQESARGRSGETSGHSGSF |
| | LYGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYGPGLCGHS |
| | SDISKQLGFSQSQRYYYYEG |
| sfGFP- | MGSKGEELFTGVVPILVELDGDVNGHKFSV |
| (r8)10-Tail | RGEGEGDATNGKLTLKFICTTGKLPVPWPT |
| (SEQ ID | LVTTLGYGVQCFSRYPDHMKRHDFFKSAMP |
| NO: 9) | EGYVQERTISFKDDGTYKTRAEVKFEGDTL |
| | VNRIELKGIDFKEDGNILGHKLEYNFNSHN |
| | VYITADKQKNGIKANFKIRHNVEDGSVQLA |
| | DHYQQNTPIGDGPVLLPDNHYLSTQSVLSK |
| | DPNEKRDHMVLLEFVTAAGITHGMDELYKS |
| | PGGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYGQVSTHEQ |
| | SESSHGWTGPSTRGRQGSRHEQAQDSSRHS |
| | ASQDGQDTIRGHPGSSRGGRQGYHHEHSVD |
| | SSGHSGSHHSHTTSQGRSDASRGQSGSRSA |
| | SRTTRNEEQSGDGSRHSGSRHHEASTHADI |
| | SRHSQAVQGQSEGSRRSRRQGSSVSQDSDS |
| | EGHSEDSERWSGSASRNHHGSAQEQLRDGS |
| | RHPRSHQEDRAGHGHSADSSRQSGTRHTQT |
| | SSGGQAASSHEQARSSAGERHGSHHQQSAD |
| | SSRHSGIGHGQASSAVRDSGHRGYSGSQAS |
| | DNEGHSEDSDTQSVSAHGQAGSHQQSHQES |
| | ARGRSGETSGHSGSFLYGQVSTHEQSESSH |
| | GWTGPSTRGRQGSRHEQAQDSSRHSASQDG |
| | QDTIRGHPGSSRGGRQGYHHEHSVDSSGHS |
| | GSHHSHTTSQGRSDASRGQSGSRSASRTTR |
| | NEEQSGDGSRHSGSRHHEASTHADISRHSQ |
| | AVQGQSEGSRRSRRQGSSVSQDSDSEGHSE |
| | DSERWSGSASRNHHGSAQEQLRDGSRHPRS |
| | HQEDRAGHGHSADSSRQSGTRHTQTSSGGQ |
| | AASSHEQARSSAGERHGSHHQQSADSSRHS |
| | GIGHGQASSAVRDSGHRGYSGSQASDNEGH |
| | SEDSDTQSVSAHGQAGSHQQSHQESARGRS |
| | GETSGHSGSFLYGQVSTHEQSESSHGWTGP |
| | STRGRQGSRHEQAQDSSRHSASQDGQDTIR |
| | GHPGSSRGGRQGYHHEHSVDSSGHSGSHHS |
| | HTTSQGRSDASRGQSGSRSASRTTRNEEQS |
| | GDGSRHSGSRHHEASTHADISRHSQAVQGQ |
| | SEGSRRSRRQGSSVSQDSDSEGHSEDSERW |
| | SGSASRNHHGSAQEQLRDGSRHPRSHQEDR |
| | AGHGHSADSSRQSGTRHTQTSSGGQAASSH |
| | EQARSSAGERHGSHHQQSADSSRHSGIGHG |
| | QASSAVRDSGHRGYSGSQASDNEGHSEDSD |
| | TQSVSAHGQAGSHQQSHQESARGRSGETSG |
| | HSGSFLYGQVSTHEQSESSHGWTGPSTRGR |
| | QGSRHEQAQDSSRHSASQDGQDTIRGHPGS |
| | SRGGRQGYHHEHSVDSSGHSGSHHSHTTSQ |
| | GRSDASRGQSGSRSASRTTRNEEQSGDGSR |
| | HSGSRHHEASTHADISRHSQAVQGQSEGSR |
| | RSRRQGSSVSQDSDSEGHSEDSERWSGSAS |
| | RNHHGSAQEQLRDGSRHPRSHQEDRAGHGH |
| | SADSSRQSGTRHTQTSSGGQAASSHEQARS |
| | SAGERHGSHHQQSADSSRHSGIGHGQASSA |
| | VRDSGHRGYSGSQASDNEGHSEDSDTQSVS |
| | AHGQAGSHQQSHQESARGRSGETSGHSGSF |
| | LYGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHT |
| | QTSSGGQAASSHEQARSSAGERHGSHHQQS |
| | ADSSRHSGIGHGQASSAVRDSGHRGYSGSQ |
| | ASDNEGHSEDSDTQSVSAHGQAGSHQQSHQ |
| | ESARGRSGETSGHSGSFLYGQVSTHEQSES |
| | SHGWTGPSTRGRQGSRHEQAQDSSRHSASQ |
| | DGQDTIRGHPGSSRGGRQGYHHEHSVDSSG |
| | HSGSHHSHTTSQGRSDASRGQSGSRSASRT |
| | TRNEEQSGDGSRHSGSRHHEASTHADISRH |
| | SQAVQGQSEGSRRSRRQGSSVSQDSDSEGH |
| | SEDSERWSGSASRNHHGSAQEQLRDGSRHP |
| | RSHQEDRAGHGHSADSSRQSGTRHTQTSSG |
| | GQAASSHEQARSSAGERHGSHHQQSADSSR |
| | HSGIGHGQASSAVRDSGHRGYSGSQASDNE |
| | GHSEDSDTQSVSAHGQAGSHQQSHQESARG |
| | RSGETSGHSGSFLYGQVSTHEQSESSHGWT |
| | GPSTRGRQGSRHEQAQDSSRHSASQDGQDT |
| | IRGHPGSSRGGRQGYHHEHSVDSSGHSGSH |
| | HSHTTSQGRSDASRGQSGSRSASRTTRNEE |
| | QSGDGSRHSGSRHHEASTHADISRHSQAVQ |
| | GQSEGSRRSRRQGSSVSQDSDSEGHSEDSE |
| | RWSGSASRNHHGSAQEQLRDGSRHPRSHQE |
| | DRAGHGHSADSSRQSGTRHTQTSSGGQAAS |
| | SHEQARSSAGERHGSHHQQSADSSRHSGIG |
| | HGQASSAVRDSGHRGYSGSQASDNEGHSED |
| | SDTQSVSAHGQAGSHQQSHQESARGRSGET |
| | SGHSGSFLYGPGLCGHSSDISKQLGFSQSQ |
| | RYYYYEG |
| sfGFP- | MGSKGEELFTGVVPILVELDGDVNGHKFSV |
| (r8)12-Tail | RGEGEGDATNGKLTLKFICTTGKLPVPWPT |
| (SEQ ID | LVTTLGYGVQCFSRYPDHMKRHDFFKSAMP |
| NO: 10) | EGYVQERTISFKDDGTYKTRAEVKFEGDTL |
| | VNRIELKGIDFKEDGNILGHKLEYNFNSHN |
| | VYITADKQKNGIKANFKIRHNVEDGSVQLA |
| | DHYQQNTPIGDGPVLLPDNHYLSTQSVLSK |
| | DPNEKRDHMVLLEFVTAAGITHGMDELYKS |
| | PGGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYGQVSTHEQ |
| | SESSHGWTGPSTRGRQGSRHEQAQDSSRHS |
| | ASQDGQDTIRGHPGSSRGGRQGYHHEHSVD |
| | SSGHSGSHHSHTTSQGRSDASRGQSGSRSA |
| | SRTTRNEEQSGDGSRHSGSRHHEASTHADI |
| | SRHSQAVQGQSEGSRRSRRQGSSVSQDSDS |
| | EGHSEDSERWSGSASRNHHGSAQEQLRDGS |
| | RHPRSHQEDRAGHGHSADSSRQSGTRHTQT |
| | SSGGQAASSHEQARSSAGERHGSHHQQSAD |
| | SSRHSGIGHGQASSAVRDSGHRGYSGSQAS |
| | DNEGHSEDSDTQSVSAHGQAGSHQQSHQES |
| | ARGRSGETSGHSGSFLYGQVSTHEQSESSH |
| | GWTGPSTRGRQGSRHEQAQDSSRHSASQDG |
| | QDTIRGHPGSSRGGRQGYHHEHSVDSSGHS |
| | GSHHSHTTSQGRSDASRGQSGSRSASRTTR |
| | NEEQSGDGSRHSGSRHHEASTHADISRHSQ |
| | AVQGQSEGSRRSRRQGSSVSQDSDSEGHSE |
| | DSERWSGSASRNHHGSAQEQLRDGSRHPRS |
| | HQEDRAGHGHSADSSRQSGTRHTQTSSGGQ |
| | AASSHEQARSSAGERHGSHHQQSADSSRHS |
| | GIGHGQASSAVRDSGHRGYSGSQASDNEGH |
| | SEDSDTQSVSAHGQAGSHQQSHQESARGRS |
| | GETSGHSGSFLYGQVSTHEQSESSHGWTGP |
| | STRGRQGSRHEQAQDSSRHSASQDGQDTIR |
| | GHPGSSRGGRQGYHHEHSVDSSGHSGSHHS |
| | HTTSQGRSDASRGQSGSRSASRTTRNEEQS |
| | GDGSRHSGSRHHEASTHADISRHSQAVQGQ |
| | SEGSRRSRRQGSSVSQDSDSEGHSEDSERW |
| | SGSASRNHHGSAQEQLRDGSRHPRSHQEDR |
| | AGHGHSADSSRQSGTRHTQTSSGGQAASSH |
| | EQARSSAGERHGSHHQQSADSSRHSGIGHG |
| | QASSAVRDSGHRGYSGSQASDNEGHSEDSD |
| | TQSVSAHGQAGSHQQSHQESARGRSGETSG |
| | HSGSFLYGQVSTHEQSESSHGWTGPSTRGR |
| | QGSRHEQAQDSSRHSASQDGQDTIRGHPGS |
| | SRGGRQGYHHEHSVDSSGHSGSHHSHTTSQ |
| | GRSDASRGQSGSRSASRTTRNEEQSGDGSR |
| | HSGSRHHEASTHADISRHSQAVQGQSEGSR |
| | RSRRQGSSVSQDSDSEGHSEDSERWSGSAS |
| | RNHHGSAQEQLRDGSRHPRSHQEDRAGHGH |
| | SADSSRQSGTRHTQTSSGGQAASSHEQARS |
| | SAGERHGSHHQQSADSSRHSGIGHGQASSA |
| | VRDSGHRGYSGSQASDNEGHSEDSDTQSVS |
| | AHGQAGSHQQSHQESARGRSGETSGHSGSF |
| | LYGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHT |
| | QTSSGGQAASSHEQARSSAGERHGSHHQQS |
| | ADSSRHSGIGHGQASSAVRDSGHRGYSGSQ |
| | ASDNEGHSEDSDTQSVSAHGQAGSHQQSHQ |
| | ESARGRSGETSGHSGSFLYGQVSTHEQSES |
| | SHGWTGPSTRGRQGSRHEQAQDSSRHSASQ |
| | DGQDTIRGHPGSSRGGRQGYHHEHSVDSSG |
| | HSGSHHSHTTSQGRSDASRGQSGSRSASRT |
| | TRNEEQSGDGSRHSGSRHHEASTHADISRH |
| | SQAVQGQSEGSRRSRRQGSSVSQDSDSEGH |
| | SEDSERWSGSASRNHHGSAQEQLRDGSRHP |
| | RSHQEDRAGHGHSADSSRQSGTRHTQTSSG |
| | GQAASSHEQARSSAGERHGSHHQQSADSSR |
| | HSGIGHGQASSAVRDSGHRGYSGSQASDNE |
| | GHSEDSDTQSVSAHGQAGSHQQSHQESARG |
| | RSGETSGHSGSFLYGQVSTHEQSESSHGWT |
| | GPSTRGRQGSRHEQAQDSSRHSASQDGQDT |
| | IRGHPGSSRGGRQGYHHEHSVDSSGHSGSH |
| | HSHTTSQGRSDASRGQSGSRSASRTTRNEE |
| | QSGDGSRHSGSRHHEASTHADISRHSQAVQ |
| | GQSEGSRRSRRQGSSVSQDSDSEGHSEDSE |
| | RWSGSASRNHHGSAQEQLRDGSRHPRSHQE |
| | DRAGHGHSADSSRQSGTRHTQTSSGGQAAS |
| | SHEQARSSAGERHGSHHQQSADSSRHSGIG |
| | HGQASSAVRDSGHRGYSGSQASDNEGHSED |
| | SDTQSVSAHGQAGSHQQSHQESARGRSGET |
| | SGHSGSFLYGQVSTHEQSESSHGWTGPSTR |
| | GRQGSRHEQAQDSSRHSASQDGQDTIRGHP |
| | GSSRGGRQGYHHEHSVDSSGHSGSHHSHTT |
| | SQGRSDASRGQSGSRSASRTTRNEEQSGDG |
| | SRHSGSRHHEASTHADISRHSQAVQGQSEG |
| | SRRSRRQGSSVSQDSDSEGHSEDSERWSGS |
| | ASRNHHGSAQEQLRDGSRHPRSHQEDRAGH |
| | GHSADSSRQSGTRHTQTSSGGQAASSHEQA |
| | RSSAGERHGSHHQQSADSSRHSGIGHGQAS |
| | SAVRDSGHRGYSGSQASDNEGHSEDSDTQS |
| | VSAHGQAGSHQQSHQESARGRSGETSGHSG |
| | SFLYGQVSTHEQSESSHGWTGPSTRGRQGS |
| | RHEQAQDSSRHSASQDGQDTIRGHPGSSRG |
| | GRQGYHHEHSVDSSGHSGSHHSHTTSQGRS |
| | DASRGQSGSRSASRTTRNEEQSGDGSRHSG |
| | SRHHEASTHADISRHSQAVQGQSEGSRRSR |
| | RQGSSVSQDSDSEGHSEDSERWSGSASRNH |
| | HGSAQEQLRDGSRHPRSHQEDRAGHGHSAD |
| | SSRQSGTRHTQTSSGGQAASSHEQARSSAG |
| | ERHGSHHQQSADSSRHSGIGHGQASSAVRD |
| | SGHRGYSGSQASDNEGHSEDSDTQSVSAHG |
| | QAGSHQQSHQESARGRSGETSGHSGSFLYG |
| | PGLCGHSSDISKQLGFSQSQRYYYYEG |
| S100-sfGFP- | MSTLLENIFAIINLFKQYSKKDKNTDTLSK |
| (r8)8-Tail | KELKELLEKEFRQILKNPDDPDMVDVFMDH |
| (SEQ ID | LDIDHNKKIDFTEFLLMVFKLAQAYYESTR |
| NO: 11) | KEGVPGSGVPGAGVPGSRSDGSKGEELFTG |
| | VVPILVELDGDVNGHKFSVRGEGEGDATNG |
| | KLTLKFICTTGKLPVPWPTLVTTLGYGVQC |
| | FSRYPDHMKRHDFFKSAMPEGYVQERTISF |
| | KDDGTYKTRAEVKFEGDTLVNRIELKGIDF |
| | KEDGNILGHKLEYNFNSHNVYITADKQKNG |
| | IKANFKIRHNVEDGSVQLADHYQQNTPIGD |
| | GPVLLPDNHYLSTQSVLSKDPNEKRDHMVL |
| | LEFVTAAGITHGMDELYKSPGGQVSTHEQS |
| | ESSHGWTGPSTRGRQGSRHEQAQDSSRHSA |
| | SQDGQDTIRGHPGSSRGGRQGYHHEHSVDS |
| | SGHSGSHHSHTTSQGRSDASRGQSGSRSAS |
| | RTTRNEEQSGDGSRHSGSRHHEASTHADIS |
| | RHSQAVQGQSEGSRRSRRQGSSVSQDSDSE |
| | GHSEDSERWSGSASRNHHGSAQEQLRDGSR |
| | HPRSHQEDRAGHGHSADSSRQSGTRHTQTS |
| | SGGQAASSHEQARSSAGERHGSHHQQSADS |
| | SRHSGIGHGQASSAVRDSGHRGYSGSQASD |
| | NEGHSEDSDTQSVSAHGQAGSHQQSHQESA |
| | RGRSGETSGHSGSFLYGQVSTHEQSESSHG |
| | WTGPSTRGRQGSRHEQAQDSSRHSASQDGQ |
| | DTIRGHPGSSRGGRQGYHHEHSVDSSGHSG |
| | SHHSHTTSQGRSDASRGQSGSRSASRTTRN |
| | EEQSGDGSRHSGSRHHEASTHADISRHSQA |
| | VQGQSEGSRRSRRQGSSVSQDSDSEGHSED |
| | SERWSGSASRNHHGSAQEQLRDGSRHPRSH |
| | QEDRAGHGHSADSSRQSGTRHTQTSSGGQA |
| | ASSHEQARSSAGERHGSHHQQSADSSRHSG |
| | IGHGQASSAVRDSGHRGYSGSQASDNEGHS |
| | EDSDTQSVSAHGQAGSHQQSHQESARGRSG |
| | ETSGHSGSFLYGQVSTHEQSESSHGWTGPS |
| | TRGRQGSRHEQAQDSSRHSASQDGQDTIRG |
| | HPGSSRGGRQGYHHEHSVDSSGHSGSHHSH |
| | TTSQGRSDASRGQSGSRSASRTTRNEEQSG |
| | DGSRHSGSRHHEASTHADISRHSQAVQGQS |
| | EGSRRSRRQGSSVSQDSDSEGHSEDSERWS |
| | GSASRNHHGSAQEQLRDGSRHPRSHQEDRA |
| | GHGHSADSSRQSGTRHTQTSSGGQAASSHE |
| | QARSSAGERHGSHHQQSADSSRHSGIGHGQ |
| | ASSAVRDSGHRGYSGSQASDNEGHSEDSDT |
| | QSVSAHGQAGSHQQSHQESARGRSGETSGH |
| | SGSFLYGQVSTHEQSESSHGWTGPSTRGRQ |
| | GSRHEQAQDSSRHSASQDGQDTIRGHPGSS |
| | RGGRQGYHHEHSVDSSGHSGSHHSHTTSQG |
| | RSDASRGQSGSRSASRTTRNEEQSGDGSRH |
| | SGSRHHEASTHADISRHSQAVQGQSEGSRR |
| | SRRQGSSVSQDSDSEGHSEDSERWSGSASR |
| | NHHGSAQEQLRDGSRHPRSHQEDRAGHGHS |
| | ADSSRQSGTRHTQTSSGGQAASSHEQARSS |
| | AGERHGSHHQQSADSSRHSGIGHGQASSAV |
| | RDSGHRGYSGSQASDNEGHSEDSDTQSVSA |
| | HGQAGSHQQSHQESARGRSGETSGHSGSFL |
| | YGQVSTHEQSESSHGWTGPSTRGRQGSRHE |
| | QAQDSSRHSASQDGQDTIRGHPGSSRGGRQ |
| | GYHHEHSVDSSGHSGSHHSHTTSQGRSDAS |
| | RGQSGSRSASRTTRNEEQSGDGSRHSGSRH |
| | HEASTHADISRHSQAVQGQSEGSRRSRRQG |
| | SSVSQDSDSEGHSEDSERWSGSASRNHHGS |
| | AQEQLRDGSRHPRSHQEDRAGHGHSADSSR |
| | QSGTRHTQTSSGGQAASSHEQARSSAGERH |
| | GSHHQQSADSSRHSGIGHGQASSAVRDSGH |
| | RGYSGSQASDNEGHSEDSDTQSVSAHGQAG |
| | SHQQSHQESARGRSGETSGHSGSFLYGQVS |
| | THEQSESSHGWTGPSTRGRQGSRHEQAQDS |
| | SRHSASQDGQDTIRGHPGSSRGGRQGYHHE |
| | HSVDSSGHSGSHHSHTTSQGRSDASRGQSG |
| | SRSASRTTRNEEQSGDGSRHSGSRHHEAST |
| | HADISRHSQAVQGQSEGSRRSRRQGSSVSQ |
| | DSDSEGHSEDSERWSGSASRNHHGSAQEQL |
| | RDGSRHPRSHQEDRAGHGHSADSSRQSGTR |
| | HTQTSSGGQAASSHEQARSSAGERHGSHHQ |
| | QSADSSRHSGIGHGQASSAVRDSGHRGYSG |
| | SQASDNEGHSEDSDTQSVSAHGQAGSHQQS |
| | HQESARGRSGETSGHSGSFLYGQVSTHEQS |
| | ESSHGWTGPSTRGRQGSRHEQAQDSSRHSA |
| | SQDGQDTIRGHPGSSRGGRQGYHHEHSVDS |
| | SGHSGSHHSHTTSQGRSDASRGQSGSRSAS |
| | RTTRNEEQSGDGSRHSGSRHHEASTHADIS |
| | RHSQAVQGQSEGSRRSRRQGSSVSQDSDSE |
| | GHSEDSERWSGSASRNHHGSAQEQLRDGSR |
| | HPRSHQEDRAGHGHSADSSRQSGTRHTQTS |
| | SGGQAASSHEQARSSAGERHGSHHQQSADS |
| | SRHSGIGHGQASSAVRDSGHRGYSGSQASD |
| | NEGHSEDSDTQSVSAHGQAGSHQQSHQESA |
| | RGRSGETSGHSGSFLYGQVSTHEQSESSHG |
| | WTGPSTRGRQGSRHEQAQDSSRHSASQDGQ |
| | DTIRGHPGSSRGGRQGYHHEHSVDSSGHSG |
| | SHHSHTTSQGRSDASRGQSGSRSASRTTRN |
| | EEQSGDGSRHSGSRHHEASTHADISRHSQA |
| | VQGQSEGSRRSRRQGSSVSQDSDSEGHSED |
| | SERWSGSASRNHHGSAQEQLRDGSRHPRSH |
| | QEDRAGHGHSADSSRQSGTRHTQTSSGGQA |
| | ASSHEQARSSAGERHGSHHQQSADSSRHSG |
| | IGHGQASSAVRDSGHRGYSGSQASDNEGHS |
| | EDSDTQSVSAHGQAGSHQQSHQESARGRSG |
| | ETSGHSGSFLYGPGLCGHSSDISKQLGFSQ |
| | SQRYYYYEG |
| mRFP1- | MASSEDVIKEFMRFKVRMEGSVNGHEFEIE |
| (r8)16 | GEGEGRPYEGTQTAKLKVTKGGPLPFAWDI |
| (SEQ ID | LSPQFQYGSKAYVKHPADIPDYLKLSFPEG |
| NO: 12) | FKWERVMNFEDGGVVTVTQDSSLQDGEFIY |
| | KVKLRGTNFPSDGPVMQKKTMGWEASTERM |
| | YPEDGALKGEIKMRLKLKDGGHYDAEVKTT |
| | YMAKKPVQLPGAYKTDIKLDITSHNEDYTI |
| | VEQYERAEGRHSTGASPGGQVSTHEQSESS |
| | HGWTGPSTRGRQGSRHEQAQDSSRHSASQD |
| | GQDTIRGHPGSSRGGRQGYHHEHSVDSSGH |
| | SGSHHSHTTSQGRSDASRGQSGSRSASRTT |
| | RNEEQSGDGSRHSGSRHHEASTHADISRHS |
| | QAVQGQSEGSRRSRRQGSSVSQDSDSEGHS |
| | EDSERWSGSASRNHHGSAQEQLRDGSRHPR |
| | SHQEDRAGHGHSADSSRQSGTRHTQTSSGG |
| | QAASSHEQARSSAGERHGSHHQQSADSSRH |
| | SGIGHGQASSAVRDSGHRGYSGSQASDNEG |
| | HSEDSDTQSVSAHGQAGSHQQSHQESARGR |
| | SGETSGHSGSFLYGQVSTHEQSESSHGWTG |
| | PSTRGRQGSRHEQAQDSSRHSASQDGQDTI |
| | RGHPGSSRGGRQGYHHEHSVDSSGHSGSHH |
| | SHTTSQGRSDASRGQSGSRSASRTTRNEEQ |
| | SGDGSRHSGSRHHEASTHADISRHSQAVQG |
| | QSEGSRRSRRQGSSVSQDSDSEGHSEDSER |
| | WSGSASRNHHGSAQEQLRDGSRHPRSHQED |
| | RAGHGHSADSSRQSGTRHTQTSSGGQAASS |
| | HEQARSSAGERHGSHHQQSADSSRHSGIGH |
| | GQASSAVRDSGHRGYSGSQASDNEGHSEDS |
| | DTQSVSAHGQAGSHQQSHQESARGRSGETS |
| | GHSGSFLYGQVSTHEQSESSHGWTGPSTRG |
| | RQGSRHEQAQDSSRHSASQDGQDTIRGHPG |
| | SSRGGRQGYHHEHSVDSSGHSGSHHSHTTS |
| | QGRSDASRGQSGSRSASRTTRNEEQSGDGS |
| | RHSGSRHHEASTHADISRHSQAVQGQSEGS |
| | RRSRRQGSSVSQDSDSEGHSEDSERWSGSA |
| | SRNHHGSAQEQLRDGSRHPRSHQEDRAGHG |
| | HSADSSRQSGTRHTQTSSGGQAASSHEQAR |
| | SSAGERHGSHHQQSADSSRHSGIGHGQASS |
| | AVRDSGHRGYSGSQASDNEGHSEDSDTQSV |
| | SAHGQAGSHQQSHQESARGRSGETSGHSGS |
| | FLYGQVSTHEQSESSHGWTGPSTRGRQGSR |
| | HEQAQDSSRHSASQDGQDTIRGHPGSSRGG |
| | RQGYHHEHSVDSSGHSGSHHSHTTSQGRSD |
| | ASRGQSGSRSASRTTRNEEQSGDGSRHSGS |
| | RHHEASTHADISRHSQAVQGQSEGSRRSRR |
| | QGSSVSQDSDSEGHSEDSERWSGSASRNHH |
| | GSAQEQLRDGSRHPRSHQEDRAGHGHSADS |
| | SRQSGTRHTQTSSGGQAASSHEQARSSAGE |
| | RHGSHHQQSADSSRHSGIGHGQASSAVRDS |
| | GHRGYSGSQASDNEGHSEDSDTQSVSAHGQ |
| | AGSHQQSHQESARGRSGETSGHSGSFLYGQ |
| | VSTHEQSESSHGWTGPSTRGRQGSRHEQAQ |
| | DSSRHSASQDGQDTIRGHPGSSRGGRQGYH |
| | HEHSVDSSGHSGSHHSHTTSQGRSDASRGQ |
| | SGSRSASRTTRNEEQSGDGSRHSGSRHHEA |
| | STHADISRHSQAVQGQSEGSRRSRRQGSSV |
| | SQDSDSEGHSEDSERWSGSASRNHHGSAQE |
| | QLRDGSRHPRSHQEDRAGHGHSADSSRQSG |
| | TRHTQTSSGGQAASSHEQARSSAGERHGSH |
| | HQQSADSSRHSGIGHGQASSAVRDSGHRGY |
| | SGSQASDNEGHSEDSDTQSVSAHGQAGSHQ |
| | QSHQESARGRSGETSGHSGSFLYGQVSTHE |
| | QSESSHGWTGPSTRGRQGSRHEQAQDSSRH |
| | SASQDGQDTIRGHPGSSRGGRQGYHHEHSV |
| | DSSGHSGSHHSHTTSQGRSDASRGQSGSRS |
| | ASRTTRNEEQSGDGSRHSGSRHHEASTHAD |
| | ISRHSQAVQGQSEGSRRSRRQGSSVSQDSD |
| | SEGHSEDSERWSGSASRNHHGSAQEQLRDG |
| | SRHPRSHQEDRAGHGHSADSSRQSGTRHTQ |
| | TSSGGQAASSHEQARSSAGERHGSHHQQSA |
| | DSSRHSGIGHGQASSAVRDSGHRGYSGSQA |
| | SDNEGHSEDSDTQSVSAHGQAGSHQQSHQE |
| | SARGRSGETSGHSGSFLYGQVSTHEQSESS |
| | HGWTGPSTRGRQGSRHEQAQDSSRHSASQD |
| | GQDTIRGHPGSSRGGRQGYHHEHSVDSSGH |
| | SGSHHSHTTSQGRSDASRGQSGSRSASRTT |
| | RNEEQSGDGSRHSGSRHHEASTHADISRHS |
| | QAVQGQSEGSRRSRRQGSSVSQDSDSEGHS |
| | EDSERWSGSASRNHHGSAQEQLRDGSRHPR |
| | SHQEDRAGHGHSADSSRQSGTRHTQTSSGG |
| | QAASSHEQARSSAGERHGSHHQQSADSSRH |
| | SGIGHGQASSAVRDSGHRGYSGSQASDNEG |
| | HSEDSDTQSVSAHGQAGSHQQSHQESARGR |
| | SGETSGHSGSFLYGQVSTHEQSESSHGWTG |
| | PSTRGRQGSRHEQAQDSSRHSASQDGQDTI |
| | RGHPGSSRGGRQGYHHEHSVDSSGHSGSHH |
| | SHTTSQGRSDASRGQSGSRSASRTTRNEEQ |
| | SGDGSRHSGSRHHEASTHADISRHSQAVQG |
| | QSEGSRRSRRQGSSVSQDSDSEGHSEDSER |
| | WSGSASRNHHGSAQEQLRDGSRHPRSHQED |
| | RAGHGHSADSSRQSGTRHTQTSSGGQAASS |
| | HEQ |
| | ARSSAGERHGSHHQQSADSSRHSGIGHGQA |
| | SSAVRDSGHRGYSGSQASDNEGHSEDSDTQ |
| | SVSAHGQAGSHQQSHQESARGRSGETSGHS |
| | GSFLYGQVSTHEQSESSHGWTGPSTRGRQG |
| | SRHEQAQDSSRHSASQDGQDTIRGHPGSSR |
| | GGRQGYHHEHSVDSSGHSGSHHSHTTSQGR |
| | SDASRGQSGSRSASRTTRNEEQSGDGSRHS |
| | GSRHHEASTHADISRHSQAVQGQSEGSRRS |
| | RRQGSSVSQDSDSEGHSEDSERWSGSASRN |
| | HHGSAQEQLRDGSRHPRSHQEDRAGHGHSA |
| | DSSRQSGTRHTQTSSGGQAASSHEQARSSA |
| | GERHGSHHQQSADSSRHSGIGHGQASSAVR |
| | DSGHRGYSGSQASDNEGHSEDSDTQSVSAH |
| | GQAGSHQQSHQESARGRSGETSGHSGSFLY |
| | GQVSTHEQSESSHGWTGPSTRGRQGSRHEQ |
| | AQDSSRHSASQDGQDTIRGHPGSSRGGRQG |
| | YHHEHSVDSSGHSGSHHSHTTSQGRSDASR |
| | GQSGSRSASRTTRNEEQSGDGSRHSGSRHH |
| | EASTHADISRHSQAVQGQSEGSRRSRRQGS |
| | SVSQDSDSEGHSEDSERWSGSASRNHHGSA |
| | QEQLRDGSRHPRSHQEDRAGHGHSADSSRQ |
| | SGTRHTQTSSGGQAASSHEQARSSAGERHG |
| | SHHQQSADSSRHSGIGHGQASSAVRDSGHR |
| | GYSGSQASDNEGHSEDSDTQSVSAHGQAGS |
| | HQQSHQESARGRSGETSGHSGSFLYGQVST |
| | HEQSESSHGWTGPSTRGRQGSRHEQAQDSS |
| | RHSASQDGQDTIRGHPGSSRGGRQGYHHEH |
| | SVDSSGHSGSHHSHTTSQGRSDASRGQSGS |
| | RSASRTTRNEEQSGDGSRHSGSRHHEASTH |
| | ADISRHSQAVQGQSEGSRRSRRQGSSVSQD |
| | SDSEGHSEDSERWSGSASRNHHGSAQEQLR |
| | DGSRHPRSHQEDRAGHGHSADSSRQSGTRH |
| | TQTSSGGQAASSHEQARSSAGERHGSHHQQ |
| | SADSSRHSGIGHGQASSAVRDSGHRGYSGS |
| | QASDNEGHSEDSDTQSVSAHGQAGSHQQSH |
| | QESARGRSGETSGHSGSFLYGQVSTHEQSE |
| | SSHGWTGPSTRGRQGSRHEQAQDSSRHSAS |
| | QDGQDTIRGHPGSSRGGRQGYHHEHSVDSS |
| | GHSGSHHSHTTSQGRSDASRGQSGSRSASR |
| | TTRNEEQSGDGSRHSGSRHHEASTHADISR |
| | HSQAVQGQSEGSRRSRRQGSSVSQDSDSEG |
| | HSEDSERWSGSASRNHHGSAQEQLRDGSRH |
| | PRSHQEDRAGHGHSADSSRQSGTRHTQTSS |
| | GGQAASSHEQARSSAGERHGSHHQQSADSS |
| | RHSGIGHGQASSAVRDSGHRGYSGSQASDN |
| | EGHSEDSDTQSVSAHGQAGSHQQSHQESAR |
| | GRSGETSGHSGSFLYGQVSTHEQSESSHGW |
| | TGPSTRGRQGSRHEQAQDSSRHSASQDGQD |
| | TIRGHPGSSRGGRQGYHHEHSVDSSGHSGS |
| | HHSHTTSQGRSDASRGQSGSRSASRTTRNE |
| | EQSGDGSRHSGSRHHEASTHADISRHSQAV |
| | QGQSEGSRRSRRQGSSVSQDSDSEGHSEDS |
| | ERWSGSASRNHHGSAQEQLRDGSRHPRSHQ |
| | EDRAGHGHSADSSRQSGTRHTQTSSGGQAA |
| | SSHEQARSSAGERHGSHHQQSADSSRHSGI |
| | GHGQASSAVRDSGHRGYSGSQASDNEGHSE |
| | DSDTQSVSAHGQAGSHQQSHQESARGRSGE |
| | TSGHSGSFLYGQVSTHEQSESSHGWTGPST |
| | RGRQGSRHEQAQDSSRHSASQDGQDTIRGH |
| | PGSSRGGRQGYHHEHSVDSSGHSGSHHSHT |
| | TSQGRSDASRGQSGSRSASRTTRNEEQSGD |
| | GSRHSGSRHHEASTHADISRHSQAVQGQSE |
| | GSRRSRRQGSSVSQDSDSEGHSEDSERWSG |
| | SASRNHHGSAQEQLRDGSRHPRSHQEDRAG |
| | HGHSADSSRQSGTRHTQTSSGGQAASSHEQ |
| | ARSSAGERHGSHHQQSADSSRHSGIGHGQA |
| | SSAVRDSGHRGYSGSQASDNEGHSEDSDTQ |
| | SVSAHGQAGSHQQSHQESARGRSGETSGHS |
| | GSFLYGQVSTHEQSESSHGWTGPSTRGRQG |
| | SRHEQAQDSSRHSASQDGQDTIRGHPGSSR |
| | GGRQGYHHEHSVDSSGHSGSHHSHTTSQGR |
| | SDASRGQSGSRSASRTTRNEEQSGDGSRHS |
| | GSRHHEASTHADISRHSQAVQGQSEGSRRS |
| | RRQGSSVSQDSDSEGHSEDSERWSGSASRN |
| | HHGSAQEQLRDGSRHPRSHQEDRAGHGHSA |
| | DSSRQSGTRHTQTSSGGQAASSHEQARSSA |
| | GERHGSHHQQSADSSRHSGIGHGQASSAVR |
| | DSGHRGYSGSQASDNEGHSEDSDTQSVSAH |
| | GQAGSHQQSHQESARGRSGETSGHSGSFLY |
| | GQVSTHEQSESSHGWTGPSTRGRQGSRHEQ |
| | AQDSSRHSASQDGQDTIRGHPGSSRGGRQG |
| | YHHEHSVDSSGHSGSHHSHTTSQGRSDASR |
| | GQSGSRSASRTTRNEEQSGDGSRHSGSRHH |
| | EASTHADISRHSQAVQGQSEGSRRSRRQGS |
| | SVSQDSDSEGHSEDSERWSGSASRNHHGSA |
| | QEQLRDGSRHPRSHQEDRAGHGHSADSSRQ |
| | SGTRHTQTSSGGQAASSHEQARSSAGERHG |
| | SHHQQSADSSRHSGIGHGQASSAVRDSGHR |
| | GYSGSQASDNEGHSEDSDTQSVSAHGQAGS |
| | HQQSHQESARGRSGETSGHSGSFLYG |
| S100- | MSTLLENIFAIINLFKQYSKKDKNTDTLSK |
| mRFP1- | KELKELLEKEFRQILKNPDDPDMVDVFMDH |
| (r8)8-Tail | LDIDHNKKIDFTEFLLMVFKLAQAYYESTR |
| (SEQ ID | KEGVPGSGVPGAGVPGSRSDASSEDVIKEF |
| NO: 13) | MRFKVRMEGSVNGHEFEIEGEGEGRPYEGT |
| | QTAKLKVTKGGPLPFAWDILSPQFQYGSKA |
| | YVKHPADIPDYLKLSFPEGFKWERVMNFED |
| | GGVVTVTQDSSLQDGEFIYKVKLRGTNFPS |
| | DGPVMQKKTMGWEASTERMYPEDGALKGEI |
| | KMRLKLKDGGHYDAEVKTTYMAKKPVQLPG |
| | AYKTDIKLDITSHNEDYTIVEQYERAEGRH |
| | STGASPGGQVSTHEQSESSHGWTGPSTRGR |
| | QGSRHEQAQDSSRHSASQDGQDTIRGHPGS |
| | SRGGRQGYHHEHSVDSSGHSGSHHSHTTSQ |
| | GRSDASRGQSGSRSASRTTRNEEQSGDGSR |
| | HSGSRHHEASTHADISRHSQAVQGQSEGSR |
| | RSRRQGSSVSQDSDSEGHSEDSERWSGSAS |
| | RNHHGSAQEQLRDGSRHPRSHQEDRAGHGH |
| | SADSSRQSGTRHTQTSSGGQAASSHEQARS |
| | SAGERHGSHHQQSADSSRHSGIGHGQASSA |
| | VRDSGHRGYSGSQASDNEGHSEDSDTQSVS |
| | AHGQAGSHQQSHQESARGRSGETSGHSGSF |
| | LYGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGQV |
| | STHEQSESSHGWTGPSTRGRQGSRHEQAQD |
| | SSRHSASQDGQDTIRGHPGSSRGGRQGYHH |
| | EHSVDSSGHSGSHHSHTTSQGRSDASRGQS |
| | GSRSASRTTRNEEQSGDGSRHSGSRHHEAS |
| | THADISRHSQAVQGQSEGSRRSRRQGSSVS |
| | QDSDSEGHSEDSERWSGSASRNHHGSAQEQ |
| | LRDGSRHPRSHQEDRAGHGHSADSSRQSGT |
| | RHTQTSSGGQAASSHEQARSSAGERHGSHH |
| | QQSADSSRHSGIGHGQASSAVRDSGHRGYS |
| | GSQASDNEGHSEDSDTQSVSAHGQAGSHQQ |
| | SHQESARGRSGETSGHSGSFLYGQVSTHEQ |
| | SESSHGWTGPSTRGRQGSRHEQAQDSSRHS |
| | ASQDGQDTIRGHPGSSRGGRQGYHHEHSVD |
| | SSGHSGSHHSHTTSQGRSDASRGQSGSRSA |
| | SRTTRNEEQSGDGSRHSGSRHHEASTHADI |
| | SRHSQAVQGQSEGSRRSRRQGSSVSQDSDS |
| | EGHSEDSERWSGSASRNHHGSAQEQLRDGS |
| | RHPRSHQEDRAGHGHSADSSRQSGTRHTQT |
| | SSGGQAASSHEQARSSAGERHGSHHQQSAD |
| | SSRHSGIGHGQASSAVRDSGHRGYSGSQAS |
| | DNEGHSEDSDTQSVSAHGQAGSHQQSHQES |
| | ARGRSGETSGHSGSFLYGQVSTHEQSESSH |
| | GWTGPSTRGRQGSRHEQAQDSSRHSASQDG |
| | QDTIRGHPGSSRGGRQGYHHEHSVDSSGHS |
| | GSHHSHTTSQGRSDASRGQSGSRSASRTTR |
| | NEEQSGDGSRHSGSRHHEASTHADISRHSQ |
| | AVQGQSEGSRRSRRQGSSVSQDSDSEGHSE |
| | DSERWSGSASRNHHGSAQEQLRDGSRHPRS |
| | HQEDRAGHGHSADSSRQSGTRHTQTSSGGQ |
| | AASSHEQARSSAGERHGSHHQQSADSSRHS |
| | GIGHGQASSAVRDSGHRGYSGSQASDNEGH |
| | SEDSDTQSVSAHGQAGSHQQSHQESARGRS |
| | GETSGHSGSFLYGQVSTHEQSESSHGWTGP |
| | STRGRQGSRHEQAQDSSRHSASQDGQDTIR |
| | GHPGSSRGGRQGYHHEHSVDSSGHSGSHHS |
| | HTTSQGRSDASRGQSGSRSASRTTRNEEQS |
| | GDGSRHSGSRHHEASTHADISRHSQAVQGQ |
| | SEGSRRSRRQGSSVSQDSDSEGHSEDSERW |
| | SGSASRNHHGSAQEQLRDGSRHPRSHQEDR |
| | AGHGHSADSSRQSGTRHTQTSSGGQAASSH |
| | EQARSSAGERHGSHHQQSADSSRHSGIGHG |
| | QASSAVRDSGHRGYSGSQASDNEGHSEDSD |
| | TQSVSAHGQAGSHQQSHQESARGRSGETSG |
| | HSGSFLYGQVSTHEQSESSHGWTGPSTRGR |
| | QGSRHEQAQDSSRHSASQDGQDTIRGHPGS |
| | SRGGRQGYHHEHSVDSSGHSGSHHSHTTSQ |
| | GRSDASRGQSGSRSASRTTRNEEQSGDGSR |
| | HSGSRHHEASTHADISRHSQAVQGQSEGSR |
| | RSRRQGSSVSQDSDSEGHSEDSERWSGSAS |
| | RNHHGSAQEQLRDGSRHPRSHQEDRAGHGH |
| | SADSSRQSGTRHTQTSSGGQAASSHEQARS |
| | SAGERHGSHHQQSADSSRHSGIGHGQASSA |
| | VRDSGHRGYSGSQASDNEGHSEDSDTQSVS |
| | AHGQAGSHQQSHQESARGRSGETSGHSGSF |
| | LYGQVSTHEQSESSHGWTGPSTRGRQGSRH |
| | EQAQDSSRHSASQDGQDTIRGHPGSSRGGR |
| | QGYHHEHSVDSSGHSGSHHSHTTSQGRSDA |
| | SRGQSGSRSASRTTRNEEQSGDGSRHSGSR |
| | HHEASTHADISRHSQAVQGQSEGSRRSRRQ |
| | GSSVSQDSDSEGHSEDSERWSGSASRNHHG |
| | SAQEQLRDGSRHPRSHQEDRAGHGHSADSS |
| | RQSGTRHTQTSSGGQAASSHEQARSSAGER |
| | HGSHHQQSADSSRHSGIGHGQASSAVRDSG |
| | HRGYSGSQASDNEGHSEDSDTQSVSAHGQA |
| | GSHQQSHQESARGRSGETSGHSGSFLYGPG |
| | LCGHSSDISKQLGFSQSQRYYYYEG |

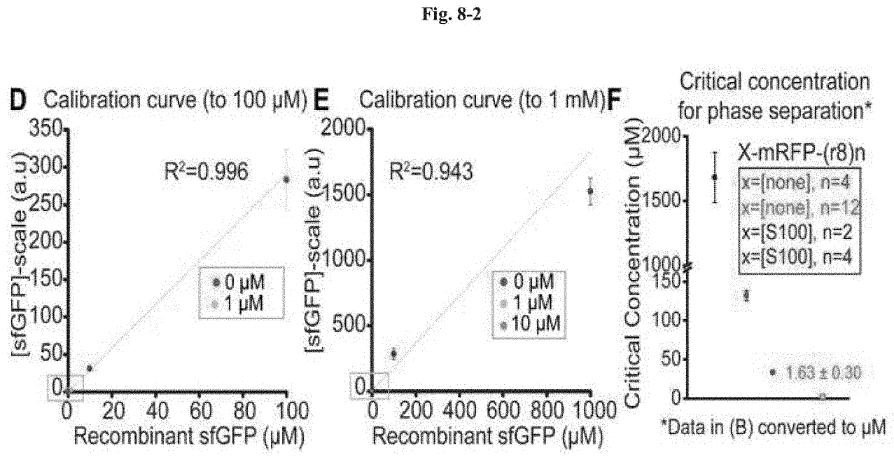

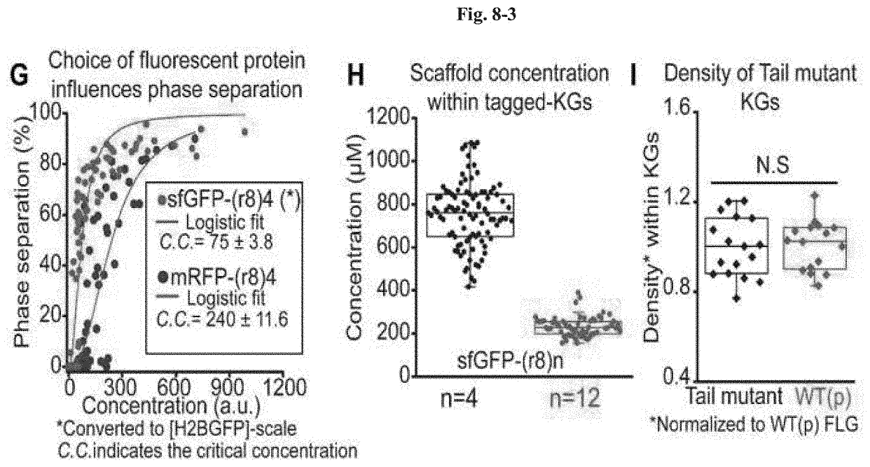

Notably, humans with filaggrin early truncation mutations fail to generate KGs. Such mutations account for >80% of cases among northern Europeans (27). To quantitatively determine how disease-associated mutations alter the critical concentration for phase separation, we incorporated a self-cleavable [p2a] sequence (28) to express equimolar amounts of mRFP-FLG variants and H2B-GFP (as a proxy for variant concentration) (FIG. 8A and TABLE 2). Live imaging of transfected HaCATs expressing comparable nuclear GFP revealed a striking relation between the number of filaggrin repeats and phase separation propensity (FIG. 1F). Over a wide range of expression levels, disease-associated mutations with ≤4 repeats exhibited a dramatic increase (˜130 to >1500 μM) in critical concentration required for phase separation behavior (FIG. 1G and FIG. S7B-F). By contrast, wild-type filaggrin (n=12) phase separated at ˜2 μM. These properties were exemplified by live imaging, exposing the rapid formation and growth of KG-like structures as filaggrin reached its critical concentration for phase separation (FIG. 1H).

Filaggrin and its paralogs belong to the S100-fused type protein family which feature two short ‘EF hand’ calcium-binding motifs (˜2% of the protein), N-terminal to the IDP domain. The S100 domain is known to dimerize (29), and when fused to filaggrin variants, it reduced the critical concentration for phase separation (FIG. 1I). Despite these favorable interactions, S100 with mut-n2 FLG mutations still failed to appreciably phase separate even at high concentration (FIG. 1I and FIG. 8B-F). Overall, when compared with mRFP fusions, sfGFP lowered the critical concentrations for phase separation of tagged-FLG variants, although the results were consistent independent of the tag (FIG. 8G-H).

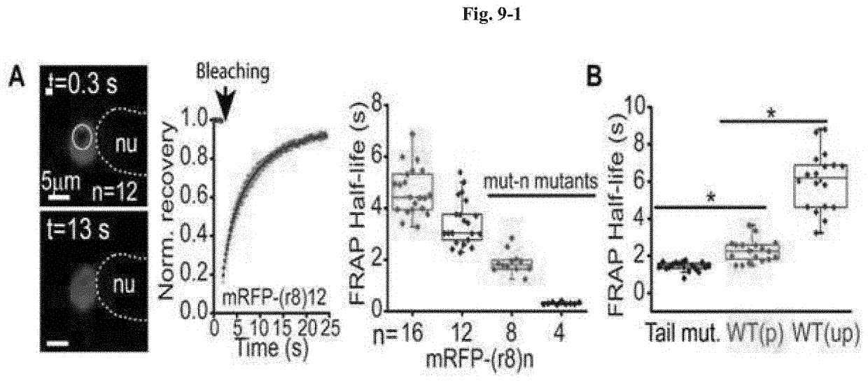

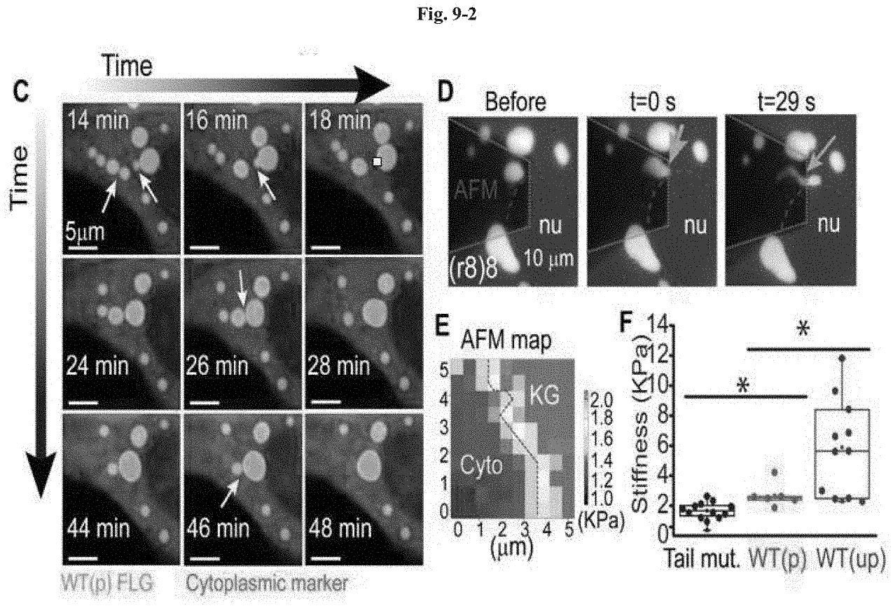

To further explore whether compromised phase separation might underlie disease-severity, we next performed fluorescence recovery after photobleaching (FRAP). As expected for a diffusive process, highly truncated, smaller FLG repeat variants exhibited more rapid recovery than WT-sized proteins (FIG. 9A). Remarkably, however, even the largest FLG variants (>430 KDa) recovered fully within a few seconds (FIG. 9A). The amino and carboxy domains flanking the repeats also affected recovery time. As predicted, the S100 dimerizing domain of FLG variants increased recovery half-life after photobleaching even further, while deletion of the carboxy tail, an intriguingly small truncation mutation seen in some patients, accelerated recovery (FIG. 8B and FIG. 8I). Overall, the dynamic FRAP behavior established filaggrin-containing KGs as bimolecular condensates, and distinguished them from mere aggregates in the cell. Moreover, since the S100 domain is cleaved during terminal differentiation, its function is likely to optimize phase separation at earlier stages when filaggrin levels are low and KGs just begin to form.

Liquid-Like Behavior of Filaggrin Granules

Live cell imaging revealed that HaCATs harboring our engineered filaggrins underwent granule rearrangements and fusion events that are hallmarks of liquid-like droplets (FIG. 9C). Individual fusion events were complete within seconds (FIG. 10).

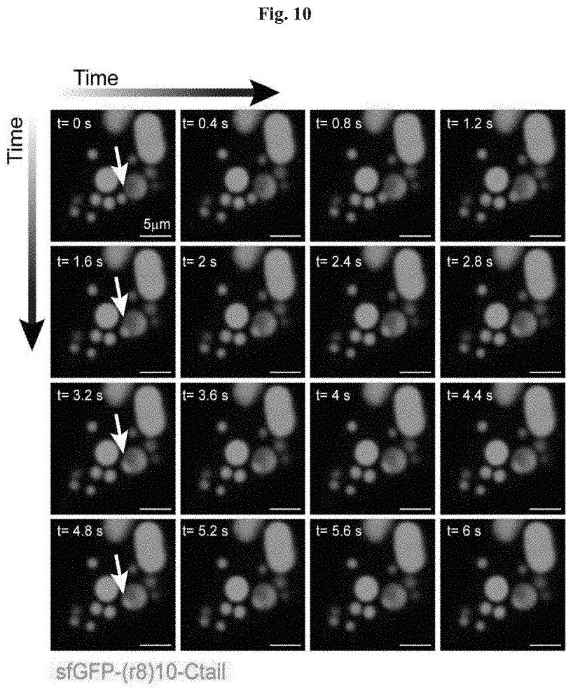

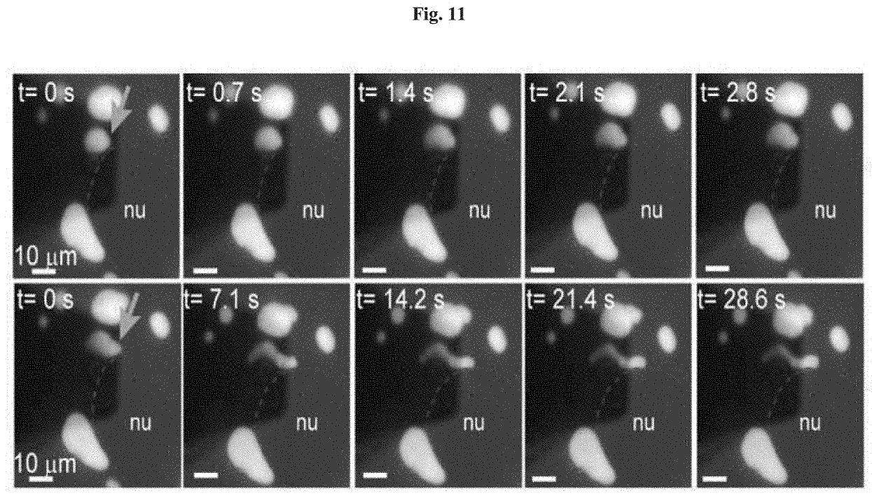

To further probe their material properties, we employed atomic-force microscopy (AFM). By applying pressure with an AFM probe directly on top of filaggrin granules, they deformed, creating liquid-like streaming around the cell's nucleus (FIG. 9D and FIG. 11). Even unprocessed (more viscous) S100-containing filaggrin granules underwent fusion when pushed into close proximity by the AFM probe (data not shown).

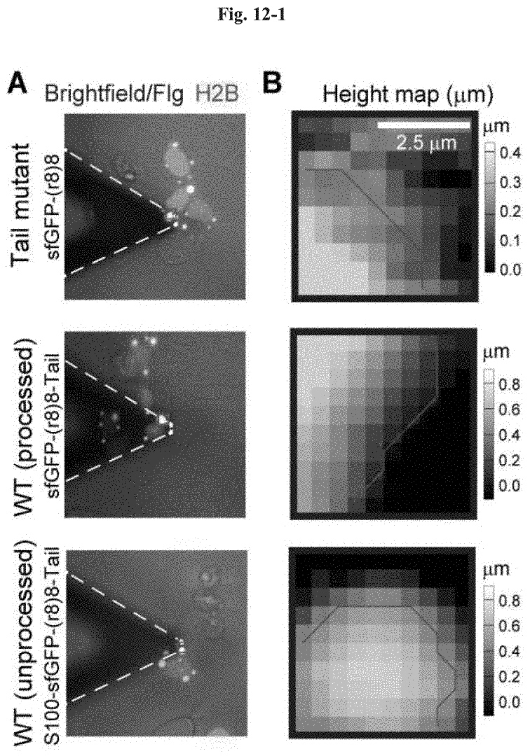

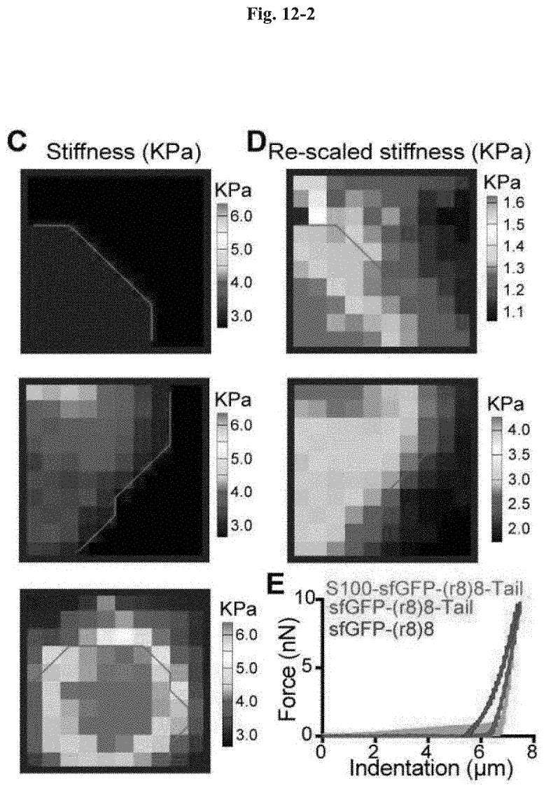

Our photobleaching data suggested that the material properties of KGs may change as a function of filaggrin processing and disease-associated mutations. To test this hypothesis, we performed serial force-indentation measurements across the granule length in HaCATs harboring different filaggrin variants (FIG. 9E and FIG. 12). Consistent with a role for the tail domain in tuning the material properties of KGs, AFM revealed a stiffening of the cellular domain spanned by tail-containing granules as compared to filaggrin counterparts that mimicked tail-deficient mutants (FIG. 9F). As suspected from the photobleaching data, unprocessed (S100-containing) filaggrin variants displayed dramatic stiffening (FIG. 9F and FIG. 12). Taken together, these data demonstrate that filaggrin granules are mechanically-responsive, liquid-like condensates in cells.

Engineering Phase Separation Sensors to Interrogate Endogenous KGs

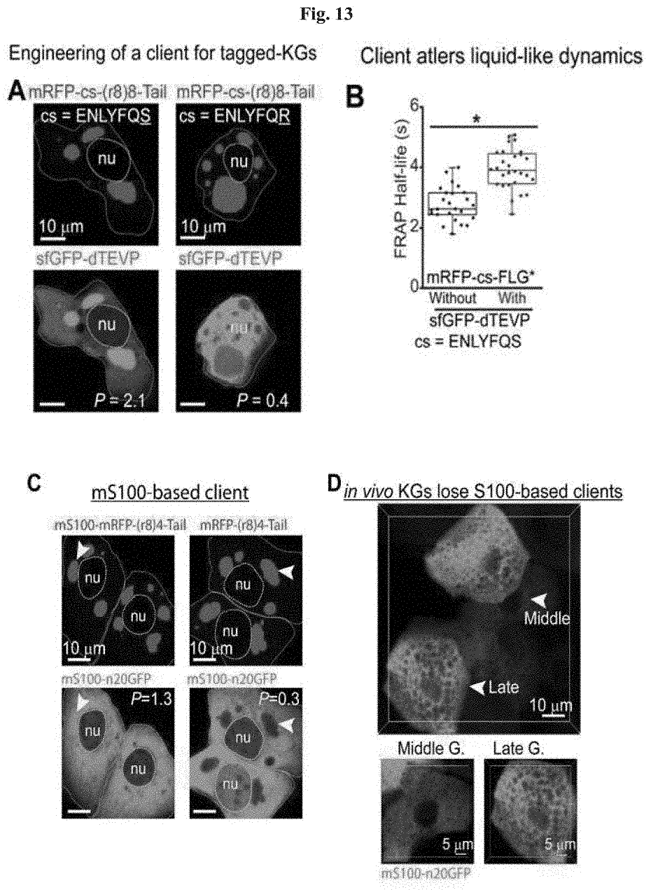

While our tagged filaggrin variants assembled de novo into KG-like structures, it was critical to address whether endogenous KGs in skin assemble through phase separation of filaggrin and if so, how their putative liquid-like properties contributed to epidermal differentiation. To do so, we could not use direct filaggrin tagging to label endogenous KGs, as it alters its biophysical properties (FIG. 8G). Similarly, oft-used client proteins that directly bind to a phase-separating protein scaffold (2, 30, 31) were not suitable, as although they can be recruited to existing liquid-like condensates and be used as carriers of fluorescence, they report scaffold localization irrespective of phase separation. Moreover, with complex differentiation programs in tissues, where processing of the scaffold can occur, the caveats of conventional clients become all the more apparent (FIG. 13).

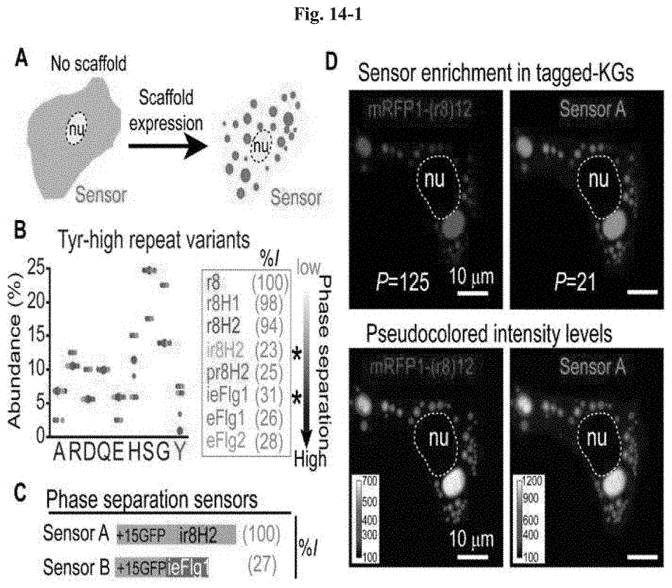

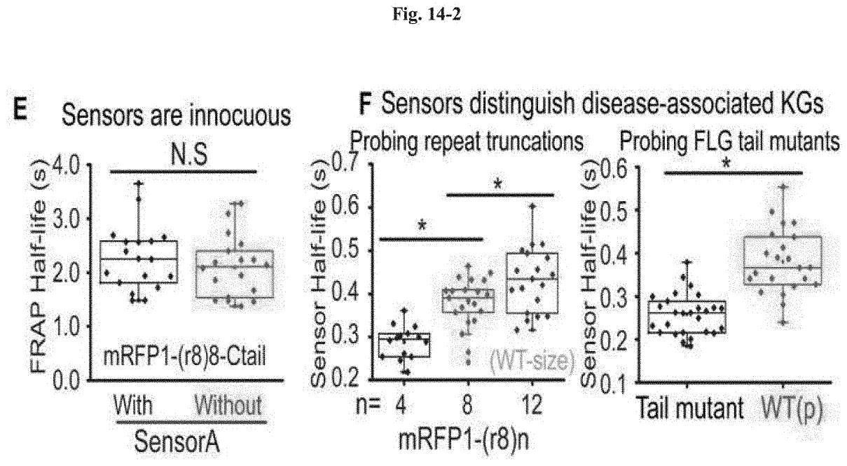

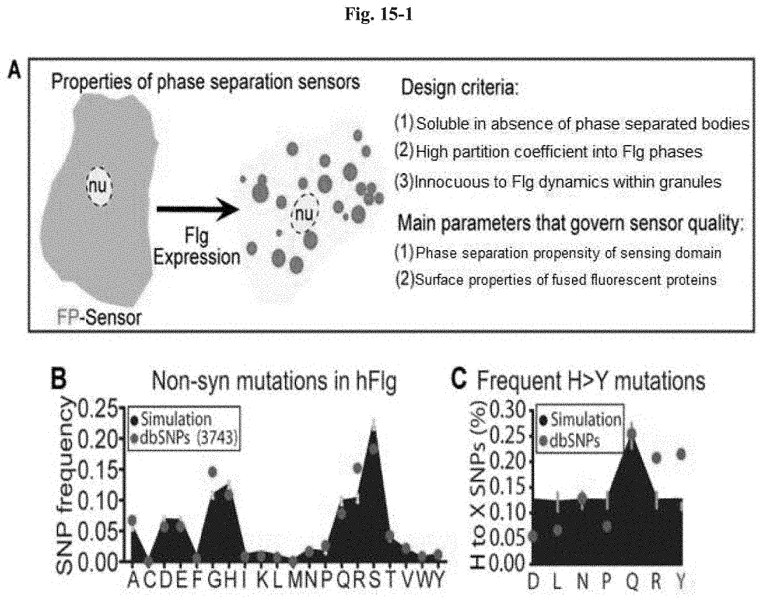

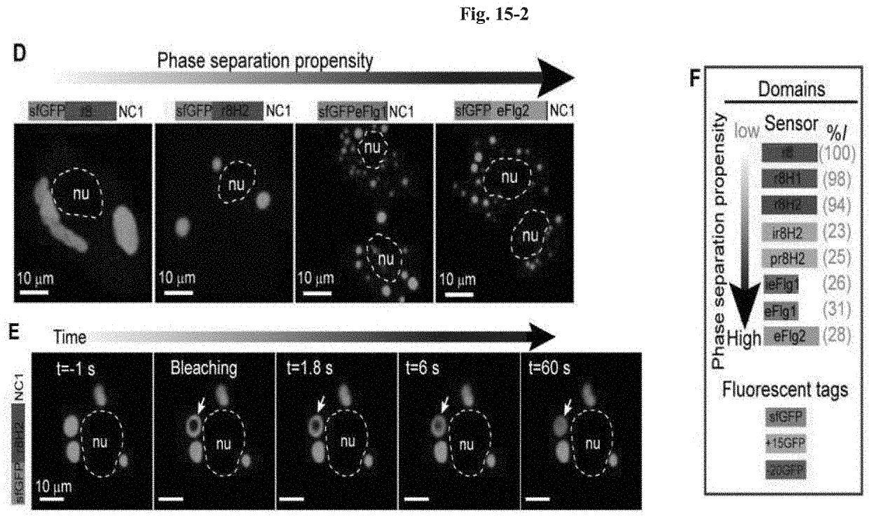

Thus, we sought to design novel clients that would permit probing the phase separation behavior of endogenous scaffold proteins as their concentration and processing change in living tissues. We aimed for soluble IDP clients that lack phase separation behavior of their own, but co-partition efficiently and innocuously into nascent phase-separated condensates by engaging in ultra-weak, phase-separation-specific (combinations of charge-charge, cation-pi, pi-pi, hydrogen-bonding and hydrophobic) interactions with the scaffold (FIG. 14A and FIG. 15A).

To engineer such ‘phase separation sensors’ for endogenous filaggrin, we exploited a) the non-pathogenic behavior of human filaggrin repeat mutants that possess His:Tyr mutations (FIG. 15B-C), and b) the inability of a sole filaggrin repeat to drive phase separation. After documenting the tuned phase-separation characteristics of Tyr-high FLG repeat #8 (r8) variants (r8H1 and r8H2) (22), we then generated variants with related sequence patterns but low sequence identity (ir8H2 and pr8H2). We also engineered proteins smaller than a filaggrin repeat, but with similar compositional biases (eFlg1, ieFlg1 and eFlg2). These proteins displayed a range of phase separation propensities (FIG. 14B, FIG. 15D-F, TABLE 3).

TABLE 3
Sequence information for synthesized phase separation sensors.

| Construct | Sequence |
|---|---|
| r8 (WT | QVSTHEQSESSHGWTGPSTRGRQGSRHEQAQDSSRHSASQDGQDTIRGHPGSS |
| repeat in | RGGRQGYHHEHSVDSSGHSGSHHSHTTSQGRSDASRGQSGSRSASRTTRNEEQ |
| human FLG) | SGDGSRHSGSRHHEASTHADISRHSQAVQGQSEGSRRSRRQGSSVSQDSDSEG |
| (SEQ ID | HSEDSERWSGSASRNHHGSAQEQLRDGSRHPRSHQEDRAGHGHSADSSRQSG |
| NO: 14) | TRHTQTSSGGQAASSHEQARSSAGERHGSHHQQSADSSRHSGIGHGQASSAVR |
| | DSGHRGYSGSQASDNEGHSEDSDTQSVSAHGQAGSHQQSHQESARGRSGETS |
| | GHSGSFLY |
| r8Hl | QVSTHEQSESSHGWTGPSTRGRQGSRYEQAQDSSRYSASQDGQDTIRGYPGSS |
| (SEQ ID | RGGRQGYHHEHSVDSSGYSGSHHSHTTSQGRSDASRGQSGSRSASRTTRNEEQ |
| NO: 15) | SGDGSRYSGSRHHEASTHADISRYSQAVQGQSEGSRRSRRQGSSVSQDSDSEG |
| | HSEDSERWSGSASRNHHGSAQEQLRDGSRHPRSHQEDRAGHGYSADSSRQSG |
| | TRHTQTSSGGQAASSHEQARSSAGERHGSHYQQSADSSRHSGIGHGQASSAVR |
| | DSGHRGYSGSQASDNEGHSEDSDTQSVSAHGQAGSHQQSHQESARGRSGETS |
| | GHSGSFLY |
| r8H2 | QVSTYEQSESSYGWTGPSTRGRQGSRYEQAQDSSRYSASQDGQDTIRGYPGSS |
| (SEQ ID | RGGRQGYYYEHSVDSSGYSGSYHSHTTSQGRSDASRGQSGSRSASRTTRNEEQ |
| NO: 16) | SGDGSRYSGSRHYEASTHADISRYSQAVQGQSEGSRRSRRQGSSVSQDSDSEG |
| | HSEDSERWSGSASRNHHGSAQEQLRDGSRYPRSHQEDRAGHGYSADSSRQSG |
| | TRYTQTSSGGQAASSHEQARSSAGERYGSHYQQSADSSRHSGIGHGQASSAVR |
| | DSGHRGYSGSQASDNEGHSEDSDTQSVSAHGQAGSHQQSYQESARGRSGETS |
| | GHSGSFLY |
| ir8H2 | YLFSGSHGSTEGSRGRASEQYSQQHSGAQGHASVSQTDSDESHGENDSAQSGS |
| (SEQ ID | YGRHGSDRVASSAQGHGIGSHRSSDASQQYHSGYREGASSRAQEHSSAAQGG |
| NO: 17) | SSTQTYRTGSQRSSDASYGHGARDEQHSRPYRSGDRLQEQASGHHNRSASGS |
| | WRESDESHGESDSDQSVSSGQRRSRRSGESQGQVAQSYRSIDAHTSAEYHRSG |
| | SYRSGDGSQEENRTTRSASRSGSQGRSADSRGQSTTHSHYSGSYGSSDVSHEY |
| | YYGQRGGRSSGPYGRITDQGDQSASYRSSDQAQEYRSGQRGRTSPGTWGYSS |
| | ESQEYTSVQ |
| pr8H2 | TRNHQGDQRSSHSSSQSYQRYPSRIHEEREDAYEHEGGSSRGGRSGGQSGGST |
| (SEQ ID | REQHSASAVGSATRQSEGGYTSYYIASQSSDSHSYGQRSSYSVGSQDQRTDGN |
| NO: 18) | AYDGTSQHDRSSASGYRGEFEQSRSAVPGASGGQDHERESQSRSRHTEWHDG |
| | DIGSGGSSESRASSGQEGARSSEDRERGAGSSSGGSRQSSTDRQHRYGGAEGS |
| | GSQGRSGSHQDDSNHQYAGQGRASYHLGYSPARQHSSSYSTRHDRYTTQYQS |
| | GQAAGSRYHSQLYTQSTDQSAASSEQADSVQVSTSSQSYRGRSWDSSGEVRR |
| | SHSYGASSH |
| eFlg1 | GRDGSHSYQGDRSGHSHQRQGYHEQSDRAGHGDSGHRGYSGRDGSHSYQGD |
| (SEQ ID | RSGHSHQRQGYHEQSDRAGHGDSGHRGYSGRDGSHSYQGDRSGHSHQRQGY |
| NO: 19) | HEQSDRAGHGDSGHRGYSGRDGSHSYQGDRSGHSHQRQGYHEQSDRAGHGD |
| | SGHRGYSGRDGSHSYQGDRSGHSHQRQGYHEQSDRAGHGDSGHRGYS |
| eFlg1 | SYGRHGSDGHGARDSQEHYGQRQHSHGSRDGQYSHSGDRGSYGRHGSDGHG |
| (SEQ ID | ARDSQEHYGQRQHSHGSRDGQYSHSGDRGSYGRHGSDGHGARDSQEHYGQR |
| NO: 20) | QHSHGSRDGQYSHSGDRGSYGRHGSDGHGARDSQEHYGQRQHSHGSRDGQY |
| | SHSGDRGSYGRHGSDGHGARDSQEHYGQRQHSHGSRDGQYSHSGDRG |
| eFlg2 | GRDGSHSYQGDRSGHSHQRQGYHEQSDRAGHGDSGHRGYSGRDGSHSYQGD |
| (SEQ ID | RSGHSHQRQGYHEQSDRAGHGDSGHRGYSGRDGSHSYQGDRSGHSHQRQGY |
| NO: 21) | HEQSDRAGHGDSGHRGYSGRDGSHSYQGDRSGHSHQRQGYHEQSDRAGHGD |
| | SGHRGYSSYGRHGSDGHGARDSQEHYGQRQHSHGSRDGQYSHSGDRGSYGR |
| | HGSDGHGARDSQEHYGQRQHSHGSRDGQYSHSGDRGSYGRHGSDGHGARDS |
| | QEHYGQRQHSHGSRDGQYSHSGDRGSYGRHGSDGHGARDSQEHYGQRQHSH |
| | GSRDGQYSHSGDRG |
| sfGFP-NES | MGASKGEELFTGVVPILVELDGDVNGHKFSVRGEGEGDATNGKLTLKFICTTG |
| (SEQ ID | KLPVPWPTLVTTLGYGVQCFSRYPDHMKRHDFFKSAMPEGYVQERTISFKDD |
| NO: 22) | GTYKTRAEVKFEGDTLVNRIELKGIDFKEDGNILGHKLEYNFNSHNVYITADK |
| | QKNGIKANFKIRHNVEDGSVQLADHYQQNTPIGDGPVLLPDNHYLSTQSVLSK |
| | DPNEKRDHMVLLEFVTAAGITHGMDELYKSGLELLEDLTLGSP |
| −20sfGFP- | MGASKGEELFTGVVPILVELDGDVNGHKFSVRGEGEGDATNGKLTLKFICTTG |
| NES | KLPVPWPTLVTTLTYGVQCFSRYPDHMDQHDFFKSAMPEGYVQERTISFKDD |
| (SEQ ID | GTYKTRAEVKFEGDTLVNRIELKGIDFKEDGNILGHKLEYNFNSHDVYITADK |
| NO: 23) | QENGIKAEFEIRHNVEDGSVQLADHYQQNTPIGDGPVLLPDDHYLSTESALSK |
| | DPNEDRDHMVLLEFVTAAGIDHGMDELYKSGLELLEDLTLGSPG |
| +15sfGFP- | MGASKGERLFTGVVPILVELDGDVNGHKFSVRGEGEGDATRGKLTLKFICTTG |
| NES | KLPVPWPTLVTTLTYGVQCFSRYPKHMKRHDFFKSAMPEGYVQERTISFKKD |
| (SEQ ID | GTYKTRAEVKFEGRTLVNRIELKGRDFKEKGNILGHKLEYNFNSHNVYITADK |
| NO: 24) | RKNGIKANFKIRHNVKDGSVQLADHYQQNTPIGRGPVLLPRNHYLSTRSALSK |
| | DPKEKRDHMVLLEFVTAAGITHGMDELYKSGLELLEDLTLGSP |
| +15sfGFPK- | MGASKGEKLFTGVVPILVELDGDVNGHKFSVRGEGEGDATKGKLTLKFICTT |
| NES | GKLPVPWPTLVTTLTYGVQCFSRYPKHMKRHDFFKSAMPEGYVQERTISFKK |
| (SEQ ID | DGTYKTRAEVKFEGKTLVNRIELKGKDFKEKGNILGHKLEYNFNSHNVYITAD |
| NO: 25) | KKKNGIKANFKIRHNVKDGSVQLADHYQQNTPIGKGPVLLPKNHYLSTKSALS |
| | KDPKEKRDHMVLLEFVTAAGITHGMDELYKSGLELLEDLTLGSPG |
| Sensor A | MGASKGERLFTGVVPILVELDGDVNGHKFSVRGEGEGDATRGKLTLKFICTTG |
| (full | KLPVPWPTLVTTLTYGVQCFSRYPKHMKRHDFFKSAMPEGYVQERTISFKKD |
| sequence) | GTYKTRAEVKFEGRTLVNRIELKGRDFKEKGNILGHKLEYNFNSHNVYITADK |
| (SEQ ID | RKNGIKANFKIRHNVKDGSVQLADHYQQNTPIGRGPVLLPRNHYLSTRSALSK |
| NO: 26) | DPKEKRDHMVLLEFVTAAGITHGMDELYKSGLELLEDLTLGSPGYLFSGSHGS |
| | TEGSRGRASEQYSQQHSGAQGHASVSQTDSDESHGENDSAQSGSYGRHGSDR |
| | VASSAQGHGIGSHRSSDASQQYHSGYREGASSRAQEHSSAAQGGSSTQTYRTG |
| | SQRSSDASYGHGARDEQHSRPYRSGDRLQEQASGHHNRSASGSWRESDESHG |
| | ESDSDQSVSSGQRRSRRSGESQGQVAQSYRSIDAHTSAEYHRSGSYRSGDGSQ |
| | EENRTTRSASRSGSQGRSADSRGQSTTHSHYSGSYGSSDVSHEYYYGQRGGRS |
| | SGPYGRITDQGDQSASYRSSDQAQEYRSGQRGRTSPGTWGYSSESQEYTSVQG |
| | s |
| Sensor B | MGASKGERLFTGVVPILVELDGDVNGHKFSVRGEGEGDATRGKLTLKFICTTG |
| (full | KLPVPWPTLVTTLTYGVQCFSRYPKHMKRHDFFKSAMPEGYVQERTISFKKD |
| sequence) | GTYKTRAEVKFEGRTLVNRIELKGRDFKEKGNILGHKLEYNFNSHNVYITADK |
| (SEQ ID | RKNGIKANFKIRHNVKDGSVQLADHYQQNTPIGRGPVLLPRNHYLSTRSALSK |
| NO: 27) | DPKEKRDHMVLLEFVTAAGITHGMDELYKSGLELLEDLTLGSPGSYGRHGSD |
| | GHGARDSQEHYGQRQHSHGSRDGQYSHSGDRGSYGRHGSDGHGARDSQEHY |
| | GQRQHSHGSRDGQYSHSGDRGSYGRHGSDGHGARDSQEHYGQRQHSHGSRD |
| | GQYSHSGDRGSYGRHGSDGHGARDSQEHYGQRQHSHGSRDGQYSHSGDRGS |
| | YGRHGSDGHGARDSQEHYGQRQHSHGSRDGQYSHSGDRGGS |
| +15GFP | MGASKGERLFTGVVPILVELDGDVNGHKFSVRGEGEGDATRGKLTLKFICTTG |
| (SEQ ID | KLPVPWPTLVTTLTYGVQCFSRYPKHMKRHDFFKSAMPEGYVQERTISFKKD |
| NO: 28) | GTYKTRAEVKFEGRTLVNRIELKGRDFKEKGNILGHKLEYNFNSHNVYITADK |
| | RKNGIKANFKIRHNVKDGSVQLADHYQQNTPIGRGPVLLPRNHYLSTRSALSK |
| | DPKEKRDHMVLLEFVTAAGITHGMDELYK |
| +15GFPK | MGASKGEKLFTGVVPILVELDGDVNGHKFSVRGEGEGDATKGKLTLKFICTT |
| (SEQ ID | GKLPVPWPTLVTTLTYGVQCFSRYPKHMKRHDFFKSAMPEGYVQERTISFKK |
| NO: 29) | DGTYKTRAEVKFEGKTLVNRIELKGKDFKEKGNILGHKLEYNFNSHNVYITAD |
| | KKKNGIKANFKIRHNVKDGSVQLADHYQQNTPIGKGPVLLPKNHYLSTKSALS |
| | KDPKEKRDHMVLLEFVTAAGITHGMDELYK |

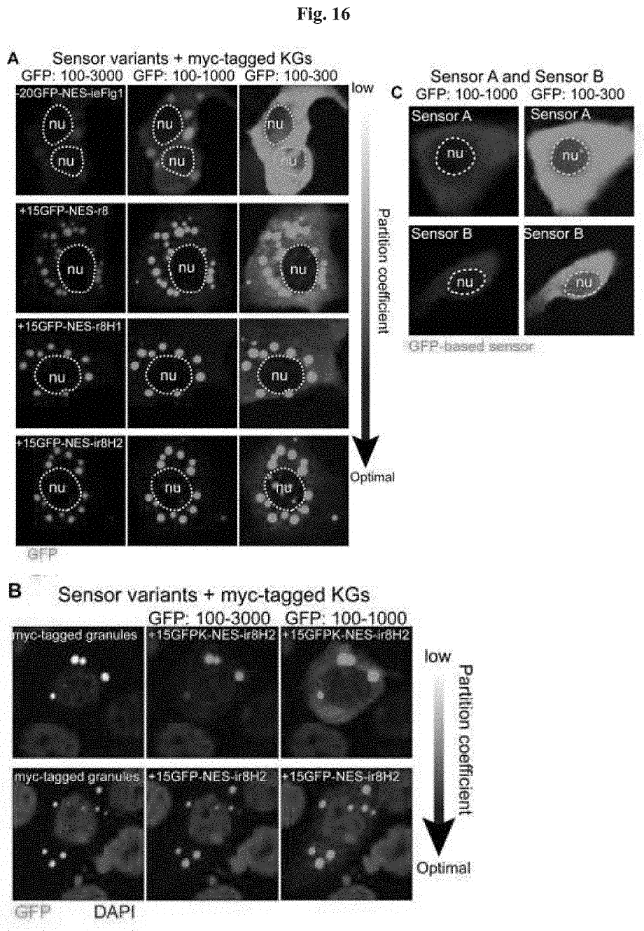

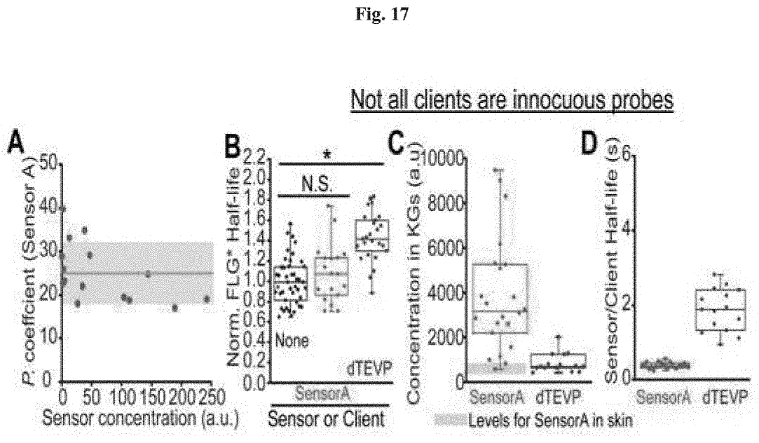

For live imaging, our sensors needed a fluorescent tag. Since surface charge of fusion proteins can affect IDP phase separation behavior (32), we first screened Tyr-high sensor variants fused to sfGFPs of varying net charges (33) for those that display high partition coefficients into KGs (FIG. 15F; FIG. 16A-B). We selected two +15GFP-based (sfGFP with net charge+15) sensor designs which shared little sequence identity among themselves or the native filaggrin repeat (FIG. 14C). The +15GFP (SEQ ID NO:28) and +15GFPK (SEQ ID NO:29) net charge+15 GFP sequences are provided above in TABLE 3. On their own, these sensors showed no phase separation (FIG. 16C), but when co-expressed with an mRFP-tagged filaggrin in HaCATs, they partitioned into the de novo assembled KGs (FIG. 14D). In particular, the exquisite relocalization of Sensor A from the cytoplasm into filaggrin granules (partition coefficient P=21) compared well to the behavior of filaggrin itself (P=125), enabling its faithful reporting of steep concentration gradients across membraneless granule boundaries. Importantly, and in contrast to a conventional client that bound stoichiometrically to a filaggrin domain (FIG. 17), FRAP dynamics of mRFP tagged-filaggrin were unaffected by Sensor A (FIG. 14E).

Due to FRAP's size-dependence, our studies in FIG. 9A were only suggestive that FLG truncating mutations accelerate liquid-like dynamics within KGs. With our new sensors as internal controls, we could now accurately determine FRAP half-lives and evaluate the liquid-like behavior of KGs formed from different-sized filaggrin mutants. As predicted, KGs assembled from truncated filaggrins differed from their full-length counterparts in displaying sensor recovery dynamics indicative of a decrease in the relative viscosity of KGs (FIG. 14F). Moreover, KGs assembled from tail mutants behaved as less viscous liquids than their tail-containing counterparts. Together, these findings underscore the power of our phase separation sensors to integrate into and innocuously report the material properties of liquid-like KGs. The data provide compelling evidence that patient disease phenotypes are linked to shifts in the liquid-like behavior of mutant KGs.

Crowding of Liquid-Like KGs within Skin Cells in Tissue.

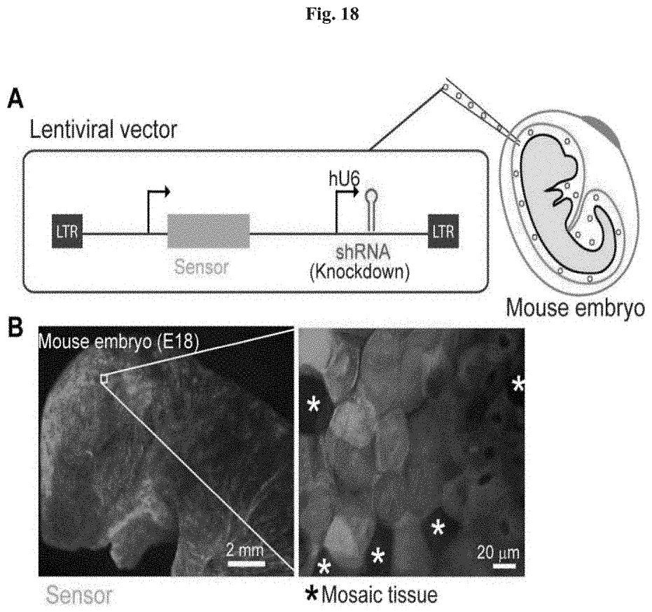

Our ultimate goal was to interrogate the dynamics of these liquid-liquid phase transitions in vivo in the skin epidermis. To this end, we employed our non-invasive in utero lentiviral delivery system to selectively, efficiently and stably transduce the single layer of embryonic E9.5 mouse skin epithelium with doxycycline-inducible transgenes encoding our sensors (FIG. 18). To induce expression during epidermal differentiation, we transduced embryos carrying a doxycycline-sensitive rtTA transactivator driven by the human Involucrin (Ivl) promoter (34).

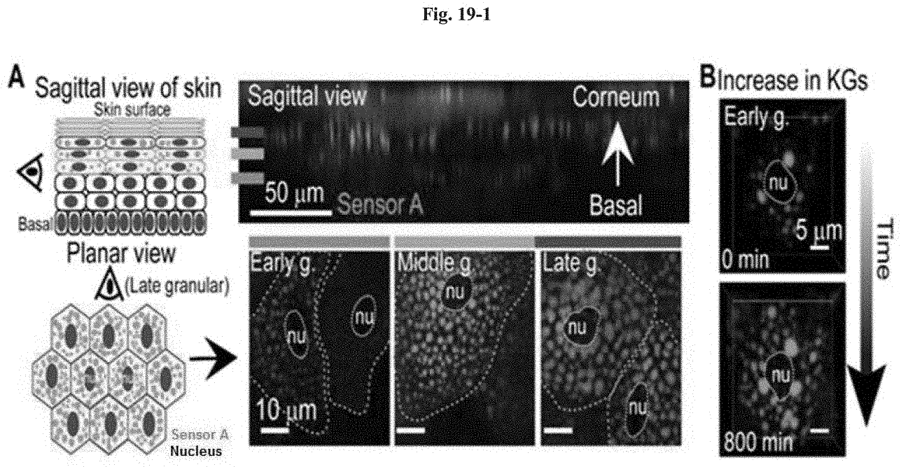

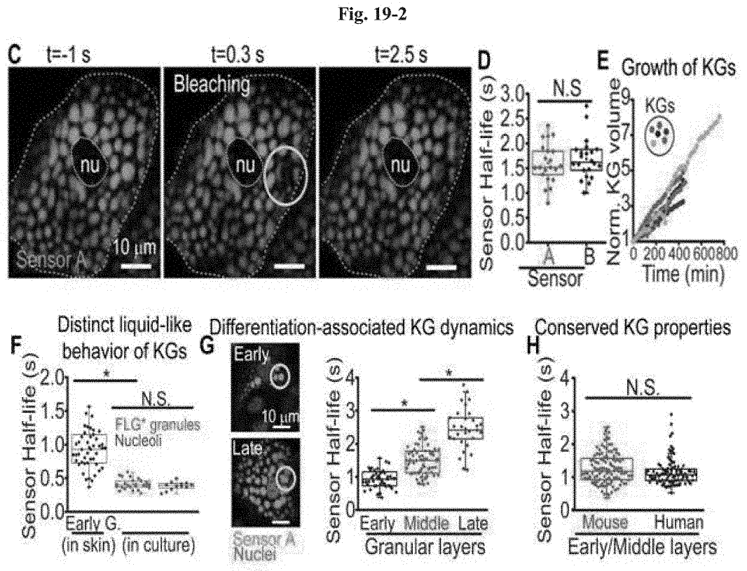

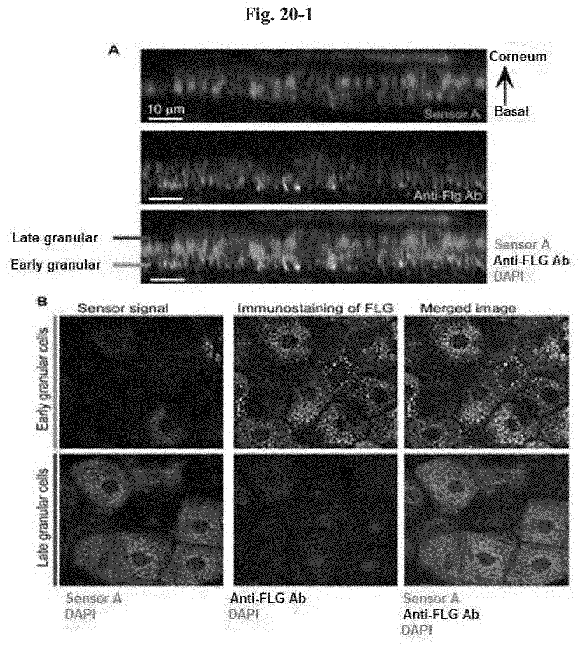

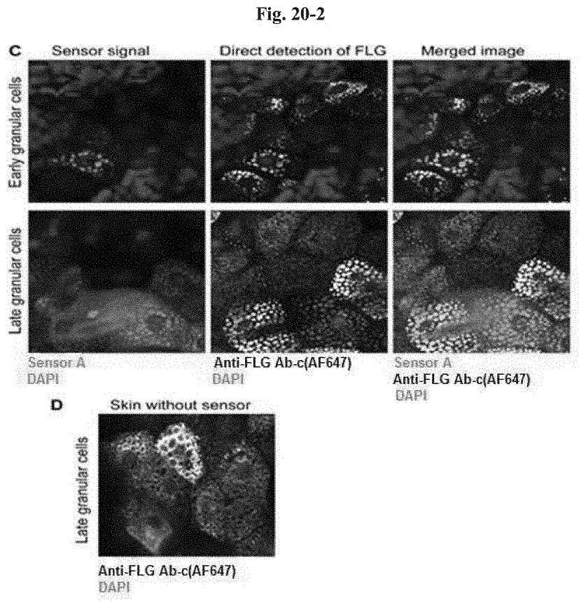

Once the skin barrier was fully mature (E18.5), doxycycline-fed embryos were subjected to live imaging and/or immunofluorescence microscopy. Sagittal confocal views revealed that bright sensor signal was confined to filaggrin-expressing granular layers, while planar views showed a robust array of sensor-labeled KGs in these cells (FIG. 19A). Moreover, and in marked contrast to conventional anti-filaggrin antibodies (35), the sensors penetrated even the large granules of the most mature (late) granular layer (FIG. 20).

The striking level of KG crowding seemed incompatible with liquid-like behavior. To gain further insights, we performed live imaging and monitored keratinocyte flux through the granular layers of skin (36). Early granular cells displayed only a few KGs, whose numbers appeared to increase through de novo granule formation (FIG. 19B). Over a half day of imaging, occasional fusions that resolved within minutes pointed to liquid-like behavior (FIG. 21). Moreover, whether Sensor A or B, signal recovery was rapid after photobleaching KGs within the mid-granular layer, further underscoring the liquid-like behavior of these endogenous KGs (FIG. 19C-D).

Despite these liquid-like features, most existing KGs grew robustly without undergoing fusion (FIG. 19E). Even KGs within the earliest granular layers in tissue exhibited liquid-like properties distinct from those of KG-like condensates that formed in HaCATs when transfected to express tagged-filaggrin (FIG. 19F). Moreover, when the sensor's nuclear export signal was removed and sensor FRAP half-life was measured within nucleoli, only filaggrin-containing KGs in tissue appeared to be relatively more viscous than the nucleolus in keratinocytes.

Probing deeper, we noticed that granular cells exhibited substantial morphological changes as they transited through the granular layers and became increasingly crowded with KGs (FIG. 19G). Correspondingly, photobleaching these KGs within early, middle and late granular cells revealed a gradual reduction in sensor dynamics as cells moved towards the skin surface. This remarkable increase in relative viscosity of skin KGs was also seen in stratifying cultures of primary human epidermal keratinocytes, which unlike immortalized keratinocytes, formed endogenous KGs that were of similar size and displayed similar liquid-like dynamics to KGs in the early- and mid-granular layers of mouse epidermis (FIG. 19H). Thus, despite pronounced species-specific divergence in filaggrin sequence, the preservation of KG's finely-tuned liquid-like behavior pointed to an underlying physiological relevance.

Stabilization of Liquid-Like Membraneless Organelles

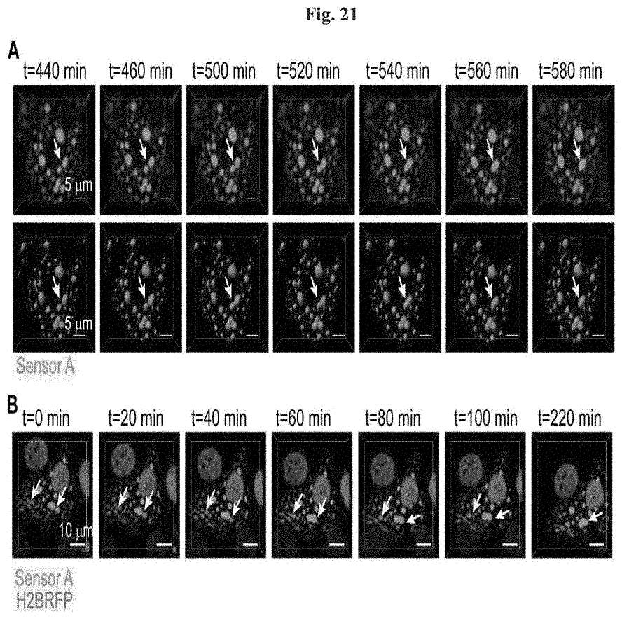

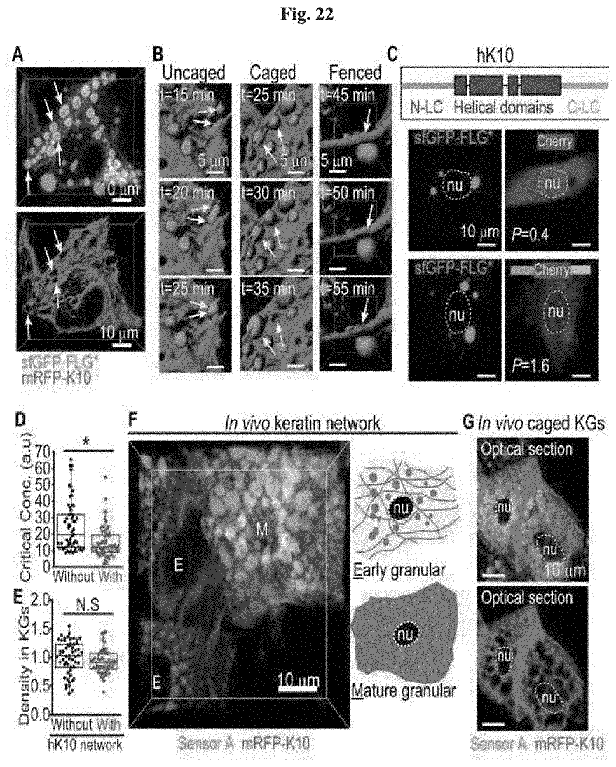

Although the rarity of fusion events among densely packed KGs might simply reflect their apparent viscosity, it was also possible that additional facets of terminal differentiation might be contributing to this puzzling behavior. Notably, the granular layer also displays an abundant network of terminal differentiation-specific keratins 1/10 (K1/K10) filaments, prompting us to test whether they might be impeding KGs from fusing and allowing them to crowd the cytoplasm as stable organelles. When HaCATs were transduced with doxycline-inducible human mRFP-K10 (TABLE 4), hK10 incorporated into the endogenous network of basal K5/K14 filaments (37). Upon co-transfection with sfGFP-FLG to drive KG formation, many of the mRFP-tagged keratin bundles encased KGs (FIG. 22A). Live imaging showed that these KGs spent prolonged periods of seemingly inert activity. Interestingly however, in regions where these KGs dislodged from filaments and became uncaged, KGs were mobile and frequently fused with other sfGFP-tagged KGs (FIG. 22B), shedding light on previous perplexing observations that unusually large KGs occur upon genetic ablation of Krt10 in mouse skin (38).

TABLE 4
Sequence information for K10-related sequences.
The underlined protein sequence is also
underlined in the name of the construct to
facilitate the identification of domains.

| Construct | Sequence |
|---|---|
| mRFP1-K10 | MASSEDVIKEFMRFKVRMEGSVNGHEFEIEGEGEGRPYEGTQTAKLKVTK |
| (SEQ ID NO: 30) | GGPLPFAWDILSPQFQYGSKAYVKHPADIPDYLKLSFPEGFKWERVMNFE |
| | DGGVVTVTQDSSLQDGEFIYKVKLRGTNFPSDGPVMQKKTMGWEASTER |
| | MYPEDGALKGEIKMRLKLKDGGHYDAEVKTTYMAKKPVQLPGAYKTDI |
| | KLDITSHNEDYTIVEQYERAEGRHSTGASGLELLEDLTLGRSDGSVRYSSS |
| | KHYSSSRSGGGGGGGGCGGGGGVSSLRISSSKGSLGGGFSSGGFSGGSFSR |
| | GSSGGGCFGGSSGGYGGLGGFGGGSFRGSYGSSSFGGSYGGSFGGGSFGG |
| | GSFGGGSFGGGGFGGGGFGGGFGGGFGGDGGLLSGNEKVTMQNLNDRLA |
| | SYLDKVRALEESNYELEGKIKEWYEKHGNSHQGEPRDYSKYYKTIDDLKN |
| | QILNLTTDNANILLQIDNARLAADDFRLKYENEVALRQSVEADINGLRRVL |
| | DELTLTKADLEMQIESLTEELAYLKKNHEEEMKDLRNVSTGDVNVEMNA |
| | APGVDLTQLLNNMRSQYEQLAEQNRKDAEAWFNEKSKELTTEIDNNIEQI |
| | SSYKSEITELRRNVQALEIELQSQLALKQSLEASLAETEGRYCVQLSQIQAQ |
| | ISALEEQLQQIRAETECQNTEYQQLLDIKIRLENEIQTYRSLLEGEGSGSSGG |
| | GGRGGGSFGGGYGGGSSGGGSSGGGHGGGHGGSSGGGYGGGSSGGGSSG |
| | GGYGGGSSSGGHGGSSSGGYGGGSSGGGGGGYGGGSSGGGSSSGGGYGG |
| | GSSSGGHKSSSSGSVGESSSKGPRY |
| N-LC(K10)- | MSVRYSSSKHYSSSRSGGGGGGGGCGGGGGVSSLRISSSKGSLGGGFSSGG |
| mCherry | FSGGSFSRGSSGGGCFGGSSGGYGGLGGFGGGSFRGSYGSSSFGGSYGGIF |
| (SEQ ID NO: 31) | GGGSFGGGSFGGGSFGGGGFGGGGFGGGFGGGFGGDGGLLSGNSPGVSK |
| | GEEDNMAIIKEFMRFKVHMEGSVNGHEFEIEGEGEGRPYEGTQTAKLKVT |
| | KGGPLPFAWDILSPQFMYGSKAYVKHPADIPDYLKLSFPEGFKWERVMNF |
| | EDGGVVTVTQDSSLQDGEFIYKVKLRGTNFPSDGPVMQKKTMGWEASSE |
| | RMYPEDGALKGEIKQRLKLKDGGHYDAEVKTTYKAKKPVQLPGAYNVNI |
| | KLDITSHNEDYTIVEQYERAEGRHSTGGMDELYKGS |
| mCherry-C- | MSVRYSSSKHYSSSRSGGGGGGGGCGGGGGVSSLRISSSKGSLGGGFSSGG |
| LC(K10) | FSGGSFSRGSSGGGCFGGSSGGYGGLGGFGGGSFRGSYGSSSFGGSYGGIF |
| (SEQ ID NO: 32) | GGGSFGGGSFGGGSFGGGGFGGGGFGGGFGGGFGGDGGLLSGNSPGVSK |
| | GEEDNMAIIKEFMRFKVHMEGSVNGHEFEIEGEGEGRPYEGTQTAKLKVT |
| | KGGPLPFAWDILSPQFMYGSKAYVKHPADIPDYLKLSFPEGFKWERVMNF |
| | EDGGVVTVTQDSSLQDGEFIYKVKLRGTNFPSDGPVMQKKTMGWEASSE |
| | RMYPEDGALKGEIKQRLKLKDGGHYDAEVKTTYKAKKPVQLPGAYNVNI |
| | KLDITSHNEDYTIVEQYERAEGRHSTGGMDELYKGSGSSGGGGRGGGSFG |
| | GGYGGGSSGGGSSGGGHGGGHGGSSGGGYGGGSSGGGSSGGGYGGGSSS |
| | GGHGGSSSGGYGGGSSGGGGGGYGGGSSGGGSSSGGGYGGGSSSGGHKS |
| | SSSGSVGESSSKGPRY |
| mCherry | MVSKGEEDNMAIIKEFMRFKVHMEGSVNGHEFEIEGEGEGRPYEGTQTAK |
| (SEQ ID NO: 33) | LKVTKGGPLPFAWDILSPQFMYGSKAYVKHPADIPDYLKLSFPEGFKWER |
| | VMNFEDGGVVTVTQDSSLQDGEFIYKVKLRGTNFPSDGPVMQKKTMGWE |
| | ASSERMYPEDGALKGEIKQRLKLKDGGHYDAEVKTTYKAKKPVQLPGAY |
| | NVNIKLDITSHNEDYTIVEQYERAEGRHSTGGMDELYKSPG |

Keratins possess a central coiled-coil ‘rod’ domain that initiates heterodimer formation and forms the backbone of the 10 nm intermediate filament (39). Whereas K5/K14 keratins of proliferative progenitors have short amino- and carboxy-LC domains, the large LC domains of K1/K10 keratins (40) are thought to protrude along the outer surface of the filament and bundle into cable-like filaments.

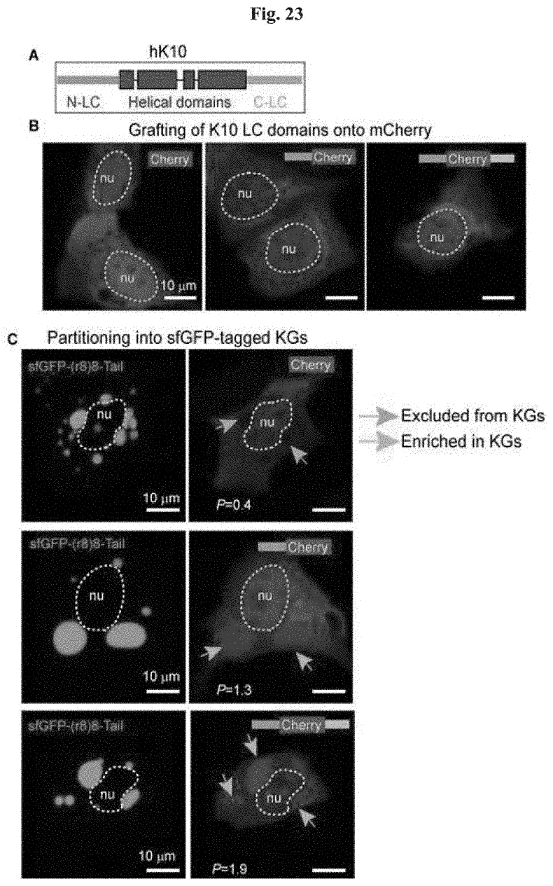

Intrigued by the packing of K10-containing filaments around filaggrin granules, we next asked whether their unique features might facilitate interactions with KGs. Examining the behaviors of mCherry fused to one, both or neither hK10 LC domains (TABLE 4), we found that in the absence of KGs, each was diffuse in the cytoplasm of cultured keratinocytes (FIG. 22C and FIG. 23). In contrast, when sfGFP-tagged filaggrin and its KGs were present, mCherry was excluded from KGs, while both mCherry constructs with hK10 LC domains partially partitioned into KGs. Moreover, the critical concentration for phase separation of sfGFP-tagged filaggrin was reduced in the presence of the K10 keratin network without altering FLG density within KGs (FIGS. 22D-E). These results suggested that weak interactions between KGs and the LC domains of terminal differentiation-associated keratins may promote the caging and stabilization of KGs in skin.

To further explore this possibility, we transduced both our phase separation sensor and suprabasal-inducible mRFP-hK10 constructs into E9.5 embryos and performed live-imaging on E18.5 skin explants. While early granular cells displayed small, relatively sparse KGs surrounded by a well-defined network of K10-containing filaments, mid-granular cells exhibited a denser keratin network interwoven among larger, more abundant KGs that remained caged and hence unable to fuse (FIGS. 22F and 22G). Our findings suggest a model whereby reciprocal density-dependent interactions between LC domains of terminal differentiation-specific keratins and KGs structure the cytoplasm to form an elaborate, interwoven network of stabilized liquid-like KGs and keratin filament bundles.

Liquid Phase KG Dynamics, Enucleation and Environmental Sensitivity

We posited that progressive crowding by keratin-stabilized KGs might distort the nucleus and other organelles in a fashion that could contribute to their destruction at the critical granular to stratum corneum transition. If so, this could explain why nuclei are often aberrantly retained in the outer skin layers of patients who also lack KGs (41).

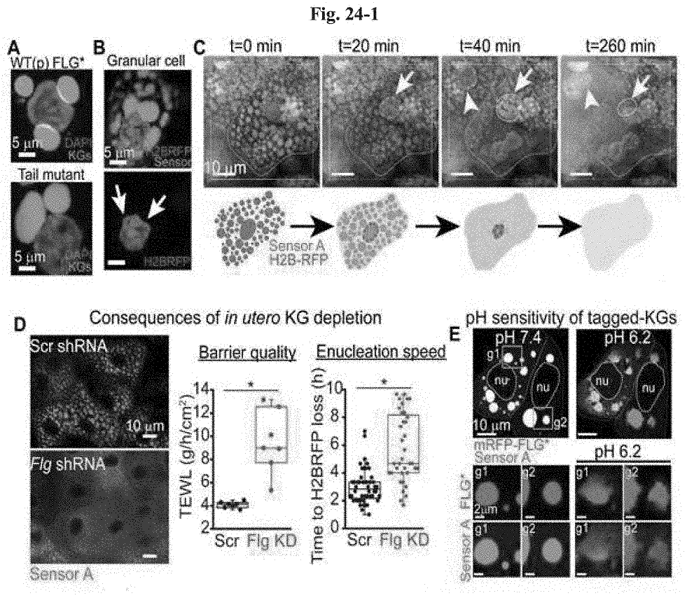

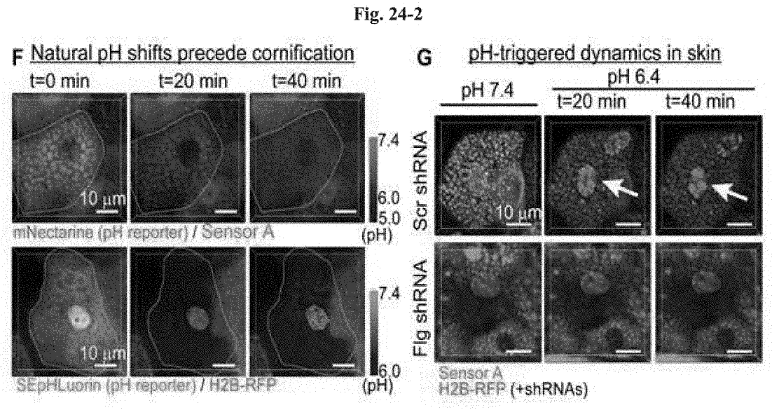

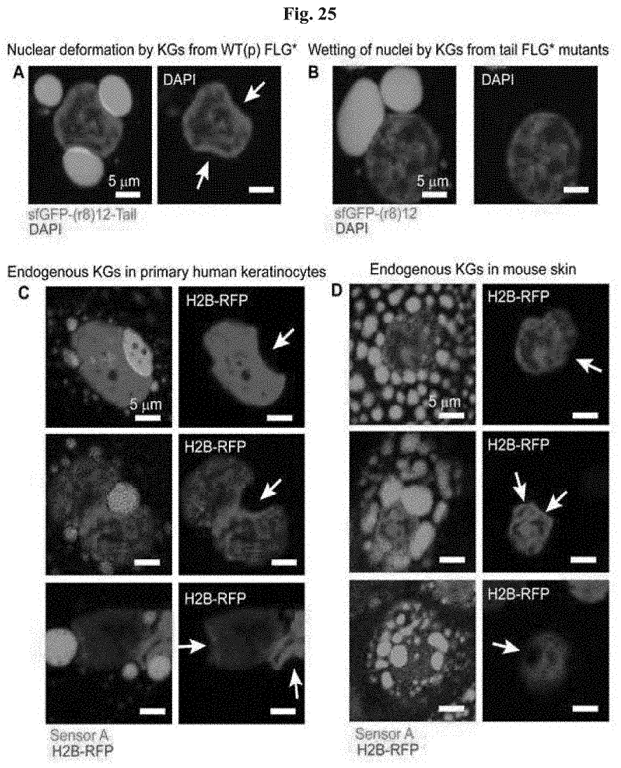

Consistent with this notion, KGs assembled de novo in HaCATs from wild-type repeat filaggrins prominently deformed nuclei, while KGs assembled from disease-associated FLG mutants instead wetted the nuclear surface without deformation (FIG. 24A and FIG. 25A-B). Endogenous KGs in primary human keratinocytes also induced prominent nuclear deformation (FIG. 25C). Similarly, when we transduced embryos with H2B-RFP and our sensors, and monitored the progression of granular cells to the stratum corneum by live imaging, we found that mature granular cell nuclei were markedly deformed as KG density increased in skin (FIG. 24B and FIG. 25D).

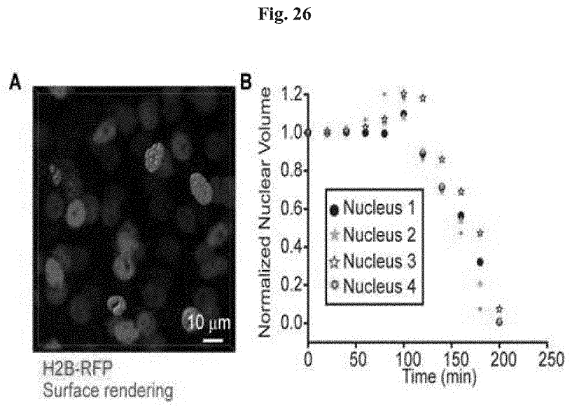

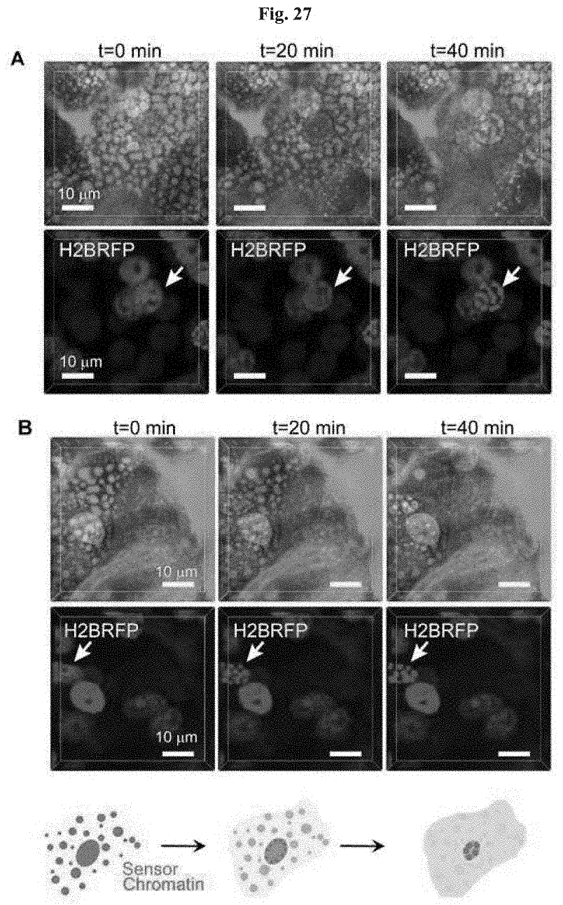

Enucleation events were difficult to capture, as the process was remarkably rapid, occurring over 2 hours (FIG. 26). Interestingly, they were always preceded by chromatin compaction and then chromatin loss/nuclear destruction (FIG. 24C and data not shown). Moreover, just as chromatin began to show signs of compaction, a marked change in the material properties of KGs occurred, reflected by a shift in the phase sensor's localization from a granular-like state to being diffuse in the cytoplasm (FIG. 24C, FIG. 27). These phenomena coincided with the loss of KGs, as granular cells transitioned to squames.

Probing further, we found that Flg knockdown in skin not only depleted KGs, but also delayed the nuclear degradation process (FIG. 24D). This was accompanied by increased transepidermal water loss (TEWL) through the skin barrier. These data suggested that KGs accelerate loss of membrane-bound organelles, an essential feature of the skin barrier.

Given the inherent environmental responsiveness of intrinsically disordered proteins (42, 43), we wondered whether the marked shift in KG dynamics late in terminal differentiation might be fueled by the environmental changes that naturally occur near or at the skin surface. In particular, while proliferative basal progenitors experience physiological pH (7.4), the skin surface is acidic (pH-5.5) (44). Since filaggrin is rich in histidine, whose physiological pKa is ˜6.1 (45), we posited that this natural difference in extracellular pH may also reflect intracellularly and in part be triggering the KG changes in material properties that we had detected at the granular to stratum corneum transition.

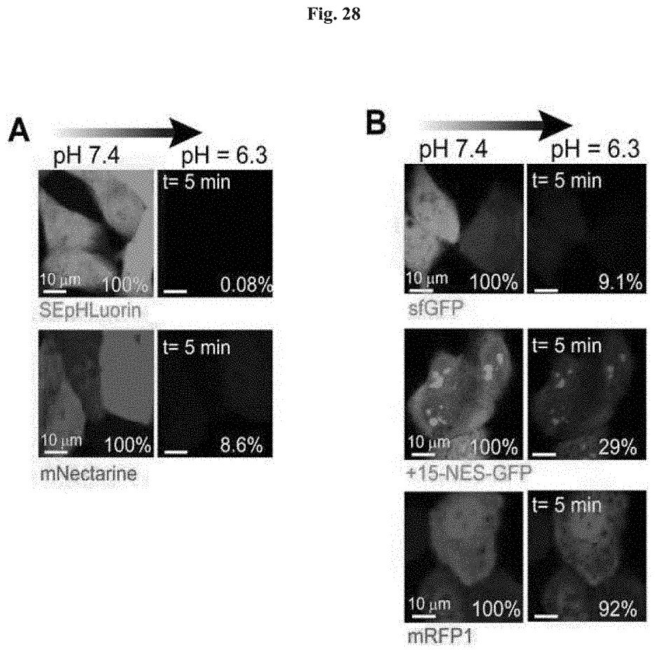

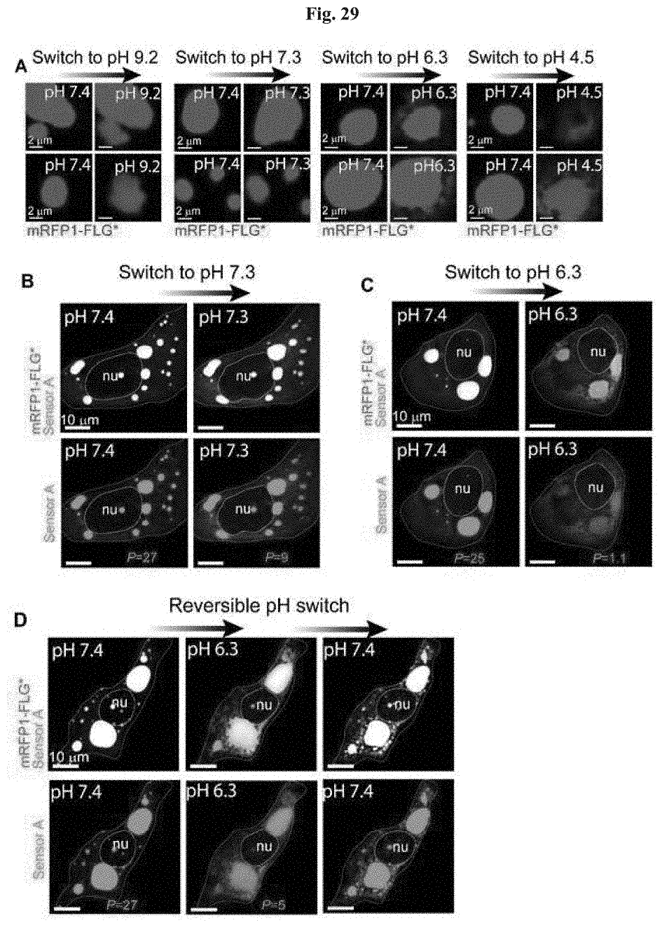

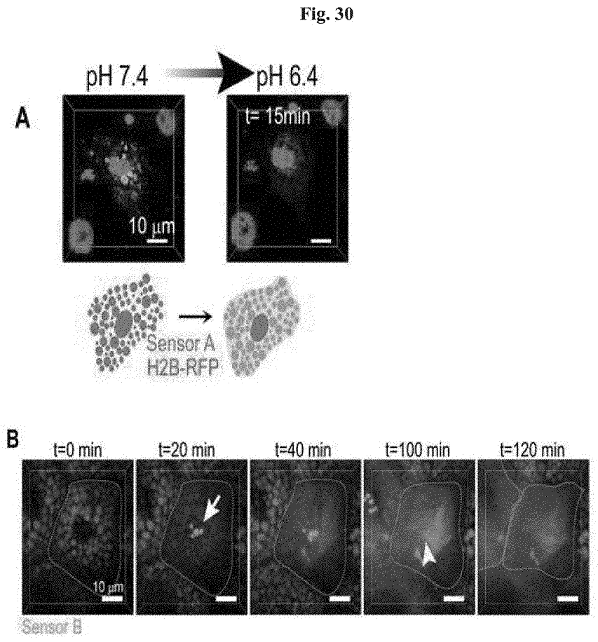

To detect intracellular pH shifts, we first transduced HaCATs with either mNectarine or SEpHlourin reporters, which rapidly lose fluorescence upon shifting from pH7.4 to pH6.3 (FIG. 28). Interestingly, when the extracellular pH was decreased to elicit an intracellular pH shift from 7.4 to ˜6.2-6.5, de novo-assembled KGs in HaCATs changed profoundly. As revealed by live imaging, both filaggrin and the sensor in particular displayed increased cytoplasmic and diminished KG-like localization concomitant with this intracellular pH shift (FIG. 24E and FIG. 29). Similar changes were seen in endogenous KGs of differentiated primary human epidermal keratinocytes when they experienced this pH shift (FIG. 30).

Given the pH sensitivity of KGs, we then turned to interrogating this process in vivo. To do so, we introduced our pH reporters into mice along with either our phase separation sensor or H2BRFP and through live imaging, monitored the natural intracellular pH shifts that we surmised would occur as granular cells approached the acidic skin surface. Over time, as each granular cell progressed to the critical granular-to-corneum transition, it experienced a sudden shift in pH, as detected by our intracellular reporters (FIG. 24F). This rapid endogenous pH shift invariably coincided with the initiation of KG dissolution (top panels) and coincided or immediately preceded an increase in chromatin compaction (bottom panels). Moreover, within 2 hr of KG dissolution and nuclear compaction, imaged granular cells within the epidermis had undergone morphological changes characteristic of enucleation and squame formation (FIG. 30B and data not shown).

Finally, we took skin explants from embryos transduced with phase-sensor, H2BRFP and either scrambled or filaggrin shRNAs, and performed live imaging immediately after shifting the extracellular pH in the medium. Accelerating the natural intracellular pH transition, granular cell KGs showed signs of disassembly, and chromatin compaction became pronounced (FIG. 24G, top panels). Intriguingly and by contrast, this pH shift did not trigger chromatin compaction in skin devoid of KGs (FIG. 24G, bottom panels). These data suggested that the pH-shift functioned specifically in altering the material properties of histidine-rich KGs, which in turn promoted chromatin compaction, enucleation and skin barrier establishment. The results further suggest that enucleation events in skin are likely driven by a combination of nuclear deformation and pH-driven release of as yet undetermined KG components.

Discussion

Our design and deployment of a new class of innocuous client protein provides a general strategy to interrogate endogenous liquid-liquid phase separation dynamics across biological systems in a non-disruptive manner. We envision that these in vivo phase separation sensors may be further functionalized to incorporate enzymes evolved for proximity proteomics (46) (47), potentially enabling—without perturbing endogenous scaffold proteins—the molecular and biophysical interrogation of endogenous liquid-liquid phase separation in organoids, tissues and living organisms.

We used this new strategy to illuminate—through the lens of phase separation—the process of skin barrier formation, which entails the appearance of enigmatic KGs in the granular layer and then their sudden disappearance as epidermal cells undergo a poorly-understood transition to the stratum corneum. These granules, long puzzling the field (48), have been viewed as inert, cytoplasmic aggregates of filaggrin, which then become cleaved into smaller fragments and amino acid derivatives that promote keratin filament bundling (21) and hydrate the stratum corneum (24). Despite decades of research, and mutations linked to atopic dermatitis (23), no clear function had been established for KGs, filaggrin or filaggrin paralogs that also accumulate as granular deposits in epithelial tissues (49, 50).

Through the engineering of filaggrins and filaggrin disease-associated variants and also our phase separation sensors, we have now shown that KGs are abundant, liquid-like membraneless organelles, which through their phase-separation-driven assembly and then disassembly, function to structure the cytoplasm and drive an environmentally sensitive program of terminal differentiation in the epidermis. By virtue of their newfound mechanical and pH-sensitive properties, KGs are ideally equipped to confer environmental-responsiveness to the rapid and adaptive process of skin barrier formation. The discovery that filaggrin-truncating mutations and loss of KGs are rooted in altered phase-separation dynamics begins to shed light on why associated skin barrier disorders are exacerbated by environmental extremes. These insights open the potential for targeting phase behavior to therapeutically treat disorders of the skin's barrier.

Liquid-phase condensates have been typically viewed as reaction centers where select components (clients) become enriched for processing or storage within cells (2). Analogously, KGs may store clients, possibly proteolytic enzymes and nucleases, that are timely (in a pH-dependent fashion) and rapidly released to promote the self-destructive phase of forming the skin barrier. Additionally, squame formation likely exploits general biophysical consequences of KG assembly, as KGs interspersed by keratin filament bundles massively crowd the keratinocyte cytoplasm and physically distort adjacent organelles prior to the ensuing environmental stimuli that trigger KG disassembly. Overall, the remarkable environmentally-sensitive dynamics of liquid-like KGs, actionable by the skin's varied environmental exposures, expose the epidermis as a tissue driven by phase separation.

Materials and Methods

Sequence Analysis of Filaggrin and its Paralogs

Full proteomes were downloaded as FASTA files from UniProt (uniprot.org). For the human proteome, we used the (non-redundant) canonical proteome. For the analysis of protein domains known to drive liquid-liquid phase separation (FIG. 4), we downloaded the complete set (>100) of proteins from the PhaSEPro database (51) and formatted them as a FASTA file. We implemented a simple Script in MATLAB R2016a (available upon request) to extract protein size, amino acid abundance and Arg-bias of all annotated proteins. Protein size values were plotted as a histogram in FIG. 3D and as a box plot in FIG. 1E. Except for human Flg (and its paralogs), most mammalian Flg and Flg paralogs in mammals remain poorly annotated or poorly sequenced in publically available genome and protein databases. TABLE 1 shows the sequences that we used as input material and details of their manual curation. Any gene assembly (manually curated) is available upon request. To calculate relevant sequence parameters (length, amino acid composition, hydropathy, Arg-bias) in FLG and its paralogs across species, we implemented a simple Script in MATLAB R2016a, but we note that any of these sequence parameters can be calculated using common online tools (web.expasy.org/protparam). Arginine-bias (Arg-bias) was calculated as the total number of arginine residues relative to the total number of positively charged residues (R+K). Hydropathy in FIG. 4 corresponds to the average hydropathy across all residues in a protein, using the Kyte-Doolittle's scale (52). To characterize non-synonymous mutations in human filaggrin (FIG. 15, we downloaded known SNPs in the human Flg gene (not annotated in ClinVar to avoid variants associated with clinical phenotypes) from NIH's dbSNP database (ncbi.nlm.nih.gov/snp/) and from the GnomAD browser (gnomad.broadinstitute.org/gene/ENSG00000143631). We used custom-made MATLAB scripts to filter for unique SNPs corresponding to non-synonymous mutations. At the time of our analysis, we annotated 3743 non-synonymous Flg SNPs in dsSNP. This script also calculated the overall percentage of mutations assigned to each of the 20 naturally-occurring amino acid residues, as shown in FIG. 15. By generating 1000 unique Flg mutant genes through random single-nucleotide mutations in Flg cDNA, we also estimated the expected random mutational burden per residue. From the total SNPs, we identified 405 SNPs involving mutations of His codons. The script then identified the mutational landscape involving these SNPs and their corresponding nonsynonymous codons (encoding Asp, Leu, Asn, Pro, Gln, Arg and Tyr), as shown in FIG. 15 and including the expected random mutational profile.

Synthesis of Repetitive DNAs Encoding Filaggrin and Filaggrin Variants

To assemble repetitive DNAs, we used a highly-efficient iterative plasmid-reconstruction approach (PRe-RDL), with minor modifications. We used a modified pET-24a(+) vector as published (53), but eliminated the terminal Tyr-stop-stop sequence to avoid altering the hydropathy of FLG sequences. Instead, the modified vector uses a terminal Gly-stop-stop-stop sequence. We refer to this modified vector as JMD2G. We purchased synthetic gblocks from IDT (Integrated DNA Technologies) encoding the eight repeat in human FLG (repeat #8, here referred as r8), sfGFP, mRFP1 and the S100 domain of human FLG (FIG. 1B)—See TABLE 2 for the corresponding protein sequences. We chose r8 as this repeat is often duplicated in humans, yielding FLG variants with 11 (this is the most common of all human FLG variants) or 12 repeats. The specific choice of a repeat (among human FLG repeats 1 to 10), however, is otherwise trivial, as individual FLG repeats are nearly identical in sequence (with >90% sequence identity in humans and typically >99% in mice). We cloned the r8 block into JMD2G and performed iterative rounds of PRe-RDL to build genes with up to 16 concatemers of r8. These genes were then modified to generate variants with the C-terminal tail domain of human FLG (TABLE 2). DNA sequences were verified by Sanger sequencing (Genewiz, N.J.) whenever possible. For long repetitive DNAs beyond the reach of Sanger sequencing, to confirm proper concatamerization of sequence-verified domains, we relied on gene size (judged by conventional DNA gel electrophoresis) and subsequent validation of expected protein properties (size and diffusion properties) upon expression in E. coli or mammalian cells. For mammalian expression, we subcloned fully-assembled repeat genes into a modified pMAX vector (Amaxa). Briefly, we added a subcloning cassette with an NheI site downstream of the chimeric CMV promoter and an EcoRI site upstream of the SV40 polyA signal. We generated pmax vectors with N-terminal proteins (sfGFP, mRFP1, S100-sfGFP, S100-mRFP1) that included a C-terminal SPG linker encoded within an XmaI site. We readily shuffled genes between JMD2G vectors and our modified pmax vectors using XmaI and EcoRI for DNA restriction. See TABLE 2 for protein sequences for all constructs. Additionally, to build genes with nuclear reporters of FLG concentration in the cell, we further modified our pMAX-based genes encoding FLG repeats to replace the N-terminal fluorescent protein (flanked by NheI and XmaI sites) with genes fragments encoding H2BGFP-[p2a]-mRFP, H2BRFP-[p2a]-sfGFP or H2BGFP-[p2a]-S100-mRFP (see TABLE 2 for sequence details) via restriction with NheI and XmaI sites. [p2a] is a codon-optimized DNA sequence (GGAAGCGGAGCGACGAATTTTAGTCTACTGAAACAAGCGGGAGACGTGGAGGAAAACC CTGGACCT) (SEQ ID NO:34) that self-cleaves during translation and so enables the synthesis of two proteins from a single transcript (28). We also built pMAX vectors encoding H2BRFP-[p2a]-H2BGFP and H2BGFP-[p2a]-H2BRFP to validate the equimolar synthesis of individual [p2a]-linked proteins—our measured GFP:RFP ratio of fluorescent signal matches the expected relative brightness (ratio=3) of these two fluorescent proteins (see FIG. 8A).

Synthesis of Genes Related to Phase Separation Sensors

TABLE 3 includes the sequence information for all sensor domains reported in FIG. 14B. The rationale for the generation of these proteins is explained in detail in the Materials and Methods. Corresponding genes were synthesized by IDT as gblocks and cloned into modified pmax vectors as described above for genes encoding FLG variants. We purchased additional gblocks encoding previously published (see references in main manuscript) super-charged variants of sfGFP: +15GFP and −20GFP. All constructs, unless indicated, included an optimized short nuclear export signal (54) (LELLEDLTL) (SEQ ID NO: 35) as linker between the N-terminal fluorescent proteins and the sensor domain. To test the intrinsic phase separation propensity of individual sensor domains (FIG. 14B), we artificially enhanced their phase separation propensity by synthesizing variants with a C-terminal trimerization domain. To create these trimerization variants, we subcloned sensor domains into a pmax vector with an N-terminal sfGFP gene and a C-terminal trimerization domain that was flanked by BamHI and EcoRI sites. Genes encoding sensor domains were readily shuffled across these vectors using XmaI and BamHI sites. We generated vectors each with one of two trimerization domains: NC1 domain from human COL18A1 (P39060, Isoform 1, residues 1442-1496) (55) or a fibritin fragment from bacteriophage T4 (so-called foldon domain) (56).

Synthesis of Genes Encoding Human K10 and its Low Complexity Domains

Because of the large low complexity domains in human K10 we were unable to successfully amplify full-length KRT10 cDNA. Instead, we first PCR-amplified a fragment of KRT10 spanning the N-terminal LC domain and the complete central coiled-coil rod domain (forward primer: TAATCATCGATCGGATGGCTCTGTTCGATACAGCTCAAGCAAGCACTACTCTT (SEQ ID NO: 36), Reverse primer: TAAGCAGGGGATCCCTCTCCTTCTAGCAGGCTGCGGTAGGTTTG (SEQ ID NO: 55)) using KRT10 cDNA (NM_000421.2) purchased from Origene. These primers added restriction sites for PvuI and BamHI at the N and C-terminus, respectively, for seamless restriction into a pmax vector harboring an N-terminal mRFP sequence and the C-terminal LC domain. The C-terminal LC domain was synthesized as a gblock by IDT (by maximizing codon usage along the length of this highly repetitive low complexity sequence). Similarly, we also obtained a gblock encoding the N-terminal LC domain flanked by NheI and XmaI sites, which we inserted into our modified pmax vector for building a gene encoding a fusion to mCherry. This vector was further modified between BamHI and EcoRI sites to introduce the C-terminal LC domain and generate mCherry fusions harboring both K10 LC domains. These constructs are listed in TABLE 4.

Characterization of Filaggrin-Like Proteins in FIG. 1F-I and FIG. 9A-B

To drive efficient expression of the relevant repetitive proteins, we transfected the corresponding pmax plasmids into HaCATs, a commonly used immortalized human keratinocyte cell line (57). First, we routinely expanded HaCATs in low calcium (50 μM) epidermal cell culture media (58). For transfection, we seeded 1.5-2×10⁵ cells per well in glass-bottom 24-well plates (P24G-1.5-10-F, Mattek) using CnT-PR media (CELLnTEC, Switzerland) supplemented with 10% epidermal media. At this seeding density cells cover the glass-bottom wells at good cell density by 15-17 h post seeding. At this time, we typically transfected cells with 0.5 μg to 3.5 μg of each plasmid, using lipofectamine 3000 (Invitrogen) and following the instructions of the manufacturer (at 1.5 μl L3000 per transfection reaction, including P3000 reagent). In a typical experiment with plasmids encoding FLG repeat variants, we scaled the amount of plasmid DNA to account for differences in gene size (e.g. 1.5 μg for a gene with 4 FLG repeats compared with 3.45 μg for a gene with 12 FLG repeats). However, as little as 0.5 μg was sufficient to induce expression of most FLG variants in TABLE 2 and we have conducted experiments in which we transfected the same amount of plasmid DNA regardless of gene size and saw the reported behaviors. We note that the amount of plasmid DNA ultimately controls transfection efficiency, that is the number of cells that robustly express the plasmid of interest, but does not play a role in defining the properties of the resulting FLG granules. One day after transfection, we changed media to a pro-differentiation media (pre-warmed to 37° C.), CnT-PR-D (CELLnTEC, Swtizerland) supplemented with 1.5 mM CaCl₂, and proceeded to image cells 6-9 h later using a spinning-disc microscope equipped with a 40× oil objective. Live imaging was conducted with cells at 37° C. and under a controlled C0₂ environment. In some specific cases and as indicated (e.g. FIG. 7A and FIG. 24A), we fixed cells, with 4% paraformaldehyde at 37° C. for 10 min, for subsequent DAPI staining and imaging. Generally, however, we show live imaging data (i.e. without fixation).

To calculate the phase separation propensity of FLG repeat proteins in FIG. 1, we operationally defined it as the percent of total (background-corrected) fluorescent signal residing within phase-separated granules and based on maximum intensity projections of live imaging data using ImageJ. In FIG. 1F we exclusively consider cells at a fixed concentration of FLG repeat variants (readily assessed by the nuclear H2BGFP signal that is equimolar to the FLG repeat proteins). In FIG. 1G, we then measured and plotted phase separation propensity for each FLG variant across the entire range of accessed expression levels. Concentration values for FLG variants were determined from the nuclear H2B reporter signal (adjusted by total cell area) in each cell to sensitively measure protein concentration even at low expression levels when FLG proteins are diffuse. Whenever we observed signs of a phase transition in our data (i.e. a concentration-dependent increase in phase separation propensity), we applied a logistic fit [y=(−100/(1+(x/x₀)\hat{ }P))+100, as expected for a phase transition] to those data using OriginPro. Using these fits (with typical adjust R²>0.9), we then approximated the critical concentration for phase separation as the EC50 of the logistic fit, that is the concentration at which most cells reach a phase separation propensity of 50%—wherein the total number of molecules in the dilute phase equals the number of molecules in the high concentration density phase. As explained in FIG. 8, while phase separation happens with a given (low) probability below the EC50 (as can be seen in our data in FIG. 1G and FIG. 8), the concentration fluctuations that potently drive phase separation near the true critical concentration of the system become dominant near the EC50, which justifies its definition as an experimental approximation to the critical value.

To calculate the density of FLG repeat within granules assembled from different FLG repeat variants (FIG. 7B), we first normalized average fluorescence within each granule to the average fluorescence of all granules assembled by mRFP1-(r8)4, which were the brightest granules. To transform changes in normalized fluorescence intensity into changes in relative density of r8 molecules within the granule, we simply scaled these data by a factor equal to the number of FLG repeats per construct divided by 4. For instance, because a single mRFP1-(r8)12 molecule carries 3 times more r8 units than a single molecule of mRFP1-(r8)4, the normalized fluorescence intensity of granules assembled from mRFP1-(r8)12 (FIG. 9A) was scaled by a factor of 3.

To study protein dynamics within granules (FIG. 9A-B), we photobleached circular regions of interest (0.54 μm in diameter) at the center of granules and imaged the process of recovery at the highest possible speed under our standard imaging conditions (200 ms intervals). For data analysis, we normalized the background-corrected fluorescence within the region of interest to the background-corrected average granule fluorescence prior to photobleaching and then corrected for loss of fluorescence in the granule area outside of the region of interest throughout the imaging process. To calculate recovery half-lives, we fitted the post-bleaching normalized data using OriginPro and a standard exponential growth curve: f=A(1−e\hat{ }(−xt)). Half-lives (time when f=0.5) were estimated as Ln(0.5)/(−t).

Characterization of Phase Separation Sensors in FIG. 14D-G

The approach to studying the behavior of phase separation sensors in HaCATs (FIG. 14D-G) is similar to the approach described for the study of FLG variants (FIG. 9A-B). In addition to vectors harboring tagged-FLG proteins, the transfection mixture included a pmax vector encoding Sensor A (at 200 ng per reaction) (see sequence information in TABLE 3). For experiments in FIG. S16A-B, we performed similar co-transfection experiments but used the sensor variants (TABLE 3) and granule-forming proteins indicated in the figure legend.

Photobleaching experiments were also as described for experiments in FIG. 9. For each measurement, we first obtained photobleaching data for the mRFP1-tagged FLG protein. We then obtained photobleaching data for (+15GFP-tagged) Sensor A in the same granule. Sensor data was processed and analyzed in the same manner as previously described for tagged-FLG proteins.

Atomic Force Microscopy (AFM) Measurements

To enable access of the AFM probe to filaggrin granules within cells, we seeded 1.5×10⁶ HaCATs into 50 mm glass-bottom dishes (Fluorodish, FD5040, WPI) using CnT-PR media supplemented with 10% epidermal media. At 15 h post seeding, we transfected HaCATs with a mixture of two pmax vectors using lipofectamine 3000 (at 7.5 μl L3000 per transfection reaction, including P3000 reagent). One vector (at 1 ug per reaction) harbored a H2B-RFP gene and was common to all transfection reactions. The second vector (at 7.5 μg per reaction) encoded one of the following FLG variants: sfGFP-(r8)8, sfGFP-(r8)8-Tail and S100-sfGFP-(r8)8-Tail (TABLE 2). One day after transfection, we washed cultures with DPBS (pre-warmed to 37° C.) and added pro-differentiation media (pre-warmed to 37° C.), CnT-PR-D (CELLnTEC, Swtizerland) supplemented with to 1.5 mM CaCl₂. Cells were transported (at 37° C.) soon after or up to 24 h later to the Molecular Cytology core facility of MSKCC for AFM measurements using a microscope stage at 37° C. AFM force measurements and manual deformations of sfGFP-tagged FLG granules were performed using an MFP-3D AFM (Asylum Research) combined with an Axio Scope inverted optical microscope (Zeiss). Silicon nitride probes with a 5 μm diameter spherical tip were used (Novascan). Cantilever spring constants were measured prior to sample analysis using the thermal fluctuation method, with nominal values of approximately 100 pN/nm. 5×5 μm force maps were acquired with 10 force points per axial dimension (0.5 μm spacing) atop sfGFP-tagged FLG granules identified using the bright-field and GFP optical images. Measurements were made using a cantilever deflection set point of 10 nN and scan rate of 1 Hz. Bright-field (AFM probe), GFP (FLG variant) and H2B-RFP (nuclei) images were acquired for each cell and granule measured to enable force map and optical image co-registration. Live-video bright-field images were also taken during force map acquisition to observe granule and cellular deformations. Force-indentation curves were analyzed using a modified Hertz model for the contact mechanics of spherical elastic bodies. The sample Poisson's ratio was 0.33 and a power law of 1.5 was used to model tip geometry. To observe granule displacement and flow following force application, the AFM tip was manually placed adjacent to sfGFP-tagged FLG granules using a micrometer. During live video-rate (14 frames/sec) image acquisition (bright-field and GFP), force was manually applied with the AFM probe in the absence of force set point feedback via micrometer manipulation.

Mice and Lentiviral Transduction

Mice were housed and cared for in an AAALAC-accredited facility, and all animal experiments were conducted in accordance with IACUC-approved protocols. We obtained the hIVL-rtTA FVB mouse line (ref. 27 of the main manuscript) from NIH as frozen embryos donated by Julie Segre at NHGRI. This line was genotyped as originally published.

For rapid generation of mice with genetically-modified skin, we used non-invasive, ultrasound-guided in utero lentiviral-mediated delivery of expression constructs and shRNAs, which selectively transduces single-layered surface ectoderm of living E9.5 mouse embryos as previously published (59). Lentiviral vectors with Scramble (not targeting) shRNAs and constitutive expression (PGK-driven) of H2B-RFP were previously reported and documented to have no adverse effects over no infection or a mock vector having no shRNA (59). We further modified this PLKO-based lentiviral vectors to replace the PGK promoter with a TRE promoter sequence (TRE3G, Clontech). We assembled the corresponding TRE repeats from small oligos synthesized by IDT and cloned them into this modified PLKO vector. PKG and TRE-based PLKO vectors were modified to include NheI and EcoRI sites downstream of the promoter. We shuffled sensor and mRFP-K10 genes from pmax vectors into PLKO-based vectors using these two restriction sites. Using these PLKO vectors we generated high titer viruses in 293FT cells as previously described (59). To induce expression of TRE-controlled genes in vivo, females fostering lentivirally-transduced embryos were fed with doxycycline starting 1 day after injection (i.e. at E10.5).

For knockdown of mouse filaggrin, we relied on our curated mouse Flg cDNA (largely based on the C57B6 mm10 genome) to identify hairpins with high intrinsic scores and no predicted off-targets using the GPP Web Portal (portals.broadinstitute.org/gpp/public). Notably, existing genome-wide shRNA libraries lack hairpins against filaggrin, likely due to the low quality of the current reference sequence and the inherent repetitiveness of most target sites. We modified our lentiviral vectors harboring H2BRFP to substitute their Scramble shRNA with two hairpins against (mouse) Flg (#01: with target sequence ATCAATCTCACAGCTATTATT (SEQ ID NO: 37) localized to the C-terminal domain, and #02: with target sequence CTCCGGATTCTACCCAGTATA (SEQ ID NO: 38) within the filaggrin repeats). We tested both in mouse skin and they efficiently depleted mFLG and its KGs (FIG. 24 shows data for hairpin #02)

To transduce human primary keratinocytes with lentiviral vectors harboring H2BRFP and phase separation sensors, we thawed frozen neonatal and adult human primary keratinocytes (i.e. two different human donors) that we purchased from Life Technologies. Expanded only for 1 passage prior to freezing, thawed cells were seeded into a 10 cm cell culture dish using Epilife media (ThermoFisher) supplemented with Human Keratinocyte Growth Supplement (ThermoFisher). At ˜80% confluency, cells were detached using StemPro Accutase (ThermoFisher) and seeded onto 6-well plates at a density of 2×10⁵ or 4×10⁵ per well. After overnight incubation, we transduced these cultures by diluting the corresponding high titer lentiviruses into supplemented Epilife media and centrifuging the 6-well plates at 1100 g for 30 min at 25-29° C. We discarded the media and added fresh media as needed as the cells expanded. Upon confluency, we detached the transduced cells using StemPro Accutase and seeded them in glass-bottom 35 mm dishes (MatTek, P35G-1.5-20-C) at about half the original cell density using CnT-PR media supplemented with 10% supplemented Epilife. Once confluent, we switched these cultures to pro-differentiation media (same as for HaCATs) and incubated them for at least 4 to 8 days prior to live imaging. We note that a similar in vitro lentiviral transduction approach was used to generate HaCATs with doxycycline-inducible expression of mRFP-K10.

Live Imaging

For live imaging of mouse skin, we harvested head and back skin from E18.5 mouse embryos that were in utero transduced as explained above. We predominantly imaged head skin as our lentiviral approach provides very high coverage in the head, though at high titer we could also routinely use back skin. Once removed from the embryos, we gently scraped off the fat leaving the dermis intact, cut out 1 cm² pieces and placed them with the stratum corneum facing down on a glass-bottom 35 mm dish (MatTek, P35G-1.5-20-C) and on top of a 50 μl drop of Phenol-red free grow-factor reduced matrigel (Corning). We pressed the tissue flat against the glass surface using a transparent porous membrane (Whatman Nuclepore Track-Etched Membranes, 13 mm diameter, 5 μm pores) and a 12 mm cover glass (Fisherbrand). Once flat, we removed the cover glass and allowed the matrigel to solidify at 37° C. for 15 min. We then added 2 ml of warm (at 37° C.) CnT-Prime Airlift, Full Thickness Skin Airlift Medium (from CELLnTEC), typically supplemented with 2 μg/ml doxycycline (unless all genes were under constitutive promoters). We imaged these samples using a spinning-disc microscope equipped with a 40× oil objective and a live imaging chamber with constant supply of CO₂ and maintained at 37° C. We imaged with up to two lasers (488 and 561 nm, at 5.2 mW) and with exposure times of 200 ms per laser. We obtained full z stacks of the suprabasal epidermis every 20 min (to limit phototoxicity) for about 16-20 h. For the pH-shift experiments in FIG. 24, we first isolated the epidermal skin layer by dispase treatment of E18.5 whole skin (37° C. for 30 min in DPBS). The tissue was mounted with the stratum corneum facing upwards in an otherwise identical manner to the approach described for long-term (about 1 day) imaging of whole skin. In this format we avoided interference and pH buffering from the dermal layer. The tissue was initially imaged using CnT-Airlift media (pH 7.4) before adding an equal volume of acidic CnT-Airlift media (regular media but supplemented for buffering of intracellular pH by adding 280 mM KCl, 20 μM Nigericin and HCl to reach a pH of ˜3.3) to set the final media pH to ˜6.2-6.4. Upon the pH-shift, we imaged the tissue every 5 min for 50 min under usual live imaging conditions. For pH-shift experiments with primary human keratinocytes, we used the same approach as described for the mouse epidermis. For HaCATs with engineered KGs, we performed the pH-shift experiments as before but with acidic CnT-PR-D supplemented with 1.5 mM CaCl₂. To analyze and present live imaging data of the thick epidermis, in the manuscript we typically present 3D projections of the raw (without rendering) fluorescent data. These 3D views were built using the Imaris software (version 8.3.1). We note that such 3D visualizations are clearly indicated in the manuscript by being displayed within a box (delimited by white lines) that provides a rough 3D guide to the viewer. Whenever indicated and if preferred for data visualization, we sometimes also present single optical sections of a larger Z stack (e.g., FIGS. 19A and 22G). Only in three instances in our manuscript we relied on 3D surface renderings, built using Imaris, of the raw fluorescence data in order to (1) quantify changes in KG volume (FIG. 19E) and (2) better visualize (FIG. 22A) their occasional fusions in tissue as well as (3) their interactions with keratins (FIG. 22A, lower panel and FIG. 22B). For live imaging of cells in culture, we typically present (and indicate so in the legends) maximum intensity projections of Z stacks spanning the entire cell volume and prepared using ImageJ. For photobleaching experiments of sensor-labeled KGs in mouse skin, we followed the procedure previously described for the analysis of sensor recovery half-lives in culture.

Selection and Synthesis of pH Reporters

For the synthesis of genetically-encoded pH reporters that sensitively respond with a pKa near 6.5 (based on the known pH range of the extracellular skin pH gradient), we chose two previously published and well-characterized pH reporters: SEpHLuorin (60) and mNectarine (61). We PCR-amplified genes encoding these proteins from Addgene plasmids (#58500 and #80151, respectively) while adding NheI and XmaI restriction sites for insertion into pMAX vectors downstream of a CMV promoter. We used these pMAX-based vectors for validation of the pH reporters using immortalized human keratinocytes (FIG. 28). For expression of pH reporters in mouse skin throughout epidermal differentiation, we subcloned genes encoding these pH reporters into our TRE3G-driven PLKO-based vectors and lentivirally-transduced embryonic mouse skin as in our previous experiments. We note that these pH reporters do not report absolute pH but rather relative changes in pH, as they are not ratiometric. However, because we use them for live imaging, we can confidently identify relative changes in intracellular pH (FIG. 28) by comparing changes in reporter fluorescence within individual cells over time. This approach accounts for the intrinsic limitation of non-ratiometric pH reporters, namely that the total fluorescent signal varies based on expression levels at the single-cell level. In our approach, rapid changes in fluorescent signal can be interpreted as relative changes in pH by correcting for the intensity of the reporter within each cell in time points immediately previous and when reporter fluorescence intensity was still unaffected.

Design and Synthesis of Conventional Client Proteins for KGs

In FIG. 13, we describe in detail the design of conventional clients—different from the new class of clients that we termed phase separation sensors in the previous section, see supplementary text—for filaggrin and its KGs. Sequence details for FLG variants that are uniquely bound (with low affinity) by each of these clients were synthesized as part of pMAX vectors and as previously described for other FLG repeat proteins—see their full sequences in TABLE 5. Briefly, these filaggrin scaffold proteins carry short unique domains recognized by the client (either the cleavage sequence for TEV protease or the murine S100 domain). Genes encoding clients were synthesized as IDT gblocks and cloned into pMAX vectors using the same cloning approach as previously described for phase separation sensors. The sequence details of each client, either a dead-variant of TEV protease (dTEVP) or a mS100 domain, are also included in TABLE 5. While the dTEVP client was exclusively studied in immortalized human keratinocytes (using transfection of corresponding pMAX vectors), for the mS100-based client, which has affinity for endogenous mouse filaggrin, we also subcloned genes encoding this client into our TRE3G-driven PLKO-vectors for lentiviral transduction of the embryonic murine epidermis (FIG. 13D).

TABLE 5
Sequence information for FLG variants recognized by conventional
clients and sequence details for their corresponding clients.
For FLG constructs, we underline the domain
that is specifically bound by the client.

| Construct | Sequence |
|---|---|
| mRFP1- | MASSED VIKEFMRFKVRMEGSVNGHEFEIEGEGEGRPYEGTQTAKLKVTK |
| ENLYFQS- | GGPLPFAWDILSPQFQYGSKAYVKHPADIPDYLKLSFPEGFKWERVMNFE |
| (r8)8-Tail | DGGVVTVTQDSSLQDGEFIYKVKLRGTNFPSDGPVMQKKTMGWEASTER |
| (SEQ ID NO: 39) | MYPEDGALKGEIKMRLKLKDGGHYDAEVKTTYMAKKPVQLPGAYKTDI |
| | KLDITSHNEDYTIVEQYERAEGRHSTGASGSENLYFQSGPGGQVSTHEQSE |
| | SSHGWTGPSTRGRQGSRHEQAQDSSRHSASQDGQDTIRGHPGSSRGGRQG |
| | YHHEHSVDSSGHSGSHHSHTTSQGRSDASRGQSGSRSASRTTRNEEQSGD |
| | GSRHSGSRHHEASTHADISRHSQAVQGQSEGSRRSRRQGSSVSQDSDSEGH |
| | SEDSERWSGSASRNHHGSAQEQLRDGSRHPRSHQEDRAGHGHSADSSRQS |
| | GTRHTQTSSGGQAASSHEQARSSAGERHGSHHQQSADSSRHSGIGHGQAS |
| | SAVRDSGHRGYSGSQASDNEGHSEDSDTQSVSAHGQAGSHQQSHQESAR |
| | GRSGETSGHSGSFLYGQVSTHEQSESSHGWTGPSTRGRQGSRHEQAQDSS |
| | RHSASQDGQDTIRGHPGSSRGGRQGYHHEHSVDSSGHSGSHHSHTTSQGR |
| | SDASRGQSGSRSASRTTRNEEQSGDGSRHSGSRHHEASTHADISRHSQAVQ |
| | GQSEGSRRSRRQGSSVSQDSDSEGHSEDSERWSGSASRNHHGSAQEQLRD |
| | GSRHPRSHQEDRAGHGHSADSSRQSGTRHTQTSSGGQAASSHEQARSSAG |
| | ERHGSHHQQSADSSRHSGIGHGQASSAVRDSGHRGYSGSQASDNEGHSED |
| | SDTQSVSAHGQAGSHQQSHQESARGRSGETSGHSGSFLYGQVSTHEQSESS |
| | HGWTGPSTRGRQGSRHEQAQDSSRHSASQDGQDTIRGHPGSSRGGRQGY |
| | HHEHSVDSSGHSGSHHSHTTSQGRSDASRGQSGSRSASRTTRNEEQSGDGS |
| | RHSGSRHHEASTHADISRHSQAVQGQSEGSRRSRRQGSSVSQDSDSEGHSE |
| | DSERWSGSASRNHHGSAQEQLRDGSRHPRSHQEDRAGHGHSADSSRQSG |
| | TRHTQTSSGGQAASSHEQARSSAGERHGSHHQQSADSSRHSGIGHGQASS |
| | AVRDSGHRGYSGSQASDNEGHSEDSDTQSVSAHGQAGSHQQSHQESARG |
| | RSGETSGHSGSFLYGQVSTHEQSESSHGWTGPSTRGRQGSRHEQAQDSSR |
| | HSASQDGQDTIRGHPGSSRGGRQGYHHEHSVDSSGHSGSHHSHTTSQGRS |
| | DASRGQSGSRSASRTTRNEEQSGDGSRHSGSRHHEASTHADISRHSQAVQ |
| | GQSEGSRRSRRQGSSVSQDSDSEGHSEDSERWSGSASRNHHGSAQEQLRD |
| | GSRHPRSHQEDRAGHGHSADSSRQSGTRHTQTSSGGQAASSHEQARSSAG |
| | ERHGSHHQQSADSSRHSGIGHGQASSAVRDSGHRGYSGSQASDNEGHSED |
| | SDTQSVSAHGQAGSHQQSHQESARGRSGETSGHSGSFLYGQVSTHEQSESS |
| | HGWTGPSTRGRQGSRHEQAQDSSRHSASQDGQDTIRGHPGSSRGGRQGY |
| | HHEHSVDSSGHSGSHHSHTTSQGRSDASRGQSGSRSASRTTRNEEQSGDGS |
| | RHSGSRHHEASTHADISRHSQAVQGQSEGSRRSRRQGSSVSQDSDSEGHSE |
| | DSERWSGSASRNHHGSAQEQLRDGSRHPRSHQEDRAGHGHSADSSRQSG |
| | TRHTQTSSGGQAASSHEQARSSAGERHGSHHQQSADSSRHSGIGHGQASS |
| | AVRDSGHRGYSGSQASDNEGHSEDSDTQSVSAHGQAGSHQQSHQESARG |
| | RSGETSGHSGSFLYGQVSTHEQSESSHGWTGPSTRGRQGSRHEQAQDSSR |
| | HSASQDGQDTIRGHPGSSRGGRQGYHHEHSVDSSGHSGSHHSHTTSQGRS |
| | DASRGQSGSRSASRTTRNEEQSGDGSRHSGSRHHEASTHADISRHSQAVQ |
| | GQSEGSRRSRRQGSSVSQDSDSEGHSEDSERWSGSASRNHHGSAQEQLRD |
| | GSRHPRSHQEDRAGHGHSADSSRQSGTRHTQTSSGGQAASSHEQARSSAG |
| | ERHGSHHQQSADSSRHSGIGHGQASSAVRDSGHRGYSGSQASDNEGHSED |
| | SDTQSVSAHGQAGSHQQSHQESARGRSGETSGHSGSFLYGQVSTHEQSESS |
| | HGWTGPSTRGRQGSRHEQAQDSSRHSASQDGQDTIRGHPGSSRGGRQGY |
| | HHEHSVDSSGHSGSHHSHTTSQGRSDASRGQSGSRSASRTTRNEEQSGDGS |
| | RHSGSRHHEASTHADISRHSQAVQGQSEGSRRSRRQGSSVSQDSDSEGHSE |
| | DSERWSGSASRNHHGSAQEQLRDGSRHPRSHQEDRAGHGHSADSSRQSG |
| | TRHTQTSSGGQAASSHEQARSSAGERHGSHHQQSADSSRHSGIGHGQASS |
| | AVRDSGHRGYSGSQASDNEGHSEDSDTQSVSAHGQAGSHQQSHQESARG |
| | RSGETSGHSGSFLYGQVSTHEQSESSHGWTGPSTRGRQGSRHEQAQDSSR |
| | HSASQDGQDTIRGHPGSSRGGRQGYHHEHSVDSSGHSGSHHSHTTSQGRS |
| | DASRGQSGSRSASRTTRNEEQSGDGSRHSGSRHHEASTHADISRHSQAVQ |
| | GQSEGSRRSRRQGSSVSQDSDSEGHSEDSERWSGSASRNHHGSAQEQLRD |
| | GSRHPRSHQEDRAGHGHSADSSRQSGTRHTQTSSGGQAASSHEQARSSAG |
| | ERHGSHHQQSADSSRHSGIGHGQASSAVRDSGHRGYSGSQASDNEGHSED |
| | SDTQSVSAHGQAGSHQQSHQESARGRSGETSGHSGSFLYGPGLCGHSSDIS |
| | KQLGFSQSQRYYYYEG |
| mRFP1- | Same as above but with a single S to R |
| ENLYFQR- | mutation as shown in bold. |
| (r8)8-Tail | |
| (SEQ ID NO: 40) | |
| mS100-mRFP1- | MSALLESITSMIEIFQQYSTSDKEEETLSKEELKELLEGQLQAVLKNPDDQD |
| (r8)4-Tail | IAEVFMQMLDVDHDDKLDFAEYLLLVLKLAKAYYEASKNEGVPGSGVPG |
| (SEQ ID NO: 41) | AGVPGSRSDASSEDVIKEFMRFKVRMEGSVNGHEFEIEGEGEGRPYEGTQT |
| | AKLKVTKGGPLPFAWDILSPQFQYGSKAYVKHPADIPDYLKLSFPEGFKW |
| | ERVMNFEDGGVVTVTQDSSLQDGEFIYKVKLRGTNFPSDGPVMQKKTMG |
| | WEASTERMYPEDGALKGEIKMRLKLKDGGHYDAEVKTTYMAKKPVQLP |
| | GAYKTDIKLDITSHNEDYTIVEQYERAEGRHSTGASPGGQVSTHEQSESSH |
| | GWTGPSTRGRQGSRHEQAQDSSRHSASQDGQDTIRGHPGSSRGGRQGYH |
| | HEHSVDSSGHSGSHHSHTTSQGRSDASRGQSGSRSASRTTRNEEQSGDGSR |
| | HSGSRHHEASTHADISRHSQAVQGQSEGSRRSRRQGSSVSQDSDSEGHSED |
| | SERWSGSASRNHHGSAQEQLRDGSRHPRSHQEDRAGHGHSADSSRQSGTR |
| | HTQTSSGGQAASSHEQARSSAGERHGSHHQQSADSSRHSGIGHGQASSAV |
| | RDSGHRGYSGSQASDNEGHSEDSDTQSVSAHGQAGSHQQSHQESARGRS |
| | GETSGHSGSFLYGQVSTHEQSESSHGWTGPSTRGRQGSRHEQAQDSSRHS |
| | ASQDGQDTIRGHPGSSRGGRQGYHHEHSVDSSGHSGSHHSHTTSQGRSDA |
| | SRGQSGSRSASRTTRNEEQSGDGSRHSGSRHHEASTHADISRHSQAVQGQS |
| | EGSRRSRRQGSSVSQDSDSEGHSEDSERWSGSASRNHHGSAQEQLRDGSR |
| | HPRSHQEDRAGHGHSADSSRQSGTRHTQTSSGGQAASSHEQARSSAGERH |
| | GSHHQQSADSSRHSGIGHGQASSAVRDSGHRGYSGSQASDNEGHSEDSDT |
| | QSVSAHGQAGSHQQSHQESARGRSGETSGHSGSFLYGQVSTHEQSESSHG |
| | WTGPSTRGRQGSRHEQAQDSSRHSASQDGQDTIRGHPGSSRGGRQGYHHE |
| | HSVDSSGHSGSHHSHTTSQGRSDASRGQSGSRSASRTTRNEEQSGDGSRHS |
| | GSRHHEASTHADISRHSQAVQGQSEGSRRSRRQGSSVSQDSDSEGHSEDSE |
| | RWSGSASRNHHGSAQEQLRDGSRHPRSHQEDRAGHGHSADSSRQSGTRH |
| | TQTSSGGQAASSHEQARSSAGERHGSHHQQSADSSRHSGIGHGQASSAVR |
| | DSGHRGYSGSQASDNEGHSEDSDTQSVSAHGQAGSHQQSHQESARGRSG |
| | ETSGHSGSFLYGQVSTHEQSESSHGWTGPSTRGRQGSRHEQAQDSSRHSAS |
| | QDGQDTIRGHPGSSRGGRQGYHHEHSVDSSGHSGSHHSHTTSQGRSDASR |
| | GQSGSRSASRTTRNEEQSGDGSRHSGSRHHEASTHADISRHSQAVQGQSEG |
| | SRRSRRQGSSVSQDSDSEGHSEDSERWSGSASRNHHGSAQEQLRDGSRHP |
| | RSHQEDRAGHGHSADSSRQSGTRHTQTSSGGQAASSHEQARSSAGERHGS |
| | HHQQSADSSRHSGIGHGQASSAVRDSGHRGYSGSQASDNEGHSEDSDTQS |
| | VSAHGQAGSHQQSHQESARGRSGETSGHSGSFLYGPGLCGHSSDISKQLGF |
| | SQSQRYYYYEG |
| sfGFP-dTEVP | MGSKGEELFTGVVPILVELDGDVNGHKFSVRGEGEGDATNGKLTLKFICT |
| (SEQ ID NO: 42) | TGKLPVPWPTLVTTLGYGVQCFSRYPDHMKRHDFFKSAMPEGYVQERTIS |
| | FKDDGTYKTRAEVKFEGDTLVNRIELKGIDFKEDGNILGHKLEYNFNSHN |
| | VYITADKQKNGIKANFKIRHNVEDGSVQLADHYQQNTPIGDGPVLLPDNH |
| | YLSTQSVLSKDPNEKRDHMVLLEFVTAAGITHGMDELYKSPGSPGSGESLF |
| | KGPRDYNPISSTICHLTNESDGHTTSLYGIGFGPFIITNKHLFRRNNGTLLVQ |
| | SLHGVFKVKNTTTLQQHLIDGRDMIIIRMPKDFPPFPQKLKFREPQREERIC |
| | LVTTNFQTKSMSSMVSDTSCTFPSSDGIFWKHWIQTKDGQAGSPLVSTRD |
| | GFIVGIHSASNFTNTNNYFTSVPKNFMELLTNQEAQQWVSGWRLNADSVL |
| | WGGHKVFMVKPEEPFQPVKEATQLMNELVYSQ |
| mS100-n20GFP | MSALLESITSMIEIFQQYSTSDKEEETLSKEELKELLEGQLQAVLKNPDDQD |
| | IAEVFMQMLDVDHDDKLDFAEYLLLVLKLAKAYYEASKNEGVPGSGVPG |
| (SEQ ID NO: 43) | AGVPGSRSDGASKGEELFTGVVPILVELDGDVNGHKFSVRGEGEGDATNG |
| | KLTLKFICTTGKLPVPWPTLVTTLTYGVQCFSRYPDHMDQHDFFKSAMPE |
| | GYVQERTISFKDDGTYKTRAEVKFEGDTLVNRIELKGIDFKEDGNILGHKL |
| | EYNFNSHDVYITADKQENGIKAEFEIRHNVEDGSVQLADHYQQNTPIGDGP |
| | VLLPDDHYLSTESALSKDPNEDRDHMVLLEFVTAAGIDHGMDELYKSGLE |
| | LLEDLTL |

Immunofluorescence of Fixed Cells and Tissues

To prepare HaCATs for immunostaining, we fixed cultures at 37° C. for 10 min using 4% paraformaldehyde in DPBS. Cultures were washed multiple times with DPBS and stored at 4° C. prior to immunostaining. To prepare murine skin for whole-mount immunostaining, we treated whole skin with dispase for 30 min at 37° C. to isolate the epidermis. We fixed the epidermis at 37° C. for 30 min in 4% paraformaldehyde. After subsequent washes in DPBS, we stored the tissue at 4C in DPBS prior to immunostaining. In all cases, we permeabilized the tissue with an antibody blocking buffer (0.3% Triton X-100, 2.4% Gelatin (Sigma, 67765-1L), 1.2% BSA and 6% normalized Donkey serum in DPBS) for 3-4 hours prior to overnight incubation with primary antibodies. The following primary antibodies were used: chicken anti-GFP (1:2000, Abcam), rabbit anti-RPTN (1:200, Sigma HPA030483), rabbit anti-mFLG (1:1000, Fuchs Lab), rabbit anti-mFLG (1:1000, Abcam ab24584) and goat anti-hFLG (1:200, Santa Cruz, sc-25897). After washing with DPBS, we typically added species-specific secondary antibodies conjugated to RRX or AF647 and incubated the cultures and tissues for 4 h at room temperature. After washing with DAPI, the samples were mounted with ProLong Gold Antifade Mountant (Invitrogen) and cured overnight prior to imaging. For filaggrin immunostaining without secondary antibodies (i.e. direct detection) in mouse skin, we first conjugated anti-mFLG (abeam) to AF647 using an Alexa Fluor™ 647 Antibody Labeling Kit (ThermoFisher) and following the instructions of the manufacturer. We refer to this conjugated antibody as anti-FLG Ab-c(AF647). For immunostaining, we permeabilized the tissue in blocking buffer overnight and added anti-FLG Ab-c(AF647) (1:50) for 4 hours at room temperature. After washing with DAPI, we mounted the tissue as described above. Cultured cells and whole-mounted fixed tissues were imaged using a spinning-disc microscope equipped with a 40× oil objective. Images were analyzed using ImageJ and Imaris 8.3.1.

Skin Barrier Assay

To measure barrier quality, we obtained trans-epidermal water loss measurements (TEWL) using a Tewameter TM 300 (Courage+Khazaka electronic GmbH) on explanted neonatal back skin. Briefly, neonates were humanely sacrificed a few hours (<12 h) after birth and their back skin was harvested and immediately spread over a clean surface. In this format, we ensured that the TM-300 probe (˜1 cm in diameter) covered a large percentage of the back skin surface. We collected four TEWL measurements per sample and consistently discarded the first measurements to limit our analysis to fully acclimatized skin. Each measurement was allowed to stabilize for 30-60 seconds. The values reported by the instrument were not further processed and corresponded to grams of lost water per hour per cm² of skin. We measured 2-3 animals in 3 independent experiments.

Statistical Analyses

Whenever we indicate statistical significance, these are cases where we reject, with a confidence greater than 0.05 (i.e., p-value<0.05). the null hypothesis that the difference in the mean values between two data sets is equal to zero. To perform this hypothesis testing we ran two-sample t-Tests using OriginPro. In all cases we verified that the statistical differences did not depend on the assumption of equal variance (Welch-correction) between samples.

Engineering of Clients that Function as Phase Separation Sensors

“Phase separation sensors” are a new class of clients optimally designed to interrogate dynamic liquid-liquid phase separation events in a way that does not perturb the process. As we define them, phase separation sensors do not bind specific domains on the scaffold protein. Rather, they engage in ultra-weak molecular interactions with key residues of the scaffold (in this case filaggrin). Consequently, only upon a liquid-liquid phase separation do we expect these proteins to become sufficiently concentrated to enable the sensors to appreciably interact with the scaffold. As our data show, these sensors can exhibit a uniquely high signal:noise ratio and participate innocuously without altering the phase separation process (FIG. 14E and FIG. 17). This design permits sensitive and innocuous probing of the evolving dynamics of liquid-phase transitions, which as we show in this manuscript, can profoundly impact tissue processes in vivo. Importantly, designing “phase separation sensors” does not require prior knowledge of scaffold protein binding domains.

By contrast, conventional clients were initially defined as macromolecules that are recruited to a condensate by binding to free sites in its protein scaffold (30). The underlying assumption has been that clients bind to specific domains within the scaffold protein and engage in specific protein-protein interactions between a domain in the client and a domain in the scaffold protein. Such clients have been successful in directing cargo to test-tube and/or phase separated compartments in cell lines in vitro. However, these traditional clients that bind domains within scaffolds have caveats as probes for endogenous phase behavior. They may, for instance, bind to the scaffold prior, regardless of the phase separation process. Moreover, as with fluorescently tagged scaffold proteins (FIG. 8G), client binding may alter both the biological features of the scaffold and its phase separation properties. Finally, one-to-one binding of a client to its target limits the client's partition coefficient as binding sites in the scaffold become saturated. This is a common scenario, since clients are often smaller than scaffolds and are thus expressed at higher levels.

We illustrate these critical differences between phase separation sensors and conventional clients by providing a direct experimental comparison with a conventional client that we engineered to bind a small domain within a filaggrin-like protein (FIGS. 13 and 17). While the conventional client was recruited into filaggrin condensates, it perturbed the normal liquid-like dynamics of the filaggrin scaffold even at concentrations that were well below those at which our phase separation sensors innocuously reported the true liquid-like dynamics of the system. Our engineered client only achieved a partition coefficient of 2.1 (similar to that of other such clients in cells as reported in the literature). By contrast, our phase separation sensors achieved an order of magnitude higher partition coefficient (P=21 for sensor A), and displayed this behavior over a wide range of expression levels (FIG. 17).

We anticipate the development and utilization of new client-based technologies to study native phase separation processes. This will be important to move beyond protein-tagging and into complex biological systems like tissues and living organisms.

Implementation of Phase Separation Sensors to Study Phase Separation Dynamics in Skin

Genetically-encoded phase separation sensors shown here feature two domains: a sensing domain proper and a fluorescent reporter consisting of a fluorescent protein with suitable surface characteristics. The overall rationale is explained above in this example and is also further depicted in FIG. 15. This example also explains in appropriate detail the rationale for the selection and optimization of the fluorescent reporter domain. Here we explain in further detail the strategy for the design of phase separation sensors that are highly sensitive to the phase separation behavior of filaggrin and filaggrin-like proteins. This or a similar strategy can be applied to design phase separation sensors that are highly sensitive to the phase separation behavior of other target phase separation proteins or component proteins in other biomolecular condensates.

Considering that a single filaggrin repeat does not drive phase separation in keratinocytes (FIG. 1G), and that non-synonymous human filaggrin mutations often involve His to Tyr mutations (FIG. 15B-C), we designed Tyr-high single repeat unit variants (denoted r8H1 and r8H2) to optimize their phase separation propensity (FIG. 15D) and tested them as potential phase separation sensors (FIG. 16). Notably, we focused on these mutations as Tyr residues have been shown to promote the phase separation behavior of IDPs (22). Specifically, to generate r8H1, we introduced into the r8 sequence (TABLE 1) all 11 His to Tyr mutations in GnomAD that specifically mapped to the r8 repeat. Then, to generate r8H2, we further introduced additional His to Tyr mutations that occurred in r9, resulting in a total of 21 His to Tyr mutations. Notably, despite the increasing mutational burden on r8H2, the sensing domain remains His-rich (5.9%) compared with the mean abundance of His in the human proteome (2.3%). We also note that mouse filaggrin has a His content of about 7.7% (significantly lower than human filaggrin or filaggrin from non-human primates). These initial variants shared high sequence identity with the original human r8 filaggrin repeat (% I in FIG. 14B). r8H1 shows 98% identity to r8 and r8H2 has 94% identity to r8 sequence. To generate sensor variants with low sequence identity as to avoid using sensor sequences that may have other potential sequence-encoded features of filaggrin—other than its potential for phase-separation-specific interactions—, we used two simple strategies: (i) sequence-reversal and (ii) scrambling of residues. An effective strategy is sequence-reversal. Sequence-reversal is interesting because the resulting sensor sequence has the same amino acids as the parent sequence and is identical to the parent sequence when the new variant is read from C- to N-terminus (as opposed to the proper N- to C-terminal direction), so that the overall composition and other physicochemical properties (e.g. the apparent side chains) remain unaltered. In our experience this strategy tends to preserve the overall biophysical properties of low complexity IDPs (22), unlike scrambling, which can introduce unusual amino acid motifs that are non-native and potentially aggregation-prone. Specifically, we generated the sensor variant ir8H2 by sequence-reversal of the r8H2 sequence and also then generated the sensor variant pr8H2 by permutation of the r8H2 sequence. As shown in FIG. 14B, these new variants have low sequence identity with respect to the original r8 repeat. In particular, ir8H2 has 23% identity to r8 and pr8H2 has 25% identity. Notably, while the phase separation propensity of ir8H2 is nearly identical to that of r8H2, pr8H2 has higher phase separation propensity and in our preliminary experiments pr8H2 showed signs of non-liquid-like behavior, unlike the canonical liquid-like behavior of r8H2-based sequences that we forced into phase separating through the addition of a trimerization domain (FIG. 13C).

We also generated additional (distant) sensor variants that are smaller than a filaggrin repeat but with similar compositional biases (eFlg1, ieFlg1 and eFlg2). Specifically, while the original filaggrin repeat is 324 amino acid resides in length and lacks a clearly discernable internal repeat structure (though low complexity in nature), we engineered a new sensor domain (eFlg1) composed of 5 repeats of a minimal filaggrin repeat that is only 40 amino acid residues in length (GRDGSHSYQGDRSGHSHQRQGYHEQSDRA GHGDSGHRGYS) (SEQ ID NO: 44). This minimal repeat does not occur in filaggrin, but some short motifs do occur within r8 (RQGYH (SEQ ID NO: 45), DRAGHG (SEQ ID NO: 46), EQS (SEQ ID NO: 47), RDGS (SEQ ID NO: 48), DSGHRGYS (SEQ ID NO: 49)) and the overall design is modeled after a canonical UCST phase transition protein-polymer with low phase separation propensity (22). We then generated ieFlg1 by sequence-reversal of eFlg1. eFlg2 corresponds to a sensor domain that approximates the size of r8 by directly fusing a 4-mer of the eFlg1 repeat with a 4-mer of the ieFlg1 repeat (TABLE 3).

Overall, we suggest that the use of naturally-occurring non-pathogenic mutations within specific domains of human phase-sensitive proteins followed by sequence-reversal constitutes a straightforward and general approach to the design of highly sensitive phase separation sensors that are tailored for specific biological systems. Other strategies are also feasible, as we also demonstrate here with the design of fully engineered sensor domains (e.g. ieFlg1 in Sensor B, FIG. 14C).

### REFERENCES

1. C. P. Brangwynne et al., Germline P granules are liquid droplets that localize by controlled dissolution/condensation. Science 324, 1729-1732 (2009).
2. S. F. Banani, H. O. Lee, A. A. Hyman, M. K. Rosen, Biomolecular condensates: organizers of cellular biochemistry. Nature reviews Molecular cell biology 18, 285 (2017).
3. Y. Shin, C. P. Brangwynne, Liquid phase condensation in cell physiology and disease. Science 357, eaaf4382 (2017).
4. M. Feric et al., Coexisting liquid phases underlie nucleolar subcompartments. Cell 165, 1686-1697 (2016).
5. X. Su et al., Phase separation of signaling molecules promotes T cell receptor signal transduction. Science 352, 595-599 (2016).
6. J. T. Wang et al., Regulation of RNA granule dynamics by phosphorylation of serine-rich, intrinsically disordered proteins in C. elegans. Elife 3, e04591 (2014).
7. A. Molliex et al., Phase separation by low complexity domains promotes stress granule assembly and drives pathological fibrillization. Cell 163, 123-133 (2015).
8. B. R. Sabari et al., Coactivator condensation at super-enhancers links phase separation and gene control. Science 361, eaar3958 (2018).
9. W.-K. Cho et al., Mediator and RNA polymerase II clusters associate in transcription-dependent condensates. Science 361, 412-415 (2018).
10. B. A. Gibson et al., Organization of chromatin by intrinsic and regulated phase separation. Cell 179, 470-484. e421 (2019).
11. G. Wan et al., Spatiotemporal regulation of liquid-like condensates in epigenetic inheritance. Nature 557, 679 (2018).
12. Y. Lin, D. S. Protter, M. K. Rosen, R. Parker, Formation and maturation of phase-separated liquid droplets by RNA-binding proteins. Molecular cell 60, 208-219 (2015).
13. H. Jiang et al., Phase transition of spindle-associated protein regulate spindle apparatus assembly. Cell 163, 108-122 (2015).
14. A. K. Rai, J.-X. Chen, M. Selbach, L. Pelkmans, Kinase-controlled phase transition of membraneless organelles in mitosis. Nature 559, 211 (2018).
15. O. Beutel, R. Maraspini, K. Pombo-Garcfa, C. Martin-Lemaitre, A. Honigmann, Phase Separation of Zonula Occludens Proteins Drives Formation of Tight Junctions. Cell 179, 923-936.e911 (2019).
16. E. S. F. Rosenzweig et al., The eukaryotic CO2-concentrating organelle is liquid-like and exhibits dynamic reorganization. Cell 171, 148-162. el 19 (2017).
17. S. Alberti, A. Gladfelter, T. Mittag, Considerations and Challenges in Studying Liquid-Liquid Phase Separation and Biomolecular Condensates. Cell 176, 419-434 (2019).
18. H. B. Schmidt, A. Barreau, R. Rohatgi, Phase separation-deficient TDP43 remains functional in splicing. Nature communications 10, 1-14 (2019).
19. D. Bracha et al., Mapping Local and Global Liquid Phase Behavior in Living Cells Using Photo-Oligomerizable Seeds. Cell 175, 1467-1480.e1413 (2018).
20. J. A. Segre, Epidermal barrier formation and recovery in skin disorders. J. Clin. Invest. 116, 1150-1158 (2006).
21. B. A. Dale, K. A. Resing, R. B. Presland, in The keratinocyte handbook, I. Leigh, E. B. Lane, F. M. Watt, Eds. (Cambridge University Press, Cambridge, U.K., 1994), chap. 17, pp. 323-350.
22. F. G. Quiroz, A. Chilkoti, Sequence heuristics to encode phase behaviour in intrinsically disordered protein polymers. Nature materials 14, 1164 (2015).
23. C. N. Palmer et al., Common loss-of-function variants of the epidermal barrier protein filaggrin are a major predisposing factor for atopic dermatitis. Nature genetics 38, 441 (2006).
24. S. J. Brown, W. I. McLean, One remarkable molecule: filaggrin. J. Invest. Derm 132, 751-762 (2012).
25. D. J. Margolis et al., Filaggrin-2 variation is associated with more persistent atopic dermatitis in African American subjects. Journal of Allergy and Clinical Immunology 133, 784-789 (2014).
26. S. Rahrig et al., Transient Epidermal Barrier Deficiency and Lowered Allergic Threshold in Filaggrin-Hornerin (FlgHrnr−/−) Double-Deficient Mice. Allergy, 1-13 (2019).
27. X. C. C. Wong et al., Array-based sequencing of filaggrin gene for comprehensive detection of disease-associated variants. Journal of Allergy and Clinical Immunology 141, 814-816 (2018).
28. C.-A. Lo et al., Quantification of protein levels in single living cells. Cell reports 13, 2634-2644 (2015).
29. C. G. Bunick et al., Crystal structure of human profilaggrin 5100 domain and identification of target proteins annexin II, stratifin, and HSP27. Journal of Investigative Dermatology 135, 1801-1809 (2015).
30. S. F. Banani et al., Compositional control of phase-separated cellular bodies. Cell 166, 651-663 (2016).
31. B. S. Schuster et al., Controllable protein phase separation and modular recruitment to form responsive membraneless organelles. Nature communications 9, 2985 (2018).
32. T. Christensen, W. Hassouneh, K. Trabbic-Carlson, A. Chilkoti, Predicting transition temperatures of elastin-like polypeptide fusion proteins. Biomacromolecules 14, 1514-1519 (2013).
33. B. R. McNaughton, J. J. Cronican, D. B. Thompson, D. R. Liu, Mammalian cell penetration, siRNA transfection, and DNA transfection by supercharged proteins. Proceedings of the National Academy of Sciences 106, 6111-6116 (2009).
34. J. Jaubert, S. Patel, J. Cheng, J. A. Segre, Tetracycline-regulated transactivators driven by the involucrin promoter to achieve epidermal conditional gene expression. Journal of Investigative Dermatology 123, 313-318 (2004).
35. C. Bonnart et al., Elastase 2 is expressed in human and mouse epidermis and impairs skin barrier function in Netherton syndrome through filaggrin and lipid misprocessing. The Journal of clinical investigation 120, 871-882 (2010).
36. P. Rompolas et al., Spatiotemporal coordination of stem cell commitment during epidermal homeostasis. Science 352, 1471-1474 (2016).
37. T. Kartasova, D. R. Roop, K. A. Holbrook, S. H. Yuspa, Mouse differentiation-specific keratins 1 and 10 require a preexisting keratin scaffold to form a filament network. The Journal of cell biology 120, 1251-1261 (1993).
38. V. Kumar et al., A keratin scaffold regulates epidermal barrier formation, mitochondrial lipid composition, and activity. J Cell Biol 211, 1057-1075 (2015).
39. C.-H. Lee, M.-S. Kim, B. M. Chung, D. J. Leahy, P. A. Coulombe, Structural basis for heteromeric assembly and perinuclear organization of keratin filaments. Nature structural & molecular biology 19, 707 (2012).
40. M. P. Hughes et al., Atomic structures of low-complexity protein segments reveal kinked (3 sheets that assemble networks. Science 359, 698-701 (2018).
41. S. Noda et al., The Asian atopic dermatitis phenotype combines features of atopic dermatitis and psoriasis with increased TH17 polarization. Journal of Allergy and Clinical Immunology 136, 1254-1264 (2015).
42. T. J. Nott et al., Phase transition of a disordered nuage protein generates environmentally responsive membraneless organelles. Molecular cell 57, 936-947 (2015).
43. F. G. Quiroz, A. Chilkoti, Sequence heuristics to encode phase behaviour in intrinsically disordered protein polymers. Nat Mater 14, 1164-1171 (2015).
44. R. Niesner et al., 3D-resolved investigation of the pH gradient in artificial skin constructs by means of fluorescence lifetime imaging. Pharmaceutical research 22, 1079-1087 (2005).
45. J. A. MacKay, D. J. Callahan, K. N. FitzGerald, A. Chilkoti, Quantitative model of the phase behavior of recombinant pH-responsive elastin-like polypeptides. Biomacromolecules 11, 2873-2879 (2010).
46. S. Alberti, Guilty by association: Mapping out the molecular sociology of droplet compartments. Molecular cell 69, 349-351 (2018).
47. S. Markmiller et al., Context-dependent and disease-specific diversity in protein interactions within stress granules. Cell 172, 590-604. e513 (2018).
48. I. Brody, An ultrastructural study on the role of the keratohyalin granules in the keratinization process. Journal of ultrastructure research 3, 84-104 (1959).
49. T. Makino, M. Takaishi, M. Morohashi, N.-h. Huh, Hornerin, a novel profilaggrin-like protein and differentiation-specific marker isolated from mouse skin. J. Biol. Chem. 276, 47445-47452 (2001).
50. P. M. Steinert, D. A. Parry, L. N. Marekov, Trichohyalin Mechanically Strengthens the Hair Follicle. J. Biol. Chem. 278, 41409-41419 (2003).
51. B. Mészáros et al., PhaSePro: the database of proteins driving liquid-liquid phase separation. Nucleic Acids Research, (2019).
52. J. Kyte, R. F. Doolittle, A simple method for displaying the hydropathic character of a protein. Journal of molecular biology 157, 105-132 (1982).
53. J. R. McDaniel, J. A. MacKay, F. G. Quiroz, A. Chilkoti, Recursive directional ligation by plasmid reconstruction allows rapid and seamless cloning of oligomeric genes. Biomacromolecules 11, 944-952 (2010).
54. A. C. Woerner et al., Cytoplasmic protein aggregates interfere with nucleocytoplasmic transport of protein and RNA. Science 351, 173-176 (2016).
55. S. P. Boudko et al., Crystal structure of human collagen XVIII trimerization domain: A novel collagen trimerization Fold. Journal of molecular biology 392, 787-802 (2009).
56. A. Ghoorchian, N. B. Holland, Molecular architecture influences the thermally induced aggregation behavior of elastin-like polypeptides. Biomacromolecules 12, 4022-4029 (2011).
57. N. Maas-Szabowski, A. Starker, N. E. Fusenig, Epidermal tissue regeneration and stromal interaction in HaCaT cells is initiated by TGF-α. Journal of cell science 116, 2937-2948 (2003).
58. J. A. Nowak, E. Fuchs, in Stem Cells in Regenerative Medicine. (Springer, 2009), pp. 215-232.
59. S. Beronja, G. Livshits, S. Williams, E. Fuchs, Rapid functional dissection of genetic networks via tissue-specific transduction and RNAi in mouse embryos. Nature medicine 16, 821 (2010).
60. S. Sankaranarayanan, D. De Angelis, J. E. Rothman, T. A. Ryan, The use of pHluorins for optical measurements of presynaptic activity. Biophysical journal 79, 2199-2208 (2000).
61. D. E. Johnson et al., Red fluorescent protein pH biosensor to detect concentrative nucleoside transport. Journal of Biological Chemistry 284, 20499-20511 (2009).
62. I. Nemoto-Hasebe et al., FLG mutation p. Lys4021X in the C-terminal imperfect filaggrin repeat in Japanese patients with atopic eczema. British Journal of Dermatology 161, 1387-1390 (2009).
63. D. T. Jones, D. Cozzetto, DISOPRED3: precise disordered region predictions with annotated protein-binding activity. Bioinformatics 31, 857-863 (2014).
64. D. J. Scott et al., A novel ultra-stable, monomeric green fluorescent protein for direct volumetric imaging of whole organs using clarity. Scientific reports 8, 667 (2018).
65. C. M. Nunn et al., Crystal structure of tobacco etch virus protease shows the protein C terminus bound within the active site. Journal of molecular biology 350, 145-155 (2005).
66. W. Wang et al., A light- and calcium-gated transcription factor for imaging and manipulating activated neurons. Nature biotechnology 35, 864 (2017).
67. M.-T. Wei et al., Phase behaviour of disordered proteins underlying low density and high permeability of liquid organelles. Nature chemistry 9, 1118 (2017).

### Example 2

### Design and Assessment of Multi-Domain Sensors

Phase separation sensors having multiple domains, particularly more than two domains have been contemplated, designed, constructed and evaluated for activity. These particularly include sensors having an artificial client protein or molecule domain and additionally at least one domain providing an accessory protein or molecule. Engineering of and exemplary phase separation sensor domains, including multi-domains, are depicted in FIG. 31.

### Design of Multi-Domain Sensors

As described above in Example 1, two-domain sensors were designed, constructed and evaluated based on a two-domain structure, with the sensors having a first domain comprising a fluorescent protein or marker and a second domain comprising an IDP sensing domain or an artificial client protein sequence. A general two-domain sensor architecture is as follows:

Two-domain: [Fluorescent marker/Enzyme/Cargo]-[Optional Linker]-[IDP sensing domain]

The two domain structure was utilized in Example 1, where exemplary two-domain sensors comprised a fluorescent marker which is exemplified by GFP, particularly a GFP with positively-charged amino acids exposed on the protein surface, such as exemplary +15GFP, linked to an IDP sensing domain, such as the artificial IDP sequence based on filaggrin sequence. The design and characteristics of the IDP sensing domain sequence(s) are described in Example 1. The Sensor A and Sensor B utilized in Example 1 are also described below. These sensors included a nuclear export signal (denoted NES) to direct the protein sensor to the cytoplasm of the cell when expressed. Each of Sensor A and Sensor B comprises a fluorescent marker protein (+15GFP was utilized), followed by a nuclear export signal (denoted NES). The NES sequence is indicated below in bold and underlined. The nuclear export signal sequence utilizes the NES sequence LELLEDLTL (SEQ ID NO: 57), which is an optimized export signal reported by Woerner A T et al (Woerner A C et al Science (2016) 351(6269):173-176. The reported LELLEDLTL was further expanded in sensors A and B to incorporate two N-terminal additional linker residues SG, to provide a full NES sequence of SGLELLEDLTL (SEQ ID NO: 58). A flexible linker sequence of 4 amino acids, particularly GSPG (SEQ ID NO: 59) was incorporated in the Sensor A and B constructs (double underlined in the sensor sequences below). Alternative or additional linker sequence GRSDGVPGSG (SEQ ID NO: 60) was also incorporated and utilized in some sensor designs and sequences (double underlined in the sensor sequences below). A suitable alternative optional linker would be from about 2-10 residues in length and lack charged residues or be zwitterionic and have equal numbers of positively-charged and negatively-charged amino acid residues.

Sensor A
Construct architecture:
[+15GFP-NES]-[linker1]-[ir8H2]
Full amino acid sequence (SEQ ID NO: 26):
MGASKGERLFTGVVPILVELDGDVNGHKFSVRGEG
EGDATRGKLTLKFICTTGKLPVPWPTLVTTLTYGV
QCFSRYPKHMKRHDFFKSAMPEGYVQERTISFKKD
GTYKTRAEVKFEGRTLVNRIELKGRDFKEKGNILG
HKLEYNFNSHNVYITADKRKNGIKANFKIRHNVKD
GSVQLADHYQONTPIGRGPVLLPRNHYLSTRSALS
KDPKEKRDHMVLLEFVTAAGITHGMDELYKSGLEL
LEDLTLGSPGYLFSGSHGSTEGSRGRASEQYSQQH
SGAQGHASVSQTDSDESHGENDSAQSGSYGRHGSD
RVASSAQGHGIGSHRSSDASQQYHSGYREGASSRA
QEHSSAAQGGSSTQTYRTGSQRSSDASYGHGARDE
QHSRPYRSGDRLQEQASGHHNRSASGSWRESDESH
GESDSDQSVSSGQRRSRRSGESQGQVAQSYRSIDA
HTSAEYHRSGSYRSGDGSQEENRTTRSASRSGSQG
RSADSRGQSTTHSHYSGSYGSSDVSHEYYYGQRGG
RSSGPYGRITDQGDQSASYRSSDQAQEYRSGQRGR
TSPGTWGYSSESQEYTSVQGS
Full amino acid sequence (SEQ ID NO: 27):
MGASKGERLFTGVVPILVELDGDVNGHKFSVRGEG
EGDATRGKLTLKFICTTGKLPVPWPTLVTTLTYGV
QCFSRYPKHMKRHDFFKSAMPEGYVQERTISFKKD
GTYKTRAEVKFEGRTLVNRIELKGRDFKEKGNILG
HKLEYNFNSHNVYITADKRKNGIKANFKIRHNVKD
GSVQLADHYQQNTPIGRGPVLLPRNHYLSTRSALS
KDPKEKRDHMVLLEFVTAAGITHGMDELYKSGLEL
LEDLTLGSPGSYGRHGSDGHGARDSQEHYGQRQHS
HGSRDGQYSHSGDRGSYGRHGSDGHGARDSQEHYG
QRQHSHGSRDGQYSHSGDRGSYGRHGSDGHGARDS
QEHYGQRQHSHGSRDGQYSHSGDRGSYGRHGSDGH
GARDSQEHYGQRQHSHGSRDGQYSHSGDRGSYGRH
GSDGHGARDSQEHYGQRQHSHGSRDGQYSHSGDRG
GS

In further studies, multi-domain sensors were designed, constructed and evaluated based on a three-domain structure (see for example FIG. 31), with the sensors having a first domain comprising a fluorescent protein or marker, a further domain comprising an enzyme or cargo protein and a final domain comprising an IDP sensing domain or an artificial client protein sequence. Linkers can optionally be utilized between the domain sequences. A general three-domain sensor architecture is as follows:

Three-domain: [Fluorescent marker]-[Optional linker]-[Enzyme/Cargo]-[Optional Linker]-[IDP sensing domain]

In an exemplary set of three-domain sensors, the Enzyme/Cargo is an enzyme and can, for example, be a peroxidase. BioID2 may be used as the enzyme domain. BioID2 biotinylates a target protein and uses endogenous or exogenously-added biotin to label a biomolecular condensate's components.

In a further example, the peroxidase Apex2, which is an engineered peroxidase enzyme developed by Ting and collaborators, was used as an accessory protein (Rhee H-W et al Science (2013) 339:1328-1331; Hung V et al Mol Cell (2014) 55:332-341). Apex2 does not function with regular native biotin (the one normally found in our bodies), but requires a chemically-modified biotin, biotin phenol (denoted BP), that is added prior to tissue processing/fixation. Addition of the small molecule BP substrate for Apex2, results in the covalent biotinylation of endogenous proteins within 1-10 nm of APEX over a 1 minute time window in living cells. Hydrogen-peroxide (H202) can be optionally added to accelerate the biotinylation reaction.

The full Apex2 sequence (including a C-terminal NES sequence) was derived from Addgene plasmid pcDNA3 APEX2-NES (#49386).

Apex2-SensorA:
Construct architecture:[+15GFP-NES]-[linker1]-
[Apex2]-[Linker2]-[ir8H2]
Full amino acid sequence (SEQ ID NO: 50)
MGASKGERLFTGVVPILVELDGDVNGHKFSVRGEG
EGDATRGKLTLKFICTTGKLPVPWPTLVTTLTYGV
QCFSRYPKHMKRHDFFKSAMPEGYVQERTISFKKD
GTYKTRAEVKFEGRTLVNRIELKGRDFKEKGNILG
HKLEYNFNSHNVYITADKRKNGIKANFKIRHNVKD
GSVQLADHYQQNTPIGRGPVLLPRNHYLSTRSALS
KDPKEKRDHMVLLEFVTAAGITHGMDELYKSGLEL
LEDLTLGRSDGVPGSGGKSYPTVSADYQDAVEKAK
KKLRGFIAEKRCAPLMLRLAFHSAGTFDKGTKTGG
PFGTIKHPAELAHSANNGLDIAVRLLEPLKAEFPI
LSYADFYQLAGVVAVEVTGGPKVPFHPGREDKPEP
PPEGRLPDPTKGSDHLRDVFGKAMGLTDQDIVALS
GGHTIGAAHKERSGFEGPWTSNPLIFDNSYFTELL
SGEKEGLLQLPSDKALLSDPVFRPLVDKYAADEDA
FFADYAEAHQKLSELGFADALQLPPLERLTLDGSP
GYLFSGSHGSTEGSRGRASEQYSQQHSGAQGHASV
SQTDSDESHGENDSAQSGSYGRHGSDRVASSAQGH
GIGSHRSSDASQQYHSGYREGASSRAQEHSSAAQG
GSSTQTYRTGSQRSSDASYGHGARDEQHSRPYRSG
DRLQEQASGHHNRSASGSWRESDESHGESDSDQSV
SSGQRRSRRSGESQGQVAQSYRSIDAHTSAEYHRS
GSYRSGDGSQEENRTTRSASRSGSQGRSADSRGQS
TTHSHYSGSYGSSDVSHEYYYGQRGGRSSGPYGRI
TDQGDQSASYRSSDQAQEYRSGQRGRTSPGTWGYS
SESQEYTSVQGS
Apex2-SensorB:
Construct architecture:
[+15GFP-NES]-[linker1]-[Apex2]-
[Linker2]-[ieFlg1]
Full amino acid sequence (SEQ ID NO: 51):
MGASKGERLFTGVVPILVELDGDVNGHKFSVRGEG
EGDATRGKLTLKFICTTGKLPVPWPTLVTTLTYGV
QCFSRYPKHMKRHDFFKSAMPEGYVQERTISFKKD
GTYKTRAEVKFEGRTLVNRIELKGRDFKEKGNILG
HKLEYNFNSHNVYITADKRKNGIKANFKIRHNVKD
GSVQLADHYQQNTPIGRGPVLLPRNHYLSTRSALS
KDPKEKRDHMVLLEFVTAAGITHGMDELYKSGLEL
LEDLTLGRSDGVPGSGGKSYPTVSADYQDAVEKAK
KKLRGFIAEKRCAPLMLRLAFHSAGTFDKGTKTGG
PFGTIKHPAELAHSANNGLDIAVRLLEPLKAEFPI
LSYADFYQLAGVVAVEVTGGPKVPFHPGREDKPEP
PPEGRLPDPTKGSDHLRDVFGKAMGLTDQDIVALS
GGHTIGAAHKERSGFEGPWTSNPLIFDNSYFTELL
SGEKEGLLQLPSDKALLSDPVFRPLVDKYAADEDA
FFADYAEAHQKLSELGFADALQLPPLERLTLDGSP
GSYGRHGSDGHGARDSQEHYGQRQHSHGSRDGQYS
HSGDRGSYGRHGSDGHGARDSQEHYGQRQHSHGSR
DGQYSHSGDRGSYGRHGSDGHGARDSQEHYGQRQH
SHGSRDGQYSHSGDRGSYGRHGSDGHGARDSQEHY
GQRQHSHGSRDGQYSHSGDRGSYGRHGSDGHGARD
SQEHYGQRQHSHGSRDGQYSHSGDRGGS

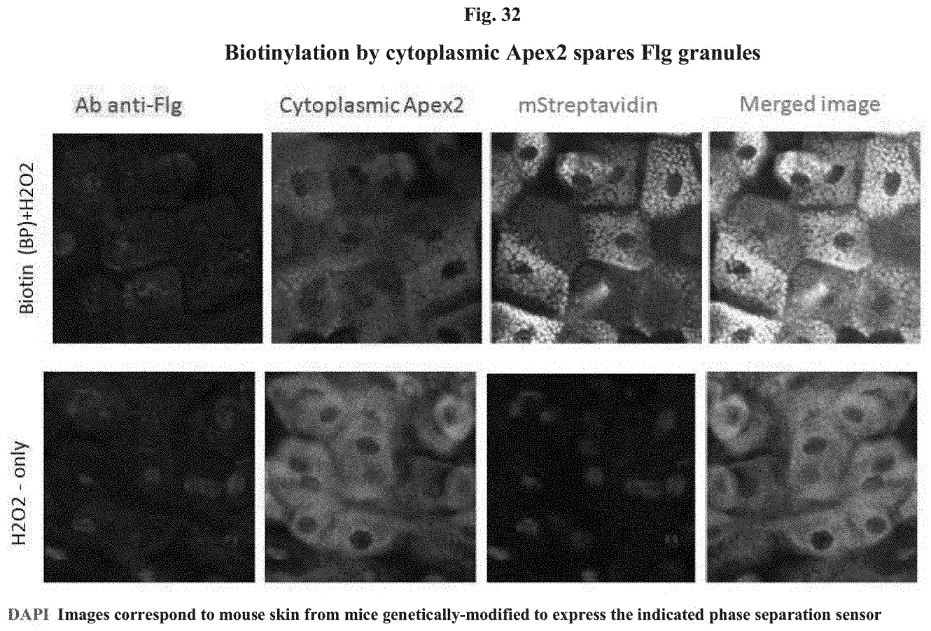

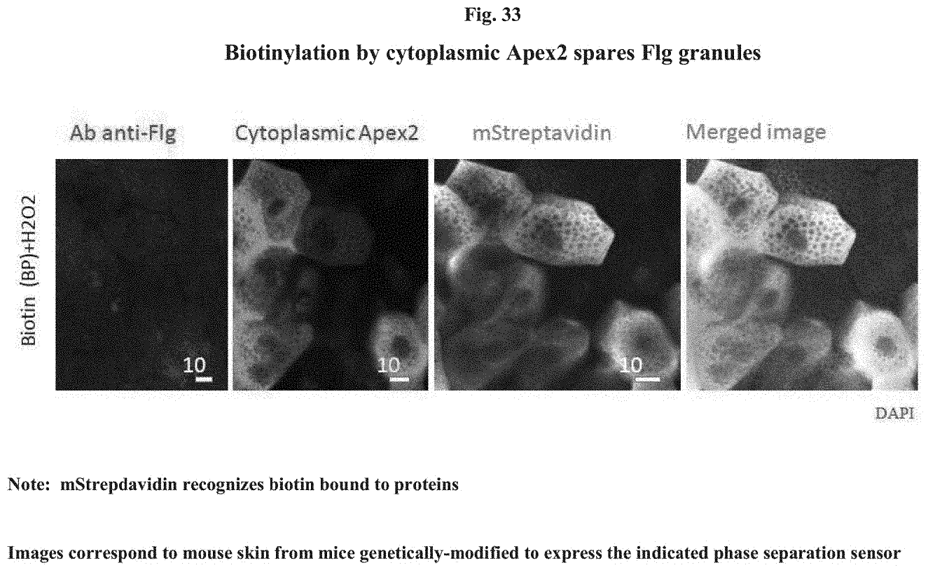

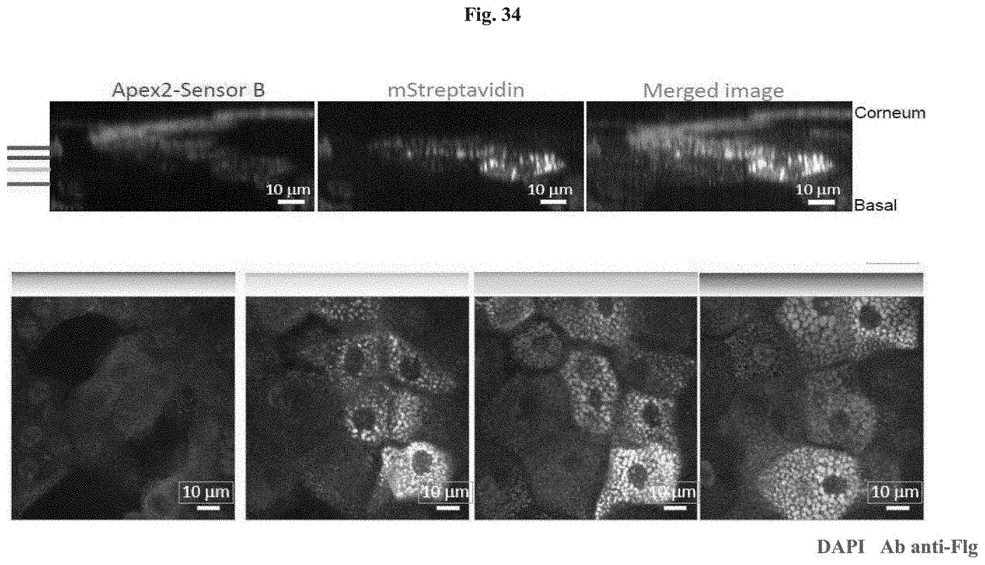

The Apex2-SensorA was evaluated in skin cells in a set of experiments in line with those outlined in Example 1. In skin from nice genetically-engineered to express the Apex2-SensorA sensor, low levels of biotinylation (above background) are observed when BP is added and H202 is omitted (not shown). Upon addition of both the substrate BP and H202 to enhance the reaction, biotinylated KG granule components are clearly visualized (FIG. 32). The advantage of Apex2 over BioID2 (which uses endogenous biotin) is that the time at which the Apex2-modified sensor becomes enzymatically-active within condensates can be controlled (that is, when both BP and H202 are exogenously provided). FIG. 33 shows that biotinylation by a cytoplasmic Apex2, which is a fluorescently-labeled Apex2 protein lacking a phase separation sensor domain, spares Flg granules. This cytoplasmic Apex2 construct may be used as a control for example in quantitative proteomics studies involving KG-targeted Apex2 sensors. Study results showing that Apex2-SensorB biotinylates early and late granules are provided in FIG. 34. We followed the same procedure as in FIG. 32, but this time the experiment involved mice whose skin was genetically-modified to express a Apex2-SensorB (SEQ ID NO:51), which features a different sensor domain (SensorB instead of SensorA). As in all other cases, the resulting tissue is mosaic, so that only a subset of cells in the epidermis express the sensor.

In addition to biotinylation, when provided with diaminobenzidine(DAB) (instead of BP), Apex2-based sensors may be used to enable visualization of condensates via electron microscopy) (Hung V et al Nat Protocol (2016) 11(3):456-475, doi.10.1038/mprot.2016.018).

In addition to Apex2 and BioiD2, experts in the art can reasonably further modify our sensor designs to include newly-evolved and more efficient enzymes, including tagging and modifying enzymes, such as alternative ligases, peroxidases, etc. Examples include TurboID and miniTurboID (Branon T C et al Nature Biotechnology (2018) 36:880-887). TurboID and miniTurboID are engineered mutants of biotin ligase which provide enzyme-catalyzed proximity labeling in living cells with much greater efficiency than BioID.

Experts in the art will further recognize that the Apex2 domain in our phase separation sensors may be further modified to include other enzymes or proteins of interest, including as to exploit the phase separation sensors as vehicles that deliver cargo to biomolecular condensates. Said cargo may include but is not limited to fluorescent proteins, proteases, nucleases, ligases, peroxidases, phosphatases, kinases and other proteins capable of modifying proteins and nucleic acids or showing a biological activity of interest.

Additional sensors in line with the tri-domain constructs above and using an alternative positively charged GFP sequence (+15GFP-Kv) are also provided. These are two-domain constructs as shown below. The fluorescent marker +15GFP-Kv is a variant of +15GFP engineered by us in which all (surface-exposed) Arg residues that contribute to +15GFP (i.e. not present in sfGFP) were replaced by Lys residues. +15GFP was engineered with eight X>R substitutions and five X>K substitutions, whereas in our +15GFP-Kv all 13 mutations are X>K substitutions.

Sensor C:
Construct architecture:
[+15GFP-Kv-NES]-[Linker1]-[ir8H2]
Full amino acid sequence (SEQ ID NO: 52):
MGASKGEKLFTGVVPILVELDGDVNGHKFSVRGEG
EGDATKGKLTLKFICTTGKLPVPWPTLVTTLTYGV
QCFSRYPKHMKRHDFFKSAMPEGYVQERTISFKKD
GTYKTRAEVKFEGKTLVNRIELKGKDFKEKGNILG
HKLEYNFNSHNVYITADKKKNGIKANFKIRHNVKD
GSVQLADHYQQNTPIGKGPVLLPKNHYLSTKSALS
KDPKEKRDHMVLLEFVTAAGITHGMDELYKSGLEL
LEDLTLGSPGYLFSGSHGSTEGSRGRASEQYSQQH
SGAQGHASVSQTDSDESHGENDSAQSGSYGRHGSD
RVASSAQGHGIGSHRSSDASQQYHSGYREGASSRA
QEHSSAAQGGSSTQTYRTGSQRSSDASYGHGARDE
QHSRPYRSGDRLQEQASGHHNRSASGSWRESDESH
GESDSDQSVSSGQRRSRRSGESQGQVAQSYRSIDA
HTSAEYHRSGSYRSGDGSQEENRTTRSASRSGSQG
RSADSRGQSTTHSHYSGSYGSSDVSHEYYYGQRGG
RSSGPYGRITDQGDQSASYRSSDQAQEYRSGQRGR
TSPGTWGYSSESQEYTSVQGS
Sensor D:
Construct architecture:
[+15GFP-Kv-NES]-[Linker1]-[ieFlg1]
Full amino acid sequence (SEQ ID NO: 53):
MGASKGEKLFTGVVPILVELDGDVNGHKFSVRGEG
EGDATKGKLTLKFICTTGKLPVPWPTL
VTTLTYGVQCFSRYPKHMKRHDFFKSAMPEGYVQE
RTISFKKDGTYKTRAEVKFEGKTLVNRIELKGKDF
KEKGNILGHKLEYNFNSHNVYITADKKKNGIKANF
KIRHNVKDGSVQLADHYQQNTPIGKGPVLLPKNHY
LSTKSALSKDPKEKRDHMVLLEFVTAAGITHGMDE
LYKSGLELLEDLTLGSPGSYGRHGSDGHGARDSQE
HYGQRQHSHGSRDGQYSHSGDRGSYGRHGSDGHGA
RDSQEHYGQRQHSHGSRDGQYSHSGDRGSYGRHGS
DGHGARDSQEHYGQRQHSHGSRDGQYSHSGDRGSY
GRHGSDGHGARDSQEHYGQRQHSHGSRDGQYSHSG
DRGSYGRHGSDGHGARDSQEHYGQRQHSHGSRDGQ
YSHSGDRGGS

### Additional Constructs and Sequences of Interest

Enzymatic-based labeling sensors such as Apex2-based sensors may be used to tag (for example with biotin using Apex2) all protein and RNA components present within biomolecular condensates. Quantitative approaches, however, will require control experiments in which tagging can also or alternatively be directed to components outside of biomolecular condensates. We engineered and validated an Apex2-based construct, Apex2-excluded, that lacks a phase separation sensing domain and which was multimerized into a trimer (via its Foldon domain) to prevent its trafficking into biomolecular condensates.

(A1): Apex2-excluded:
Construct architecture:
[sfGFP]-[NES]-[Linker1]-[Apex2]-
[Linker2]-[Foldon]
Full amino acid sequence (SEQ ID NO: 54):
MGASKGEELFTGVVPILVELDGDVNGHKFSVRGEG
EGDATNGKLTLKFICTTGKLPVPWPTLVTTLGYGV
QCFSRYPDHMKRHDFFKSAMPEGYVQERTISFKDD
GTYKTRAEVKFEGDTLVNRIELKGIDFKEDGNILG
HKLEYNFNSHNVYITADKQKNGIKANFKIRHNVED
GSVQLADHYQQNTPIGDGPVLLPDNHYLSTQSVLS
KDPNEKRDHMVLLEFVTAAGITHGMDELYKSGLEL
LEDLTLGRSDGVPGSGGKSYPTVSADYQDAVEKAK
KKLRGFIAEKRCAPLMLRLAFHSAGTFDKGTKTGG
PFGTIKHPAELAHSANNGLDIAVRLLEPLKAEFPI
LSYADFYQLAGVVAVEVTGGPKVPFHPGREDKPEP
PPEGRLPDPTKGSDHLRDVFGKAMGLTDQDIVALS
GGHTIGAAHKERSGFEGPWTSNPLIFDNSYFTELL
SGEKEGLLQLPSDKALLSDPVFRPLVDKYAADEDA
FFADYAEAHQKLSELGFADALQLPPLERLTLDGSP
GSPGSGYIPEAPRDGQAYVRKDGEWVLLSTFL

This invention may be embodied in other forms or carried out in other ways without departing from the spirit or essential characteristics thereof. The present disclosure is therefore to be considered in all aspects and embodiments illustrative and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. A phase separation sensor capable of targeting or associating with a biomolecular condensate and comprising at least two protein domains, wherein the first domain comprises one or more accessory protein and the second domain comprises an artificial client protein having intrinsic disorder and capable of engaging in ultra-weak phase separation-specific amino acid interactions with one or more component protein in the condensate.

2. The sensor of claim 1 wherein
(a) the sensor lacks independent phase separation behavior when expressed in the cell; and/or
(b) the sensor associates with the biomolecular condensate without disrupting the condensate.

3. (canceled)

4. The sensor of claim 1 wherein the artificial client protein is an intrinsically disordered protein having low complexity sequence and wherein the artificial client protein sequence comprises similar compositional bias or comprises related sequence patterns with low sequence identity to amino acid sequence of a naturally-occurring intrinsically disordered protein or protein region within a larger protein and which is responsible for driving assembly of said biomolecular condensate.

5. (canceled)

6. The sensor of claim 4 wherein the artificial client protein sequence is designed based on reversing the amino acid sequence of a target component's sequence reading C-terminal to N-terminal so as to provide a distinct and non-natural amino acid sequence having similar amino acid composition.

7. The sensor of claim 1 wherein at least one accessory protein provides a detectable or functional label, or is an enzyme.

8. The sensor of claim 1 wherein at least one accessory protein is selected from fluorescent protein, protease, nuclease, ligase, peroxidase, phosphatase, kinase and protein capable of modifying a protein or nucleic acid.

9. (canceled)

10. The sensor of claim 8 wherein at least one accessory protein is a fluorescent protein and wherein the fluorescent protein is a GFP protein with positively-charged amino acids exposed on the protein surface.

11. (canceled)

12. The sensor of claim 1 wherein at least one accessory protein is capable of tagging one or more biomolecular condensate component with a detectable or functional molecule, peptide or marker.

13. The sensor of claim 1 wherein the sensor is a functionalized sensor and at least one accessory protein is capable of modifying a target component protein in the condensate or is capable of delivering a compound or agent to the condensate or to a target component protein in the condensate.

14. (canceled)

15. The sensor of claim 1 wherein the one or more accessory protein(s) and/or the accessory protein(s) and the artificial client protein are separated by a flexible linker sequence.

16. The sensor of claim 1 wherein the target component protein is a filaggrin family protein or paralog protein.

17. The sensor of claim 16 wherein the artificial client protein comprises a sequence selected from TABLE 3 and SEQ ID NOs: 17-21.

18. The sensor of claim 1 wherein the biomolecular condensate is selected from a keratohyalin granule (KG), P granule, Germ granule, Lewy bodies, synaptic condensates, stress granule, P bodies, T cell signalosome, crystalline condensates of the lens fibers, Nucleoli, Paraspeckles, Histone Locus Bodies, Cajal Bodies, Heterochromatin and other cytoplasmic or nuclear condensates or membraneless organelles assembled through liquid-liquid phase separation.

19. The sensor of claim 1 wherein the biomolecular condensate is a cytoplasmically-located condensate or wherein the biomolecular condensate is located in the nucleus.

20. (canceled)

21. A composition comprising the sensor of claim 1, optionally further comprising one or more vehicle, carrier or diluent.

22. A nucleic acid encoding the sensor of claim 1.

23. A vector comprising the nucleic acid of claim 22.

24. A method for targeting a biomolecular condensate or of detecting or visualizing a biomolecular condensate in a cell or tissue comprising administering to the cell or tissue or otherwise expressing in the cell or tissue the sensor of claim 1 or transfecting or transducing the cell with the vector of claim 23.

25. (canceled)

26. (canceled)

27. The method of claim 24 wherein the sensor comprises at least one accessory protein selected from a fluorescent protein, a protein that creates contrast suitable for electron microscopy, or a protein capable of tagging or labeling the condensate with a detectable or functional label or marker.

28. A method for monitoring or manipulating biomolecular condensates in a cell comprising administering to the cell or otherwise expressing in the cell or tissue the sensor of claim 1, wherein the sensor is capable of tagging the condensate with a detectable or functional label or marker or wherein the sensor or its cargo is capable of altering or tuning the material properties of the condensate.

29. (canceled)

30. A method for evaluating or screening a drug, compound or agent for modifying or altering a biomolecular condensate in a cell comprising administering to the cell or otherwise expressing in the cell or tissue the sensor of claim 1 wherein the sensor is capable of targeting or associating with the condensate so as to evaluate the condensate in the presence and absence of the drug, compound or agent.

31. The method of claim 24, wherein the biomolecular condensate is selected from a keratohyalin granule (KG), P granule, Germ granule, Lewy bodies, synaptic condensates, stress granule, P bodies, T cell signalosome, crystalline condensates of the lens fibers, Nucleoli, Paraspeckles, Histone Locus Bodies, Cajal Bodies, Heterochromatin and other cytoplasmic or nuclear condensates or membraneless organelles assembled through liquid-liquid phase separation.

32. A kit for evaluation of biomolecular condensates in cells or tissues comprising the sensor of claim 1, the nucleic acid of claim 22 or the vector of claim 23.


SEQUENCE LISTING (60 sequences)

SEQ ID NO: 1 - protein, 4654 aa, Mus musculus
MSALLESITSMIEIFQQYSTSDKEEETLSKEELKELLEGQLQAVLKNPDDQDIAEVFMQMLDVDHDDKLDFAEYLLLVLKLAKAYYEASKNESFQTHGSNGRSKTDYKGLEEEGEEGNKQNLRRRHGGTDGKRKSDRTRSPNGKRGKRQESRCRSEGKDKHRREPEKHRHQQDSKRKQRHGSGSTERKDNRNKKNRQSKERNYDEIYDNGKYNEDWEASYNNCYYKTQNTTLDQREGNRRPRADSQKEPQSSHGQADNSDSEGGRQQSHSKPSPVRADQRRSRAGQAGSSKVSARSGSGGRGQSPDGSGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSRYYYEQEHSEEESDSQHQHGHQHEQQRGHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGALQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGTSAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGNQGAHQKQGRDSARSRGSNQGHSSSRHQADSPRVSARSGSGGRGQSPDASGRNSTKRDRPRQPSPSQSSDSHVHSGAPDQGPRGTPSSVNRRAGSISGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSRYYYEQEHSEEESDSQHQHGHQHEQQRGHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRIGSSADRQGRRGVSESQASDSEGHSDFSEGQAVGAHRQSEAGQRHEQRSSRGQHGSGYYYEQEHSEEESDSQHQHGHQHEQQRGTQHQHEHQQPESGHRQQQSSGRGHQGTHQEQGRDSARSRGSNQGHSSSRHQADSPRVSARSGSGGRGQSPDASGRSSNRRDRPRQPSPSQSSDSQVHSGVQVEGRRRQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGALQGQASAQGRAGSQGQAQGRVGSSADRQGRRGVSGSQASDSEGHSDFSEGQAVGAHRQSGAGQRHEQRSSRGQHGSGYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEESDSQHQHSHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRSQHGSGYYYEQEHSEEESDSQHQHSHQHEQQRGHQHQHQHQHEHEQPESGHRQQQSSGRGNQGAHQEQGRDSARSRGSNQGHSSSRHQADSPRVSARSGSGGRGQSPDASGRSSNRRDRPRQPSPSQSSDSHVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRSRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGALQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSGSQASDSEGHSDFSEGQAVGAHRQSGAGQRHEQRSSRGQHGSGYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSSSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEESDSQHQQGHQHEQQRGHQHQHQHQHEHEQPESGHRQQQSSGRGHQGAHQEQGRDSARSRGSNQGHSSSRHQADSPRVSARSGSGGRGQSPDGSGRSSNRRDRPRQPSASQSSDSQVHSGVQVEAQRGQSSSANRRAGSSSGSGVQSAAASGQGGYESIFTAKHLDFNQSHSYYYY

SEQ ID NO: 2 - protein, 568 aa, Artificial Sequence
  sfGFP-(r8)
MGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYG

SEQ ID NO: 3 - protein, 893 aa, Artificial Sequence
  sfGFP-(r8)2
MGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYG

SEQ ID NO: 4 - protein, 1543 aa, Artificial Sequence
  sfGFP-(r8)4
MGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYG

SEQ ID NO: 5 - protein, 2843 aa, Artificial Sequence
  sfGFP-(r8)8
MGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYG

SEQ ID NO: 6 - protein, 3493 aa, Artificial Sequence
  sfGFP-(r8)10
MGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYG

SEQ ID NO: 7 - protein, 4143 aa, Artificial Sequence
  sfGFP-(r8)12
MGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYG

SEQ ID NO: 8 - protein, 2870 aa, Artificial Sequence
  sfGFP-(r8)-Tail
MGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGPGLCGHSSDISKQLGFSQSQRYYYYEG

SEQ ID NO: 9 - protein, 3520 aa, Artificial Sequence
  sfGFP-(r8)10-Tail
MGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGPGLCGHSSDISKQLGFSQSQRYYYYEG

SEQ ID NO: 10 - protein, 4170 aa, Artificial Sequence
  sfGFP-(r8)12-Tail
MGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGPGLCGHSSDISKQLGFSQSQRYYYYEG

SEQ ID NO: 11 - protein, 2979 aa, Artificial Sequence
  S100-sfGFP-(r8)8-Tail
MSTLLENIFAIINLFKQYSKKDKNTDTLSKKELKELLEKEFRQILKNPDDPDMVDVFMDHLDIDHNKKIDFTEFLLMVFKLAQAYYESTRKEGVPGSGVPGAGVPGSRSDGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGPGLCGHSSDISKQLGFSQSQRYYYYEG

SEQ ID NO: 12 - protein, 5429 aa, Artificial Sequence
  mRFP-(r8)16
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGASPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYG

SEQ ID NO: 13 - protein, 2965 aa, Artificial Sequence
  S100-mRFP-(r8)8-Tail
MSTLLENIFAIINLFKQYSKKDKNTDTLSKKELKELLEKEFRQILKNPDDPDMVDVFMDHLDIDHNKKIDFTEFLLMVFKLAQAYYESTRKEGVPGSGVPGAGVPGSRSDASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGASPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGPGLCGHSSDISKQLGFSQSQRYYYYEG

SEQ ID NO: 14 - protein, 324 aa, Artificial Sequence
  r8
QVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLY

SEQ ID NO: 15 - protein, 324 aa, Artificial Sequence
  r8H1
QVSTHEQSESSHGWTGPSTRGRQGSRYEQAQDSSRYSASQDGQDTIRGYPGSSRGGRQGYHHEHSVDSSGYSGSHHSHTTSQGRSDASRGQSGSRSASRTTRNEEQSGDGSRYSGSRHHEASTHADISRYSQAVQGQSEGSRRSRRQGSSVSQDSDSEGHSEDSERWSGSASRNHHGSAQEQLRDGSRHPRSHQEDRAGHGYSADSSRQSGTRHTQTSSGGQAASSHEQARSSAGERHGSHYQQSADSSRHSGIGHGQASSAVRDSGHRGYSGSQASDNEGHSEDSDTQSVSAHGQAGSHQQSHQESARGRSGETSGHSGSFLY

SEQ ID NO: 16 - protein, 324 aa, Artificial Sequence
  r8H2
QVSTYEQSESSYGWTGPSTRGRQGSRYEQAQDSSRYSASQDGQDTIRGYPGSSRGGRQGYYYEHSVDSSGYSGSYHSHTTSQGRSDASRGQSGSRSASRTTRNEEQSGDGSRYSGSRHYEASTHADISRYSQAVQGQSEGSRRSRRQGSSVSQDSDSEGHSEDSERWSGSASRNHHGSAQEQLRDGSRYPRSHQEDRAGHGYSADSSRQSGTRYTQTSSGGQAASSHEQARSSAGERYGSHYQQSADSSRHSGIGHGQASSAVRDSGHRGYSGSQASDNEGHSEDSDTQSVSAHGQAGSHQQSYQESARGRSGETSGHSGSFLY

SEQ ID NO: 17 - protein, 324 aa, Artificial Sequence
  ir8H2
YLFSGSHGSTEGSRGRASEQYSQQHSGAQGHASVSQTDSDESHGENDSAQSGSYGRHGSDRVASSAQGHGIGSHRSSDASQQYHSGYREGASSRAQEHSSAAQGGSSTQTYRTGSQRSSDASYGHGARDEQHSRPYRSGDRLQEQASGHHNRSASGSWRESDESHGESDSDQSVSSGQRRSRRSGESQGQVAQSYRSIDAHTSAEYHRSGSYRSGDGSQEENRTTRSASRSGSQGRSADSRGQSTTHSHYSGSYGSSDVSHEYYYGQRGGRSSGPYGRITDQGDQSASYRSSDQAQEYRSGQRGRTSPGTWGYSSESQEYTSVQ

SEQ ID NO: 18 - protein, 324 aa, Artificial Sequence
  pr8H2
TRNHQGDQRSSHSSSQSYQRYPSRIHEEREDAYEHEGGSSRGGRSGGQSGGSTREQHSASAVGSATRQSEGGYTSYYIASQSSDSHSYGQRSSYSVGSQDQRTDGNAYDGTSQHDRSSASGYRGEFEQSRSAVPGASGGQDHERESQSRSRHTEWHDGDIGSGGSSESRASSGQEGARSSEDRERGAGSSSGGSRQSSTDRQHRYGGAEGSGSQGRSGSHQDDSNHQYAGQGRASYHLGYSPARQHSSSYSTRHDRYTTQYQSGQAAGSRYHSQLYTQSTDQSAASSEQADSVQVSTSSQSYRGRSWDSSGEVRRSHSYGASSH

SEQ ID NO: 19 - protein, 200 aa, Artificial Sequence
  eFlg1
GRDGSHSYQGDRSGHSHQRQGYHEQSDRAGHGDSGHRGYSGRDGSHSYQGDRSGHSHQRQGYHEQSDRAGHGDSGHRGYSGRDGSHSYQGDRSGHSHQRQGYHEQSDRAGHGDSGHRGYSGRDGSHSYQGDRSGHSHQRQGYHEQSDRAGHGDSGHRGYSGRDGSHSYQGDRSGHSHQRQGYHEQSDRAGHGDSGHRGYS

SEQ ID NO: 20 - protein, 200 aa, Artificial Sequence
  ieFlg1
SYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRG

SEQ ID NO: 21 - protein, 320 aa, Artificial Sequence
  eFlg2
GRDGSHSYQGDRSGHSHQRQGYHEQSDRAGHGDSGHRGYSGRDGSHSYQGDRSGHSHQRQGYHEQSDRAGHGDSGHRGYSGRDGSHSYQGDRSGHSHQRQGYHEQSDRAGHGDSGHRGYSGRDGSHSYQGDRSGHSHQRQGYHEQSDRAGHGDSGHRGYSSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRG

SEQ ID NO: 22 - protein, 254 aa, Artificial Sequence
  sfGFP-NES
MGASKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSGLELLEDLTLGSP

SEQ ID NO: 23 - protein, 255 aa, Artificial Sequence
  -20sfGFP-NES
MGASKGEELFTGVVPILVELDGDVNGHKFSVRGEGEGDATNGKLTLKFICTTGKLPVPWPTLVTTLTYGVQCFSRYPDHMDQHDFFKSAMPEGYVQERTISFKDDGTYKTRAEVKFEGDTLVNRIELKGIDFKEDGNILGHKLEYNFNSHDVYITADKQENGIKAEFEIRHNVEDGSVQLADHYQQNTPIGDGPVLLPDDHYLSTESALSKDPNEDRDHMVLLEFVTAAGIDHGMDELYKSGLELLEDLTLGSPG

SEQ ID NO: 24 - protein, 254 aa, Artificial Sequence
  +15sfGFP-NES
MGASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKSGLELLEDLTLGSP

SEQ ID NO: 25 - protein, 255 aa, Artificial Sequence
  +15sfGFPK-NES
MGASKGEKLFTGVVPILVELDGDVNGHKFSVRGEGEGDATKGKLTLKFICTTGKLPVPWPTLVTTLTYGVQCFSRYPKHMKRHDFFKSAMPEGYVQERTISFKKDGTYKTRAEVKFEGKTLVNRIELKGKDFKEKGNILGHKLEYNFNSHNVYITADKKKNGIKANFKIRHNVKDGSVQLADHYQQNTPIGKGPVLLPKNHYLSTKSALSKDPKEKRDHMVLLEFVTAAGITHGMDELYKSGLELLEDLTLGSPG

SEQ ID NO: 26 - protein, 581 aa, Artificial Sequence
  Sensor A
MGASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKSGLELLEDLTLGSPGYLFSGSHGSTEGSRGRASEQYSQQHSGAQGHASVSQTDSDESHGENDSAQSGSYGRHGSDRVASSAQGHGIGSHRSSDASQQYHSGYREGASSRAQEHSSAAQGGSSTQTYRTGSQRSSDASYGHGARDEQHSRPYRSGDRLQEQASGHHNRSASGSWRESDESHGESDSDQSVSSGQRRSRRSGESQGQVAQSYRSIDAHTSAEYHRSGSYRSGDGSQEENRTTRSASRSGSQGRSADSRGQSTTHSHYSGSYGSSDVSHEYYYGQRGGRSSGPYGRITDQGDQSASYRSSDQAQEYRSGQRGRTSPGTWGYSSESQEYTSVQGS

SEQ ID NO: 27 - protein, 457 aa, Artificial Sequence
  Sensor B
MGASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKSGLELLEDLTLGSPGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGGS

SEQ ID NO: 28 - protein, 240 aa, Artificial Sequence
  +15GFP
MGASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYK

SEQ ID NO: 29 - protein, 240 aa, Artificial Sequence
  +15GFPK
MGASKGEKLFTGVVPILVELDGDVNGHKFSVRGEGEGDATKGKLTLKFICTTGKLPVPWPTLVTTLTYGVQCFSRYPKHMKRHDFFKSAMPEGYVQERTISFKKDGTYKTRAEVKFEGKTLVNRIELKGKDFKEKGNILGHKLEYNFNSHNVYITADKKKNGIKANFKIRHNVKDGSVQLADHYQQNTPIGKGPVLLPKNHYLSTKSALSKDPKEKRDHMVLLEFVTAAGITHGMDELYK

SEQ ID NO: 30 - protein, 826 aa, Artificial Sequence
  mRFP1-K10
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGASGLELLEDLTLGRSDGSVRYSSSKHYSSSRSGGGGGGGGCGGGGGVSSLRISSSKGSLGGGFSSGGFSGGSFSRGSSGGGCFGGSSGGYGGLGGFGGGSFRGSYGSSSFGGSYGGSFGGGSFGGGSFGGGSFGGGGFGGGGFGGGFGGGFGGDGGLLSGNEKVTMQNLNDRLASYLDKVRALEESNYELEGKIKEWYEKHGNSHQGEPRDYSKYYKTIDDLKNQILNLTTDNANILLQIDNARLAADDFRLKYENEVALRQSVEADINGLRRVLDELTLTKADLEMQIESLTEELAYLKKNHEEEMKDLRNVSTGDVNVEMNAAPGVDLTQLLNNMRSQYEQLAEQNRKDAEAWFNEKSKELTTEIDNNIEQISSYKSEITELRRNVQALEIELQSQLALKQSLEASLAETEGRYCVQLSQIQAQISALEEQLQQIRAETECQNTEYQQLLDIKIRLENEIQTYRSLLEGEGSGSSGGGGRGGGSFGGGYGGGSSGGGSSGGGHGGGHGGSSGGGYGGGSSGGGSSGGGYGGGSSSGGHGGSSSGGYGGGSSGGGGGGYGGGSSGGGSSSGGGYGGGSSSGGHKSSSSGSVGESSSKGPRY

SEQ ID NO: 31 - protein, 385 aa, Artificial Sequence
  N-LC(K10)mCherry
MSVRYSSSKHYSSSRSGGGGGGGGCGGGGGVSSLRISSSKGSLGGGFSSGGFSGGSFSRGSSGGGCFGGSSGGYGGLGGFGGGSFRGSYGSSSFGGSYGGIFGGGSFGGGSFGGGSFGGGGFGGGGFGGGFGGGFGGDGGLLSGNSPGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGS

SEQ ID NO: 32 - protein, 513 aa, Artificial Sequence
  mCherry-C-LC(K10)
MSVRYSSSKHYSSSRSGGGGGGGGCGGGGGVSSLRISSSKGSLGGGFSSGGFSGGSFSRGSSGGGCFGGSSGGYGGLGGFGGGSFRGSYGSSSFGGSYGGIFGGGSFGGGSFGGGSFGGGGFGGGGFGGGFGGGFGGDGGLLSGNSPGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGSSGGGGRGGGSFGGGYGGGSSGGGSSGGGHGGGHGGSSGGGYGGGSSGGGSSGGGYGGGSSSGGHGGSSSGGYGGGSSGGGGGGYGGGSSGGGSSSGGGYGGGSSSGGHKSSSSGSVGESSSKGPRY

SEQ ID NO: 33 - protein, 239 aa, Artificial Sequence
  mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSPG

SEQ ID NO: 34 - DNA, 66 bp, Artificial Sequence
  p2a sequence
ggaagcggagcgacgaattttagtctactgaaacaagcgggagacgtggaggaaaaccctggacct

SEQ ID NO: 35 - protein, 9 aa, Artificial Sequence
  nuclear export sequence
LELLEDLTL

SEQ ID NO: 36 - DNA, 53 bp, Artificial Sequence
  forward primer
taatcatcgatcggatggctctgttcgatacagctcaagcaagcactactctt

SEQ ID NO: 37 - DNA, 21 bp, Artificial Sequence
  hairpin mouse Flg1
atcaatctcacagctattatt

SEQ ID NO: 38 - DNA, 21 bp, Artificial Sequence
  hairpin mouse Flg
ctccggattctacccagtata

SEQ ID NO: 39 - protein, 2866 aa, Artificial Sequence
  mRFP1-ENLYFQS-(r8)8-Tail
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGASGSENLYFQSGPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGPGLCGHSSDISKQLGFSQSQRYYYYEG

SEQ ID NO: 40 - protein, 2866 aa, Artificial Sequence
  mRFP1-ENLYFQR-(r8)8-Tail
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGASGSENLYFQRGPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGPGLCGHSSDISKQLGFSQSQRYYYYEG

SEQ ID NO: 41 - protein, 1665 aa, Artificial Sequence
  mS100-mRFP1-(r8)4-Tail
MSALLESITSMIEIFQQYSTSDKEEETLSKEELKELLEGQLQAVLKNPDDQDIAEVFMQMLDVDHDDKLDFAEYLLLVLKLAKAYYEASKNEGVPGSGVPGAGVPGSRSDASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGASPGGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYGPGLCGHSSDISKQLGFSQSQRYYYYEG

SEQ ID NO: 42 - protein, 488 aa, Artificial Sequence
  sfGFP-dTEVP
MGSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSPGSPGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQAGSPLVSTRDGFIVGIHSASNFTNTNNYFTSVPKNFMELLTNQEAQQWVSGWRLNADSVLWGGHKVFMVKPEEPFQPVKEATQLMNELVYSQ

SEQ ID NO: 43 - protein, 360 aa, Artificial Sequence
  mS100-n20GFP
MSALLESITSMIEIFQQYSTSDKEEETLSKEELKELLEGQLQAVLKNPDDQDIAEVFMQMLDVDHDDKLDFAEYLLLVLKLAKAYYEASKNEGVPGSGVPGAGVPGSRSDGASKGEELFTGVVPILVELDGDVNGHKFSVRGEGEGDATNGKLTLKFICTTGKLPVPWPTLVTTLTYGVQCFSRYPDHMDQHDFFKSAMPEGYVQERTISFKDDGTYKTRAEVKFEGDTLVNRIELKGIDFKEDGNILGHKLEYNFNSHDVYITADKQENGIKAEFEIRHNVEDGSVQLADHYQQNTPIGDGPVLLPDDHYLSTESALSKDPNEDRDHMVLLEFVTAAGIDHGMDELYKSGLELLEDLTL

SEQ ID NO: 44 - protein, 40 aa, Artificial Sequence
  designed Flg repeat
GRDGSHSYQGDRSGHSHQRQGYHEQSDRAGHGDSGHRGYS

SEQ ID NO: 45 - protein, 5 aa, Artificial Sequence
  r8 repeat motif
RQGYH

SEQ ID NO: 46 - protein, 6 aa, Artificial Sequence
  r8 repeat motif
DRAGHG

SEQ ID NO: 47 - protein, 3 aa, Artificial Sequence
  r8 repeat motif
EQS

SEQ ID NO: 48 - protein, 4 aa, Artificial Sequence
  r8 repeat motif
RDGS

SEQ ID NO: 49 - protein, 8 aa, Artificial Sequence
  r8 repeat motif
DSGHRGYS

SEQ ID NO: 50 - protein, 852 aa, Artificial Sequence
  Apex2-Sensor A
MGASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKSGLELLEDLTLGRSDGVPGSGGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADALQLPPLERLTLDGSPGYLFSGSHGSTEGSRGRASEQYSQQHSGAQGHASVSQTDSDESHGENDSAQSGSYGRHGSDRVASSAQGHGIGSHRSSDASQQYHSGYREGASSRAQEHSSAAQGGSSTQTYRTGSQRSSDASYGHGARDEQHSRPYRSGDRLQEQASGHHNRSASGSWRESDESHGESDSDQSVSSGQRRSRRSGESQGQVAQSYRSIDAHTSAEYHRSGSYRSGDGSQEENRTTRSASRSGSQGRSADSRGQSTTHSHYSGSYGSSDVSHEYYYGQRGGRSSGPYGRITDQGDQSASYRSSDQAQEYRSGQRGRTSPGTWGYSSESQEYTSVQGS

SEQ ID NO: 51 - protein, 728 aa, Artificial Sequence
  Apex2-Sensor B
MGASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYKSGLELLEDLTLGRSDGVPGSGGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADALQLPPLERLTLDGSPGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGGS

SEQ ID NO: 52 - protein, 581 aa, Artificial Sequence
  Sensor C
MGASKGEKLFTGVVPILVELDGDVNGHKFSVRGEGEGDATKGKLTLKFICTTGKLPVPWPTLVTTLTYGVQCFSRYPKHMKRHDFFKSAMPEGYVQERTISFKKDGTYKTRAEVKFEGKTLVNRIELKGKDFKEKGNILGHKLEYNFNSHNVYITADKKKNGIKANFKIRHNVKDGSVQLADHYQQNTPIGKGPVLLPKNHYLSTKSALSKDPKEKRDHMVLLEFVTAAGITHGMDELYKSGLELLEDLTLGSPGYLFSGSHGSTEGSRGRASEQYSQQHSGAQGHASVSQTDSDESHGENDSAQSGSYGRHGSDRVASSAQGHGIGSHRSSDASQQYHSGYREGASSRAQEHSSAAQGGSSTQTYRTGSQRSSDASYGHGARDEQHSRPYRSGDRLQEQASGHHNRSASGSWRESDESHGESDSDQSVSSGQRRSRRSGESQGQVAQSYRSIDAHTSAEYHRSGSYRSGDGSQEENRTTRSASRSGSQGRSADSRGQSTTHSHYSGSYGSSDVSHEYYYGQRGGRSSGPYGRITDQGDQSASYRSSDQAQEYRSGQRGRTSPGTWGYSSESQEYTSVQGS

SEQ ID NO: 53 - protein, 457 aa, Artificial Sequence
  Sensor D
MGASKGEKLFTGVVPILVELDGDVNGHKFSVRGEGEGDATKGKLTLKFICTTGKLPVPWPTLVTTLTYGVQCFSRYPKHMKRHDFFKSAMPEGYVQERTISFKKDGTYKTRAEVKFEGKTLVNRIELKGKDFKEKGNILGHKLEYNFNSHNVYITADKKKNGIKANFKIRHNVKDGSVQLADHYQQNTPIGKGPVLLPKNHYLSTKSALSKDPKEKRDHMVLLEFVTAAGITHGMDELYKSGLELLEDLTLGSPGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGSYGRHGSDGHGARDSQEHYGQRQHSHGSRDGQYSHSGDRGGS

SEQ ID NO: 54 - protein, 557 aa, Artificial Sequence
  Apex2-excluded sensor
MGASKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKSGLELLEDLTLGRSDGVPGSGGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADALQLPPLERLTLDGSPGSPGSGYIPEAPRDGQAYVRKDGEWVLLSTFL

SEQ ID NO: 55 - DNA, 44 bp, Artificial Sequence
  reverse primer
taagcaggggatccctctccttctagcaggctgcggtaggtttg

SEQ ID NO: 56 - protein, 4061 aa, Homo sapiens
MSTLLENIFAIINLFKQYSKKDKNTDTLSKKELKELLEKEFRQILKNPDDPDMVDVFMDHLDIDHNKKIDFTEFLLMVFKLAQAYYESTRKENLPISGHKHRKHSHHDKHEDNKQEENKENRKRPSSLERRNNRKGNKGRSKSPRETGGKRHESSSEKKERKGYSPTHREEEYGKNHHNSSKKEKNKTENTRLGDNRKRLSERLEEKEDNEEGVYDYENTGRMTQKWIQSGHIATYYTIQDEAYDTTDSLLEENKIYERSRSSDGKSSSQVNRSRHENTSQVPLQESRTRKRRGSRVSQDRDSEGHSEDSERHSGSASRNHHGSAWEQSRDGSRHPRSHDEDRASHGHSADSSRQSGTRHAETSSRGQTASSHEQARSSPGERHGSGHQQSADSSRHSATGRGQASSAVSDRGHRGSSGSQASDSEGHSENSDTQSVSGHGKAGLRQQSHQESTRGRSGERSGRSGSSLYQVSTHEQPDSAHGRTGTSTGGRQGSHHEQARDSSRHSASQEGQDTIRGHPGSSRGGRQGSHHEQSVNRSGHSGSHHSHTTSQGRSDASHGQSGSRSASRQTRNEEQSGDGTRHSGSRHHEASSQADSSRHSQVGQGQSSGPRTSRNQGSSVSQDSDSQGHSEDSERWSGSASRNHHGSAQEQSRDGSRHPRSHHEDRAGHGHSADSSRKSGTRHTQNSSSGQAASSHEQARSSAGERHGSRHQLQSADSSRHSGTGHGQASSAVRDSGHRGSSGSQATDSEGHSEDSDTQSVSGHGQAGHHQQSHQESARDRSGERSRRSGSFLYQVSTHKQSESSHGWTGPSTGVRQGSHHEQARDNSRHSASQDGQDTIRGHPGSSRRGRQGSHHEQSVDRSGHSGSHHSHTTSQGRSDASRGQSGSRSASRTTRNEEQSRDGSRHSGSRHHEASSHADISRHSQAGQGQSEGSRTSRRQGSSVSQDSDSEGHSEDSERWSGSASRNHRGSAQEQSRHGSRHPRSHHEDRAGHGHSADSSRQSGTPHAETSSGGQAASSHEQARSSPGERHGSRHQQSADSSRHSGIPRRQASSAVRDSGHWGSSGSQASDSEGHSEESDTQSVSGHGQDGPHQQSHQESARDWSGGRSGRSGSFIYQVSTHEQSESAHGRTRTSTGRRQGSHHEQARDSSRHSASQEGQDTIRAHPGSRRGGRQGSHHEQSVDRSGHSGSHHSHTTSQGRSDASHGQSGSRSASRQTRKDKQSGDGSRHSGSRHHEAASWADSSRHSQVGQEQSSGSRTSRHQGSSVSQDSDSERHSDDSERLSGSASRNHHGSSREQSRDGSRHPGFHQEDRASHGHSADSSRQSGTHHTESSSHGQAVSSHEQARSSPGERHGSRHQQSADSSRHSGIGHRQASSAVRDSGHRGSSGSQVTNSEGHSEDSDTQSVSAHGQAGPHQQSHKESARGQSGESSGRSRSFLYQVSSHEQSESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQSESAHGRTGPSTGGRQRSRHEQARDSSRHSASQEGQDTIRGHPGSSRGGRQGSHYEQSVDSSGHSGSHHSHTTSQERSDVSRGQSGSRSVSRQTRNEKQSGDGSRHSGSRHHEASSRADSSRHSQVGQGQSSGPRTSRNQGSSVSQDSDSQGHSEDSERWSGSASRNHLGSAWEQSRDGSRHPGSHHEDRAGHGHSADSSRQSGTRHTESSSRGQAASSHEQARSSAGERHGSHHQLQSADSSRHSGIGHGQASSAVRDSGHRGYSGSQASDSEGHSEDSDTQSVSAQGKAGPHQQSHKESARGQSGESSGRSGSFLYQVSTHEQSESTHGQSAPSTGGRQGSHYDQAQDSSRHSASQEGQDTIRGHPGPSRGGRQGSHQEQSVDRSGHSGSHHSHTTSQGRSDASRGQSGSRSASRKTYDKEQSGDGSRHSGSHHHEASSWADSSRHSLVGQGQSSGPRTSRPRGSSVSQDSDSEGHSEDSERRSGSASRNHHGSAQEQSRDGSRHPRSHHEDRAGHGHSAESSRQSGTHHAENSSGGQAASSHEQARSSAGERHGSHHQQSADSSRHSGIGHGQASSAVRDSGHRGSSGSQASDSEGHSEDSDTQSVSAHGQAGPHQQSHQESTRGRSAGRSGRSGSFLYQVSTHEQSESAHGRTGTSTGGRQGSHHKQARDSSRHSTSQEGQDTIHGHPGSSSGGRQGSHYEQLVDRSGHSGSHHSHTTSQGRSDASHGHSGSRSASRQTRNDEQSGDGSRHSGSRHHEASSRADSSGHSQVGQGQSEGPRTSRNWGSSFSQDSDSQGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYQVSTHEQSESSHGWTGPSTRGRQGSRHEQAQDSSRHSASQYGQDTIRGHPGSSRGGRQGYHHEHSVDSSGHSGSHHSHTTSQGRSDASRGQSGSRSASRTTRNEEQSGDSSRHSVSRHHEASTHADISRHSQAVQGQSEGSRRSRRQGSSVSQDSDSEGHSEDSERWSGSASRNHRGSVQEQSRHGSRHPRSHHEDRAGHGHSADRSRQSGTRHAETSSGGQAASSHEQARSSPGERHGSRHQQSADSSRHSGIPRGQASSAVRDSRHWGSSGSQASDSEGHSEESDTQSVSGHGQAGPHQQSHQESARDRSGGRSGRSGSFLYQVSTHEQSESAHGRTRTSTGRRQGSHHEQARDSSRHSASQEGQDTIRGHPGSSRRGRQGSHYEQSVDRSGHSGSHHSHTTSQGRSDASRGQSGSRSASRQTRNDEQSGDGSRHSWSHHHEASTQADSSRHSQSGQGQSAGPRTSRNQGSSVSQDSDSQGHSEDSERWSGSASRNHRGSAQEQSRDGSRHPTSHHEDRAGHGHSAESSRQSGTHHAENSSGGQAASSHEQARSSAGERHGSHHQQSADSSRHSGIGHGQASSAVRDSGHRGSSGSQASDSEGHSEDSDTQSVSAHGQAGPHQQSHQESTRGRSAGRSGRSGSFLYQVSTHEQSESAHGRAGPSTGGRQGSRHEQARDSSRHSASQEGQDTIRGHPGSRRGGRQGSYHEQSVDRSGHSGSHHSHTTSQGRSDASHGQSGSRSASRETRNEEQSGDGSRHSGSRHHEASTQADSSRHSQSGQGESAGSRRSRRQGSSVSQDSDSEAYPEDSERRSESASRNHHGSSREQSRDGSRHPGSSHRDTASHVQSSPVQSDSSTAKEHGHFSSLSQDSAYHSGIQSRGSPHSSSSYHYQSEGTERQKGQSGLVWRHGSYGSADYDYGESGFRHSQHGSVSYNSNPVVFKERSDICKASAFGKDHPRYYATYINKDPGLCGHSSDISKQLGFSQSQRYYYYE

SEQ ID NO: 57 - protein, 9 aa, Artificial Sequence
  nuclear export signal (NES)
LELLEDLTL

SEQ ID NO: 58 - protein, 11 aa, Artificial sequence
  nuclear export signal (NES)
SGLELLEDLTL

SEQ ID NO: 59 - protein, 4 aa, Artificial sequence
  linker sequence
GSPG

SEQ ID NO: 60 - protein, 10 aa, Artificial sequence
  linker sequence
GRSDGVPGSG